(19) United States
(12) Patent Application Publication
(10) Pub. No.: US 2024/0336915 A1
(43) Pub. Date: Oct. 10, 2024

(54) COMPOUNDS AND METHODS FOR REDUCING DUX4 EXPRESSION

(71) Applicant: Ionis Pharmaceuticals, Inc., Carlsbad, CA (US)

(72) Inventors: Ruben E. Valas, Oceanside, CA (US); Paymaan Jafar-nejad, Carlsbad, CA (US); Frank Rigo, Carlsbad, CA (US); Susan M. Freier, San Diego, CA (US); Huynh-Hoa Bui, San Diego, CA (US); Priyam Singh, San Diego, CA (US)

(73) Assignee: Ionis Pharmaceuticals, Inc., Carlsbad, CA (US)

(21) Appl. No.: 18/262,438

(22) PCT Filed: Jan. 21, 2022
(86) PCT No.: PCT/US2022/013323

§ 371 Date: Jul. 21, 2023

Related U.S. Application Data
(60) Provisional application No. 63/231,559, filed on Aug. 10, 2021.
(60) Provisional application No. 63/217,624, filed on Jul. 1, 2021.
(60) Provisional application No. 63/140,674, filed on Jan. 22, 2021.

(51) Int. Cl. C12N 15/113

(52) U.S. Cl. CPC C12N 15/113; C12N 2310/14; C12N 2310/315; C12N 2310/3231; C12N 2310/3341

(57) ABSTRACT

Provided are compounds, pharmaceutical compositions, and methods of use for reducing the amount or activity of DUX4 RNA in a cell or animal, and in certain instances reducing the amount of DUX4 protein in a cell or animal. Such compounds, methods, and pharmaceutical compositions are useful to ameliorate at least one symptom or hallmark of a muscular dystrophy. Such symptoms and hallmarks include muscle weakness and/or muscle wasting in facio, scapula, and/or humeral muscle that can progress to the muscles of the trunk and/or lower limbs. Such muscular dystrophies include Facioscapulohumeral muscular dystrophy (FSHD).

### SEQUENCE LISTING

The present application is being filed along with a Sequence Listing in electronic format. The Sequence Listing is provided as a file entitled BIOL0415WOSEQ_ST25.txt, created on Jan. 11, 2022, which is 356 KB in size. The information in the electronic format of the sequence listing is incorporated herein by reference in its entirety.

### FIELD

Provided are compounds, methods, and pharmaceutical compositions for reducing the amount or activity of DUX4 RNA in a cell or animal, and in certain instances reducing the amount of DUX4 protein in a cell or animal. Such compounds, methods, and pharmaceutical compositions are useful to ameliorate at least one symptom or hallmark of a muscular dystrophy or a neuromuscular disorder. Such symptoms and hallmarks include muscle weakness and muscle wasting in facio, scapula, and/or humeral muscle that can progress to the muscles of the trunk and/or lower limbs.

Such muscular dystrophies include Facioscapulohumeral muscular dystrophy (FSHD).

### BACKGROUND

Facioscapulohumeral muscular dystrophy (FSHD) is a progressive skeletal muscle disorder affecting the facial, scapular, and/or humeral muscles. FSHD is characterized by a clinical variety of symptoms, including but not limited to muscle weakness and muscle wasting in facio, scapula, and/or humeral muscles that can progress to the muscles of the trunk and lower limbs. The disorder is caused by the aberrant expression of double homeobox 4 (DUX4) in skeletal muscle cells.

Currently there is a lack of acceptable options for treating muscular dystrophies such as FSHD. It is therefore an object herein to provide compounds, methods, and pharmaceutical compositions for the treatment of such diseases or disorders.

### SUMMARY

Provided herein are compounds, pharmaceutical compositions, and methods of use for reducing the amount or activity of DUX4 RNA, and in certain embodiments reducing the amount of DUX4 protein in a cell or animal. In certain embodiments, the animal has a disease or disorder associated with DUX4. In certain embodiments, the animal has a muscular dystrophy. In certain embodiments, the animal has a neuromuscular disorder. In certain embodiments, the animal has facioscapulohumeral muscular dystrophy (FSHD). In certain embodiments, compounds useful for reducing the amount or activity of DUX4 RNA are oligomeric compounds. In certain embodiments, compounds useful for reducing the amount or activity of DUX4 RNA are modified oligonucleotides.

Also provided are methods useful for ameliorating at least one symptom or hallmark of a disease or disorder associated with DUX4. In certain embodiments, the disease or disorder associated with DUX4 is a neuromuscular disorder. In certain embodiments, the disease or disorder associated with DUX4 is a muscular dystrophy. In certain embodiments, the muscular dystrophy is Facioscapulohumeral muscular dystrophy (FSHD). In certain embodiments, the symptom or hallmark includes muscle weakness and/or muscle wasting in facio, scapula, and/or humeral muscle that can progress to the muscles of the trunk and/or lower limbs.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/of” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout the disclosure are incorporated by reference herein in their entirety.

Unless otherwise indicated, the following terms have the following meanings:

As used herein, “2′-deoxynucleoside” means a nucleoside comprising a 2′-H(H) deoxyribosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside or a nucleoside comprising an unmodified 2′-deoxyribosyl sugar moiety may comprise a modified nucleobase or may comprise an RNA nucleobase (uracil).

As used herein, “2′-MOE” means a 2′-OCH₂CH₂OCH₃ group in place of the 2′—OH group of a ribosyl sugar moiety. A “2′-MOE sugar moiety” or a “2′-O-methoxyethyl sugar moiety” means a sugar moiety with a 2′-OCH₂CH₂OCH₃ group in place of the 2′—OH group of a ribosyl sugar moiety. Unless otherwise indicated, a 2′-MOE sugar moiety is in the f-D configuration. “MOE” means O-methoxyethyl.

As used herein, “2′-MOE nucleoside” or “2′-O(CH₂)₂OCH₃ nucleoside” means a nucleoside comprising a 2′-MOE sugar moiety (or 2′-O(CH₂)₂OCH₃ ribosyl sugar moiety).

As used herein, “2′-OMe” means a 2′-OCH₃ group in place of the 2′—OH group of a ribosyl sugar moiety. A “2′-O-methyl sugar moiety” means a sugar moiety with a 2′-OCH₃ group in place of the 2′—OH group of a ribosyl sugar moiety. Unless otherwise indicated, a 2′-OMe has the β-D ribosyl stereochemical configuration.

As used herein, “2′-OMe nucleoside” means a nucleoside comprising a 2′-OMe sugar moiety.

As used herein, “2′-F” means a 2′-fluoro group in place of the 2′—OH group of a ribosyl sugar moiety. A “2′-F sugar moiety” or “2′-fluororibosyl sugar moiety” means a sugar moiety with a 2′—F group in place of the 2′-OH group of a ribosyl sugar moiety. Unless otherwise indicated, a 2′-F has the β-D ribosyl stereochemical configuration.

As used herein, “2′-F nucleoside” means a nucleoside comprising a 2′-F sugar moiety.

As used herein, “2′-substituted nucleoside” means a nucleoside comprising a 2′-substituted furanosyl sugar moiety. As used herein, “2′-substituted” in reference to a sugar moiety means a sugar moiety comprising at least one 2′-substituent group other than H or OH.

As used herein, “3′ target site” refers to the 3′-most nucleotide of a target nucleic acid which is complementary to an antisense oligonucleotide, when the antisense oligonucleotide is hybridized to the target nucleic acid.

As used herein, “5′ target site” refers to the 5′-most nucleotide of a target nucleic acid which is complementary to an antisense oligonucleotide, when the antisense oligonucleotide is hybridized to the target nucleic acid.

As used herein, “5-methylcytosine” means a cytosine modified with a methyl group attached to the 5 position. A 5-methylcytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “administration” or “administering” means providing a pharmaceutical agent or composition to an animal.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark or the delayed onset or slowing of progression in the severity or frequency of a symptom or hallmark. In certain embodiments, the symptom or hallmark is muscle weakness or muscle wasting in facio, scapula, and/or humeral muscle that can progress to the muscles of the trunk and lower limbs. The progression or severity of indicators may be determined by subjective or objective measures, which are known to those skilled in the art.

As used herein, “animal” means a human or non-human animal.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense agent” means an antisense compound and optionally one or more additional features, such as a sense compound.

As used herein, “antisense compound” means an antisense oligonucleotide and optionally one or more additional features, such as a conjugate group.

As used herein, “sense compound” means a sense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides.

As used herein, “sense oligonucleotide” means an oligonucleotide, including the oligonucleotide portion of a sense compound, that is capable of hybridizing to an antisense oligonucleotide.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl sugar moiety. In certain embodiments, the furanosyl sugar moiety is a ribosyl sugar moiety. In certain embodiments, the bicyclic sugar moiety does not comprise a furanosyl sugar moiety.

As used herein, “blunt” or “blunt ended” in reference to an oligomeric duplex formed by two oligonucleotides means that there are no terminal unpaired nucleotides (i.e. no overhanging nucleotides). One or both ends of a double-stranded RNAi agent can be blunt.

As used herein, “cell-targeting moiety” means a conjugate group or portion of a conjugate group that is capable of binding to a particular cell type or particular cell types.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) similar to cerebrospinal fluid and is biocompatible with CSF.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide or one or more portions thereof and the nucleobases of another nucleic acid or one or more portions thereof are capable of hydrogen bonding with one another when the nucleobase sequence of the oligonucleotide and the other nucleic acid are aligned in opposing directions. As used herein, “complementary nucleobases” means nucleobases that are capable of forming hydrogen bonds with one another. Complementary nucleobase pairs include adenine (A) and thymine (T), adenine (A) and uracil (U), cytosine (C) and guanine (G), 5-methylcytosine (^{m}C) and guanine (G). Certain modified nucleobases that pair with natural nucleobases or with other modified nucleobases are known in the art. For example, inosine can pair with adenosine, cytosine, or uracil. Complementary oligonucleotides and/or nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or nucleic acid at each nucleobase of the shorter of the two oligonucleotides, or at each nucleoside if the oligonucleotides are the same length.

As used herein, “complementary region” in reference to an oligonucleotide is the range of nucleobases of the oligonucleotide that is complementary with a second oligonucleotide or target nucleic acid.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt modified sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt modified sugar moiety.

As used herein, “deoxy region” means a region of 5-12 contiguous nucleotides, wherein at least 70% of the nucleosides are 2′-β-D-deoxynucleosides. In certain embodiments, each nucleoside is selected from a 2′-β-D-deoxynucleoside, a bicyclic nucleoside, and a 2′-substituted nucleoside. In certain embodiments, a deoxy region supports RNase H activity. In certain embodiments, a deoxy region is the gap or internal region of a gapmer.

As used herein, “diluent” means an ingredient in a composition that lacks pharmacological activity, but is pharmaceutically necessary or desirable. For example, the diluent in an injected composition can be a liquid, e.g. aCSF, PBS, or saline solution.

As used herein, “double-stranded” in reference to a region or an oligonucleotide means a duplex formed by complementary strands of nucleic acids (including, but not limited to oligonucleotides) hybridized to one another. In certain embodiments, the two strands of a double-stranded region are separate molecules. In certain embodiments, the two strands are regions of the same molecule that has folded onto itself (e.g., a hairpin structure).

As used herein, “duplex” or “duplex region” means the structure formed by two oligonucleotides or portions thereof that are hybridized to one another.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings” or “wing segments.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE nucleosides. As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid that is amenable to oligomeric agent or oligomeric compound-mediated reduction of the amount or activity of the target nucleic acid.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “internucleoside linkage” means the covalent linkage between contiguous nucleosides in an oligonucleotide. As used herein, “modified internucleoside linkage” means any internucleoside linkage other than a phosphodiester internucleoside linkage. “Phosphorothioate internucleoside linkage” or “PS internucleoside linkage” is a modified internucleoside linkage in which one of the non-bridging oxygen atoms of a phosphodiester internucleoside linkage is replaced with a sulfur atom.

As used herein, “inverted nucleoside” means a nucleotide having a 3′ to 3′ and/or 5′ to 5′ internucleoside linkage, as shown herein.

As used herein, “inverted sugar moiety” means the sugar moiety of an inverted nucleoside or an abasic sugar moiety having a 3′ to 3′ and/or 5′ to 5′ internucleoside linkage.

As used herein, “linked nucleosides” are nucleosides that are connected in a contiguous sequence (i.e., no additional nucleosides are presented between those that are linked).

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “mismatch” or “non-complementary” means a nucleobase of a first nucleic acid sequence that is not complementary with the corresponding nucleobase of a second nucleic acid sequence or target nucleic acid when the first and second nucleic acid sequences are aligned in opposing directions.

As used herein, “motif” means the pattern of unmodified and/or modified sugar moieties, nucleobases, and/or internucleoside linkages, in an oligonucleotide.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methylcytosine” is a modified nucleobase. A universal base is a modified nucleobase that can pair with any one of the five unmodified nucleobases.

As used herein, “nucleobase sequence” means the order of contiguous nucleobases in a target nucleic acid or oligonucleotide independent of any sugar or internucleoside linkage modification.

As used herein, “nucleoside” means a compound, or fragment of a compound, comprising a nucleobase and a sugar moiety. The nucleobase and sugar moiety are each, independently, unmodified or modified.

As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. An oligonucleotide may be paired with a second oligonucleotide that is complementary to the oligonucleotide or it may be unpaired. A “single-stranded oligonucleotide” is an unpaired oligonucleotide. A “double-stranded oligonucleotide” is an oligonucleotide that is paired with a second oligonucleotide.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by an animal. In certain embodiments, a pharmaceutically acceptable carrier or diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein “prodrug” means a therapeutic agent in a first form outside the body that is converted to a second form within an animal or cells thereof. Typically, conversion of a prodrug within the animal is facilitated by the action of an enzymes (e.g., endogenous or viral enzyme) or chemicals present in cells or tissues and/or by physiologic conditions. In certain embodiments, the first form of the prodrug is less active than the second form.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “RNA” means an RNA transcript and includes pre-mRNA and mature mRNA unless otherwise specified.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNA (ssRNAi), and microRNA, including microRNA mimics. RNAi agents may comprise conjugate groups and/or terminal groups. In certain embodiments, an RNAi agent modulates the amount, activity, and/or splicing of a target nucleic acid. The term RNAi agent excludes antisense agents that act through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H agents may comprise conjugate groups and/or terminal groups. In certain embodiments, an RNase H agent modulates the amount and/or activity of a target nucleic acid. The term RNase H agent excludes antisense agents that act principally through RISC/Ago2.

As used herein, “antisense RNase H oligonucleotide” means an oligonucleotide comprising a region that is complementary to a target sequence, and which includes at least one chemical modification suitable for RNase H-mediated nucleic acid reduction.

As used herein, “antisense RNAi oligonucleotide” means an oligonucleotide comprising a region that is complementary to a target sequence, and which includes at least one chemical modification suitable for RNAi-mediated nucleic acid reduction.

As used herein, “self-complementary” in reference to an oligonucleotide means an oligonucleotide that at least partially hybridizes to itself.

As used herein, “single-stranded” means a nucleic acid (including but not limited to an oligonucleotide) that is unpaired and is not part of a duplex. Single-stranded compounds are capable of hybridizing with complementary nucleic acids to form duplexes, at which point they are no longer single-stranded.

As used herein, “stabilized phosphate group” means a 5′-phosphate analog that is metabolically more stable than a 5′-phosphate as naturally occurs on DNA or RNA.

As used herein, “standard in vitro assay” means the assay described in Examples 1, 2, or 13 and reasonable variations thereof. In certain embodiments, “standard in vitro RNase H assay” means an in vitro assay for use with RNase H agents, and can include the assay described in Example 1 or Example 2 and reasonable variations thereof. In certain embodiments, “standard in vitro RNAi assay” means an in vitro assay for use with RNAi agents, and can include the assay described in Example 13 and reasonable variations thereof.

As used herein, “standard in vivo assay” means the assay described in Example 6 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal. In certain embodiments, the subject is a human.

As used herein, “sugar moiety” means an unmodified sugar moiety or a modified sugar moiety. As used herein, “unmodified sugar moiety” means a 2′-OH(H) β-D-ribosyl sugar moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests. In certain embodiments, symptoms and hallmarks include muscle weakness and muscle wasting in facio, scapula, and/or humeral muscle that can progress to the muscles of the trunk and lower limbs.

As used herein, “target nucleic acid” and “target RNA” mean a nucleic acid that an antisense compound is designed to affect. Target RNA means an RNA transcript and includes pre-mRNA and mature mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “treating” means improving a subject's disease or condition by administering an oligomeric agent or oligomeric compound described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a pharmaceutical agent or composition that provides a therapeutic benefit to an animal. For example, a therapeutically effective amount improves a symptom of a disease.

### CERTAIN EMBODIMENTS

The present disclosure provides the following non-limiting numbered embodiments:

Embodiment 1. An oligomeric compound comprising a modified oligonucleotide consisting of 12 to 50 linked nucleosides wherein the nucleobase sequence of the modified oligonucleotide is at least 80% complementary to an equal length portion of a DUX4 RNA, and wherein the modified oligonucleotide comprises at least one modification selected from a modified sugar moiety and a modified internucleoside linkage.

Embodiment 2. An oligomeric compound comprising a modified oligonucleotide consisting of 12 to 50 linked nucleosides and having a nucleobase sequence comprising at least 12, at least 13, at least 14, at least 15, at least 16, at least 17, at least 18, at least 19, or 20 nucleobases of any of SEQ ID NOs: 20-172, wherein the modified oligonucleotide comprises at least one modification selected from a modified sugar moiety and a modified internucleoside linkage.

Embodiment 3. An oligomeric compound comprising a modified oligonucleotide consisting of 12 to 50 linked nucleosides and having a nucleobase sequence comprising at least 12, at least 13, at least 14, at least 15, or 16 nucleobases of any of SEQ ID NOs: 173-1171, wherein the modified oligonucleotide comprises at least one modification selected from a modified sugar moiety and a modified internucleoside linkage.

Embodiment 4. An oligomeric compound comprising a modified oligonucleotide consisting of 12 to 50 linked nucleosides and having a nucleobase sequence comprising at least 12, at least 13, at least 14, at least 15, at least 16, at least 17, at least 18, at least 19, at least 20, at least 21, at least 22, or 23 nucleobases of any of SEQ ID NOs: 1176-1241, 1308, 1310-1473, or 1638, wherein the modified oligonucleotide comprises at least one modification selected from a modified sugar moiety and a modified internucleoside linkage.

Embodiment 5. An oligomeric compound comprising a modified oligonucleotide consisting of 12 to 50 linked nucleosides and having a nucleobase sequence comprising at least 8, at least 9, at least 10, at least 11, at least 12, at least 13, at least 14, at least 15, at least 16, at least 17, at least 18, at least 19, at least 20, at least 21, at least 22, or 23 nucleobases complementary to:
nucleobases 2697-2730 of SEQ ID NO: 1;
nucleobases 2756-2778 of SEQ ID NO: 1;
nucleobases 2732-2760 of SEQ ID NO: 1;
nucleobases 2783-2806 of SEQ ID NO: 1 and/or nucleobases 10-33 of SEQ ID NO: 2;
nucleobases 2833-2853 of SEQ ID NO: 1 and/or nucleobases 60-80 of SEQ ID NO: 2;
nucleobases 2953-2975 of SEQ ID NO: 1 and/or nucleobases 180-202 of SEQ ID NO: 2;
nucleobases 3097-3136 of SEQ ID NO: 1 and/or nucleobases 324-363 of SEQ ID NO: 2;
nucleobases 3174-3210 of SEQ ID NO: 1 and/or nucleobases 401-437 of SEQ ID NO: 2;
nucleobases 3361-3400 of SEQ ID NO: 1 and/or nucleobases 588-627 of SEQ ID NO: 2;
nucleobases 3883-3926 of SEQ ID NO: 1 and/or nucleobases 1110-1153 of SEQ ID NO: 2;
nucleobases 4007-4029 of SEQ ID NO: 1 and/or nucleobases 1234-1256 of SEQ ID NO: 2;
nucleobases 4103-4134 of SEQ ID NO: 1 and/or nucleobases 1330-1361 of SEQ ID NO: 2;
nucleobases 4503-4522 of SEQ ID NO: 1;
nucleobases 4509-4530 of SEQ ID NO: 1;
nucleobases 4667-4694 of SEQ ID NO: 1 and/or nucleobases 1532-1559 of SEQ ID NO: 2;
nucleobases 4828-4850 of SEQ ID NO: 1 and/or nucleobases 1693-1710 of SEQ ID NO: 2;
nucleobases 768-787 of SEQ ID NO: 2;
nucleobases 2955-2984 of SEQ ID NO: 1 and/or nucleobases 182-211 of SEQ ID NO: 2;
nucleobases 3099-3128 of SEQ ID NO: 1 and/or nucleobases 326-355 of SEQ ID NO: 2;
nucleobases 3171-3209 of SEQ ID NO: 1 and/or nucleobases 398-436 of SEQ ID NO: 2; or
nucleobases 3859-3888 of SEQ ID NO: 1 and/or nucleobases 1086-1115 of SEQ ID NO: 2;
wherein the modified oligonucleotide comprises at least one modification selected from a modified sugar moiety and a modified internucleoside linkage.

Embodiment 6. An oligomeric compound comprising a modified oligonucleotide consisting of 12 to 50 linked nucleosides and having a nucleobase sequence comprising at least 8, at least 9, at least 10, at least 11, at least 12, at least 13, at least 14, at least 15, at least 16, at least 17, at least 18, at least 19, at least 20, at least 21, at least 22, or 23 nucleobases of a sequence selected from:
SEQ ID NOs:24, 98, 318-320, 359-361, 398-400, 438-440, 852, and 857;
SEQ ID NOs: 323, 324, 364, 404, 443, and 444;
SEQ ID NOs: 73, 148, 327, 367, 407, 408, 208, and 447;
SEQ ID NOs: 299, 340, 379, and 420-421;
SEQ ID NOs: 300-301, 341-342, 380, 422, and 1002;
SEQ ID NOs: 331, 332, 370, 371, 412, 413, 451, and 452;
SEQ ID NOs: 326, 366, 406, 446, and 777;
SEQ ID NOs: 100, 186, 321, 362, 645, 719, 791, 796, 867, 873, 943, 946, and 1019;
SEQ ID NOs: 802, 880, and 1088-1090;
SEQ ID NOs: 668, 742, 815, 893, 966, and 1040;
SEQ ID NOs: 673, 747-748, 822, 898, 971, and 1045;
SEQ ID NOs: 39, 678-681, 753-755, 827-829, 903-905, 976-978, 1050-1052, and 1135-1136;
SEQ ID NOs: 117, 684, 758, 1056, and 1141-1144;
SEQ ID NOs: 123, 695-697, 769-771, 843, 844, 919, 920, 992, 993, 1066, and 1067;
SEQ ID NOs: 67, 141, 646-647, 720-721, 793-794, 870-871, 945, 947-948, and 1020-1021;
SEQ ID NOs: 652, 726, 799, 876, 953, 1026, and 1079;
SEQ ID NOs: 163, 658, 732, 883, 957, 1031, and 1105-1112;
SEQ ID NOs: 1424 and 1425;
SEQ ID NOs: 1214 and 1410;
SEQ ID NOs: 1405, 1209, and 1210; and
SEQ ID NOs: 1197 and 1335.

Embodiment 7. The oligomeric compound of any of embodiments 1-6, wherein the modified oligonucleotide has a nucleobase sequence that is at least 85%, at least 90%, at least 95%, or 100% complementary to any of the nucleobase sequences of SEQ ID NOs: 1-4 when measured across the entire nucleobase sequence of the modified oligonucleotide.

Embodiment 8. The oligomeric compound of any of embodiments 1-7, wherein the modified oligonucleotide consists of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

Embodiment 9. The oligomeric compound of any of embodiments 1-8, wherein the modified oligonucleotide comprises at least one modified nucleoside.

Embodiment 10. The oligomeric compound of embodiment 9, wherein the modified oligonucleotide comprises at least one modified nucleoside comprising a modified sugar moiety.

Embodiment 11. The oligomeric compound of embodiment 10, wherein the modified sugar moiety comprises a bicyclic sugar moiety.

Embodiment 12. The oligomeric compound of embodiment 11, wherein the bicyclic sugar moiety comprises a 2′-4′ bridge selected from —O—CH₂—; and —O—CH(CH₃)—.

Embodiment 13. The oligomeric compound of any of embodiments 10-12, wherein the modified oligonucleotide comprises at least one modified nucleoside comprising a non-bicyclic modified sugar moiety.

Embodiment 14. The oligomeric compound of embodiment 13, wherein the non-bicyclic modified sugar moiety is a 2′-O(CH₂)₂OCH₃ ribosyl sugar moiety, a cEt sugar moiety, a 2′-OMe sugar moiety, or a 2′-F sugar moiety.

Embodiment 15. The oligomeric compound of any of embodiments 10-14, wherein the modified oligonucleotide comprises at least one modified nucleoside comprising a sugar surrogate.

Embodiment 16. The oligomeric compound of embodiment 15, wherein the sugar surrogate is any of morpholino, modified morpholino, PNA, THP, and F-HNA.

Embodiment 17. The oligomeric compound of any of embodiments 1-16, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides; wherein each of the 5′-region nucleosides and each of the 3′-region nucleosides comprises a modified sugar moiety and each of the central region nucleosides comprises a 2′-β-D-deoxyribosyl sugar moiety.

Embodiment 18. The oligomeric compound of any of embodiments 1-16, wherein the modified oligonucleotide has a sugar motif comprising:
a 5′-region consisting of 3 linked 5′-region nucleosides;
a central region consisting of 10 linked central region nucleosides; and
a 3′-region consisting of 3 linked 3′-region nucleosides; wherein each of the 5′-region nucleosides and each of the 3′-region nucleosides comprises a modified sugar moiety and each of the central region nucleosides comprises a 2′-β-D-deoxyribosyl sugar moiety.

Embodiment 19. The oligomeric compound of any of embodiments 1-16, wherein the modified oligonucleotide has a sugar motif comprising:
a 5′-region consisting of 5 linked 5′-region nucleosides;
a central region consisting of 10 linked central region nucleosides; and
a 3′-region consisting of 5 linked 3′-region nucleosides; wherein each of the 5′-region nucleosides and each of the 3′-region nucleosides comprises a modified sugar moiety and each of the central region nucleosides comprises a 2′-β-D-deoxyribosyl sugar moiety.

Embodiment 20. The oligomeric compound of embodiment 17, wherein the modified oligonucleotide has a 5′-region consisting of 3 linked 5′-region nucleosides;
a central region consisting of 10 linked central region nucleosides; and
a 3′-region consisting of 3 linked 3′-region nucleosides; wherein
each of the 5′-region nucleosides and each of the 3′-region nucleosides comprises a cEt sugar moiety and each of the central region nucleosides comprises a 2′-β-D-deoxyribosyl sugar moiety.

Embodiment 21. The oligomeric compound of embodiment 17, wherein the modified oligonucleotide has
a 5′-region consisting of 5 linked 5′-region nucleosides;
a central region consisting of 10 linked central region nucleosides; and
a 3′-region consisting of 5 linked 3′-region nucleosides; wherein
each of the 5′-region nucleosides and each of the 3′-region nucleosides comprises a 2′-O(CH₂)₂OCH₃ ribosyl sugar moiety and each of the central region nucleosides comprises a 2′-β-D-deoxyribosyl sugar moiety.

Embodiment 22. The oligomeric compound of any of embodiments 1-21, wherein the modified oligonucleotide comprises at least one modified internucleoside linkage.

Embodiment 23. The oligomeric compound of embodiment 22, wherein each internucleoside linkage of the modified oligonucleotide is a modified internucleoside linkage.

Embodiment 24. The oligomeric compound of embodiment 22 or embodiment 23, wherein at least one internucleoside linkage is a phosphorothioate internucleoside linkage.

Embodiment 25. The oligomeric compound of embodiment 23, wherein each internucleoside linkage of the modified oligonucleotide is a phosphorothioate internucleoside linkage.

Embodiment 26. The oligomeric compound of embodiment 22 or embodiment 24, wherein each internucleoside linkage is independently selected from a phosphodiester internucleoside linkage and a phosphorothioate internucleoside linkage.

Embodiment 27. The oligomeric compound of any of embodiments 22-26, wherein the internucleoside linkage motif of the modified oligonucleotide is selected from: 5′-sssssssssssssssssss-3′ and 5′-sssssssssssssss-3′, wherein each “s” represents a phosphorothioate internucleoside linkage.

Embodiment 28. The oligomeric compound of any of embodiments 1-27, wherein the modified oligonucleotide comprises a modified nucleobase.

Embodiment 29. The oligomeric compound of embodiment 28, wherein the modified nucleobase is a 5-methylcytosine.

Embodiment 30. The oligomeric compound of any of embodiments 1-29, wherein the modified oligonucleotide consists of 12-30, 12-22, 12-20,14-18, 14-20, 15-17, 15-25, 16-20, 18-22 or 18-20 linked nucleosides, or a pharmaceutically acceptable salt thereof.

Embodiment 31. The oligomeric compound of embodiment 30, which is a pharmaceutically acceptable salt comprising one or more cations selected from sodium, potassium, calcium, and magnesium.

Embodiment 32. The oligomeric compound of any of embodiments 1-31, wherein the modified oligonucleotide consists of 16 linked nucleosides.

Embodiment 33. The oligomeric compound of any of embodiments 1-31, wherein the modified oligonucleotide consists of 20 linked nucleosides.

Embodiment 34. The oligomeric compound of any of embodiments 1-31, wherein the modified oligonucleotide consists of 23 linked nucleosides.

Embodiment 35. The oligomeric compound of any of embodiments 1-33, wherein the oligomeric compound activates RNase H.

Embodiment 36. The oligomeric compound of any of embodiments 1-35, consisting of the modified oligonucleotide.

Embodiment 37. The oligomeric compound of any of embodiments 1-35, consisting of the modified oligonucleotide and a conjugate group.

Embodiment 38. The oligomeric compound of embodiment 37, wherein the conjugate group comprises a conjugate moiety and a conjugate linker.

Embodiment 39. The oligomeric compound of embodiment 38, wherein the conjugate moiety is a lipophilic group.

Embodiment 40. The oligomeric compound of embodiment 38, wherein the conjugate moiety is selected from a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, and C5 alkenyl.

Embodiment 41. The oligomeric compound of embodiment 38, wherein the conjugate moiety is a 6-palmitamidohexyl conjugate moiety.

Embodiment 42. The oligomeric compound of any of embodiments 38-41, wherein the conjugate linker is a phosphodiester linker.

Embodiment 43. The oligomeric compound of any of embodiments 37-42, wherein the conjugate group has the following structure:

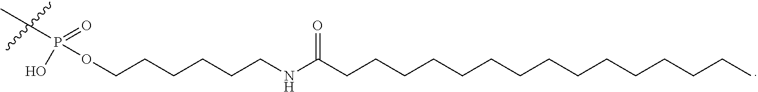 [SMILES: *P(=O)(O)OCCCCCCNC(=O)CCCCCCCCCCCCCCC]

Embodiment 44. The oligomeric compound of any of embodiments 38-43, wherein the conjugate linker consists of a single bond.

Embodiment 45. The oligomeric compound of any of embodiments 38-44, wherein the conjugate linker is cleavable.

Embodiment 46. The oligomeric compound of any of embodiments 38-45, wherein the conjugate linker comprises 1-3 linker-nucleosides.

Embodiment 47. The oligomeric compound of any of embodiments 37-46, wherein the conjugate group is attached to the modified oligonucleotide at the 5′-end of the modified oligonucleotide.

Embodiment 48. The oligomeric compound of any of embodiments 37-46, wherein the conjugate group is attached to the modified oligonucleotide at the 3′-end of the modified oligonucleotide.

Embodiment 49. The oligomeric compound of any of embodiments 1-48, comprising a terminal group.

Embodiment 50. The oligomeric compound of any of embodiments 1-45 or 47-49, wherein the oligomeric compound does not comprise linker-nucleosides.

Embodiment 51. An oligomeric compound according to the following chemical structure:

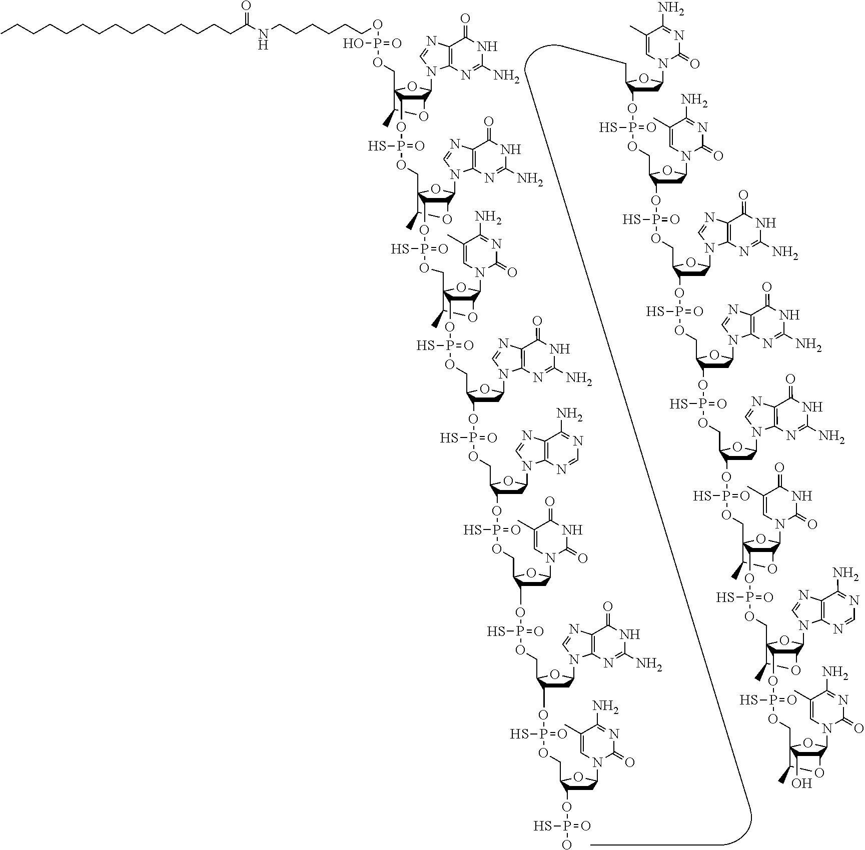

Embodiment 52. The oligomeric compound of embodiment 51, which is a pharmaceutically acceptable salt comprising one or more cations selected from sodium, potassium, calcium, and magnesium.

Embodiment 53. An oligomeric compound according to the following chemical structure:

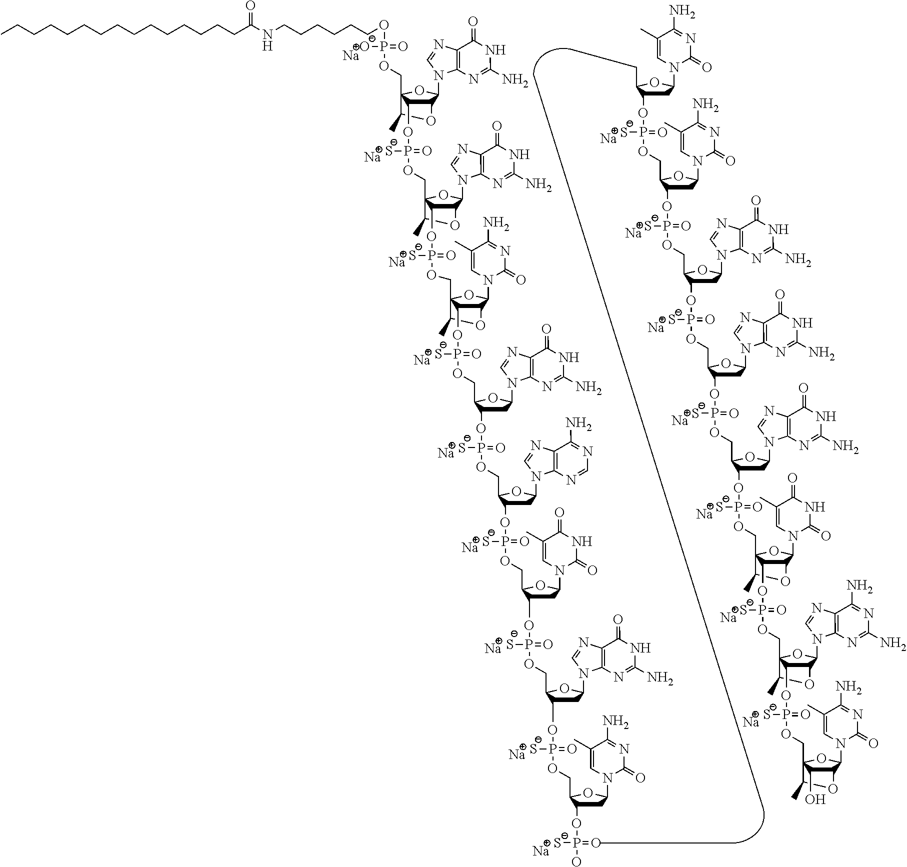

Embodiment 54. An oligomeric compound comprising a modified oligonucleotide and conjugate group according to the following chemical notation: (6-palmitamidohexyl)-GₖₛGₖₛ^{m}CₖₛG_{ds}A_{ds}T_{ds}G_{ds}^{m}C_{ds}^{m}C_{ds}^{m}C_{ds}G_{ds}G_{ds}G_{ds}TₖₛAₖₛ^{m}Cₖ (SEQ ID NO:248), wherein:
A=an adenine nucleobase,
^{m}C=a 5-methylcytosine nucleobase,
G=a guanine nucleobase,
T=a thymine nucleobase,
k=a cEt sugar moiety,
d=a 2′-β-D-deoxyribosyl sugar moiety, and
s=a phosphorothioate internucleoside linkage.

Embodiment 55. A chirally enriched population of oligomeric compounds of any of embodiments 1-54, wherein the population is enriched for modified oligonucleotides comprising at least one particular phosphorothioate internucleoside linkage having a particular stereochemical configuration.

Embodiment 56. The chirally enriched population of embodiment 55, wherein the population is enriched for modified oligonucleotides comprising at least one particular phosphorothioate internucleoside linkage having the (Sp) configuration.

Embodiment 57. The chirally enriched population of embodiment 55, wherein the population is enriched for modified oligonucleotides comprising at least one particular phosphorothioate internucleoside linkage having the (Rp) configuration.

Embodiment 58. The chirally enriched population of embodiment 55, wherein the population is enriched for modified oligonucleotides having a particular, independently selected stereochemical configuration at each phosphorothioate internucleoside linkage.

Embodiment 59. The chirally enriched population of embodiment 55, wherein the population is enriched for modified oligonucleotides having the (Sp) configuration at each phosphorothioate internucleoside linkage or for modified oligonucleotides having the (Rp) configuration at each phosphorothioate internucleoside linkage.

Embodiment 60. The chirally enriched population of embodiment 55, wherein the population is enriched for modified oligonucleotides having the (Rp) configuration at one particular phosphorothioate internucleoside linkage and the (Sp) configuration at each of the remaining phosphorothioate internucleoside linkages.

Embodiment 61. The chirally enriched population of embodiment 55, wherein the population is enriched for modified oligonucleotides having at least 3 contiguous phosphorothioate internucleoside linkages in the Sp, Sp, and Rp configurations, in the 5′ to 3′ direction.

Embodiment 62. A population of oligomeric compounds of any of embodiments 1-54, wherein all of the phosphorothioate internucleoside linkages of the modified oligonucleotide are stereorandom.

Embodiment 63. An oligomeric duplex comprising a first oligomeric compound and a second oligomeric compound comprising a second modified oligonucleotide, wherein the first oligomeric compound is an oligomeric compound of any of embodiments 1-54.

Embodiment 64. The oligomeric duplex of embodiment 63, wherein the second modified oligonucleotide consists of 12 to 50 linked nucleosides, and wherein the nucleobase sequence of the second modified oligonucleotide comprises a complementary region of at least 12 nucleobases that is at least 90% complementary to an equal length portion of the first modified oligonucleotide.

Embodiment 65. An oligomeric duplex comprising:
a first oligomeric compound comprising a first modified oligonucleotide consisting of 19 to 30 linked nucleosides, wherein the nucleobase sequence of the first modified oligonucleotide comprises at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 1176-1241, 1308, 1310-1473, or 1638; and
a second oligomeric compound comprising a second modified oligonucleotide consisting of 15 to 30 linked nucleosides wherein the nucleobase sequence of the second modified oligonucleotide comprises a complementary region of at least 12 nucleobases that is at least 90% complementary to an equal length portion of the first modified oligonucleotide.

Embodiment 66. An oligomeric duplex comprising:
a first oligomeric compound comprising a first modified oligonucleotide consisting of 19 to 30 linked nucleosides, wherein the nucleobase sequence of the first modified oligonucleotide comprises at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 1176-1241, 1308, 1310-1473, or 1638; and
a second oligomeric compound comprising a second modified oligonucleotide consisting of 15 to 30 linked nucleosides wherein the nucleobase sequence of the second modified oligonucleotide comprises at least 12, at least 13, at least 14, at least 15, at least 16, at least 17, at least 18, at least 19, at least 20, or 21 contiguous nucleobases of the nucleobase sequence of any of SEQ ID NOs: 1242-1307, 1309, 1474-1637, or 1639, wherein the nucleobase sequence of the second modified oligonucleotide is at least 90% complementary to an equal length portion of the first modified oligonucleotide.

Embodiment 67. An oligomeric duplex comprising:
a first oligomeric compound comprising a first modified oligonucleotide consisting of 23 linked nucleosides, wherein the nucleobase sequence of the first modified oligonucleotide comprises the nucleobase sequence of any of SEQ ID NOs: 1176-1241, 1308, 1310-1473, or 1638; and
a second oligomeric compound comprising a second modified oligonucleotide consisting of 21 linked nucleosides, wherein the nucleobase sequence of the second modified oligonucleotide comprises the nucleobase sequence of any of SEQ ID NOs: 1242-1307, 1309, 1474-1637, or 1639, wherein the nucleobase sequence of the second modified oligonucleotide is at least 90% complementary to an equal length portion of the first modified oligonucleotide.

Embodiment 68. The oligomeric duplex of any of embodiments 63-67, wherein the modified oligonucleotide of the first oligomeric compound comprises a 5′-stabilized phosphate group.

Embodiment 69. The oligomeric duplex of embodiment 68, wherein the 5′-stabilized phosphate group comprises a cyclopropyl phosphonate or a vinyl phosphonate.

Embodiment 70. The oligomeric duplex of any of embodiments 63-69, wherein at least one nucleoside of the first modified oligonucleotide comprises a modified sugar moiety.

Embodiment 71. The oligomeric duplex of embodiment 70, wherein the modified sugar moiety of the first modified oligonucleotide comprises a bicyclic sugar moiety.

Embodiment 72. The oligomeric duplex of embodiment 71, wherein the bicyclic sugar moiety comprises a 2′-4′ bridge selected from —O—CH₂—; and —O—CH(CH₃)—.

Embodiment 73. The oligomeric duplex of embodiment 70, wherein the modified sugar moiety of the first modified oligonucleotide comprises a non-bicyclic modified sugar moiety.

Embodiment 74. The oligomeric duplex of embodiment 73, wherein the non-bicyclic modified sugar moiety of the first modified oligonucleotide is a 2′-OMe sugar moiety or a 2′-F sugar moiety.

Embodiment 75. The oligomeric duplex of any of embodiments 63-74, wherein at least one nucleoside of the first modified oligonucleotide comprises a sugar surrogate.

Embodiment 76. The oligomeric duplex of any of embodiments 63-75, wherein the first modified oligonucleotide comprises at least one modified internucleoside linkage.

Embodiment 77. The oligomeric duplex of embodiment 76, wherein at least one modified internucleoside linkage of the first modified oligonucleotide is a phosphorothioate internucleoside linkage.

Embodiment 78. The oligomeric duplex of embodiment 76, wherein each internucleoside linkage of the first modified oligonucleotide is independently selected from a phosphodiester and a phosphorothioate internucleoside linkage.

Embodiment 79. The oligomeric duplex of any of embodiments 63-78, wherein at least one nucleoside of the second modified oligonucleotide comprises a modified sugar moiety.

Embodiment 80. The oligomeric duplex of embodiment 79, wherein the modified sugar moiety of the second modified oligonucleotide comprises a bicyclic sugar moiety.

Embodiment 81. The oligomeric duplex of embodiment 80, wherein the bicyclic sugar moiety comprises a 2′-4′ bridge selected from —O—CH₂—; and —O—CH(CH₃)—.

Embodiment 82. The oligomeric duplex of embodiment 79, wherein the modified sugar moiety of the second modified oligonucleotide comprises a non-bicyclic modified sugar moiety.

Embodiment 83. The oligomeric duplex of embodiment 82, wherein the non-bicyclic modified sugar moiety of the second modified oligonucleotide is a 2′-OMe sugar moiety or a 2′-F sugar moiety.

Embodiment 84. The oligomeric duplex of any of embodiments 63-83, wherein at least one nucleoside of the second modified oligonucleotide comprises a sugar surrogate.

Embodiment 85. The oligomeric duplex of any of embodiments 63-84, wherein the second modified oligonucleotide comprises at least one modified internucleoside linkage.

Embodiment 86. The oligomeric duplex of embodiment 85, wherein at least one modified internucleoside linkage of the second modified oligonucleotide is a phosphorothioate internucleoside linkage.

Embodiment 87. The oligomeric duplex of embodiment 85, wherein each internucleoside linkage of the second modified oligonucleotide is independently selected from a phosphodiester and a phosphorothioate internucleoside linkage.

Embodiment 88. The oligomeric duplex of any of embodiments 63-87, wherein the internucleoside linkage motif of the first modified oligonucleotide is ssooooooooooooooooooss and the internucleoside linkage motif of the second modified oligonucleotide is ssooooooooooooooooss, wherein each “o” represents a phosphodiester internucleoside linkage and each “s” represents a phosphorothioate internucleoside linkage.

Embodiment 89. The oligomeric duplex of any of embodiments 63-88, wherein the first modified oligonucleotide has a sugar motif of 5′-yfyfyfyfyfyfyfyfyfyfyyy-3′ and the second modified oligonucleotide has a sugar motif of 5′-fyfyfyfyfyfyfyfyfyfyf-3′, wherein each “y” represents a 2′-OMe sugar moiety and each “f” represents a 2′-F sugar moiety.

Embodiment 90. The oligomeric duplex of any of embodiments 63-89, wherein the first modified oligonucleotide and the second modified oligonucleotide each independently comprises at least one modified nucleobase.

Embodiment 91. The oligomeric duplex of embodiment 90, wherein the at least one modified nucleobase is 5-methylcytosine.

Embodiment 92. The oligomeric duplex of any of embodiments 63-91, wherein 1-4 3′-most nucleosides of the first modified oligonucleotide are overhanging nucleosides.

Embodiment 93. The oligomeric duplex of any of embodiments 63-92, wherein the duplex is blunt ended at the 5′-end of the first modified oligonucleotide.

Embodiment 94. The oligomeric duplex of any of embodiments 63-93, wherein the second oligomeric compound comprises a conjugate group comprising a conjugate moiety and a conjugate linker.

Embodiment 95. The oligomeric duplex of embodiment 94, wherein the conjugate linker consists of a single bond.

Embodiment 96. The oligomeric duplex of embodiment 94, wherein the conjugate linker is cleavable.

Embodiment 97. The oligomeric duplex of embodiment 94, wherein the conjugate linker comprises 1-3 linker-nucleosides.

Embodiment 98. The oligomeric duplex of any of embodiments 94-97, wherein the conjugate group is attached to the 5′-end of the second modified oligonucleotide.

Embodiment 99. The oligomeric duplex of any of embodiments 94-97, wherein the conjugate group is attached to the 3′-end of the second modified oligonucleotide.

Embodiment 100. The oligomeric duplex of any of embodiments 94-97, wherein the conjugate group is attached via the 2′ position of a ribosyl sugar moiety at an internal position of the second modified oligonucleotide.

Embodiment 101. The oligomeric duplex of any of embodiments 94-101, wherein the conjugate group comprises a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

Embodiment 102. The oligomeric duplex of any of embodiments 94-101, wherein the conjugate moiety is a 6-palmitamidohexyl conjugate moiety.

Embodiment 103. The oligomeric duplex of any of embodiments 94 or 96-102, wherein the conjugate linker is a phosphodiester linker.

Embodiment 104. The oligomeric duplex of any one of embodiments 94 or 96-100, wherein the conjugate group has the following structure:

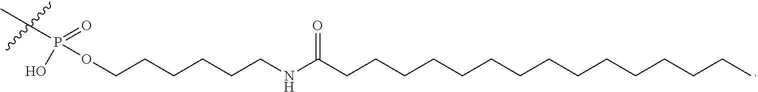 [SMILES: *P(=O)(O)OCCCCCCNC(=O)CCCCCCCCCCCCCCC]

Embodiment 105. The oligomeric duplex of any of embodiments 94-104, wherein the conjugate group comprises a cell-targeting moiety.

Embodiment 106. The oligomeric duplex of any of embodiments 63-105, wherein the second modified oligonucleotide comprises a terminal group.

Embodiment 107. The oligomeric duplex of embodiment 106, wherein the terminal group is an abasic sugar moiety.

Embodiment 108. The oligomeric duplex of any of embodiments 63-107, wherein the second modified oligonucleotide consists of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

Embodiment 109. The oligomeric duplex of any of embodiments 63-66 or 68-108, wherein the first modified oligonucleotide consists of 23 linked nucleosides and the second modified oligonucleotide consists of 21 linked nucleosides.

Embodiment 110. An antisense agent comprising or consisting of an antisense compound, wherein the antisense compound is the oligomeric compound of any of embodiments 1-54.

Embodiment 111. An antisense agent, wherein the antisense agent is the oligomeric duplex of any of embodiments 63-109.

Embodiment 112. The antisense agent of embodiment 110 or embodiment 111, wherein the antisense agent is:
i) an RNase H agent capable of reducing the amount of DUX4 nucleic acid through the activation of RNase H; or
ii) an RNAi agent capable of reducing the amount of DUX4 nucleic acid through the activation of RISC/Ago2.

Embodiment 113. The antisense agent of any of embodiments 110-112, wherein the antisense agent comprises a conjugate group, and wherein the conjugate group comprises a cell-targeting moiety.

Embodiment 114. A pharmaceutical composition comprising an oligomeric compound of any of embodiments 1-54, a population of oligomeric compounds of any of embodiments 55-62, an oligomeric duplex of any of embodiments 63-109, or an antisense agent of any of embodiments 110-113, and a pharmaceutically acceptable diluent.

Embodiment 115. The pharmaceutical composition of embodiment 114, wherein the pharmaceutically acceptable diluent is phosphate buffered saline (PBS).

Embodiment 116. The pharmaceutical composition of embodiment 115, wherein the pharmaceutical composition consists essentially of the oligomeric compound and PBS.

Embodiment 117. The pharmaceutical composition of embodiment 115, wherein the pharmaceutical composition consists essentially of the population of oligomeric compounds and PBS.

Embodiment 118. The pharmaceutical composition of embodiment 115, wherein the pharmaceutical composition consists essentially of the oligomeric duplex or the antisense agent and PBS.

Embodiment 119. A method comprising administering to a subject an oligomeric compound of any of embodiments 1-54, a population of oligomeric compounds of any of embodiments 55-62, an oligomeric duplex of any of embodiments 63-109, an antisense agent of any of embodiments 110-113, or a pharmaceutical composition of any of embodiments 114-118.

Embodiment 120. A method of treating a disease or disorder associated with DUX4 comprising administering to a subject having or at risk for developing a disease or disorder associated with DUX4 a therapeutically effective amount of an oligomeric compound of any of embodiments 1-54, a population of oligomeric compounds of any of embodiments 55-62, an oligomeric duplex of any of embodiments 63-109, an antisense agent of any of embodiments 110-113, or a pharmaceutical composition of any of embodiments 114-118; and thereby treating the disease or disorder associated with DUX4.

Embodiment 121. The method of embodiment 120, where the disease or disorder associated with DUX4 is a muscle dystrophy.

Embodiment 122. The method of embodiment 120, wherein the disease or disorder associated with DUX4 is facioscapulohumeral muscular dystrophy (FSHD).

Embodiment 123. The method of any of embodiments 120-122, wherein at least one symptom or hallmark of the disease or disorder associated with DUX4 is ameliorated.

Embodiment 124. The method of embodiment 123, wherein the symptom or hallmark is muscle weakness or muscle wasting in facio, scapula, and/or humeral muscle.

Embodiment 125. The method of embodiment 124, wherein administering the oligomeric compound of any of embodiments 1-54, the population of oligomeric compounds of any of embodiments 55-62, the oligomeric duplex of any of embodiments 63-109, the antisense agent of any of embodiments 110-113, or the pharmaceutical composition of any of embodiments 114-118 reduces or delays the onset or progression of muscle weakness or muscle wasting in the subject.

Embodiment 126. The method of any of embodiments 119-125, wherein the oligomeric compound of any of embodiments 1-54, the population of oligomeric compounds of any of embodiments 55-62, the oligomeric duplex of any of embodiments 63-109, the antisense agent of any of embodiments 110-113, or the pharmaceutical composition of any of embodiments 114-118 is administered systemically.

Embodiment 127. The method of any of embodiments 119-126, wherein the subject is a human.

Embodiment 128. A method of reducing expression of DUX4 in a cell comprising contacting the cell with an oligomeric compound of any of embodiments 1-54, a population of oligomeric compounds of any of embodiments 55-62, an oligomeric duplex of any of embodiments 63-109, an antisense agent of any of embodiments 110-113, or a pharmaceutical composition of any of embodiments 114-118.

Embodiment 129. The method of embodiment 128, wherein the cell is a muscle cell.

Embodiment 130. The method of embodiment 128 or embodiment 129, wherein the cell is a human cell.

Embodiment 131. Use of the oligomeric compound of any of embodiments 1-54, the population of oligomeric compounds of any of embodiments 55-62, the oligomeric duplex of any of embodiments 63-109, the antisense agent of any of embodiments 110-113, or the pharmaceutical composition of any of embodiments 114-118 for treating a disease or disorder associated with DUX4.

Embodiment 132. Use of the oligomeric compound of any of embodiments 1-54, the population of oligomeric compounds of any of embodiments 55-62, the oligomeric duplex of any of embodiments 63-109, the antisense agent of any of embodiments 110-113, or the pharmaceutical composition of any of embodiments 114-118 in the manufacture of a medicament for treating a disease or disorder associated with DUX4.

Embodiment 133. The use of embodiment 131 or embodiment 132, wherein the disease or disorder associated with DUX4 is FSHD.

Certain Oligomeric Agents and Oligomeric Compounds Certain embodiments provide oligomeric agents targeted to a DUX4 nucleic acid. In certain embodiments, the DUX4 nucleic acid has the sequence set forth in SEQ ID NO: 1 (GENBANK Accession No. NC_000004.12, truncated from nucleotides 190171001 to 190187000), SEQ ID NO: 2 (GENBANK Accession No. NM_001306068.2), SEQ ID NO: 3 (GENBANK Accession No. FJ439133.1), or SEQ ID NO: 4 (GENBANK Accession No. NM_001293798.2), each of which is incorporated by reference in its entirety. In certain embodiments, the oligomeric agent is a single-stranded oligomeric compound. In certain embodiments, the oligomeric agent is oligomeric duplex.

Certain embodiments provide an oligomeric compound comprising a modified oligonucleotide consisting of 12 to 50 linked nucleosides wherein the nucleobase sequence of the modified oligonucleotide is at least 80% complementary to an equal length portion of a DUX4 RNA, and wherein the modified oligonucleotide comprises at least one modification selected from a modified sugar moiety and a modified internucleoside linkage. In certain embodiments, the DUX4 nucleic acid has the nucleobase sequence of SEQ ID NO: 1, 2, 3, or 4.

Certain embodiments provide an oligomeric compound comprising a modified oligonucleotide consisting of 12 to 50 linked nucleosides and having a nucleobase sequence comprising at least 12, at least 13, at least 14, at least 15, at least 16, at least 17, at least 18, at least 19, or 20 nucleobases of any of SEQ ID NOs: 20-172.

Certain embodiments provide an oligomeric compound comprising a modified oligonucleotide consisting of 12 to 50 linked nucleosides and having a nucleobase sequence comprising at least 12, at least 13, at least 14, at least 15, or 16 nucleobases of any of SEQ ID NOs: 173-1171.

Certain embodiments provide an oligomeric compound comprising a modified oligonucleotide consisting of 12 to 50 linked nucleosides and having a nucleobase sequence comprising at least 12, at least 13, at least 14, at least 15, at least 16, at least 17, at least 18, at least 19, at least 20, at least 21, at least 22, or 23 nucleobases of any of SEQ ID NOs: 1176-1241, 1308, 1310-1473, or 1638.

In any of the oligomeric compounds provided herein, the nucleobase sequence of the modified oligonucleotide can be at least 85%, at least 90%, at least 95%, or 100% complementary to an equal length portion of a DUX4 nucleic acid, wherein the DUX4 nucleic acid has the nucleobase sequences of any of SEQ ID NOs: 1-4.

In any of the oligomeric compounds provided herein, the modified oligonucleotide consists of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

In any of the oligomeric compounds provided herein, at least one nucleoside of the modified oligonucleotide can comprise a modified sugar moiety. In certain embodiments, the modified sugar moiety comprises a bicyclic sugar moiety, such as a 2′-4′ bridge selected from —O—CH₂—; and —O—CH(CH₃)—. In certain embodiments, the modified sugar moiety comprises a non-bicyclic sugar moiety, such as a 2′-MOE sugar moiety, a cEt sugar moiety, a 2′-OMe sugar moiety, or a 2′-F sugar moiety.

In any of the oligomeric compounds provided herein, at least one nucleoside of the modified oligonucleotide compound can comprise a sugar surrogate.

In any of the oligomeric compounds provided herein, at least one internucleoside linkage of the modified oligonucleotide can comprise a modified internucleoside linkage, such as a phosphorothioate internucleoside linkage. In certain embodiments, each internucleoside linkage of the modified oligonucleotide can be a modified internucleoside linkage or each internucleoside linkage of the modified oligonucleotide can be a phosphorothioate internucleoside linkage. In certain embodiments, at least one internucleoside linkage of the modified oligonucleotide can be a phosphodiester internucleoside linkage. In certain embodiments, each internucleoside linkage of the modified oligonucleotide can be independently selected from a phosphodiester or a phosphorothioate internucleoside linkage. In certain embodiments, at least 2, at least 3, at least 4, at least 5, or at least 6 internucleoside linkages of the modified oligonucleotide can be phosphodiester internucleoside linkages. In certain embodiments, at least 10, at least 11, at least 12, at least 13, at least 14, at least 15, at least 16, at least 17, at least 18, at least 19, or 20 internucleoside linkages of the modified oligonucleotide can be phosphorothioate internucleoside linkages.

In any of the oligomeric compounds provided herein, at least one nucleobase of the modified oligonucleotide can be a modified nucleobase, such as 5-methylcytosine. In certain embodiments, each cytosine is 5-methylcytosine.

In any of the oligomeric compounds provided herein, the modified oligonucleotide can comprise a deoxy region consisting of 5-12 contiguous 2′-deoxynucleosides. In certain embodiments, each nucleoside of the deoxy region is a 2′-β-D-deoxynucleoside. In certain embodiments, the deoxy region consists of 7, 8, 9, 10, or 7-10 linked nucleosides. In certain embodiments, each nucleoside immediately adjacent to the deoxy region comprises a modified sugar moiety. In certain embodiments, the deoxy region is flanked on the 5′-side by a 5′-region consisting of 1-6 linked 5′-region nucleosides and on the 3′-side by a 3′-region consisting of 1-6 linked 3′-region nucleosides; wherein the 3′- most nucleoside of the 5′-region comprises a modified sugar moiety; and the 5′-most nucleoside of the 3′-region comprises a modified sugar moiety. In certain embodiments, each nucleoside of the 3′-region comprises a modified sugar moiety. In certain embodiments, each nucleoside of the 5′-region comprises a modified sugar moiety.

In certain embodiments, a compound comprises or consists of a modified oligonucleotide consisting of 16 to 50 linked nucleobases and having a nucleobase sequence comprising the nucleobase sequence recited in any one of SEQ ID NOs: 20-172, wherein the modified oligonucleotide has:
a 5′-region consisting of 5 linked 5′-region nucleosides;
a central region consisting of 10 linked central region nucleosides; and a 3′-region consisting of 5 linked 3′-region nucleosides;
wherein each of the 5′-region nucleosides and each of the 3′-region nucleosides comprises a 2′-MOE sugar moiety, each of the central region nucleosides comprises a 2′-β-D-deoxyribosyl sugar moiety, and wherein each cytosine is a 5-methylcytosine. In certain embodiments, the modified oligonucleotide consists of 15 to 30 linked nucleosides. In certain embodiments, the modified oligonucleotide consists of 20 linked nucleosides.

In certain embodiments, a compound comprises or consists of a modified oligonucleotide consisting of 16 to 50 linked nucleobases and having a nucleobase sequence comprising the nucleobase sequence recited in any one of SEQ ID NOs: 173-1171, wherein the modified oligonucleotide has:
a 5′-region consisting of 3 linked 5′-region nucleosides;
a central region consisting of 10 linked central region nucleosides; and
a 3′-region consisting of 3 linked 3′-region nucleosides;
   wherein each of the 5′-region nucleosides and each of the 3′-region nucleosides comprises a 2′-cEt sugar moiety, each of the central region nucleosides comprises a 2′-β-D-deoxyribosyl sugar moiety, and wherein each cytosine is a 5-methylcytosine. In certain embodiments, the modified oligonucleotide consists of 15 to 30 linked nucleosides. In certain embodiments, the modified oligonucleotide consists of 16 linked nucleosides.

In certain embodiments, an oligomeric compound comprises a conjugate group. In certain embodiments, the conjugate group comprises a conjugate linker and a conjugate moiety. In certain embodiments, the conjugate linker consists of a single bond, the conjugate linker is cleavable, the conjugate linker comprises 1-3 linker-nucleosides, the conjugate linker does not comprise any linker nucleosides, the conjugate group is attached to the modified oligonucleotide at the 5′-end of the modified oligonucleotide, or the conjugate group is attached to the modified oligonucleotide at the 3′-end of the modified oligonucleotide.

In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In certain embodiments, the conjugate group has the following structure:

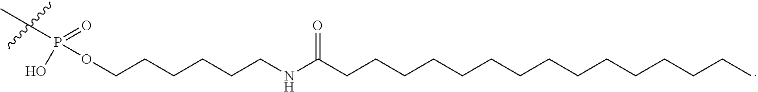 [SMILES: *P(=O)(O)OCCCCCCNC(=O)CCCCCCCCCCCCCCC]

### Certain Oligomeric Duplexes

Certain embodiments are directed to oligomeric duplexes comprising a first oligomeric compound and a second oligomeric compound.

In certain embodiments, an oligomeric duplex comprises:
a first oligomeric compound comprising a first modified oligonucleotide consisting of 12 to 50 linked nucleosides, wherein the nucleobase sequence of the first modified oligonucleotide is at least 80% complementary to an equal length portion within nucleobases sequence of any of SEQ ID NOs: 20-1171; and
a second oligomeric compound comprising a second modified oligonucleotide consisting of 12 to 50 linked nucleosides wherein the nucleobase sequence of the second modified oligonucleotide comprises a complementary region of at least 8 nucleobases that is at least 90% complementary to an equal length portion of the first modified oligonucleotide.

In certain embodiments, an oligomeric duplex comprises:
a first oligomeric compound comprising a first modified oligonucleotide consisting of 12 to 50 linked nucleosides wherein the nucleobase sequence of the first modified oligonucleotide comprises at least 8, at least 9, at least 10, at least 11, at least 12, at least 13, at least 14, at least 15, or at least 16 contiguous nucleobases of the nucleobase sequence of any of SEQ ID NOs: 20-1171, wherein each thymine is replaced by uracil; and
a second oligomeric compound comprising a second modified oligonucleotide consisting of 12 to 50 linked nucleosides wherein the nucleobase sequence of the second modified oligonucleotide comprises a complementary region of at least 8 nucleobases that is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:
a first oligomeric compound comprising a first modified oligonucleotide consisting of 16 to 50 linked nucleosides wherein the nucleobase sequence of the first modified oligonucleotide comprises the nucleobase sequence of any of SEQ ID NOs: 20-1171, wherein each thymine is replaced by uracil; and
a second oligomeric compound comprising a second modified oligonucleotide consisting of 16 to 50 linked nucleosides wherein the nucleobase sequence of the second modified oligonucleotide comprises a complementary region of at least 16 nucleobases that is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:
a first oligomeric compound comprising a first modified oligonucleotide consisting of 19 to 30 linked nucleosides wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 1176-1241, 1308, 1310-1473, or 1638; and
a second oligomeric compound comprising a second modified oligonucleotide consisting of 15 to 30 linked nucleosides wherein the nucleobase sequence of the second modified oligonucleotide comprises a complementary region of at least 8 nucleobases that is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:
a first oligomeric compound comprising a first modified oligonucleotide consisting of 19 to 30 linked nucleosides wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 1176-1241, 1308, 1310-1473, or 1638; and
a second oligomeric compound comprising a second modified oligonucleotide consisting of 15 to 30 linked nucleosides wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or 21 contiguous nucleobases of the nucleobase sequence of any of SEQ ID NOs: 1242-1307, 1309, 1474-1637, or 1639, wherein the nucleobase sequence of the second modified oligonucleotide is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:
a first oligomeric compound comprising a first modified oligonucleotide consisting of 23 linked nucleosides wherein the nucleobase sequence of the first modified oligonucleotide comprises the nucleobase sequence of any of SEQ ID NOs: 1176-1241, 1308, 1310-1473, or 1638; and
a second oligomeric compound comprising a second modified oligonucleotide consisting of 21 linked nucleosides wherein the nucleobase sequence of the second modified oligonucleotide comprises the nucleobase sequence of any of SEQ ID NOs: 1242-1307, 1309, 1474-1637, or 1639, wherein the nucleobase sequence of the second modified oligonucleotide is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises a first oligomeric compound comprising a first modified oligonucleotide consisting of 19 to 30 linked nucleosides and a second oligomeric compound comprising a second modified oligonucleotide consisting of 15 to 30 linked nucleosides, wherein the nucleobase sequence of the first modified oligonucleotide and the nucleobase sequence of the second modified oligonucleotide each comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any of the following pairs of nucleobase sequences recited in: SEQ ID NOs: 1176-1639, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises a first oligomeric compound comprising a first modified oligonucleotide consisting of 19 to 30 linked nucleosides and a second oligomeric compound comprising a second modified oligonucleotide consisting of 15 to 30 linked nucleosides, wherein the nucleobase sequences of the first modified oligonucleotide and second modified oligonucleotide comprise any of the following pairs of nucleobase sequences recited in: SEQ ID NOs: 1176-1639, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises a first oligomeric compound comprising a first modified oligonucleotide consisting of 23 linked nucleosides and a second oligomeric compound comprising a second modified oligonucleotide consisting of 21 linked nucleosides, wherein the nucleobase sequences of the first modified oligonucleotide and second modified oligonucleotide consist of any of the following pairs of nucleobase sequences recited in: SEQ ID NOs: 1176-1639, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In any of the oligomeric duplexes described herein, at least one nucleoside of the first modified oligonucleotide and/or the second modified oligonucleotide can comprise a modified sugar moiety. Examples of suitable modified sugar moieties include, but are not limited to, a bicyclic sugar moiety, such as a 2′-4′ bridge selected from —O—CH2-; and —O—CH(CH3)-, and a non-bicyclic sugar moiety, such as a 2′-MOE sugar moiety, a 2′-F sugar moiety, a 2′-OMe sugar moiety, or a 2′-NMA sugar moiety. In certain embodiments, at least 80%, at least 90%, or 100% of the nucleosides of the first modified oligonucleotide and/or the second modified oligonucleotide comprises a modified sugar moiety selected from 2′-F and 2′-OMe.

In any of the oligomeric duplexes described herein, at least one nucleoside of the first modified oligonucleotide and/or the second modified oligonucleotide can comprise a sugar surrogate. Examples of suitable sugar surrogates include, but are not limited to, morpholino, peptide nucleic acid (PNA), glycol nucleic acid (GNA), and unlocked nucleic acid (UNA). In certain embodiments, at least one nucleoside of the first modified oligonucleotide comprises a sugar surrogate, which can be a GNA.

In any of the oligomeric duplexes described herein, at least one internucleoside linkage of the first modified oligonucleotide and/or the second modified oligonucleotide can comprise a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage.

In any of the oligomeric duplexes described herein, at least one internucleoside linkage of the first modified oligonucleotide and/or the second modified oligonucleotide can comprise a phosphodiester internucleoside linkage.

In any of the oligomeric duplexes described herein, each internucleoside linkage of the first modified oligonucleotide and/or the second modified oligonucleotide can be independently selected from a phosphodiester or a phosphorothioate internucleoside linkage.

In any of the oligomeric duplexes described herein, the internucleoside linkage motif of the first modified oligonucleotide can be ssooooooooooooooooooss and the internucleoside linkage motif of the second modified oligonucleotide can be ssooooooooooooooooss, wherein each “o” represents a phosphodiester internucleoside linkage and each “s” represents a phosphorothioate internucleoside linkage.

In any of the oligomeric duplexes described herein, at least one nucleobase of the first modified oligonucleotide and/or the second modified oligonucleotide can be modified nucleobase. In certain embodiments, the modified nucleobase is 5-methylcytosine.

In any of the oligomeric duplexes described herein, the first modified oligonucleotide can comprise a stabilized phosphate group attached to the 5′ position of the 5′-most nucleoside. In certain embodiments, the stabilized phosphate group comprises a cyclopropyl phosphonate or an (E)-vinyl phosphonate.

In any of the oligomeric duplexes described herein, the first modified oligonucleotide can comprise a conjugate group. In certain embodiments, the conjugate group comprises a conjugate linker and a conjugate moiety. In certain embodiments, the conjugate group is attached to the first modified oligonucleotide at the 5′-end of the modified oligonucleotide. In certain embodiments, the conjugate group is attached to the first modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, CIt alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In any of the oligomeric duplexes described herein, the second modified oligonucleotide can comprise a conjugate group. In certain embodiments, the conjugate group comprises a conjugate linker and a conjugate moiety. In certain embodiments, the conjugate group is attached to the second modified oligonucleotide at the 5′-end of the modified oligonucleotide. In certain embodiments, the conjugate group is attached to the second modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In certain embodiments, an antisense agent comprises an antisense compound, which comprises an oligomeric compound or an oligomeric duplex described herein. In certain embodiments, an antisense agent, which can comprise an oligomeric compound or an oligomeric duplex described herein, is an RNAi agent capable of reducing the amount of DUX4 nucleic acid through the activation of RISC/Ago2.

Certain embodiments provide an oligomeric agent comprising two or more oligomeric duplexes. In certain embodiments, an oligomeric agent comprises two or more of any of the oligomeric duplexes described herein. In certain embodiments, an oligomeric agent comprises two or more of the same oligomeric duplex, which can be any of the oligomeric duplexes described herein. In certain embodiments, the two or more oligomeric duplexes are linked together. In certain embodiments, the two or more oligomeric duplexes are covalently linked together. In certain embodiments, the second modified oligonucleotides of two or more oligomeric duplexes are covalently linked together. In certain embodiments, the second modified oligonucleotides of two or more oligomeric duplexes are covalently linked together at their 3′ ends. In certain embodiments, the two or more oligomeric duplexes are covalently linked together by a glycol linker, such as a tetraethylene glycol linker. Certain such compounds are described in, e.g., Alterman, et al., Nature Biotech., 37:844-894, 2019.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage. Certain modified nucleosides and modified internucleoside linkages suitable for use in modified oligonucleotides are described below.

### A. Certain Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modified nucleobases may be incorporated into antisense oligonucleotides.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including, but not limited, to substituents at the 2′, 3′, 4′, and/or 5′ positions. In certain embodiments, the furanosyl sugar moiety is a ribosyl sugar moiety. In certain embodiments, one or more acyclic substituent of non-bicyclic modified sugar moieties is branched. In certain embodiments, non-bicyclic modified sugar moieties comprise a substituent group at the 2′-position. Examples of substituent groups suitable for the 2′-position of modified sugar moieties include but are not limited to: —F, —OCH₃ (“OMe” or “O-methyl”), and —O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂₀N(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂ON(CH₃)₂ (“DMAOE”), 2′-O(CH₂)₂O(CH₂)₂N(CH₃)₂ (“DMAEOE”), and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂₀N(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂₀N(CH₃)₂, O(CH₂)₂O(CH₂)₂N(CH₃)₂, O(CH₂)₂ON(CH₃)₂ (“DMAOE”), O(CH₂)₂O(CH₂)₂N(CH₃)₂ (“DMAEOE”) and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, OCH₂CH₂OCH₃, O(CH₂)₂SCH₃, O(CH₂)₂₀N(CH3)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the ρ3-D-ribosyl isomeric configuration unless otherwise specified.

In certain embodiments, non-bicyclic modified sugar moieties comprise a substituent group at the 4′-position. Examples of substituent groups suitable for the 4′-position of modified sugar moieties include but are not limited to alkoxy (e.g., methoxy), alkyl, and those described in Manoharan et al., WO 2015/106128.

In certain embodiments, non-bicyclic modified sugar moieties comprise a substituent group at the 3′-position. Examples of substituent groups suitable for the 3′-position of modified sugar moieties include but are not limited to alkoxy (e.g., methoxy), alkyl (e.g., methyl, ethyl).

In certain embodiments, non-bicyclic modified sugar moieties comprise a substituent group at the 5′-position. Examples of substituent groups suitable for the 5′-position of modified sugar moieties include but are not limited to vinyl, alkoxy (e.g., methoxy), alkyl (e.g., methyl (R or S), ethyl).

In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In naturally occurring nucleic acids, sugars are linked to one another 3′ to 5′. In certain embodiments, oligonucleotides include one or more nucleoside or sugar moiety linked at an alternative position, for example at the 2′ position or inverted 5′ to 3′. For example, where the linkage is at the 2′ position, the 2′-substituent groups may instead be at the 3′-position.

Certain modified sugar moieties comprise a substituent that bridges two atoms of the furanosyl ring to form a second ring, resulting in a bicyclic sugar moiety. In certain embodiments, the bicyclic sugar moiety comprises abridge between the 4′ and the 2′ furanose ring atoms. Examples of such 4′ to 2′ bridging sugar substituents include but are not limited to: 4′-CH₂—2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂—2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)—2′ (see, e.g., Allerson et al., U.S. Pat. No. 7,696,345 and Allerson et al., U.S. Pat. No. 8,124,745), 4′-CH₂—C(H)(CH₃)—2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJᵢ, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

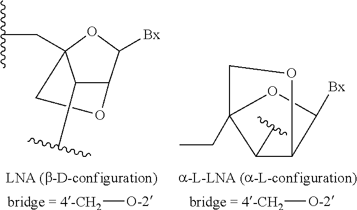

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mal Cane Ther 6(3):833-843; Gmunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, CJ. Bioorg. & Med. Chem. 2002, 10, 841-854), fluoro HNA:

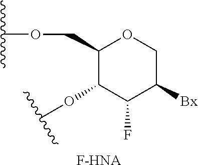

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

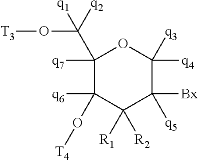

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

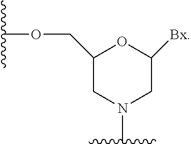

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876. In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378. Representative U.S. patents that teach the preparation of PNA compounds include, but are not limited to, U.S. Pat. Nos. 5,539,082; 5,714,331; and 5,719,262. Additional PNA compounds suitable for use in the oligonucleotides of the invention are described in, for example, in Nielsen et al., Science, 1991, 254, 1497-1500.

In certain embodiments, sugar surrogates are the “unlocked” sugar structure of UNA (unlocked nucleic acid) nucleosides. UNA is an unlocked acyclic nucleic acid, wherein any of the bonds of the sugar has been removed, forming an unlocked sugar surrogate. Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

In certain embodiments, sugar surrogates are the glycerol as found in GNA (glycol nucleic acid) nucleosides as depicted below:

### (S)-GNA

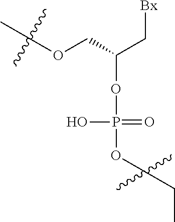

where Bx represents any nucleobase.

Many other bicyclic and tricyclic sugar and sugar surrogates are known in the art that can be used in modified nucleosides.

### 2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside. In certain embodiments, modified oligonucleotides comprise one or more inosine nucleosides (i.e., nucleosides comprising a hypoxanthine nucleobase).

In certain embodiments, modified nucleobases are selected from: 5-substituted pyrimidines, 6-azapyrimi-dines, alkyl or alkynyl substituted pyrimidines, alkyl substituted purines, and N-2, N-6 and O-6 substituted purines. In certain embodiments, modified nucleobases are selected from: 5-methylcytosine, 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphodiesters, which contain a phosphodiester bond (“P═O”) (also referred to as unmodified or naturally occurring linkages), phosphotriesters, methylphosphonates, phosphoramidates, phosphorothioates (“P═S”), and phosphorodithioates (“HS-P═S”). Representative non-phosphorous containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphodiester internucleoside linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO/2021/030778, incorporated by reference herein. In certain embodiments, a modified internucleoside linkage comprises the formula:

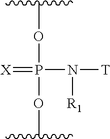

wherein independently for each internucleoside linking group of the modified oligonucleotide:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

In certain embodiments, a modified internucleoside linkage comprises a mesyl phosphoramidate linking group having a formula:

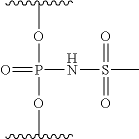 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

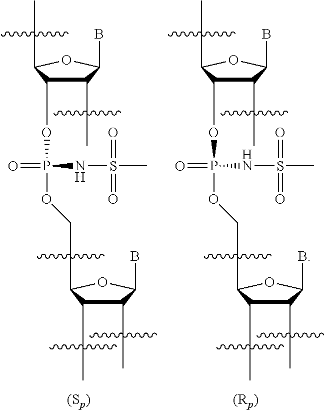

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

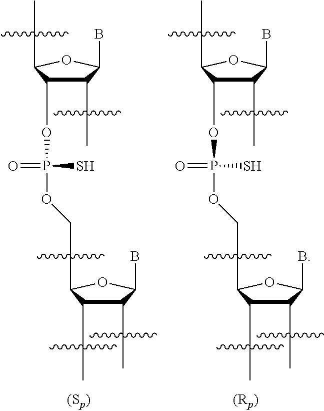

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′—CH₂—N(CH₃)—O-5′), amide-3 (3′—CH₂—C(═O)—N(H)-5′), amide-4 (3′—CH₂—N(H)—C(═O)-5′), formacetal (3′-O—CH₂-0-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

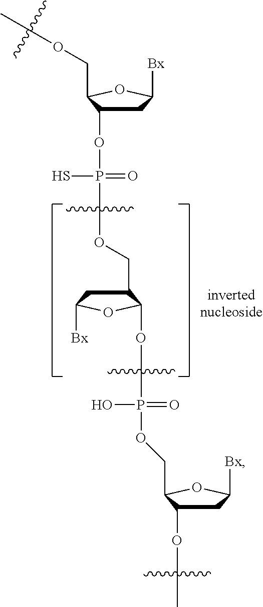

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

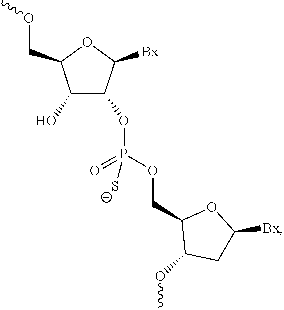

wherein each Bx represents any nucleobase.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

### Uniformly Modified Oligonucleotides

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified nucleotide comprises the same 2′-modification.

### Gapmer Oligonucleotides

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety. In certain embodiments, at least five nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety.

In certain embodiments, the gapmer is a deoxy gapmer. In certain embodiments, the nucleosides on the gap side of each wing/gap junction comprise 2′-β-D-deoxyribosyl sugar moieties and the nucleosides on the wing sides of each wing/gap junction comprise modified sugar moieties. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, at least one nucleoside of the gap of a gapmer comprises a modified sugar moiety. In certain embodiments, at least one nucleoside of the gap of a gapmer comprises a 2′-OMe sugar moiety.

In certain embodiments, modified oligonucleotides comprise or consist of a portion having a fully modified sugar motif. In such embodiments, each nucleoside of the fully modified portion of the modified oligonucleotide comprises a modified sugar moiety. In certain embodiments, each nucleoside of the entire modified oligonucleotide comprises a modified sugar moiety. In certain embodiments, modified oligonucleotides comprise or consist of a portion having a fully modified sugar motif, wherein each nucleoside within the fully modified portion comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing and the gap nucleosides comprise 2′-β-D-deoxyribosyl sugar moieties. Thus, a 5-10-5 MOE gapmer consists of 5 linked 2′-MOE nucleosides in the 5′-wing, 10 linked 2′-β-D-deoxynucleosides in the gap, and 5 linked 2′-MOE nucleosides in the 3′-wing. A 3-10-3 cEt gapmer consists of 3 linked cEt nucleosides in the 5′-wing, 10 linked 2′-β-D-deoxynucleosides in the gap, and 3 linked cEt nucleosides in the 3′-wing. A 5-8-5 gapmer consists of 5 linked nucleosides comprising a modified sugar moiety in the 5′-wing, 8 linked 2′-β-D-deoxynucleosides in the gap, and 5 linked nucleosides comprising a modified sugar moiety in the 3′-wing. A 5-8-5 mixed gapmer has at least two different modified sugar moieties in the 5′- and/or the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers.

In certain embodiments, modified oligonucleotides have a sugar motif selected from 5′-eeeeeddddddddddeeeee-3′ or 5′-kkkddddddddddkkk-3′, wherein each “d” represents a 2′-β-D-deoxyribosyl sugar moiety, each “e” represents a 2′-MOE sugar moiety, and each “k” represents a cEt sugar moiety. In certain embodiments, modified oligonucleotides have the following sugar motif: 5′-eeeeeddddddddddeeeee-3′, wherein each “d” represents a 2′-β-D-deoxyribosyl sugar moiety and each “e” represents a 2′-MOE sugar moiety. In certain embodiments, modified oligonucleotides have the following sugar motif: 5′-kkkddddddddddkkk-3′, wherein each “d” represents a 2′-β-D-deoxyribosyl sugar moiety and each “k” represents a cEt sugar moiety.

### Antisense RNAi Oligonucleotides

In certain embodiments, the sugar moiety of at least one nucleoside of an antisense RNAi oligonucleotide is a modified sugar moiety.

In certain embodiments, at least one nucleoside of the antisense RNAi oligonucleotide comprises a 2′-OMe sugar moiety. In certain embodiments, at least 2 nucleosides comprise 2′-OMe sugar moieties. In certain embodiments, at least 3 nucleosides comprise 2′-OMe sugar moieties. In certain embodiments, at least 4 nucleosides comprise 2′-OMe sugar moieties. In certain embodiments, at least 5 nucleosides comprise 2′-OMe sugar moieties. In certain embodiments, at least 6 nucleosides comprise 2′-OMe sugar moieties. In certain embodiments, at least 7 nucleosides comprise 2′-OMe sugar moieties. In certain embodiments, at least 8 nucleosides comprise 2′-OMe sugar moieties. In certain embodiments, at least 9 nucleosides comprise 2′-OMe sugar moieties. In certain embodiments, at least 10 nucleosides comprise 2′-OMe sugar moieties. In certain embodiments, at least 11 nucleosides comprise 2′-OMe sugar moieties. In certain embodiments, at least 12 nucleosides comprise 2′-OMe sugar moieties. In certain embodiments, at least 13 nucleosides comprise 2′-OMe sugar moieties. In certain embodiments, at least 14 nucleosides comprise 2′-OMe sugar moieties. In certain embodiments, at least 15 nucleosides comprise 2′-OMe sugar moieties. In certain embodiments, at least 17 nucleosides comprise 2′-OMe sugar moieties. In certain such embodiments, at least 18 nucleosides comprise 2′-OMe sugar moieties. In certain such embodiments, at least 20 nucleosides comprise 2′-OMe sugar moieties. In certain embodiments, at least 21 nucleosides comprise 2′-OMe sugar moieties. In certain embodiments, 1 or 2 nucleosides comprise 2′-OMe sugar moieties. In certain embodiments, 1-3 nucleosides comprise 2′-OMe sugar moieties. In certain embodiments, 1-4 nucleosides comprise 2′-OMe sugar moieties. In certain embodiments, 1-5 nucleosides comprise 2′-OMe sugar moieties. In certain embodiments, 1-7 nucleosides comprise 2′-OMe sugar moieties. In certain embodiments, 1-10 nucleosides comprise 2′-OMe sugar moieties. In certain embodiments, 1-12 nucleosides comprise 2′-OMe sugar moieties. In certain embodiments, 1-13 nucleosides comprise 2′-OMe sugar moieties. In certain embodiments, 13 nucleosides comprise 2′-OMe sugar moieties and 3 of those 2′-OMe nucleosides are contiguous. In certain such embodiments, the remainder of the nucleosides are 2′-F modified.

In certain embodiments, at least one nucleoside of the antisense RNAi oligonucleotide comprises a 2′-F sugar moiety. In certain embodiments, at least 2 nucleosides comprise 2′-F sugar moieties. In certain embodiments, at least 3 nucleosides comprise 2′-F sugar moieties. In certain embodiments, at least 4 nucleosides comprise 2′-F sugar moieties. In certain embodiments, at least 5 nucleosides comprise 2′-F sugar moieties. In certain embodiments, at least 6 nucleosides comprise 2′-F sugar moieties. In certain embodiments, at least 7 nucleosides comprise 2′-F sugar moieties. In certain embodiments, at least 8 nucleosides comprise 2′-F sugar moieties. In certain embodiments, at least 9 nucleosides comprise 2′-F sugar moieties. In certain embodiments, at least 10 nucleosides comprise 2′-F sugar moieties. In certain embodiments, at least 11 nucleosides comprise 2′-F sugar moieties. In certain embodiments, at least 12 nucleosides comprise 2′-F sugar moieties. In certain embodiments, at least 13 nucleosides comprise 2′-F sugar moieties. In certain embodiments, at least 14 nucleosides comprise 2′-F sugar moieties. In certain embodiments, at least 16 nucleosides comprise 2′-F sugar moieties. In certain embodiments, at least 18 nucleosides comprise 2′-F sugar moieties. In certain embodiments, at least 20 nucleosides comprise 2′-F sugar moieties. In certain embodiments, at least 21 nucleosides comprise 2′-F sugar moieties. In certain embodiments, 1 or 2 nucleosides comprise 2′-F sugar moieties. In certain embodiments, 1-3 nucleosides comprise 2′-F sugar moieties. In certain embodiments, at least 1-4 nucleosides comprise 2′-F sugar moieties. In certain embodiments, 1-5 nucleosides comprise 2′-F sugar moieties. In certain embodiments, 1-7 nucleosides comprise 2′-F sugar moieties. In certain embodiments, 1-10 nucleosides comprise 2′-F sugar moieties. In certain embodiments, every other nucleosides of an antisense RNAi oligonucleotide are 2′-F nucleosides. In certain such embodiments, the remainder of the nucleosides are 2′-OMe modified.

In certain embodiments, at least one nucleoside of the antisense RNAi oligonucleotide comprises a 2′-OMe sugar moiety and at least one nucleoside comprises a 2′-F sugar moiety. In certain embodiments, at least 1, 2, 3, 4, 5, 6, 7, 8, 9, 10, 11, 12, or 13 nucleosides comprises a 2′-OMe sugar moiety and at least 1, 2, 3, 4, 5, 6, 7, 8, 9, or 10 nucleosides comprises a 2′-F sugar moiety. In certain embodiments, the antisense RNAi oligonucleotide comprises a sugar motif of fyf or yfy, wherein each “f” represents a 2′-F sugar moiety and each “y” represents a 2′-OMe sugar moiety. In certain embodiments, the antisense RNAi oligonucleotide has a sugar motif of yfyfyfyfyfyfyfyfyfyfyyy, wherein each “f” represents a 2′-F sugar moiety and each “y” represents a 2′-OMe sugar moiety.

In certain embodiments, one nucleoside of an antisense RNAi oligonucleotide is a UNA. In certain embodiments, one nucleoside of an antisense RNAi oligonucleotide is a GNA. In certain embodiments, 1-4 nucleosides of an antisense RNAi oligonucleotide is/are DNA. In certain such embodiments, the 1-4 DNA nucleosides are at one or both ends of the antisense RNAi oligonucleotide.

### Sense RNAi Oligonucleotides

In certain embodiments, the sugar moiety of at least one nucleoside of a sense RNAi oligonucleotide is a modified sugar moiety.

In certain such embodiments, at least one nucleoside of the sense RNAi oligonucleotide comprises a 2′-OMe sugar moiety. In certain embodiments, at least 2 nucleosides comprise 2′-OMe sugar moieties. In certain embodiments, at least 3 nucleosides comprise 2′-OMe sugar moieties. In certain embodiments, at least 4 nucleosides comprise 2′-OMe sugar moieties. In certain embodiments, at least 5 nucleosides comprise 2′-OMe sugar moieties. In certain embodiments, at least 6 nucleosides comprise 2′-OMe sugar moieties. In certain embodiments, at least 7 nucleosides comprise 2′-OMe sugar moieties. In certain embodiments, at least 8 nucleosides comprise 2′-OMe sugar moieties. In certain embodiments, at least 9 nucleosides comprise 2′-OMe sugar moieties. In certain embodiments, at least 10 nucleosides comprise 2′-OMe sugar moieties. In certain embodiments, at least 12 nucleosides comprise 2′-OMe sugar moieties. In certain embodiments, at least 14 nucleosides comprise 2′-OMe sugar moieties. In certain embodiments, at least 15 nucleosides comprise 2′-OMe sugar moieties. In certain embodiments, at least 17 nucleosides comprise 2′-OMe sugar moieties. In certain such embodiments, at least 18 nucleosides comprise 2′-OMe sugar moieties. In certain such embodiments, at least 20 nucleosides comprise 2′-OMe sugar moieties. In certain embodiments, 1 or 2 nucleosides comprise 2′-OMe sugar moieties. In certain embodiments, 1-3 nucleosides comprise 2′-OMe sugar moieties. In certain embodiments, 1-4 nucleosides comprise 2′-OMe sugar moieties. In certain embodiments, 1-5 nucleosides comprise 2′-OMe sugar moieties. In certain embodiments, 1-7 nucleosides comprise 2′-OMe sugar moieties. In certain embodiments, 1-10 nucleosides comprise 2′-OMe sugar moieties. In certain embodiments, every other nucleosides of a sense RNAi oligonucleotide are 2′-OMe nucleosides. In certain such embodiments, the remainder of the nucleosides are 2′-F modified.

In certain embodiments, at least one nucleoside of the sense RNAi oligonucleotide comprises a 2′-F sugar moiety. In certain embodiments, at least 2 nucleosides comprise 2′-F sugar moieties. In certain embodiments, at least 3 nucleosides comprise 2′-F sugar moieties. In certain embodiments, at least 4 nucleosides comprise 2′-F sugar moieties. In certain embodiments, at least 5 nucleosides comprise 2′-F sugar moieties. In certain embodiments, at least 6 nucleosides comprise 2′-F sugar moieties. In certain embodiments, at least 7 nucleosides comprise 2′-F sugar moieties. In certain embodiments, at least 8 nucleosides comprise 2′-F sugar moieties. In certain embodiments, at least 9 nucleosides comprise 2′-F sugar moieties. In certain embodiments, at least 10 nucleosides comprise 2′-F sugar moieties. In certain embodiments, at least 11 nucleosides comprise 2′-F sugar moieties. In certain embodiments, 1 or 2 nucleosides comprise 2′-F sugar moieties. In certain embodiments, 1-3 nucleosides comprise 2′-F sugar moieties. In certain embodiments, at least 1-4 nucleosides comprise 2′-F sugar moieties. In certain embodiments, at least 1-5 nucleosides comprise 2′-F sugar moieties. In certain embodiments, at least 1-7 nucleosides comprise 2′-F sugar moieties. In certain embodiments, at least 1-10 nucleosides comprise 2′-F sugar moieties. In certain embodiments, at least 1-11 nucleosides comprise 2′-F sugar moieties. In certain embodiments, every other nucleosides of a sense RNAi oligonucleotide are 2′-F nucleosides. In certain embodiments, the remainder of the nucleosides are 2′OMe modified.

In certain embodiments, at least one nucleoside of the sense RNAi oligonucleotide comprises a 2′-OMe sugar moiety and at least one nucleoside comprises a 2′-F sugar moiety. In certain embodiments, at least 1, 2, 3, 4, 5, 6, 7, 8, 9, or 10 nucleosides comprises a 2′-OMe sugar moiety and at least 1, 2, 3, 4, 5, 6, 7, 8, 9, 10, or 11 nucleosides comprises a 2′-F sugar moiety. In certain embodiments, the sense RNAi oligonucleotide comprises a sugar motif of fyf or yfy, wherein each “f” represents a 2′-F sugar moiety and each “y” represents a 2′-OMe sugar moiety. In certain embodiments, the sense RNAi oligonucleotide has a sugar motif of fyfyfyfyfyfyfyfyfyf, wherein each “f” represents a 2′-F sugar moiety and each “y” represents a 2′-OMe sugar moiety.

In certain embodiments, one nucleoside of a sense RNAi oligonucleotide is a UNA. In certain embodiments, one nucleoside of a sense RNAi oligonucleotide is a GNA. In certain embodiments, 1-4 nucleosides of a sense RNAi oligonucleotide is/are DNA. In certain such embodiments, the 1-4 DNA nucleosides are at one or both ends of the sense RNAi oligonucleotide.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines. In certain embodiments, all of the cytosine nucleobases are 5-methylcytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

### Gapmer Oligonucleotides

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-β-D-deoxyribosyl sugar moiety. In certain embodiments, the modified nucleobase is selected from: a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate.

### Gapmer Oligonucleotides

In certain embodiments, the sugar motif of a modified oligonucleotide is a gapmer and the internucleoside linkages within the gap are all modified. In certain embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, or Rp motif. In certain embodiments, populations of modified oligonucleotides are enriched for modified oligonucleotides comprising such internucleoside linkage motifs.

In certain embodiments, modified oligonucleotides have an internucleoside linkage motif of sssssssssssssss or sssssssssssssssssss, wherein each “s” represents a phosphorothioate internucleoside linkage. In certain embodiments, modified oligonucleotides have an internucleoside linkage motif of sssssssssssssss. In certain embodiments, modified oligonucleotides have an internucleoside linkage motif of sssssssssssssssssss.

In certain embodiments, modified oligonucleotides have an internucleoside linkage motif comprising one or more mesyl phosphoramidate linking groups. In certain embodiments, one or more phosphorothioate internucleoside linkages or one or more phosphodiester internucleoside linkages of the internucleoside linkage motifs herein is substituted with a mesyl phosphoramidates linking group.

### Antisense RNAi Oligonucleotides

In certain embodiments, at least one linkage of the antisense RNAi oligonucleotide is a modified linkage. In certain embodiments, the 5′-most linkage (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain embodiments, the modified linkage is a phosphorothioate linkage. In certain embodiments, the remaining linkages are all unmodified phosphodiester linkages. In certain embodiments, antisense RNAi oligonucleotides have an internucleoside linkage motif of ssooooooooooooooooooss, wherein each “s” represents a phosphorothioate internucleoside linkage and each “o” represents a phosphodiester internucleoside linkage.

In certain embodiments, at least one linkage of the antisense RNAi oligonucleotide is an inverted linkage.

### Sense RNAi Oligonucleotides

In certain embodiments, at least one linkage of the sense RNAi oligonucleotides is a modified linkage. In certain embodiments, the 5′-most linkage (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain embodiments, the modified linkage is a phosphorothioate linkage. In certain embodiments, the remaining linkages are all unmodified phosphodiester linkages. In certain embodiments, sense RNAi oligonucleotides have an internucleoside linkage motif of ssooooooooooooooooss, wherein each “s” represents a phosphorothioate internucleoside linkage and each “o” represents a phosphodiester internucleoside linkage.

In certain embodiments, at least one linkage of the sense RNAi oligonucleotides is an inverted linkage.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides (including modified oligonucleotides) consist of 16 linked nucleosides. In certain embodiments, oligonucleotides (including modified oligonucleotides) consist of 17 linked nucleosides. In certain embodiments, oligonucleotides (including modified oligonucleotides) consist of 18 linked nucleosides. In certain embodiments, oligonucleotides (including modified oligonucleotides) consist of 19 linked nucleosides. In certain embodiments, oligonucleotides (including modified oligonucleotides) consist of 20 linked nucleosides.

### Gapmer Oligonucleotides

In certain embodiments, a modified oligonucleotide is a gapmer. In certain embodiments, the gapmer consists of X to Y linked nucleosides, where X represents the fewest number of nucleosides in the range and Y represents the largest number nucleosides in the range. In certain embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, gapmers consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, a gapmer consists of 16 linked nucleosides. In certain embodiments, a gapmer consists of 17 linked nucleosides. In certain embodiments, a gapmer consists consist of 18 linked nucleosides. In certain embodiments, a gapmer consists of 19 linked nucleosides. In certain embodiments, a gapmer consists of 20 linked nucleosides.

### Antisense RNAi Oligonucleotides

In certain embodiments, antisense RNAi oligonucleotides consist of 17-30 linked nucleosides. In certain embodiments, antisense RNAi oligonucleotides consist of 17-25 linked nucleosides. In certain embodiments, antisense RNAi oligonucleotides consist of 17-23 linked nucleosides. In certain embodiments, antisense RNAi oligonucleotides consist of 17-21 linked nucleosides. In certain embodiments, antisense RNAi oligonucleotides consist of 18-30 linked nucleosides. In certain embodiments, antisense RNAi oligonucleotides consist of 20-30 linked nucleosides. In certain embodiments, antisense RNAi oligonucleotides consist of 21-30 linked nucleosides. In certain embodiments, antisense RNAi oligonucleotides consist of 23-30 linked nucleosides. In certain embodiments, antisense RNAi oligonucleotides consist of 18-25 linked nucleosides. In certain embodiments, antisense RNAi oligonucleotides consist of 20-22 linked nucleosides. In certain embodiments, antisense RNAi oligonucleotides consist of 21-23 linked nucleosides. In certain embodiments, antisense RNAi oligonucleotides consist of 23-24 linked nucleosides. In certain embodiments, antisense RNAi oligonucleotides consist of 20 linked nucleosides. In certain embodiments, antisense RNAi oligonucleotides consist of 21 linked nucleosides. In certain embodiments, antisense RNAi oligonucleotides consist of 22 linked nucleosides. In certain embodiments, antisense RNAi oligonucleotides consist of 23 linked nucleosides.

### Sense RNAi Oligonucleotides

In certain embodiments, sense RNAi oligonucleotides consist of 17-30 linked nucleosides. In certain embodiments, sense RNAi oligonucleotides consist of 17-25 linked nucleosides. In certain embodiments, sense RNAi oligonucleotides consist of 17-23 linked nucleosides. In certain embodiments, sense RNAi oligonucleotides consist of 17-21 linked nucleosides. In certain embodiments, sense RNAi oligonucleotides consist of 18-30 linked nucleosides. In certain embodiments, sense RNAi oligonucleotides consist of 20-30 linked nucleosides. In certain embodiments, sense RNAi oligonucleotides consist of 21-30 linked nucleosides. In certain embodiments, sense RNAi oligonucleotides consist of 23-30 linked nucleosides. In certain embodiments, sense RNAi oligonucleotides consist of 18-25 linked nucleosides. In certain embodiments, sense RNAi oligonucleotides consist of 20-22 linked nucleosides. In certain embodiments, sense RNAi oligonucleotides consist of 21-23 linked nucleosides. In certain embodiments, sense RNAi oligonucleotides consist of 23-24 linked nucleosides. In certain embodiments, sense RNAi oligonucleotides consist of 20 linked nucleosides. In certain embodiments, sense RNAi oligonucleotides consist of 21 linked nucleosides. In certain embodiments, sense RNAi oligonucleotides consist of 22 linked nucleosides. In certain embodiments, sense RNAi oligonucleotides consist of 23 linked nucleosides.

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both R-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In certain embodiments, a conjugate group is a lipid having the following structure:

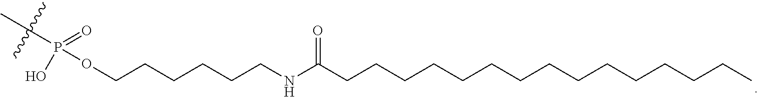 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates (e.g., GalNAc), antibodies, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Cell-Targeting Moieties

In certain embodiments, a conjugate group comprises a cell-targeting moiety. In certain embodiments, a conjugate group has the general formula:

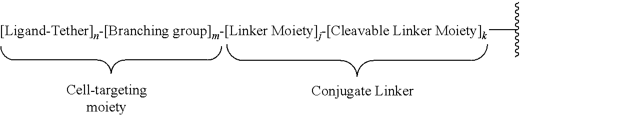

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, a conjugate group comprises a cell-targeting conjugate moiety. In certain embodiments, a conjugate group has the general formula:

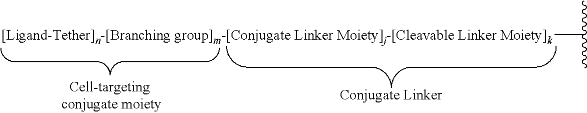

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety targets neurons. In certain embodiments, the cell-targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

In certain embodiments, conjugate groups comprise cell-targeting moieties that have affinities for transferrin receptor (TfR) (also referred to herein as TfR1 and CD71). In certain embodiments, a conjugate group described herein comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the anti-TfR1 antibody or fragment thereof can be any known in the art including but not limited to those described in WO1991/004753; WO2013/103800; WO2014/144060; WO2016/081643; WO2016/179257; WO2016/207240; WO2017/221883; WO2018/129384; WO2018/124121; WO2019/151539; WO2020/132584; WO2020/028864; U.S. Pat. Nos. 7,208,174; 9,034,329; and 10,550,188. In certain embodiments, a fragment of an anti-TfR1 antibody is F(ab′)₂, Fab, Fab′, Fv, or scFv.

In certain embodiments, the conjugate group comprises a protein or peptide capable of binding TfR1. In certain embodiments, the protein or peptide capable of binding TfR1 can be any known in the art including but not limited to those described in WO2019/140050; WO2020/037150; WO2020/124032; and U.S. Pat. No. 10,138,483.

In certain embodiments, the conjugate group comprises an aptamer capable of binding TfR1. In certain embodiments, the aptamer capable of binding TfR1 can be any known in the art including but not limited to those described in WO2013/163303; WO2019/033051; and WO2020/245198.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic sugar moieties and/or inverted nucleosides. In certain embodiments, terminal groups comprise one or more 2′-linked nucleosides or sugar moieties. In certain such embodiments, the 2′-linked group is an abasic sugar moiety.

### III. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in the standard in vitro assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi agents. RNAi agents may be double-stranded (siRNA or dsRNAi) or single-stranded (ssRNAi).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### IV. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain embodiments, the target non-coding RNA is selected from: a long non-coding RNA, a short non-coding RNA, an intronic RNA molecule.

### A. Complementarity/Mismatches to the Target Nucleic Acid and Duplex Complementarity

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved.

### Gapmer Oligonucleotides

In certain embodiments, a mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### Antisense RNAi Oligonucleotides

In certain embodiments, antisense RNAi oligonucleotides comprise one or more mismatched nucleobases relative to the target nucleic acid. In certain embodiments, RNAi activity against the target is reduced by such mismatch, but activity against a non-target is reduced by a greater amount. Thus, in certain embodiments selectivity of the antisense RNAi oligonucleotides is improved.

In certain embodiments, antisense RNAi oligonucleotides comprise a targeting region complementary to the target nucleic acid. In certain embodiments, the targeting region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides. In certain embodiments, the targeting region constitutes 70%, 80%, 85%, 90%, or 95% of the nucleosides of the antisense RNAi oligonucleotide. In certain embodiments, the targeting region constitutes all of the nucleosides of the antisense RNAi oligonucleotide. In certain embodiments, the targeting region of the antisense RNAi oligonucleotide is at least 99%, 95%, 90%, 85%, or 80% complementary to the target nucleic acid. In certain embodiments, the targeting region of the antisense RNAi oligonucleotide is 100% complementary to the target nucleic acid.

### Sense RNAi Oligonucleotides

In certain embodiments, RNAi agents comprise a sense RNAi oligonucleotide. In such embodiments, sense RNAi oligonucleotides comprise a region complementary to the antisense RNAi oligonucleotide. In certain embodiments, the complementary region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides. In certain embodiments, the complementary region constitutes 70%, 80%, 85%, 90%, or 95% of the nucleosides of the sense RNAi oligonucleotide. In certain embodiments, the complementary region constitutes all of the nucleosides of the sense RNAi oligonucleotide. In certain embodiments, the complementary region of the sense RNAi oligonucleotide is at least 99%, 95%, 90%, 85%, or 80% complementary to the antisense RNAi oligonucleotide. In certain embodiments, the complementary region of the sense RNAi oligonucleotide is 100% complementary to the antisense RNAi oligonucleotide.

The complementary region of a sense RNAi oligonucleotide hybridizes with the antisense RNAi oligonucleotide to form a duplex region. In certain embodiments, such duplex region consists of 7 hybridized pairs of nucleosides (one of each pair being on the antisense RNAi oligonucleotide and the other of each pair bien on the sense RNAi oligonucleotide). In certain embodiments, a duplex region comprises at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 hybridized pairs. In certain embodiments, each nucleoside of antisense RNAi oligonucleotide is within the duplex region (i.e., the antisense RNAi oligonucleotide has no overhanging nucleosides). In certain embodiments, the antisense RNAi oligonucleotide includes unpaired nucleosides at the 3′-end and/or the 5′end (overhanging nucleosides). In certain embodiments, each nucleoside of sense RNAi oligonucleotide is within the duplex region (i.e., the sense RNAi oligonucleotide has no overhanging nucleosides). In certain embodiments, the sense RNAi oligonucleotide includes unpaired nucleosides at the 3′-end and/or the 5′end (overhanging nucleosides). In certain embodiments, duplexes formed by the antisense RNAi oligonucleotide and the sense RNAi oligonucleotide do not include any overhangs at one or both ends. Such ends without overhangs are referred to as blunt ends. In certain embodiments wherein the antisense RNAi oligonucleotide has overhanging nucleosides, one or more of those overhanging nucleosides are complementary to the target nucleic acid. In certain embodiments wherein the antisense RNAi oligonucleotide has overhanging nucleosides, one or more of those overhanging nucleosides are not complementary to the target nucleic acid.

### B. DUX4

In certain embodiments, oligomeric compounds comprise or consist of an oligonucleotide comprising a region that is complementary to a target nucleic acid, wherein the target nucleic acid is DUX4. In certain embodiments, DUX4 nucleic acid has the sequence set forth SEQ ID NO: 1 (GENBANK Accession No. NC_000004.12, truncated from nucleotides 190171001 to 190187000) or SEQ ID NO: 2 (GENBANK Accession No. NM_001306068.2). In certain embodiments, DUX4 nucleic acid has the sequence set forth in any of the known splice variants of DUX4, including but not limited to SEQ ID NO: 3 (GENBANK Accession No. FJ439133.1) and SEQ ID NO: 4 (GENBANK Accession No. NM_001293798.2). In certain embodiments, contacting a cell with an oligomeric compound complementary to SEQ ID NO: 1, SEQ ID NO: 2, SEQ ID NO: 3, or SEQ ID NO: 4 reduces the amount of DUX4 RNA, and in certain embodiments reduces the amount of DUX4 protein. In certain embodiments, the oligomeric compound consists of a modified oligonucleotide. In certain embodiments, the oligomeric compound consists of a modified oligonucleotide and a conjugate group.

In certain embodiments, contacting a cell with an oligomeric compound complementary to any one of SEQ ID NOs: 1-4 reduces the amount of DUX4 RNA in a cell. In certain embodiments, contacting a cell with an oligomeric compound complementary to any one of SEQ ID NOs: 1-4 reduces the amount of DUX4 protein in a cell. In certain embodiments, the cell is in vitro. In certain embodiments, the cell is in a subject. In certain embodiments, the oligomeric compound consists of a modified oligonucleotide. In certain embodiments, contacting a cell in a subject with an oligomeric compound complementary to any one of SEQ ID NOs: 1-4 ameliorates one or more symptoms or hallmarks of a disease or disorder associated with DUX4. In certain embodiments, the disease or disorder associated DUX4 is a neuromuscular disorder. In certain embodiments, the disease or disorder associated DUX4 is a muscular dystrophy. In certain embodiments, the muscular dystrophy is Facioscapulohumeral muscular dystrophy (FSHD).

In certain embodiments, an oligomeric compound complementary to any one of SEQ ID NOs: 1-4 is capable of reducing the among of DUX4 RNA in vitro by at least 10%, at least 20%, at least 30%, at least 40%, at least 50%, at least 60%, at least 70%, at least 80%, or at least 90% when administered according to the standard in vitro assay. In certain embodiments, an oligomeric compound complementary to any one of SEQ ID NOs: 1-4 is capable of reducing the amount of DUX4 RNA in vivo by at least 10%, at least 20%, at least 30%, at least 40%, at least 50%, at least 60%, at least 70%, at least 80%, or at least 90% when administered according to the standard in vivo assay. In certain embodiments, an oligomeric compound complementary to any one of SEQ ID NOs: 1-4 is capable of reducing the among of DUX4 protein in vitro by at least 10%, at least 20%, at least 30%, at least 40%, at least 50%, at least 60%, at least 70%, at least 80%, or at least 90% when administered according to the standard in vitro assay. In certain embodiments, an oligomeric compound complementary to any one of SEQ ID NOs: 1-4 is capable of reducing the amount of DUX4 protein in vivo by at least 10%, at least 20%, at least 30%, at least 40%, at least 50%, at least 60%, at least 70%, at least 80%, or at least 90% when administered according to the standard in vivo assay. In certain embodiments, an oligomeric compound complementary to any one of SEQ ID NOs: 1-4 is capable of reducing the among of DUX4 RNA in the muscle of a subject by at least 10%, at least 20%, at least 30%, at least 40%, at least 50%, at least 60%, at least 70%, at least 80%, or at least 90%. In certain embodiments, an oligomeric compound complementary to any one of SEQ ID NOs: 1-4 is capable of reducing the amount of DUX4 protein in the muscle of a subject by at least 10%, at least 20%, at least 30%, at least 40%, at least 50%, at least 60%, at least 70%, at least 80%, or at least 90%.

### C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissues are muscle cells and muscle tissues. Such muscle tissues include all skeletal muscles including, but not limited to, upper and lower limbs, trunk, head, and neck.

### V. Certain Methods and Uses

Certain embodiments provided herein relate to methods of reducing or inhibiting DUX4 expression or activity, which can be useful for treating, preventing, or ameliorating a disease or disorder associated with DUX4. In certain embodiments, the disease or disorder associated DUX4 is a neuromuscular disorder. In certain embodiments, the disease or disorder associated DUX4 is a muscular dystrophy. In certain embodiments, the muscular dystrophy is Facioscapulohumeral muscular dystrophy (FSHD).

In certain embodiments, a method comprises administering to a subject an oligomeric compound, a modified oligonucleotide, an oligomeric duplex, or an antisense agent, any of which having a nucleobase sequence complementary to a DUX4 nucleic acid. In certain embodiments, the subject has or is at risk for developing a disease or disorder associated with DUX4. In certain embodiments, the subject has a neuromuscular disorder. In certain embodiments, the subject has a muscular dystrophy. In certain embodiments, the subject has Facioscapulohumeral muscular dystrophy (FSHD).

In certain embodiments, a method for treating a disease or disorder associated with DUX4 comprises administering to a subject an oligomeric compound, a modified oligonucleotide, an oligomeric duplex, or an antisense agent, any of which having a nucleobase sequence complementary to a DUX4 nucleic acid. In certain embodiments, the subject has or is at risk for developing a disease or disorder associated with DUX4. In certain embodiments, the subject has a neuromuscular disorder. In certain embodiments, the subject has a muscular dystrophy. In certain embodiments, the subject has Facioscapulohumeral muscular dystrophy (FSHD). In certain embodiments, at least one symptom or hallmark of the disease or disorder associated with DUX4 is ameliorated. In certain embodiments, the at least one symptom or hallmark is muscle weakness or muscle wasting in facio, scapula, and/or humeral muscle that can progress to the muscles of the trunk and/or lower limbs. In certain embodiments, administration of the oligomeric compound, the modified oligonucleotide, the oligomeric duplex, or the antisense agent to the subject reduces or delays the onset or progression of muscle weakness or muscle wasting in facio, scapula, and/or humeral muscle or further reduces or delays the onset or progression of muscle weakness or muscle wasting into the muscles of the trunk and/or lower limbs.

In certain embodiments, a method of reducing expression of DUX4 nucleic acid, for example RNA, or reducing expression of DUX4 protein in a cell comprises contacting the cell with an oligomeric compound, a modified oligonucleotide, an oligomeric duplex, or an antisense agent, any of which having a nucleobase sequence complementary to a DUX4 nucleic acid. In certain embodiments, the subject has or is at risk for developing a disease or disorder associated with DUX4. In certain embodiments, the subject has or is at risk for developing a neuromuscular disorder. In certain embodiments, the subject has or is at risk for developing a muscular dystrophy. In certain embodiments, the subject has or is at risk for developing Facioscapulohumeral muscular dystrophy (FSHD). In certain embodiments, the cell is a muscle cell. In certain embodiments, the cell is a human cell.

Certain embodiments are drawn to an oligomeric compound, a modified oligonucleotide, an oligomeric duplex, or an antisense agent, any of which having a nucleobase sequence complementary to a DUX4 nucleic acid, for use in treating a disease or disorder associated with DUX4 or for use in the manufacture of a medicament for treating a disease or disorder associated with DUX4. In certain embodiments, the disease or disorder associated with DUX4 is a neuromuscular disorder. In certain embodiments, the disease or disorder associated with DUX4 is a muscular dystrophy. In certain embodiments, the disease or disorder associated with DUX4 is Facioscapulohumeral muscular dystrophy (FSHD).

In any of the methods or uses described herein, the oligomeric compound, the modified oligonucleotide, the oligomeric duplex, or the antisense agent can be any described herein.

### VI. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid (“artificial CSF” or “aCSF”). In certain embodiments, the artificial cerebrospinal fluid is pharmaceutical grade.

In certain embodiments, a pharmaceutical composition comprises a modified oligonucleotide and artificial cerebrospinal fluid (aCSF). In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts. In certain embodiments, prodrugs comprise one or more conjugate group attached to an oligonucleotide, wherein the conjugate group is cleaved by endogenous nucleases within the body.

In certain embodiments, oligomeric compounds are lyophilized and isolated as sodium salts. In certain embodiments, the sodium salt of an oligomeric compound is mixed with a pharmaceutically acceptable diluent. In certain embodiments, the pharmaceutically acceptable diluent comprises sterile saline, sterile water, PBS, or aCSF. In certain embodiments, the sodium salt of an oligomeric compound is mixed with PBS. In certain embodiments, the sodium salt of an oligomeric compound is mixed with aCSF.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphodiester linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a pharmaceutically acceptable salt thereof” expressly includes all such forms that may be fully or partially protonated/de-protonated/in association with a cation or a combination of cations. In certain embodiments, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid.

In certain embodiments, where a modified oligonucleotide or an oligomeric compound is in solution comprising sodium (e.g., saline), the modified oligonucleotide or oligomeric compound may be partially or fully de-protonated and in association with sodium ions. However, the mass of the protons is nevertheless counted toward the weight of the dose, and the mass of the sodium ions is not counted toward the weight of the dose. Thus, for example, a dose, or dosage unit, of 10 mg of Compound No. 541106 or Compound No. 613801 equals the number of fully protonated molecules that weighs 10 mg. This would be equivalent to 10.58 mg of solvent-free, sodium acetate-free, anhydrous sodiated Compound No. 541106 or 10.61 mg of solvent-free, sodium acetate-free, anhydrous sodiated Compound No. 613801.

In certain embodiments, where a modified oligonucleotide or oligomeric compound is in a solution, such as aCSF, comprising sodium, potassium, calcium, and magnesium, the modified oligonucleotide or oligomeric compound may be partially or fully de-protonated and in association with sodium, potassium, calcium, and/or magnesium. However, the mass of the protons is nevertheless counted toward the weight of the dose, and the mass of the sodium, potassium, calcium, and magnesium ions is not counted toward the weight of the dose.

In certain embodiments, when an oligomeric compound comprises a conjugate group, the mass of the conjugate group may be included in calculating the dose of such oligomeric compound. If the conjugate group also has an acid, the conjugate group is likewise assumed to be fully protonated for the purpose of calculating dose.

### VII. Certain Hotspot Regions

### 1. Nucleobases 2697-2730 of SEQ ID NO: 1

In certain embodiments, nucleobases 2697-2730 of SEQ ID NO: 1 comprise a hotspot region. In certain embodiments, modified oligonucleotides are complementary within nucleobases 2697-2730 of SEQ ID NO: 1. In certain embodiments, modified oligonucleotides are 16 nucleobases in length. In certain embodiments, modified oligonucleotides are 20 nucleobases in length. In certain embodiments, modified oligonucleotides are gapmers. In certain embodiments, modified oligonucleotides are 5-10-5 or 3-10-3 gapmers. In certain embodiments, the gapmers are MOE gapmers. In certain embodiments, the gapmers are cEt gapmers. In certain embodiments, the sugar motif for the gapmers is (from 5′ to 3′): eeeeeddddddddddeeeee; wherein each “d” represents a 2′-β-D-deoxyribosyl sugar moiety, and each “e” represents a 2′-MOE sugar moiety. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 24, 98, 318-320, 359-361, 398-400, 438-440, 852, and 857 are complementary within nucleobases 2697-2730 of SEQ ID NO: 1.

Compounds 541110, 541187, 1099107-1099118, 1582921, and 1582472 are complementary within nucleobases 2697-2730 of SEQ ID NO: 1.

In certain embodiments, modified oligonucleotides complementary within nucleobases 2697-2730 of SEQ ID NO: 1 achieve at least 27% reduction of DUX4 RNA in the standard in vitro RNase H assay. In certain embodiments, modified oligonucleotides complementary within nucleobases 2697-2730 of SEQ ID NO: 1 achieve an average of 56% reduction of DUX4 RNA in the standard in vitro RNase H assay.

### 2. Nucleobases 2756-2778 of SEQ ID NO: 1

In certain embodiments, nucleobases 2756-2778 of SEQ ID NO: 1 comprise a hotspot region. In certain embodiments, modified oligonucleotides are complementary within nucleobases 2756-2778 of SEQ ID NO: 1. In certain embodiments, modified oligonucleotides are 16 nucleobases in length. In certain embodiments, modified oligonucleotides are gapmers. In certain embodiments, modified oligonucleotides are 3-10-3 gapmers. In certain embodiments, the gapmers are cEt gapmers. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 323, 324, 364, 404, 443, and 444 are complementary within nucleobases 2756-2778 of SEQ ID NO: 1.

Compounds 1099128-1099133 are complementary within nucleobases 2756-2778 of SEQ ID NO: 1.

In certain embodiments, modified oligonucleotides complementary within nucleobases 2756-2778 of SEQ ID NO: 1 achieve at least 25% reduction of DUX4 RNA in the standard in vitro RNase H assay. In certain embodiments, modified oligonucleotides complementary within nucleobases 2756-2778 of SEQ ID NO: 1 achieve an average of 57% reduction of DUX4 RNA in the standard in vitro RNase H assay.

3. Nucleobases 4103-4134 of SEQ ID NO: 1 and/or 1330-1361 of SEO ID NO: 2

In certain embodiments, 4103-4134 of SEQ ID NO: 1 and/or 1330-1361 of SEQ ID NO: 2 comprise a hotspot region. In certain embodiments, modified oligonucleotides are complementary within nucleobases 4103-4134 of SEQ ID NO: 1 and/or 1330-1361 of SEQ ID NO: 2. In certain embodiments, modified oligonucleotides are 16 nucleobases in length. In certain embodiments, modified oligonucleotides are 20 nucleobases in length. In certain embodiments, modified oligonucleotides are gapmers. In certain embodiments, modified oligonucleotides are 5-10-5 or 3-10-3 gapmers. In certain embodiments, the gapmers are MOE gapmers. In certain embodiments, the gapmers are cEt gapmers. In certain embodiments, the sugar motif for the gapmers is (from 5′ to 3′): eeeeeddddddddddeeeee; wherein each “d” represents a 2′-β-D-deoxyribosyl sugar moiety, and each “e” represents a 2′-MOE sugar moiety. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 73, 148, 327, 367, 407, 408, 208, and 447 are complementary within nucleobases 4103-4134 of SEQ ID NO: 1 and/or 1330-1361 of SEQ ID NO: 2.

Compounds 541160, 541238, 613853, and 1099147-1099151 are complementary within nucleobases 4103-4134 of SEQ ID NO: 1 and/or 1330-1361 of SEQ ID NO: 2.

In certain embodiments, modified oligonucleotides complementary within nucleobases 4103-4134 of SEQ ID NO: 1 and/or 1330-1361 of SEQ ID NO: 2 achieve at least 14% reduction of DUX4 RNA in the standard in vitro RNase H assay. In certain embodiments, modified oligonucleotides complementary within nucleobases 4103-4134 of SEQ ID NO: 1 and/or 1330-1361 of SEQ ID NO: 2 achieve an average of 52% reduction of DUX4 RNA in the standard in vitro RNase H assay.

### 4. Nucleobases 4503-4522 of SEQ ID NO: 1

In certain embodiments, nucleobases 4503-4522 of SEQ ID NO: 1 comprise a hotspot region. In certain embodiments, modified oligonucleotides are complementary within nucleobases 4503-4522 of SEQ ID NO: 1. In certain embodiments, modified oligonucleotides are 16 nucleobases in length. In certain embodiments, modified oligonucleotides are gapmers. In certain embodiments, modified oligonucleotides are 3-10-3 gapmers. In certain embodiments, the gapmers are cEt gapmers. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 299, 340, 379, and 420-421 are complementary within nucleobases 4503-4522 of SEQ ID NO: 1.

Compounds 1098903-1098907 are complementary within nucleobases 4503-4522 of SEQ ID NO: 1.

In certain embodiments, modified oligonucleotides complementary within nucleobases 4503-4522 of SEQ ID NO: 1 achieve at least 34% reduction of DUX4 RNA in the standard in vitro RNase H assay. In certain embodiments, modified oligonucleotides complementary within nucleobases 4503-4522 of SEQ ID NO: 1 achieve an average of 55% reduction of DUX4 RNA in the standard in vitro RNase H assay.

### 5. Nucleobases 4509-4530 of SEQ ID NO: 1

In certain embodiments, nucleobases 4509-4530 of SEQ ID NO: 1 comprise a hotspot region. In certain embodiments, modified oligonucleotides are complementary within nucleobases 4509-4530 of SEQ ID NO: 1. In certain embodiments, modified oligonucleotides are 16 nucleobases in length. In certain embodiments, modified oligonucleotides are gapmers. In certain embodiments, modified oligonucleotides are 3-10-3 gapmers. In certain embodiments, the gapmers are cEt gapmers. In In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 300-301, 341-342, 380, 422, and 1002 are complementary within nucleobases 4509-4530 of SEQ ID NO: 1.

Compounds 1098908-1098913 and 1582464 are complementary within nucleobases 4509-4530 of SEQ ID NO: 1.

In certain embodiments, modified oligonucleotides complementary within nucleobases 4509-4530 of SEQ ID NO: 1 achieve at least 43% reduction of DUX4 RNA in the standard in vitro RNase H assay. In certain embodiments, modified oligonucleotides complementary within nucleobases 4509-4530 of SEQ ID NO: 1 achieve an average of 61% reduction of DUX4 RNA in the standard in vitro RNase H assay.

6. Nucleobases 4828-4850 of SEQ ID NO: 1 and/or 1693-1710 of SEQ ID NO: 2

In certain embodiments, 4828-4850 of SEQ ID NO: 1 and/or 1693-1710 of SEQ ID NO: 2 comprise a hotspot region. In certain embodiments, modified oligonucleotides are complementary within nucleobases 4828-4850 of SEQ ID NO: 1 and/or 1693-1710 of SEQ ID NO: 2. In certain embodiments, modified oligonucleotides are 16 nucleobases in length. In certain embodiments, modified oligonucleotides are gapmers. In certain embodiments, modified oligonucleotides are 3-10-3 gapmers. In certain embodiments, the gapmers are cEt gapmers. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 331, 332, 370, 371, 412, 413, 451, and 452 are complementary within nucleobases 4828-4850 of SEQ ID NO: 1 and/or 1693-1710 of SEQ ID NO: 2.

Compounds 1099177-1099184 are complementary within nucleobases 4828-4850 of SEQ ID NO: 1 and/or 1693-1710 of SEQ ID NO: 2.

In certain embodiments, modified oligonucleotides complementary within nucleobases 4828-4850 of SEQ ID NO: 1 and/or 1693-1710 of SEQ ID NO: 2 achieve at least 44% reduction of DUX4 RNA in the standard in vitro RNase H assay. In certain embodiments, modified oligonucleotides complementary within nucleobases 4828-4850 of SEQ ID NO: 1 and/or 1693-1710 of SEQ ID NO: 2 achieve an average of 55% reduction of DUX4 RNA in the standard in vitro RNase H assay.

### 7. Nucleobases 768-787 of SEO ID NO: 2

In certain embodiments, nucleobases 768-787 of SEQ ID NO: 2 comprise a hotspot region. In certain embodiments, modified oligonucleotides are complementary within nucleobases 768-787 of SEQ ID NO: 2. In certain embodiments, modified oligonucleotides are 16 nucleobases in length. In certain embodiments, modified oligonucleotides are gapmers. In certain embodiments, the gapmers are cEt gapmers. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 326, 366, 406, 446, and 777 are complementary within nucleobases 768-787 of SEQ ID NO: 2.

Compounds 1099138-1099141 and 1582913 are complementary within nucleobases 768-787 of SEQ ID NO: 2.

In certain embodiments, modified oligonucleotides complementary within nucleobases 768-787 of SEQ ID NO: 2 achieve at least 53% reduction of DUX4 RNA in the standard in vitro RNase H assay. In certain embodiments, modified oligonucleotides complementary within nucleobases 768-787 of SEQ ID NO: 2 achieve an average of 65% reduction of DUX4 RNA in the standard in vitro RNase H assay.

### 8. Nucleobases 2732-2760 of SEQ ID NO: 1

In certain embodiments, nucleobases 2732-2760 of SEQ ID NO: 1 comprise a hotspot region. In certain embodiments, modified oligonucleotides are complementary within nucleobases 2732-2760 of SEQ ID NO: 1. In certain embodiments, modified oligonucleotides are 16 nucleobases in length. In certain embodiments, modified oligonucleotides are 20 nucleobases in length. In certain embodiments, modified oligonucleotides are gapmers. In certain embodiments, modified oligonucleotides are 5-10-5 or 3-10-3 gapmers. In certain embodiments, the gapmers are MOE gapmers. In certain embodiments, the gapmers are cEt gapmers. In certain embodiments, the sugar motif for the gapmers is (from 5′ to 3′): eeeeeddddddddddeeeee; wherein each “d” represents a 2′-β-D-deoxyribosyl sugar moiety, and each “e” represents a 2′-MOE sugar moiety. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 100, 186, 321, 362, 645, 719, 791, 796, 867, 873, 943, 946, and 1019 are complementary within nucleobases 2732-2760 of SEQ ID NO: 1.

Compounds 541189, 613830, 1099121-1099122, 1582534-1582535, 1582543, 1582548-1582549, 1582551, 1582557, 1582566, and 1582568 are complementary within nucleobases 2732-2760 of SEQ ID NO: 1.

In certain embodiments, modified oligonucleotides complementary within nucleobases 2732-2760 of SEQ ID NO: 1 achieve at least 13% reduction of DUX4 RNA in the standard in vitro RNase H assay. In certain embodiments, modified oligonucleotides complementary within nucleobases 2732-2760 of SEQ ID NO: 1 achieve an average of 69% reduction of DUX4 RNA in the standard in vitro RNase H assay.

9. Nucleobases 2783-2806 of SEO ID NO: 1 and/or nucleobases 10-33 of SEO ID NO: 2

In certain embodiments, nucleobases 2783-2806 of SEQ ID NO: 1 and/or nucleobases 10-33 of SEQ ID NO: 2 comprise a hotspot region. In certain embodiments, modified oligonucleotides are complementary within nucleobases 2783-2806 of SEQ ID NO: 1 and/or nucleobases 10-33 of SEQ ID NO: 2. In certain embodiments, modified oligonucleotides are 16 nucleobases in length. In certain embodiments, modified oligonucleotides are gapmers. In certain embodiments, the gapmers are cEt gapmers. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 802, 880, 1088-1090 are complementary within nucleobases 2783-2806 of SEQ ID NO: 1 and/or nucleobases 10-33 of SEQ ID NO: 2.

Compounds 1582611-1582612 and 1604089-1604091 are complementary within nucleobases 2783-2806 of SEQ ID NO: 1 and/or nucleobases 10-33 of SEQ ID NO: 2.

In certain embodiments, modified oligonucleotides complementary within nucleobases 2783-2806 of SEQ ID NO: 1 and/or nucleobases 10-33 of SEQ ID NO: 2 achieve at least 71% reduction of DUX4 RNA in the standard in vitro RNase H assay. In certain embodiments, modified oligonucleotides complementary within nucleobases 2783-2806 of SEQ ID NO: 1 and/or nucleobases 10-33 of SEQ ID NO: 2 achieve an average of 79% reduction of DUX4 RNA in the standard in vitro RNase H assay.

10. Nucleobases 2833-2853 of SEO ID NO: 1 and/or nucleobases 60-80 of SEO ID NO: 2

In certain embodiments, nucleobases 2833-2853 of SEQ ID NO: 1 and/or nucleobases 60-80 of SEQ ID NO: 2 comprise a hotspot region. In certain embodiments, modified oligonucleotides are complementary within nucleobases 2833-2853 of SEQ ID NO: 1 and/or nucleobases 60-80 of SEQ ID NO: 2. In certain embodiments, modified oligonucleotides are 16 nucleobases in length. In certain embodiments, modified oligonucleotides are gapmers. In certain embodiments, the gapmers are cEt gapmers. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 668, 742, 815, 893, 966, and 1040 are complementary within nucleobases 2833-2853 of SEQ ID NO: 1 and/or nucleobases 60-80 of SEQ ID NO: 2.

Compounds 1582702-1582707 are complementary within nucleobases 2833-2853 of SEQ ID NO: 1 and/or nucleobases 60-80 of SEQ ID NO: 2.

In certain embodiments, modified oligonucleotides complementary within nucleobases 2833-2853 of SEQ ID NO: 1 and/or nucleobases 60-80 of SEQ ID NO: 2 achieve at least 59% reduction of DUX4 RNA in the standard in vitro RNase H assay. In certain embodiments, modified oligonucleotides complementary within nucleobases 2833-2853 of SEQ ID NO: 1 and/or nucleobases 60-80 of SEQ ID NO: 2 achieve an average of 79% reduction of DUX4 RNA in the standard in vitro RNase H assay.

11. Nucleobases 2953-2975 of SEQ ID NO: 1 and/or nucleobases 180-202 of SEQ ID NO: 2

In certain embodiments, nucleobases 2953-2975 of SEQ ID NO: 1 and/or nucleobases 180-202 of SEQ ID NO: 2 comprise a hotspot region. In certain embodiments, modified oligonucleotides are complementary within nucleobases 2953-2975 of SEQ ID NO: 1 and/or nucleobases 180-202 of SEQ ID NO: 2. In certain embodiments, modified oligonucleotides are 16 nucleobases in length. In certain embodiments, modified oligonucleotides are gapmers. In certain embodiments, the gapmers are cEt gapmers. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 673, 747-748, 822, 898, 971, and 1045 are complementary within nucleobases 2953-2975 of SEQ ID NO: 1 and/or nucleobases 180-202 of SEQ ID NO: 2.

Compounds 1582734-1582740 are complementary within nucleobases 2953-2975 of SEQ ID NO: 1 and/or nucleobases 180-202 of SEQ ID NO: 2.

In certain embodiments, modified oligonucleotides complementary within nucleobases 2953-2975 of SEQ ID NO: 1 and/or nucleobases 180-202 of SEQ ID NO: 2 achieve at least 73% reduction of DUX4 RNA in the standard in vitro RNase H assay. In certain embodiments, modified oligonucleotides complementary within nucleobases 2953-2975 of SEQ ID NO: 1 and/or nucleobases 180-202 of SEQ ID NO: 2 achieve an average of 78% reduction of DUX4 RNA in the standard in vitro RNase H assay.

12. Nucleobases 3097-3136 of SEQ ID NO: 1 and/or nucleobases 324-363 of SEQ ID NO: 2

In certain embodiments, nucleobases 3097-3136 of SEQ ID NO: 1 and/or nucleobases 324-363 of SEQ ID NO: 2 comprise a hotspot region. In certain embodiments, modified oligonucleotides are complementary within nucleobases 3097-3136 of SEQ ID NO: 1 and/or nucleobases 324-363 of SEQ ID NO: 2. In certain embodiments, modified oligonucleotides are 16 nucleobases in length. In certain embodiments, modified oligonucleotides are 20 nucleobases in length. In certain embodiments, modified oligonucleotides are gapmers. In certain embodiments, modified oligonucleotides are 5-10-5 or 3-10-3 gapmers. In certain embodiments, the gapmers are MOE gapmers. In certain embodiments, the gapmers are cEt gapmers. In certain embodiments, the sugar motif for the gapmers is (from 5′ to 3′): eeeeeddddddddddeeeee; wherein each “d” represents a 2′-β-D-deoxyribosyl sugar moiety, and each “e” represents a 2′-MOE sugar moiety. In certain embodiments, the sugar motif for the gapmers is (from 5′ to 3′): kkkddddddddddkkk; wherein each “d” represents a 2′-D-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 39, 678-681, 753-755, 827-829, 903-905, 976-978, 1050-1052, and 1135-1136 are complementary within nucleobases 3097-3136 of SEQ ID NO: 1 and/or nucleobases 324-363 of SEQ ID NO: 2.

Compounds 541125, 1582765-1582783, and 1604144-1604145 are complementary within nucleobases 3097-3136 of SEQ ID NO: 1 and/or nucleobases 324-363 of SEQ ID NO: 2.

In certain embodiments, modified oligonucleotides complementary within nucleobases 3097-3136 of SEQ ID NO: 1 and/or nucleobases 324-363 of SEQ ID NO: 2 achieve at least 30% reduction of DUX4 RNA in the standard in vitro RNase H assay. In certain embodiments, modified oligonucleotides complementary within nucleobases 3097-3136 of SEQ ID NO: 1 and/or nucleobases 324-363 of SEQ ID NO: 2 achieve an average of 76% reduction of DUX4 RNA in the standard in vitro RNase H assay.

13. Nucleobases 3174-3210 of SEO ID NO: 1 and/or nucleobases 401-437 of SEO ID NO: 2

In certain embodiments, nucleobases 3174-3210 of SEQ ID NO: 1 and/or nucleobases 401-437 of SEQ ID NO: 2 comprise a hotspot region. In certain embodiments, modified oligonucleotides are complementary within nucleobases 3174-3210 of SEQ ID NO: 1 and/or nucleobases 401-437 of SEQ ID NO: 2. In certain embodiments, modified oligonucleotides are 16 nucleobases in length. In certain embodiments, modified oligonucleotides are 20 nucleobases in length. In certain embodiments, modified oligonucleotides are gapmers. In certain embodiments, modified oligonucleotides are 5-10-5 or 3-10-3 gapmers. In certain embodiments, the gapmers are MOE gapmers. In certain embodiments, the gapmers are cEt gapmers. In certain embodiments, the sugar motif for the gapmers is (from 5′ to 3′): eeeeeddddddddddeeeee; wherein each “d” represents a 2′-β-D-deoxyribosyl sugar moiety, and each “e” represents a 2′-MOE sugar moiety. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 117, 684, 758, 1056, and 1141-1144 are complementary within nucleobases 3174-3210 of SEQ ID NO: 1 and/or nucleobases 401-437 of SEQ ID NO: 2.

Compounds 541206, 1582800-1582802, and 1604150-1604153 are complementary within nucleobases 3174-3210 of SEQ ID NO: 1 and/or nucleobases 401-437 of SEQ ID NO: 2.

In certain embodiments, modified oligonucleotides complementary within nucleobases 3174-3210 of SEQ ID NO: 1 and/or nucleobases 401-437 of SEQ ID NO: 2 achieve at least 53% reduction of DUX4 RNA in the standard in vitro RNase H assay. In certain embodiments, modified oligonucleotides complementary within nucleobases 3174-3210 of SEQ ID NO: 1 and/or nucleobases 401-437 of SEQ ID NO: 2 achieve an average of 84% reduction of DUX4 RNA in the standard in vitro RNase H assay.

14. Nucleobases 3361-3400 of SEO ID NO: 1 and/or nucleobases 588-627 of SEO ID NO: 2

In certain embodiments, nucleobases 3361-3400 of SEQ ID NO: 1 and/or nucleobases 588-627 of SEQ ID NO: 2 comprise a hotspot region. In certain embodiments, modified oligonucleotides are complementary within nucleobases 3361-3400 of SEQ ID NO: 1 and/or nucleobases 588-627 of SEQ ID NO: 2. In certain embodiments, modified oligonucleotides are 16 nucleobases in length. In certain embodiments, modified oligonucleotides are 20 nucleobases in length. In certain embodiments, modified oligonucleotides are gapmers. In certain embodiments, modified oligonucleotides are 5-10-5 or 3-10-3 gapmers. In certain embodiments, the gapmers are MOE gapmers. In certain embodiments, the gapmers are cEt gapmers. In certain embodiments, the sugar motif for the gapmers is (from 5′ to 3′): eeeeeddddddddddeeeee; wherein each “d” represents a 2′-β-D-deoxyribosyl sugar moiety, and each “e” represents a 2′-MOE sugar moiety. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 123, 695-697, 769-771, 843, 844, 919, 920, 992, 993, 1066, and 1067 are complementary within nucleobases 3361-3400 of SEQ ID NO: 1 and/or nucleobases 588-627 of SEQ ID NO: 2.

Compounds 541212, 1582863-1582873, and 1582875-1582877 are complementary within nucleobases 3361-3400 of SEQ ID NO: 1 and/or nucleobases 588-627 of SEQ ID NO: 2.

In certain embodiments, modified oligonucleotides complementary within nucleobases 3361-3400 of SEQ ID NO: 1 and/or nucleobases 588-627 of SEQ ID NO: 2 achieve at least 37% reduction of DUX4 RNA in the standard in vitro RNase H assay. In certain embodiments, modified oligonucleotides complementary within nucleobases 3361-3400 of SEQ ID NO: 1 and/or nucleobases 588-627 of SEQ ID NO: 2 achieve an average of 70% reduction of DUX4 RNA in the standard in vitro RNase H assay.

15. Nucleobases 3883-3926 of SEQ ID NO: 1 and/or nucleobases 1110-1153 of SEQ ID NO: 2

In certain embodiments, nucleobases 3883-3926 of SEQ ID NO: 1 and/or nucleobases 1110-1153 of SEQ ID NO: 2 comprise a hotspot region. In certain embodiments, modified oligonucleotides are complementary within nucleobases 3883-3926 of SEQ ID NO: 1 and/or nucleobases 1110-1153 of SEQ ID NO: 2. In certain embodiments, modified oligonucleotides are 16 nucleobases in length. In certain embodiments, modified oligonucleotides are 20 nucleobases in length. In certain embodiments, modified oligonucleotides are gapmers. In certain embodiments, modified oligonucleotides are 5-10-5 or 3-10-3 gapmers. In certain embodiments, the gapmers are MOE gapmers. In certain embodiments, the gapmers are cEt gapmers. In certain embodiments, the sugar motif for the gapmers is (from 5′ to 3′): eeeeeddddddddddeeeee; wherein each “d” represents a 2′-β-D-deoxyribosyl sugar moiety, and each “e” represents a 2′-MOE sugar moiety. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 67, 141, 646-647, 720-721, 793-794, 870-871, 945, 947-948, and 1020-1021 are complementary within nucleobases 3883-3926 of SEQ ID NO: 1 and/or nucleobases 1110-1153 of SEQ ID NO: 2.

Compounds 541153, 541230, 1582550, 1582552-1582556, and 1582558-1582564 are complementary within nucleobases 3883-3926 of SEQ ID NO: 1 and/or nucleobases 1110-1153 of SEQ ID NO: 2.

In certain embodiments, modified oligonucleotides complementary within nucleobases 3883-3926 of SEQ ID NO: 1 and/or nucleobases 1110-1153 of SEQ ID NO: 2 achieve at least 27% reduction of DUX4 RNA in the standard in vitro RNase H assay. In certain embodiments, modified oligonucleotides complementary within nucleobases 3883-3926 of SEQ ID NO: 1 and/or nucleobases 1110-1153 of SEQ ID NO: 2 achieve an average of 71% reduction of DUX4 RNA in the standard in vitro RNase H assay.

16. Nucleobases 4007-4029 of SEO ID NO: 1 and/or nucleobases 1234-1256 of SEO ID NO: 2

In certain embodiments, nucleobases 4007-4029 of SEQ ID NO: 1 and/or nucleobases 1234-1256 of SEQ ID NO: 2 comprise a hotspot region. In certain embodiments, modified oligonucleotides are complementary within nucleobases 4007-4029 of SEQ ID NO: 1 and/or nucleobases 1234-1256 of SEQ ID NO: 2. In certain embodiments, modified oligonucleotides are 16 nucleobases in length. In certain embodiments, modified oligonucleotides are gapmers. In certain embodiments, the gapmers are cEt gapmers. In certain embodiments, the sugar motif for the gapmers is (from 5′ to 3′): kkkddddddddddkkk; wherein each “d” represents a 2′-D-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 652, 726, 799, 876, 953, 1026, and 1079 are complementary within nucleobases 4007-4029 of SEQ ID NO: 1 and/or nucleobases 1234-1256 of SEQ ID NO: 2.

Compounds 1582589-1582594 and 1604080 are complementary within nucleobases 4007-4029 of SEQ ID NO: 1 and/or nucleobases 1234-1256 of SEQ ID NO: 2.

In certain embodiments, modified oligonucleotides complementary within nucleobases 4007-4029 of SEQ ID NO: 1 and/or nucleobases 1234-1256 of SEQ ID NO: 2 achieve at least 65% reduction of DUX4 RNA in the standard in vitro RNase H assay. In certain embodiments, modified oligonucleotides complementary within nucleobases 4007-4029 of SEQ ID NO: 1 and/or nucleobases 1234-1256 of SEQ ID NO: 2 achieve an average of 82% reduction of DUX4 RNA in the standard in vitro RNase H assay.

17. Nucleobases 4667-4694 of SEQ ID NO: 1 and/or nucleobases 1532-1559 of SEQ ID NO: 2

In certain embodiments, nucleobases 4667-4694 of SEQ ID NO: 1 and/or nucleobases 1532-1559 of SEQ ID NO: 2 comprise a hotspot region. In certain embodiments, modified oligonucleotides are complementary within nucleobases 4667-4694 of SEQ ID NO: 1 and/or nucleobases 1532-1559 of SEQ ID NO: 2. In certain embodiments, modified oligonucleotides are 16 nucleobases in length. In certain embodiments, modified oligonucleotides are 20 nucleobases in length. In certain embodiments, modified oligonucleotides are gapmers. In certain embodiments, modified oligonucleotides are 5-10-5 or 3-10-3 gapmers. In certain embodiments, the gapmers are MOE gapmers. In certain embodiments, the gapmers are cEt gapmers. In certain embodiments, the sugar motif for the gapmers is (from 5′ to 3′): eeeeeddddddddddeeeee; wherein each “d” represents a 2′-D-D-deoxyribosyl sugar moiety, and each “e” represents a 2′-MOE sugar moiety. In certain embodiments, the sugar motif for the gapmers is (from 5′ to 3′): kkkddddddddddkkk; wherein each “d” represents a 2′-β-D-deoxyribosyl sugar moiety, and each “k” represents a cEt sugar moiety. Each cytosine residue is a 5-methylcytosine.

In certain embodiments, the nucleosides of the modified oligonucleotides are linked by phosphorothioate internucleoside linkages.

The nucleobase sequences of SEQ ID NOs: 163, 658, 732, 883, 957, 1031, and 1105-1112 are complementary within nucleobases 4667-4694 of SEQ ID NO: 1 and/or nucleobases 1532-1559 of SEQ ID NO: 2.

Compounds 541256, 1582645-1582649, and 1604114-1604121 are complementary within nucleobases 4667-4694 of SEQ ID NO: 1 and/or nucleobases 1532-1559 of SEQ ID NO: 2.

In certain embodiments, modified oligonucleotides complementary within nucleobases 4667-4694 of SEQ ID NO: 1 and/or nucleobases 1532-1559 of SEQ ID NO: 2 achieve at least 63% reduction of DUX4 RNA in the standard in vitro RNase H assay. In certain embodiments, modified oligonucleotides complementary within nucleobases 4667-4694 of SEQ ID NO: 1 and/or nucleobases 1532-1559 of SEQ ID NO: 2 achieve an average of 83% reduction of DUX4 RNA in the standard in vitro RNase H assay.

18. Nucleobases 2955-2984 of SEQ ID NO: 1 and/or nucleobases 182-211 of SEQ ID NO: 2

In certain embodiments, nucleobases 2955-2984 of SEQ ID NO: 1 and/or nucleobases 182-211 of SEQ ID NO: 2 comprise a hotspot region. In certain embodiments, modified oligonucleotides (e.g., antisense RNAi oligonucleotides) are complementary within nucleobases 2955-2984 of SEQ ID NO: 1 and/or nucleobases 182-211 of SEQ ID NO: 2. In certain embodiments, modified oligonucleotides (e.g., antisense RNAi oligonucleotides) are 23 nucleobases in length. In certain embodiments, modified oligonucleotides (e.g., antisense RNAi oligonucleotides) comprise one or more sugar modifications. In certain embodiments, modified oligonucleotides (e.g., antisense RNAi oligonucleotides) comprise one or more 2′-OMe sugar moieties, one or more 2′-F sugar moieties, or a combination thereof. In certain embodiments, the sugar motif of the modified oligonucleotides (e.g., antisense RNAi oligonucleotides) is (from 5′ to 3′): yfyfyfyfyfyfyfyfyfyfyyy, wherein each “y” represents a 2′-OMe sugar moiety and each “f” represents a 2′-F sugar moiety. Each cytosine residue is a 5-methylcytosine.

In certain embodiments, the nucleosides of the modified oligonucleotides (e.g., antisense RNAi oligonucleotides) are linked by an internucleoside linkage selected from a phosphodiester or a phosphorothioate internucleoside linkage.

The nucleobase sequences of SEQ ID NOs: 1425 and 1424 (antisense RNAi oligonucleotides) are complementary within nucleobases 2955-2984 of SEQ ID NO: 1 and/or nucleobases 182-211 of SEQ ID NO: 2.

Compounds 1588626 and 1588623 (RNAi agents) are complementary within nucleobases 2955-2984 of SEQ ID NO: 1 and/or nucleobases 182-211 of SEQ ID NO: 2.

In certain embodiments, modified oligonucleotides (e.g., antisense RNAi oligonucleotides) within nucleobases 2955-2984 of SEQ ID NO: 1 and/or nucleobases 182-211 of SEQ ID NO: 2 achieve at least 87% reduction of DUX4 RNA in the standard in vitro RNAi assay. In certain embodiments, modified oligonucleotides (e.g., antisense RNAi oligonucleotides) within nucleobases 2955-2984 of SEQ ID NO: 1 and/or nucleobases 182-211 of SEQ ID NO: 2 achieve an average of 90% reduction of DUX4 RNA in the standard in vitro RNAi assay.

19. Nucleobases 3099-3128 of SEQ ID NO: 1 and/or nucleobases 326-355 of SEQ ID NO: 2

In certain embodiments, nucleobases 3099-3128 of SEQ ID NO: 1 and/or nucleobases 326-355 of SEQ ID NO: 2 comprise a hotspot region. In certain embodiments, modified oligonucleotides (e.g., antisense RNAi oligonucleotides) are complementary within nucleobases 3099-3128 of SEQ ID NO: 1 and/or nucleobases 326-355 of SEQ ID NO: 2. In certain embodiments, modified oligonucleotides (e.g., antisense RNAi oligonucleotides) are 23 nucleobases in length. In certain embodiments, modified oligonucleotides (e.g., antisense RNAi oligonucleotides) comprise one or more sugar modifications. In certain embodiments, modified oligonucleotides (e.g., antisense RNAi oligonucleotides) comprise one or more 2′-OMe sugar moieties, one or more 2′-F sugar moieties, or a combination thereof. In certain embodiments, the sugar motif of the modified oligonucleotides (e.g., antisense RNAi oligonucleotides) is (from 5′ to 3′): yfyfyfyfyfyfyfyfyfyfyyy, wherein each “y” represents a 2′-OMe sugar moiety and each “f” represents a 2′-F sugar moiety. Each cytosine residue is a 5-methylcytosine.

In certain embodiments, the nucleosides of the modified oligonucleotides (e.g., antisense RNAi oligonucleotides) are linked by an internucleoside linkage selected from a phosphodiester or a phosphorothioate internucleoside linkage.

The nucleobase sequences of SEQ ID NOs: 1214 and 1410 (antisense RNAi oligonucleotides) are complementary within nucleobases 3099-3128 of SEQ ID NO: 1 and/or nucleobases 326-355 of SEQ ID NO: 2.

Compounds 1588572 and 1588569 (RNAi agents) are complementary within nucleobases 3099-3128 of SEQ ID NO: 1 and/or nucleobases 326-355 of SEQ ID NO: 2.

In certain embodiments, modified oligonucleotides (e.g., antisense RNAi oligonucleotides) within nucleobases 3099-3128 of SEQ ID NO: 1 and/or nucleobases 326-355 of SEQ ID NO: 2 achieve at least 63% reduction of DUX4 RNA in the standard in vitro RNAi assay. In certain embodiments, modified oligonucleotides (e.g., antisense RNAi oligonucleotides) within nucleobases 3099-3128 of SEQ ID NO: 1 and/or nucleobases 326-355 of SEQ ID NO: 2 achieve an average of 77% reduction of DUX4 RNA in the standard in vitro RNAi assay.

20. Nucleobases 3171-3209 of SEO ID NO: 1 and/or nucleobases 398-436 of SEO ID NO: 2

In certain embodiments, nucleobases 3171-3209 of SEQ ID NO: 1 and/or nucleobases 398-436 of SEQ ID NO: 2 comprise a hotspot region. In certain embodiments, modified oligonucleotides (e.g., antisense RNAi oligonucleotides) are complementary within nucleobases 3171-3209 of SEQ ID NO: 1 and/or nucleobases 398-436 of SEQ ID NO: 2. In certain embodiments, modified oligonucleotides (e.g., antisense RNAi oligonucleotides) are 23 nucleobases in length. In certain embodiments, modified oligonucleotides (e.g., antisense RNAi oligonucleotides) comprise one or more sugar modifications. In certain embodiments, modified oligonucleotides (e.g., antisense RNAi oligonucleotides) comprise one or more 2′-OMe sugar moieties, one or more 2′-F sugar moieties, or a combination thereof. In certain embodiments, the sugar motif of the modified oligonucleotides (e.g., antisense RNAi oligonucleotides) is (from 5′ to 3′): yfyfyfyfyfyfyfyfyfyyy, wherein each “y” represents a 2′-OMe sugar moiety and each “f” represents a 2′-F sugar moiety. Each cytosine residue is a 5-methylcytosine.

In certain embodiments, the nucleosides of the modified oligonucleotides (e.g., antisense RNAi oligonucleotides) are linked by an internucleoside linkage selected from a phosphodiester or a phosphorothioate internucleoside linkage.

The nucleobase sequences of SEQ ID NOs: 1405, 1210, and 1209 (antisense RNAi oligonucleotides) are complementary within nucleobases 3171-3209 of SEQ ID NO: 1 and/or nucleobases 398-436 of SEQ ID NO: 2.

Compounds 1588545, 1588542, and 1588539 (RNAi agents) are complementary within nucleobases 3171-3209 of SEQ ID NO: 1 and/or nucleobases 398-436 of SEQ ID NO: 2.

In certain embodiments, modified oligonucleotides (e.g., antisense RNAi oligonucleotides) within nucleobases 3171-3209 of SEQ ID NO: 1 and/or nucleobases 398-436 of SEQ ID NO: 2 achieve at least 64% reduction of DUX4 RNA in the standard in vitro RNAi assay. In certain embodiments, modified oligonucleotides (e.g., antisense RNAi oligonucleotides) within nucleobases 3171-3209 of SEQ ID NO: 1 and/or nucleobases 398-436 of SEQ ID NO: 2 achieve an average of 83% reduction of DUX4 RNA in the standard in vitro RNAi assay.

21. Nucleobases 3859-3888 of SEO ID NO: 1 and/or nucleobases 1086-1115 of SEO ID NO: 2

In certain embodiments, nucleobases 3859-3888 of SEQ ID NO: 1 and/or nucleobases 1086-1115 of SEQ ID NO: 2 comprise a hotspot region. In certain embodiments, modified oligonucleotides (e.g., antisense RNAi oligonucleotides) are complementary within nucleobases 3859-3888 of SEQ ID NO: 1 and/or nucleobases 1086-1115 of SEQ ID NO: 2. In certain embodiments, modified oligonucleotides (e.g., antisense RNAi oligonucleotides) are 23 nucleobases in length. In certain embodiments, modified oligonucleotides (e.g., antisense RNAi oligonucleotides) comprise one or more sugar modifications. In certain embodiments, modified oligonucleotides (e.g., antisense RNAi oligonucleotides) comprise one or more 2′-OMe sugar moieties, one or more 2′-F sugar moieties, or a combination thereof. In certain embodiments, the sugar motif of the modified oligonucleotides (e.g., antisense RNAi oligonucleotides) is (from 5′ to 3′): yfyfyfyfyfyfyfyfyfyfyyy, wherein each “y” represents a 2′-OMe sugar moiety and each “f” represents a 2′-F sugar moiety. Each cytosine residue is a 5-methylcytosine.

In certain embodiments, the nucleosides of the modified oligonucleotides (e.g., antisense RNAi oligonucleotides) are linked by an internucleoside linkage selected from a phosphodiester or a phosphorothioate internucleoside linkage.

The nucleobase sequences of SEQ ID NOs: 1197 and 1335 (antisense RNAi oligonucleotides) are complementary within nucleobases 3859-3888 of SEQ ID NO: 1 and/or nucleobases 1086-1115 of SEQ ID NO: 2.

Compounds 1588286 and 1588283 (RNAi agents) are complementary within nucleobases 3859-3888 of SEQ ID NO: 1 and/or nucleobases 1086-1115 of SEQ ID NO: 2.

In certain embodiments, modified oligonucleotides (e.g., antisense RNAi oligonucleotides) within nucleobases 3859-3888 of SEQ ID NO: 1 and/or nucleobases 1086-1115 of SEQ ID NO: 2 achieve at least 60% reduction of DUX4 RNA in the standard in vitro RNAi assay. In certain embodiments, modified oligonucleotides (e.g., antisense RNAi oligonucleotides) within nucleobases 3859-3888 of SEQ ID NO: 1 and/or nucleobases 1086-1115 of SEQ ID NO: 2 achieve an average of 70% reduction of DUX4 RNA in the standard in vitro RNAi assay.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine(methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or 3 such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Effect of 5-10-5 MOE Uniform Phosphorothioate Modified Oligonucleotides on Human DUX4 RNA In Vitro, Single Dose

Modified oligonucleotides complementary to human DUX4 nucleic acid were designed and tested for their single dose effects on DUX4 RNA in vitro. The modified oligonucleotides were tested in a series of experiments that had the same culture conditions.

The modified oligonucleotides in the table below are 5-10-5 MOE gapmers with uniform phosphorothioate internucleoside linkages. The gapmers are 20 nucleosides in length, wherein the central gap segment consists of ten 2′-β-D-deoxynucleosides, and wherein the 5′ and 3′ wing segments each consist of five 2′-MOE nucleosides. The sugar motif for the gapmers is (from 5′ to 3′): eeeeeddddddddddeeeee; wherein each “d” represents a 2′-β-D-deoxyribosyl sugar moiety, and each “e” represents a 2′-MOE sugar moiety. The internucleoside linkage motif for the gapmers is (from 5′ to 3′): sssssssssssssssssss; wherein each “s” represents a phosphorothioate internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (GENBANK Accession No. NC_000004.12, truncated from nucleotides 190171001 to 190187000), to SEQ ID NO: 2 (GENBANK Accession No. NM_001306068.2), to SEQ ID NO: 3 (GENBANK Accession No. FJ439133.1), or to any combination of these SEQ ID NOs. “N/A” indicates that the modified oligonucleotide is not 100% complementary to that particular target nucleic acid sequence.

54-2 cells (Resnick et al., 2019, Cell Reports 29, 1812-1820) were cultured in F10 nutrient media (Gibco #11550) containing 20% FBS, 1 μM dexamethasone, and 10 ng/mL human FGF (Promega rhFGF G5071). The cells were plated at 6,000 cells per well and allowed to come to 100% confluence before media was changed to F-10 media containing 1% horse serum, 10 μg/mL insulin, and 10 μg/mL holo-transferrin to induce differentiation. Cells were allowed to differentiate for 72 hours before transfection of modified oligonucleotides. Differentiated 54-2 cells were treated with modified oligonucleotide at a concentration of 100 nM using cytofectin. After a treatment period of approximately 24 hours, total RNA was isolated from the cells and DUX4 RNA levels were measured by quantitative real-time RTPCR. DUX4 RNA levels were measured by human primer-probe set RTS3502 (forward sequence CCCGGCTGACGTGCAA, designated herein as SEQ ID NO: 11; reverse sequence AGCCAGAATTTCACGGAAGAAC, designated herein as SEQ ID NO: 12; probe sequence AGCTCGCTGGCCTCTCTGTGCC, designated herein as SEQ ID NO: 13). DUX4 RNA levels were normalized to total RNA content, as measured by RIBOGREEN®. Reduction of DUX4 RNA is presented in the tables below as percent DUX4 RNA relative to the amount of DUX4 RNA in untreated control cells (% UTC). The values marked with an “t” indicate that the modified oligonucleotide is complementary to the amplicon region of the primer probe set. Additional assays may be used to measure the potency and efficacy of the modified oligonucleotides complementary to the amplicon region.

Each separate experimental analysis described in this example is identified by a letter ID from A through B in the table column labeled “Analysis ID”.

TABLE 1
Reduction of DUX4 RNA by 5-10-5 MOE gapmers with uniform phosphorothioate internucleoside
linkages in differentiated 54-2 cells

| | SEQ | SEQ | SEQ | SEQ | | | | |
|---|---|---|---|---|---|---|---|---|
| | ID | ID | ID | ID | | | | |
| | No: 1 | No: 1 | No: 2 | No: 2 | | | | SEQ |
| Compound | Start | Stop | Start | Stop | | DUX4 | Analysis | ID |
| Number | Site | Site | Site | Site | Sequence (5′ to 3′) | (% UTC) | ID | NO |
| 541106 | 2616 | 2635 | N/A | N/A | CCAGCCGCCCTTGTAAAGGC | 89 | A | 20 |
| 541107 | 2642 | 2661 | N/A | N/A | GCCCGGACAGCCAGCCAGCC | 71 | A | 21 |
| 541108 | 2664 | 2683 | N/A | N/A | GGCAGGTGCAGCCAGGAGGC | 72 | A | 22 |
| 541109 | 2686 | 2705 | N/A | N/A | CCTCAGCCGGACTGTGCACT | 51 | A | 23 |
| 541110 | 2708 | 2727 | N/A | N/A | GAGGCCGGCGGGCTCCCGTG | 67 | A | 24 |
| 541111 | 2730 | 2749 | N/A | N/A | CACGGACGGACGCGGGCAGA | 66 | A | 25 |
| 541112 | 2752 | 2771 | N/A | N/A | CGGTGAGCCCCGGCCGGAAT | 64 | A | 26 |
| 541113 | 2808 | 2827 | 35 | 54 | TCGTCCCCGGGCTTCCGCGG | 69 | A | 27 |
| 541114 | 2830 | 2849 | 57 | 76 | AAACGAGTCTCCGTCGCCGT | 52 | A | 28 |
| 541115 | 2866 | 2885 | 93 | 112 | AGCAGGCTCGCAGGGCCTCG | 103 | A | 29 |
| 541116 | 2897 | 2916 | 124 | 143 | GTGGCGATGCCCGGGTACGG | 67 | A | 30 |
| 541117 | 2919 | 2938 | 146 | 165 | GGCCTGGGCCAGCCGTTCTC | 48 | A | 31 |
| 541118 | 2941 | 2960 | 168 | 187 | CCCTGGGCTCCGGAATGCCG | 72 | A | 32 |
| 541119 | 2963 | 2982 | 190 | 209 | TCATTCTGAAACCAAATCTG | 93 | A | 33 |
| 541120 | 2985 | 3004 | 212 | 231 | CTGCCTCAGCTGGCGTGACC | 56 | A | 34 |
| 541121 | 3007 | 3026 | 234 | 253 | AGGGCCGAGATTCCCGCCGG | 55 | A | 35 |
| 541122 | 3035 | 3054 | 262 | 281 | CCTTCTGGCGGGCCGCGTCT | 83 | A | 36 |
| 541123 | 3057 | 3076 | 284 | 303 | GACGGCGGTCCGCTTTCGCC | 76 | A | 37 |
| 541124 | 3085 | 3104 | 312 | 331 | GGAGGAGCAGGGCGGTCTGG | 113 | A | 38 |
| 541125 | 3107 | 3126 | 334 | 353 | AAGCGATCCTTCTCAAAGGC | 70 | A | 39 |
| 541126 | 3129 | 3148 | 356 | 375 | CTCCCGGGCGGCGATGCCTG | 66 | A | 40 |
| 541127 | 3151 | 3170 | 378 | 397 | GGCCCGTCTCTCTGGCCAGC | 64 | A | 41 |
| 541128 | 3173 | 3192 | 400 | 419 | ATCTGAATCCTGGACTCCGG | 63 | A | 42 |
| 541129 | 3195 | 3214 | 422 | 441 | GGCCCTTCGATTCTGAAACC | 61 | A | 43 |
| 541130 | 3217 | 3236 | 444 | 463 | TGCCACCCTGTCCCGGGTGC | 77 | A | 44 |
| 541131 | 3239 | 3258 | 466 | 485 | CCGCCTGCCTGCGCGGGCGC | 83 | A | 45 |
| 541132 | 3290 | 3309 | 517 | 536 | GCGACCCACGAGGGAGCAGG | 93 | A | 46 |
| 541133 | 3312 | 3331 | 539 | 558 | CCACGCGCCGGTGTGGGCGA | 82 | A | 47 |
| 541134 | 3352 | 3371 | 579 | 598 | CAGGCGCGCAGGGCACGTGG | 79 | A | 48 |
| 541135 | 3385 | 3404 | 612 | 631 | CCTGGCTCACGAAAGCCCCC | 51 | A | 49 |
| 541136 | 3420 | 3439 | 647 | 666 | CTGGCTGGGCTGCAGCGCGG | 59 | A | 50 |
| 541137 | 3442 | 3461 | 669 | 688 | AGATCCCCTCTGCCGGCGCG | 65 | A | 51 |
| 541138 | 3472 | 3491 | 699 | 718 | CGAAATCCCCGCGCGCCGGG | 79 | A | 52 |
| 541139 | 3502 | 3521 | 729 | 748 | CCCCGTCCGGAGGAGCCGGG | 90 | A | 53 |
| 541140 | N/A | N/A | 756 | 775 | AGCGAGGAGCCTGAGGGTGG | 83 | A | 54 |
| 541141 | 3551 | 3570 | 778 | 797 | CTTTTGCCCGGGTGCGGAGG | 85 | A | 55 |
| 541142 | 3573 | 3592 | 800 | 819 | CTGCGGGTCCCGGTCCTCCC | 53 | A | 56 |
| 541143 | 3611 | 3630 | 838 | 857 | GGCTGTGCCACCGCGCAGGG | 61 | A | 57 |
| 541144 | 3633 | 3652 | 860 | 879 | CGGCCCCGCTTGAGCGGGCC | 75 | A | 58 |
| 541145 | 3655 | 3674 | 882 | 901 | GCGCAAGCACCCCTTGGCCC | 67 | A | 59 |
| 541146 | 3678 | 3697 | 905 | 924 | CGGACTCCCCTGGGACGTGG | 52 | A | 60 |
| 541147 | 3700 | 3719 | 927 | 946 | GACCCCGGCCCCAGCCCCAC | 99 | A | 61 |
| 541148 | 3747 | 3766 | 974 | 993 | TGGAGCTGCCCCGGCTTGGG | 62 | A | 62 |
| 541149 | 3795 | 3814 | 1022 | 1041 | CCCCTGCCGCGCGGAGGCGG | 71 | A | 63 |
| 541150 | 3817 | 3836 | 1044 | 1063 | GCGCCGGGATGCCTTGCATC | 61 | A | 64 |
| 541151 | 3848 | 3867 | 1075 | 1094 | CAGGGCGCCGGCTCCTGGAG | 64 | A | 65 |
| 541152 | 3878 | 3897 | 1105 | 1124 | TCCAGCAGCAGGCCGCAGGG | 61 | A | 66 |
| 541153 | 3900 | 3919 | 1127 | 1146 | CTCCGGGCTCGCCAGGAGCT | 73 | A | 67 |
| 541154 | 3922 | 3941 | 1149 | 1168 | GAGGTTGCGCCTGCTGCAGA | 68 | A | 68 |
| 541155 | 3958 | 3977 | 1185 | 1204 | AGGCCTCCAGCTCCCCCGGG | 57 | A | 69 |
| 541156 | 4016 | 4035 | 1243 | 1262 | AGCAGAGCCCGGTATTCTTC | 57 | A | 70 |
| 541157 | 4038 | 4057 | 1265 | 1284 | CCCCGCGTCCTAAAGCTCCT | 65 | A | 71 |
| 541158 | 4060 | 4079 | 1287 | 1306 | GAACCACCCGACCCCGTCCC | 89 | A | 72 |
| 541160 | 4104 | 4123 | 1331 | 1350 | CCAGCCAGGTGTTCCCCGCG | 53 | A | 73 |
| 541161 | 4126 | 4145 | 1353 | 1372 | GCGGAGACACGCCCCTCCGT | 116 | A | 74 |
| 541162 | 4163 | 4182 | 1390 | 1409 | GGAATCCCAGGCCGGTCAGC | 106 | A | 75 |
| 541163 | 4185 | 4204 | 1412 | 1431 | CCGGGCCTAGACCTAGAAGG | 95 | A | 76 |
| 541164 | N/A | N/A | 1434 | 1453 | CTCCGCGGTGTGGAGTCTCT | 71 | A | 77 |
| 541165 | N/A | N/A | 1456 | 1475 | TGCCCAGGAAAGAATGGCAG | 86 | A | 78 |
| 541166 | 4251 | 4270 | 1478 | 1497 | GCCGGCTCTGGGATCCCCGG | 68 | A | 79 |
| 541167 | 4284 | 4303 | N/A | N/A | TGCGCAGTAGGCGGCCCACC | 65 | A | 80 |
| 541168 | 4306 | 4325 | N/A | N/A | CGGCTGCCCGCAAACCCGCG | 58 | A | 81 |
| 541169 | 4328 | 4347 | N/A | N/A | CGGGCTGCTCCCACAGCCCA | 85 | A | 82 |
| 541170 | 4350 | 4369 | N/A | N/A | GGAGAGGCAGGAGAGCTCTG | 57 | A | 83 |
| 541171 | 4439 | 4458 | N/A | N/A | GGTCTCCACCCAGCCCAGGG | 66 | A | 84 |
| 541172 | 4481 | 4500 | N/A | N/A | AGGCCCGGACGCTGCGCGGG | 73 | A | 85 |
| 541174 | 4535 | 4554 | N/A | N/A | GGCGGGCGACGGTGGCGCGG | 66 | A | 86 |
| 541176 | 4598 | 4617 | N/A | N/A | AGGTATGCTTTTGACCGCCA | 50 | A | 87 |
| 541177 | 4620 | 4639 | N/A | N/A | GGAAGCGGGCAAAGACAGAC | 95 | A | 88 |
| 541178 | 4642 | 4661 | N/A | N/A | GCACTGCGCGCAGGTCTAGC | 66 | A | 89 |
| 541179 | 4677 | 4696 | 1542 | 1561 | GGCCAGCGAGCTCCCTTGCA | 48† | A | 90 |
| 541180 | 4699 | 4718 | 1564 | 1583 | CGGAAGAACAAGGGCACAGA | 52† | A | 91 |
| 541181 | 4743 | 4762 | 1608 | 1627 | AGACAGCGTCGGAAGGTGGG | 69 | A | 92 |
| 541182 | 4765 | 4784 | 1630 | 1649 | TAACTCTAATCCAGGTTTGC | 56 | A | 93 |
| 541183 | 4787 | 4806 | 1652 | 1671 | TGAACTAATCATCCAGGAGA | 54 | A | 94 |
| 541184 | 2627 | 2646 | N/A | N/A | CAGCCAGCCAGCCAGCCGCC | 66 | B | 95 |
| 541185 | 2653 | 2672 | N/A | N/A | CCAGGAGGCCTGCCCGGACA | 65 | B | 96 |
| 541186 | 2675 | 2694 | N/A | N/A | CTGTGCACTGCGGCAGGTGC | 63 | B | 97 |
| 541187 | 2697 | 2716 | N/A | N/A | GCTCCCGTGCACCTCAGCCG | 56 | B | 98 |
| 541188 | 2719 | 2738 | N/A | N/A | GCGGGCAGAGAGAGGCCGGC | 84 | B | 99 |
| 541189 | 2741 | 2760 | N/A | N/A | GGCCGGAATTTCACGGACGG | 49 | B | 100 |
| 541190 | 2763 | 2782 | N/A | N/A | GAGGGCCATCGCGGTGAGCC | 66 | B | 101 |
| 541191 | 2819 | 2838 | 46 | 65 | CGTCGCCGTCCTCGTCCCCG | 61 | B | 102 |
| 541192 | 2855 | 2874 | 82 | 101 | AGGGCCTCGCTTTGGCTCGG | 56 | B | 103 |
| 541193 | 2877 | 2896 | 104 | 123 | GTTCCGCTCAAAGCAGGCTC | 62 | B | 104 |
| 541194 | 2908 | 2927 | 135 | 154 | GCCGTTCTCTGGTGGCGATG | 53 | B | 105 |
| 541195 | 2930 | 2949 | 157 | 176 | GGAATGCCGATGGCCTGGGC | 70 | B | 106 |
| 541196 | 2952 | 2971 | 179 | 198 | CCAAATCTGGACCCTGGGCT | 59 | B | 107 |
| 541197 | 2974 | 2993 | 201 | 220 | GGCGTGACCTCTCATTCTGA | 49 | B | 108 |
| 541198 | 2996 | 3015 | 223 | 242 | TCCCGCCGGTGCTGCCTCAG | 51 | B | 109 |
| 541199 | 3024 | 3043 | 251 | 270 | GCCGCGTCTCCCGGGCCAGG | 53 | B | 110 |
| 541200 | 3046 | 3065 | 273 | 292 | GCTTTCGCCGGCCTTCTGGC | 51 | B | 111 |
| 541201 | 3068 | 3087 | 295 | 314 | TGGGATCCGGTGACGGCGGT | 76 | B | 112 |
| 541202 | 3096 | 3115 | 323 | 342 | CTCAAAGGCTCGGAGGAGCA | 69 | B | 113 |
| 541203 | 3118 | 3137 | 345 | 364 | CGATGCCTGGAAAGCGATCC | 60 | B | 114 |
| 541204 | 3140 | 3159 | 367 | 386 | CTGGCCAGCTCCTCCCGGGC | 40 | B | 115 |
| 541205 | 3162 | 3181 | 389 | 408 | GGACTCCGGGAGGCCCGTCT | 33 | B | 116 |
| 541206 | 3184 | 3203 | 411 | 430 | TCTGAAACCAGATCTGAATC | 47 | B | 117 |
| 541207 | 3206 | 3225 | 433 | 452 | CCCGGGTGCCTGGCCCTTCG | 54 | B | 118 |
| 541208 | 3228 | 3247 | 455 | 474 | CGCGGGCGCCCTGCCACCCT | 59 | B | 119 |
| 541209 | 3276 | 3295 | 503 | 522 | AGCAGGGTGACCCCCGCCGG | 51 | B | 120 |
| 541210 | 3301 | 3320 | 528 | 547 | TGTGGGCGAAGGCGACCCAC | 73 | B | 121 |
| 541211 | 3323 | 3342 | 550 | 569 | AGCCCCGTTCCCCACGCGCC | 50 | B | 122 |
| 541212 | 3374 | 3393 | 601 | 620 | AAAGCCCCCTGTGGGAGAGC | 63 | B | 123 |
| 541213 | 3396 | 3415 | 623 | 642 | GGCCCTCGCTGCCTGGCTCA | 54 | B | 124 |
| 541214 | 3431 | 3450 | 658 | 677 | GCCGGCGCGGCCTGGCTGGG | 65 | B | 125 |
| 541215 | 3453 | 3472 | 680 | 699 | GGCAGGTTGGGAGATCCCCT | 50 | B | 126 |
| 541216 | 3483 | 3502 | 710 | 729 | GGCGGCGTAGGCGAAATCCC | 53 | B | 127 |
| 541217 | 3513 | 3532 | 740 | 759 | GTGGGAGAGCGCCCCGTCCG | 46 | B | 128 |
| 541218 | N/A | N/A | 767 | 786 | GTGCGGAGGCCAGCGAGGAG | 75 | B | 129 |
| 541219 | 3562 | 3581 | 789 | 808 | GGTCCTCCCGGCTTTTGCCC | 56 | B | 130 |
| 541220 | 3584 | 3603 | 811 | 830 | AGGCCGTCGCGCTGCGGGTC | 73 | B | 131 |
| 541221 | 3622 | 3641 | 849 | 868 | GAGCGGGCCCAGGCTGTGCC | 57 | B | 132 |
| 541222 | 3644 | 3663 | 871 | 890 | CCTTGGCCCTGCGGCCCCGC | 39 | B | 133 |
| 541223 | 3666 | 3685 | 893 | 912 | GGACGTGGGTGGCGCAAGCA | 58 | B | 134 |
| 541224 | 3689 | 3708 | 916 | 935 | CAGCCCCACCACGGACTCCC | 68 | B | 135 |
| 541225 | 3720 | 3739 | 947 | 966 | CGCCGCCCCGGCGACCTGGG | 57 | B | 136 |
| 541226 | 3784 | 3803 | 1011 | 1030 | CGGAGGCGGAGGCGTCCGGG | 79 | B | 137 |
| 541227 | 3806 | 3825 | 1033 | 1052 | CCTTGCATCTGCCCCTGCCG | 60 | B | 138 |
| 541228 | 3837 | 3856 | 1064 | 1083 | CTCCTGGAGCGCCTGGGAGG | 82 | B | 139 |
| 541229 | 3859 | 3878 | 1086 | 1105 | GGAGTGCAGACCAGGGCGCC | 67 | B | 140 |
| 541230 | 3889 | 3908 | 1116 | 1135 | CCAGGAGCTCATCCAGCAGC | 56 | B | 141 |
| 541231 | 3911 | 3930 | 1138 | 1157 | TGCTGCAGAAACTCCGGGCT | 71 | B | 142 |
| 541232 | 3933 | 3952 | 1160 | 1179 | CGTTTCTAGGAGAGGTTGCG | 54 | B | 143 |
| 541233 | 4005 | 4024 | 1232 | 1251 | GTATTCTTCCTCGCTGAGGG | 56 | B | 144 |
| 541234 | 4027 | 4046 | 1254 | 1273 | AAAGCTCCTCCAGCAGAGCC | 66 | B | 145 |
| 541235 | 4049 | 4068 | 1276 | 1295 | CCCCGTCCCAACCCCGCGTC | 80 | B | 146 |
| 541237 | 4093 | 4112 | 1320 | 1339 | TTCCCCGCGAAAGAGAGGCC | 46 | B | 147 |
| 541238 | 4115 | 4134 | 1342 | 1361 | CCCCTCCGTAGCCAGCCAGG | 53 | B | 148 |
| 541239 | 4152 | 4171 | 1379 | 1398 | CCGGTCAGCCCGGTGGAGGG | 104 | B | 149 |
| 541240 | 4174 | 4193 | 1401 | 1420 | CCTAGAAGGCAGGAATCCCA | 97 | B | 150 |
| 541241 | 4196 | 4215 | 1423 | 1442 | GGAGTCTCTCACCGGGCCTA | 45 | B | 151 |
| 541242 | N/A | N/A | 1445 | 1464 | GAATGGCAGTTCTCCGCGGT | 67 | B | 152 |
| 541243 | 4240 | 4259 | 1467 | 1486 | GATCCCCGGGATGCCCAGGA | 63 | B | 153 |
| 541244 | 4273 | 4292 | N/A | N/A | CGGCCCACCTGCTGGTACCT | 80 | B | 154 |
| 541245 | 4295 | 4314 | N/A | N/A | AAACCCGCGCGTGCGCAGTA | 63 | B | 155 |
| 541246 | 4317 | 4336 | N/A | N/A | CACAGCCCAGGCGGCTGCCC | 83 | B | 156 |
| 541247 | 4339 | 4358 | N/A | N/A | AGAGCTCTGCCCGGGCTGCT | 77 | B | 157 |
| 541248 | 4361 | 4380 | N/A | N/A | GGTGGGCTGGTGGAGAGGCA | 80 | B | 158 |
| 541249 | 4460 | 4479 | N/A | N/A | CCCGGTGTTTCGCGGGACGG | 63 | B | 159 |
| 541252 | 4565 | 4584 | N/A | N/A | GGCAGCTGGGAGGCTGCAGG | 97 | B | 160 |
| 541254 | 4609 | 4628 | N/A | N/A | AAGACAGACAGAGGTATGCT | 80 | B | 161 |
| 541255 | 4631 | 4650 | N/A | N/A | AGGTCTAGCCAGGAAGCGGG | 46 | B | 162 |
| 541256 | 4666 | 4685 | 1531 | 1550 | TCCCTTGCACGTCAGCCGGG | 44† | B | 163 |
| 541257 | 4688 | 4707 | 1553 | 1572 | GGGCACAGAGAGGCCAGCGA | 49† | B | 164 |
| 541258 | 4710 | 4729 | 1575 | 1594 | CCAGAATTTCACGGAAGAAC | 62† | B | 165 |
| 541259 | 4754 | 4773 | 1619 | 1638 | CAGGTTTGCCTAGACAGCGT | 41 | B | 166 |
| 541260 | 4776 | 4795 | 1641 | 1660 | TCCAGGAGATGTAACTCTAA | 45 | B | 167 |
| 541261 | 4798 | 4817 | 1663 | 1682 | TAATATATCTCTGAACTAAT | 87 | B | 168 |

TABLE 2
Reduction of DUX4 RNA by 5-10-5 MOE gapmers with uniform phosphorothioate internucleoside
linkages in differentiated 54-2 cells

| | SEQ | SEQ | | | | |
|---|---|---|---|---|---|---|
| | ID | ID | | | | |
| | No: | No: | | | | |
| | 3 | 3 | | DUX4 | | SEQ |
| Compound | Start | Stop | | (% | Analysis | ID |
| Number | Site | Site | Sequence (5′ to 3′) | UTC) | ID | NO |
| 541159 | 7314 | 7333 | AGAGAGGCCACGGCCCTGCC | 86 | A | 169 |
| 541175 | 7808 | 7827 | AGCTCCGCGCTGGCAGCTGG | 62 | A | 170 |
| 541236 | 7303 | 7322 | GGCCCTGCCCCGAACCACCC | 81 | B | 171 |
| 541253 | 7819 | 7838 | TGACCGCCAGGAGCTCCGCG | 68 | B | 172 |

### Example 2: Effect of 3-10-3 cEt Uniform Phosphorothioate Modified Oligonucleotides on Human DUX4 RNA In Vitro, Single Dose

Modified oligonucleotides complementary to human DUX4 nucleic acid were designed and tested for their single dose effects on DUX4 RNA in vitro. The modified oligonucleotides were tested in a series of experiments that had the same culture conditions.

The modified oligonucleotides in the tables below are 3-10-3 cEt gapmers with uniform phosphorothioate internucleoside linkages. The gapmers are 16 nucleosides in length, wherein the central gap segment consists of ten 2′-β-D-deoxynucleosides, and wherein the 5′ and 3′ wing segments each consist of three cEt nucleosides. The sugar motif for the gapmers is (from 5′ to 3′): kkkddddddddddkkk; wherein each “d” represents a 2′-β-D-deoxyribosyl sugar moiety, and each “k” represents a cEt sugar moiety. The internucleoside linkage motif for the gapmers is (from 5′ to 3′): sssssssssssssss; wherein each “s” represents a phosphorothioate internucleoside linkage. Each cytosine residue is a 5-methylcytosine.

“Stat site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above), to SEQ ID NO: 2 (described herein above), to SEQ ID NO: 3 (described herein above), or to any combination of these SEQ ID NOs. “N/A” indicates that the modified oligonucleotide is not 100% complementary to that particular target nucleic acid sequence.

54-2 cells plated at a density of 10,000 cells per well, were differentiated as described herein above, and were treated with modified oligonucleotide at a concentration of 100 nM using cytofectin. After a treatment period of approximately 24 hours, total RNA was isolated from the cells and DUX4 RNA levels were measured by quantitative real-time RTPCR. DUX4 RNA levels were measured by human primer-probe set RTS3502 (described herein in Example 1). DUX4 RNA levels were normalized to total RNA content, as measured by RIBOGREEN®. Reduction of DUX4 RNA is presented in the tables below as percent DUX4 RNA relative to the amount of DUX4 RNA in untreated control cells (% UTC). The values marked with an “t” indicate that the modified oligonucleotide is complementary to the amplicon region of the primer probe set. Additional assays may be used to measure the potency and efficacy of the modified oligonucleotides complementary to the amplicon region.

Each separate experimental analysis described in this example is identified by a letter ID from C through H in the table column labeled “Analysis ID”.

In the tables below, Compound No. 541114 (described herein above) was used as a benchmark.

TABLE 3
Reduction of DUX4 RNA by 3-10-3 cEt gapmers with uniform phosphorothioate internucleoside
linkages in differentiated 54-2 cells

| | SEQ | SEQ | SEQ | SEQ | | | | |
|---|---|---|---|---|---|---|---|---|
| | ID | ID | ID | ID | | | | |
| | No: 1 | No: 1 | No: 2 | No: 2 | | | | SEQ |
| Compound | Start | Stop | Start | Stop | | DUX4 (% | Analysis | ID |
| Number | Site | Site | Site | Site | Sequence (5′ to 3′) | UTC) | ID | NO |
| 541114 | 2830 | 2849 | 57 | 76 | AAACGAGTCTCCGTCGCCGT | 69 | C | 28 |
| 613801 | 4276 | 4291 | N/A | N/A | GGCCCACCTGCTGGTA | 68 | C | 173 |
| 613802 | 4321 | 4336 | N/A | N/A | CACAGCCCAGGCGGCT | 76 | C | 174 |
| 613803 | 4365 | 4380 | N/A | N/A | GGTGGGCTGGTGGAGA | 106 | C | 175 |
| 613804 | 4460 | 4475 | N/A | N/A | GTGTTTCGCGGGACGG | 53 | C | 176 |
| 613805 | 4508 | 4523 | N/A | N/A | AGGCGAGCCGCCGGAG | 98 | C | 177 |
| 613822 | 2172 | 2187 | N/A | N/A | CGTTGCCGGGACGGTC | 115 | C | 178 |
| 613823 | 2232 | 2247 | N/A | N/A | GGGAGTCTTGAGTGTG | 78 | C | 179 |
| 613824 | 2292 | 2307 | N/A | N/A | TCGGCAGCAGGGAGAA | 124 | C | 180 |
| 613825 | 2322 | 2337 | N/A | N/A | ACGGGAAGCCGCTCTC | 90 | C | 181 |
| 613826 | 2416 | 2431 | N/A | N/A | CGGAGAGACGAAGAGG | 89 | C | 182 |
| 613827 | 2570 | 2585 | N/A | N/A | AATGGCGGTGAGCCCC | 100 | C | 183 |
| 613828 | 2622 | 2637 | N/A | N/A | AGCCAGCCGCCCTTGT | 91 | C | 184 |
| 613829 | 2669 | 2684 | N/A | N/A | CGGCAGGTGCAGCCAG | 115 | C | 185 |
| 613830 | 2739 | 2754 | N/A | N/A | AATTTCACGGACGGAC | 87 | C | 186 |
| 613831 | 2832 | 2847 | 59 | 74 | ACGAGTCTCCGTCGCC | 70 | C | 187 |
| 613832 | 2876 | 2891 | 103 | 118 | GCTCAAAGCAGGCTCG | 88 | C | 188 |
| 613833 | 2914 | 2929 | 141 | 156 | CAGCCGTTCTCTGGTG | 106 | C | 189 |
| 613834 | 2935 | 2950 | 162 | 177 | CGGAATGCCGATGGCC | 81 | C | 190 |
| 613835 | 3042 | 3057 | 269 | 284 | CGGCCTTCTGGCGGGC | 101 | C | 191 |
| 613836 | 3096 | 3111 | 323 | 338 | AAGGCTCGGAGGAGCA | 122 | C | 192 |
| 613837 | 3142 | 3157 | 369 | 384 | GGCCAGCTCCTCCCGG | 84 | C | 193 |
| 613838 | 3164 | 3179 | 391 | 406 | ACTCCGGGAGGCCCGT | 98 | C | 194 |
| 613839 | 3246 | 3261 | 473 | 488 | AGGCCGCCTGCCTGCG | 85 | C | 195 |
| 613840 | 3331 | 3346 | 558 | 573 | GGGAAGCCCCGTTCCC | 111 | C | 196 |
| 613841 | 3446 | 3461 | 673 | 688 | AGATCCCCTCTGCCGG | 77 | C | 197 |
| 613842 | 3505 | 3520 | 732 | 747 | CCCGTCCGGAGGAGCC | 104 | C | 198 |
| 613843 | 3553 | 3568 | 780 | 795 | TTTGCCCGGGTGCGGA | 86 | C | 199 |
| 613844 | 3611 | 3626 | 838 | 853 | GTGCCACCGCGCAGGG | 77 | C | 200 |
| 613845 | 3646 | 3661 | 873 | 888 | TTGGCCCTGCGGCCCC | 107 | C | 201 |
| 613846 | 3720 | 3735 | 947 | 962 | GCCCCGGCGACCTGGG | 108 | C | 202 |
| 613847 | 3816 | 3831 | 1043 | 1058 | GGGATGCCTTGCATCT | 88 | C | 203 |
| 613848 | 3882 | 3897 | 1109 | 1124 | TCCAGCAGCAGGCCGC | 89 | C | 204 |
| 613849 | 3936 | 3951 | 1163 | 1178 | GTTTCTAGGAGAGGTT | 79 | C | 205 |
| 613850 | 3988 | 4003 | 1215 | 1230 | TGCTTCCAGCGAGGCG | 100 | C | 206 |
| 613851 | 4051 | 4066 | 1278 | 1293 | CCGTCCCAACCCCGCG | 73 | C | 207 |
| 613853 | 4116 | 4131 | 1343 | 1358 | CTCCGTAGCCAGCCAG | 86 | C | 208 |
| 613854 | 4155 | 4170 | 1382 | 1397 | CGGTCAGCCCGGTGGA | 116 | C | 209 |
| 613855 | 4195 | 4210 | 1422 | 1437 | CTCTCACCGGGCCTAG | 69 | C | 210 |
| 613856 | 4239 | 4254 | 1466 | 1481 | CCGGGATGCCCAGGAA | 84 | C | 211 |
| 613857 | 4275 | 4290 | N/A | N/A | GCCCACCTGCTGGTAC | 115 | C | 212 |
| 613858 | 4311 | 4326 | N/A | N/A | GCGGCTGCCCGCAAAC | 110 | C | 213 |
| 613859 | 4332 | 4347 | N/A | N/A | CGGGCTGCTCCCACAG | 97 | C | 214 |
| 613860 | 4463 | 4478 | N/A | N/A | CCGGTGTTTCGCGGGA | 98 | C | 215 |
| 613862 | 4606 | 4621 | N/A | N/A | ACAGAGGTATGCTTTT | 113 | C | 216 |
| 613863 | 4647 | 4662 | 1512 | 1527 | CGCACTGCGCGCAGGT | 81 | C | 217 |
| 613864 | 4706 | 4721 | 1571 | 1586 | TCACGGAAGAACAAGG | 84† | C | 218 |
| 613865 | 4755 | 4770 | 1620 | 1635 | GTTTGCCTAGACAGCG | 84 | C | 219 |
| 613866 | 4778 | 4793 | 1643 | 1658 | CAGGAGATGTAACTCT | 93 | C | 220 |
| | 6419 | 6434 | | | | | | |
| 613867 | 4826 | 4841 | 1691 | 1706 | AGGATCCACAGGGAGG | 72 | C | 221 |
| 613868 | 4876 | 4891 | N/A | N/A | TATTGTGACATATCTC | 110 | C | 222 |
| | 8835 | 8850 | | | | | | |
| | 10001 | 10016 | | | | | | |
| 613870 | 4975 | 4990 | N/A | N/A | TAACCATTCTCTAGGT | 100 | C | 223 |
| 613871 | 5026 | 5041 | N/A | N/A | CTGTCTACACGAGAAT | 93 | C | 224 |
| 613872 | 5096 | 5111 | N/A | N/A | CTCTGCCTACAGGAGG | 117 | C | 225 |
| | 11107 | 11122 | | | | | | |
| 613873 | 5148 | 5163 | N/A | N/A | TTTGTGAGATATCTCT | 104 | C | 226 |
| 613874 | 5181 | 5196 | N/A | N/A | TGTAACCCTTGTCAAG | 121 | C | 227 |
| 613875 | 5219 | 5234 | N/A | N/A | GGCTTTGTGATATATA | 84 | C | 228 |
| 613876 | 5273 | 5288 | N/A | N/A | CTCTCCAATGCTCACT | 125 | C | 229 |
| 613877 | 5316 | 5331 | N/A | N/A | TAACACTTGTCTAAGC | 119 | C | 230 |
| 613878 | 5378 | 5393 | N/A | N/A | CTGTCTAGGTTCAGAC | 89 | C | 231 |
| 541114 | 2830 | 2849 | 57 | 76 | AAACGAGTCTCCGTCGCCGT | 79 | D | 28 |
| 613879 | 4289 | 4304 | N/A | N/A | GTGCGCAGTAGGCGGC | 73 | D | 232 |
| 613880 | 4343 | 4358 | N/A | N/A | AGAGCTCTGCCCGGGC | 99 | D | 233 |
| 613881 | 4380 | 4395 | N/A | N/A | GCGGTCAGGCGGCGGG | 86 | D | 234 |
| 613882 | 4493 | 4508 | N/A | N/A | GCGGTGTCAGGCCCGG | 76 | D | 235 |
| 613883 | 4534 | 4549 | N/A | N/A | GCGACGGTGGCGCGGG | 89 | D | 236 |
| 613899 | 2158 | 2173 | N/A | N/A | TCTCGCACACGCAGGC | 157 | D | 237 |
| 613900 | 2207 | 2222 | N/A | N/A | CTCTCCGTGAAGGAGG | 126 | D | 238 |
| 613901 | 2251 | 2266 | N/A | N/A | TGTGGAACTGAACCTC | 100 | D | 239 |
| 613902 | 2308 | 2323 | N/A | N/A | TCTGGGCTCCCACGCG | 89 | D | 240 |
| 613903 | 2349 | 2364 | N/A | N/A | CCGGACCTCTCCAGGG | 100 | D | 241 |
| 613904 | 2533 | 2548 | N/A | N/A | CCGGAAGGGACCCAGG | 120 | D | 242 |
| 613905 | 2607 | 2622 | N/A | N/A | TAAAGGCCCACAGGCA | 115 | D | 243 |
| 613906 | 2647 | 2662 | N/A | N/A | TGCCCGGACAGCCAGC | 127 | D | 244 |
| 613907 | 2692 | 2707 | N/A | N/A | CACCTCAGCCGGACTG | 110 | D | 245 |
| 613908 | 2754 | 2769 | N/A | N/A | GTGAGCCCCGGCCGGA | 127 | D | 246 |
| 613909 | 2859 | 2874 | 86 | 101 | AGGGCCTCGCTTTGGC | 87 | D | 247 |
| 613910 | 2899 | 2914 | 126 | 141 | GGCGATGCCCGGGTAC | 74 | D | 248 |
| 613911 | 2921 | 2936 | 148 | 163 | CCTGGGCCAGCCGTTC | 84 | D | 249 |
| 613912 | 2997 | 3012 | 224 | 239 | CGCCGGTGCTGCCTCA | 83 | D | 250 |
| 613913 | 3073 | 3088 | 300 | 315 | CTGGGATCCGGTGACG | 74 | D | 251 |
| 613914 | 3122 | 3137 | 349 | 364 | CGATGCCTGGAAAGCG | 128 | D | 252 |
| 613915 | 3160 | 3175 | 387 | 402 | CGGGAGGCCCGTCTCT | 98 | D | 253 |
| 613916 | 3199 | 3214 | 426 | 441 | GGCCCTTCGATTCTGA | 82 | D | 254 |
| 613917 | 3303 | 3318 | 530 | 545 | TGGGCGAAGGCGACCC | 101 | D | 255 |
| 613918 | 3388 | 3403 | 615 | 630 | CTGGCTCACGAAAGCC | 111 | D | 256 |
| 613919 | 3480 | 3495 | 707 | 722 | TAGGCGAAATCCCCGC | 110 | D | 257 |
| 613920 | N/A | N/A | 761 | 776 | CAGCGAGGAGCCTGAG | 121 | D | 258 |
| 613921 | 3596 | 3611 | 823 | 838 | GGCCCGGCAGGCCGTC | 149 | D | 259 |
| 613922 | 3628 | 3643 | 855 | 870 | TTGAGCGGGCCCAGGC | 83 | D | 260 |
| 613923 | 3657 | 3672 | 884 | 899 | GCAAGCACCCCTTGGC | 91 | D | 261 |
| 613924 | 3748 | 3763 | 975 | 990 | AGCTGCCCCGGCTTGG | 85 | D | 262 |
| 613925 | 3860 | 3875 | 1087 | 1102 | GTGCAGACCAGGGCGC | 93 | D | 263 |
| 613926 | 3921 | 3936 | 1148 | 1163 | TGCGCCTGCTGCAGAA | 116 | D | 264 |
| 613927 | 3973 | 3988 | 1200 | 1215 | GGCCTCTTCCGAGGCC | 135 | D | 265 |
| 613928 | 4015 | 4030 | 1242 | 1257 | AGCCCGGTATTCTTCC | 111 | D | 266 |
| 613929 | 4066 | 4081 | 1293 | 1308 | CCGAACCACCCGACCC | 115 | D | 267 |
| 613930 | 4098 | 4113 | 1325 | 1340 | GTTCCCCGCGAAAGAG | 110 | D | 268 |
| 613931 | 4131 | 4146 | 1358 | 1373 | GGCGGAGACACGCCCC | 134 | D | 269 |
| 613932 | 4176 | 4191 | 1403 | 1418 | TAGAAGGCAGGAATCC | 132 | D | 270 |
| 613933 | N/A | N/A | 1451 | 1466 | AAGAATGGCAGTTCTC | 104 | D | 271 |
| 613934 | 4260 | 4275 | 1487 | 1502 | CCTGGGCCGGCTCTGG | 104 | D | 272 |
| 613935 | 4288 | 4303 | N/A | N/A | TGCGCAGTAGGCGGCC | 196 | D | 273 |
| 613936 | 4312 | 4327 | N/A | N/A | GGCGGCTGCCCGCAAA | 131 | D | 274 |
| 613937 | 4490 | 4505 | N/A | N/A | GTGTCAGGCCCGGACG | 108 | D | 275 |
| 613938 | 4600 | 4615 | N/A | N/A | GTATGCTTTTGACCGC | 107 | D | 276 |
| 613940 | 4684 | 4699 | 1549 | 1564 | AGAGGCCAGCGAGCTC | 76† | D | 277 |
| 613941 | 4721 | 4736 | 1586 | 1601 | CATTCAGCCAGAATTT | 86† | D | 278 |
| 613942 | 4770 | 4785 | 1635 | 1650 | GTAACTCTAATCCAGG | 57 | D | 279 |
| 613943 | 4802 | 4817 | 1667 | 1682 | TAATATATCTCTGAAC | 120 | D | 280 |
| 613944 | 4842 | 4857 | N/A | N/A | GATGCAAATCTTCTAT | 130 | D | 281 |
| 613946 | 4960 | 4975 | N/A | N/A | TTCAGTCTACTATGGA | 112 | D | 282 |
| 613947 | 4998 | 5013 | N/A | N/A | CTACACTGATCACCTA | 120 | D | 283 |
| 613948 | 5041 | 5056 | N/A | N/A | ACAATTGTCTAGGCTC | 96 | D | 284 |
| 613949 | 5111 | 5126 | N/A | N/A | GAACACTTGCCTACAC | 83 | D | 285 |
| 613950 | 5166 | 5181 | N/A | N/A | GGTTTGGCTTATAGGG | 121 | D | 286 |
| 613951 | 5207 | 5222 | N/A | N/A | TATATTTCCACTGCTC | 107 | D | 287 |
| 613952 | 5238 | 5253 | N/A | N/A | CTGGGCTTTGTCTACA | 108 | D | 288 |
| 613953 | 5284 | 5299 | N/A | N/A | TTGTGACAGATCTCTC | 124 | D | 289 |
| 613955 | 5394 | 5409 | N/A | N/A | CAAGGTGATGTAACTC | 145 | D | 290 |
| 613956 | 2764 | 2779 | N/A | N/A | GGCCATCGCGGTGAGC | 63 | D | 291 |
| 613910 | 2899 | 2914 | 126 | 141 | GGCGATGCCCGGGTAC | 32 | E | 248 |
| 1098874 | 4304 | 4319 | N/A | N/A | CCCGCAAACCCGCGCG | 51 | E | 292 |
| 1098878 | 4308 | 4323 | N/A | N/A | GCTGCCCGCAAACCCG | 46 | E | 293 |
| 1098882 | 4314 | 4329 | N/A | N/A | CAGGCGGCTGCCCGCA | 30 | E | 294 |
| 1098887 | 4382 | 4397 | N/A | N/A | GGGCGGTCAGGCGGCG | 51 | E | 295 |
| 1098891 | 4467 | 4482 | N/A | N/A | GGGCCCGGTGTTTCGC | 76 | E | 296 |
| 1098896 | 4495 | 4510 | N/A | N/A | GAGCGGTGTCAGGCCC | 41 | E | 297 |
| 1098900 | 4500 | 4515 | N/A | N/A | CGCCGGAGCGGTGTCA | 47 | E | 298 |
| 1098904 | 4504 | 4519 | N/A | N/A | GAGCCGCCGGAGCGGT | 32 | E | 299 |
| 1098908 | 4509 | 4524 | N/A | N/A | GAGGCGAGCCGCCGGA | 36 | E | 300 |
| 1098912 | 4513 | 4528 | N/A | N/A | AGAGGAGGCGAGCCGC | 25 | E | 301 |
| 1098916 | 4536 | 4551 | N/A | N/A | GGGCGACGGTGGCGCG | 46 | E | 302 |
| 1099049 | 2167 | 2182 | N/A | N/A | CCGGGACGGTCTCGCA | 80 | E | 303 |
| 1099053 | 2176 | 2191 | N/A | N/A | TCGCCGTTGCCGGGAC | 56 | E | 304 |
| 1099057 | 2214 | 2229 | N/A | N/A | AGGCCCTCTCTCCGTG | 61 | E | 305 |
| 1099061 | 2355 | 2370 | N/A | N/A | GGCTCTCCGGACCTCT | 50 | E | 306 |
| 1099065 | 2359 | 2374 | N/A | N/A | GGCCGGCTCTCCGGAC | 80 | E | 307 |
| 1099069 | 2538 | 2553 | N/A | N/A | CCACCCCGGAAGGGAC | 78 | E | 308 |
| 1099073 | 2543 | 2558 | N/A | N/A | CCGCCCCACCCCGGAA | 53 | E | 309 |
| 1099077 | 2560 | 2575 | N/A | N/A | AGCCCCCCTGGGACAG | 55 | E | 310 |
| 1099081 | 2566 | 2581 | N/A | N/A | GCGGTGAGCCCCCCTG | 35 | E | 311 |
| 1099085 | 2609 | 2624 | N/A | N/A | TGTAAAGGCCCACAGG | 83 | E | 312 |
| 1099089 | 2613 | 2628 | N/A | N/A | CCCTTGTAAAGGCCCA | 66 | E | 313 |
| 1099093 | 2617 | 2632 | N/A | N/A | GCCGCCCTTGTAAAGG | 83 | E | 314 |
| 1099097 | 2646 | 2661 | N/A | N/A | GCCCGGACAGCCAGCC | 45 | E | 315 |
| 1099101 | 2652 | 2667 | N/A | N/A | AGGCCTGCCCGGACAG | 58 | E | 316 |
| 1099105 | 2695 | 2710 | N/A | N/A | GTGCACCTCAGCCGGA | 60 | E | 317 |
| 1099109 | 2704 | 2719 | N/A | N/A | CGGGCTCCCGTGCACC | 33 | E | 318 |
| 1099113 | 2708 | 2723 | N/A | N/A | CCGGCGGGCTCCCGTG | 44 | E | 319 |
| 1099117 | 2713 | 2728 | N/A | N/A | AGAGGCCGGCGGGCTC | 37 | E | 320 |
| 1099121 | 2732 | 2747 | N/A | N/A | CGGACGGACGCGGGCA | 35 | E | 321 |
| 1099125 | 2752 | 2767 | N/A | N/A | GAGCCCCGGCCGGAAT | 75 | E | 322 |
| 1099129 | 2759 | 2774 | N/A | N/A | TCGCGGTGAGCCCCGG | 41 | E | 323 |
| 1099133 | 2763 | 2778 | N/A | N/A | GCCATCGCGGTGAGCC | 28 | E | 324 |
| 1099137 | 2768 | 2783 | N/A | N/A | GGAGGGCCATCGCGGT | 55 | E | 325 |
| 1099141 | N/A | N/A | 772 | 787 | GGTGCGGAGGCCAGCG | 39 | E | 326 |
| 1099149 | 4105 | 4120 | 1332 | 1347 | GCCAGGTGTTCCCCGC | 34 | E | 327 |
| 1099153 | 4192 | 4207 | 1419 | 1434 | TCACCGGGCCTAGACC | 36 | E | 328 |
| 1099169 | 4649 | 4664 | 1514 | 1529 | TGCGCACTGCGCGCAG | 112 | E | 329 |
| 1099174 | 4681 | 4696 | 1546 | 1561 | GGCCAGCGAGCTCCCT | 36† | E | 330 |
| 1099178 | 4829 | 4844 | 1694 | 1709 | TATAGGATCCACAGGG | 38 | E | 331 |
| 1099182 | 4833 | 4848 | N/A | N/A | CTTCTATAGGATCCAC | 43 | E | 332 |
| 613910 | 2899 | 2914 | 126 | 141 | GGCGATGCCCGGGTAC | 34 | F | 248 |
| 1098875 | 4305 | 4320 | N/A | N/A | GCCCGCAAACCCGCGC | 47 | F | 333 |
| 1098879 | 4309 | 4324 | N/A | N/A | GGCTGCCCGCAAACCC | 43 | F | 334 |
| 1098883 | 4340 | 4355 | N/A | N/A | GCTCTGCCCGGGCTGC | 61 | F | 335 |
| 1098888 | 4464 | 4479 | N/A | N/A | CCCGGTGTTTCGCGGG | 53 | F | 336 |
| 1098892 | 4489 | 4504 | N/A | N/A | TGTCAGGCCCGGACGC | 41 | F | 337 |
| 1098897 | 4496 | 4511 | N/A | N/A | GGAGCGGTGTCAGGCC | 59 | F | 338 |
| 1098901 | 4501 | 4516 | N/A | N/A | CCGCCGGAGCGGTGTC | 42 | F | 339 |
| 1098905 | 4505 | 4520 | N/A | N/A | CGAGCCGCCGGAGCGG | 38 | F | 340 |
| 1098909 | 4510 | 4525 | N/A | N/A | GGAGGCGAGCCGCCGG | 33 | F | 341 |
| 1098913 | 4515 | 4530 | N/A | N/A | GCAGAGGAGGCGAGCC | 53 | F | 342 |
| 1098917 | 4537 | 4552 | N/A | N/A | CGGGCGACGGTGGCGC | 53 | F | 343 |
| 1099050 | 2173 | 2188 | N/A | N/A | CCGTTGCCGGGACGGT | 50 | F | 344 |
| 1099054 | 2177 | 2192 | N/A | N/A | GTCGCCGTTGCCGGGA | 57 | F | 345 |
| 1099058 | 2315 | 2330 | N/A | N/A | GCCGCTCTCTGGGCTC | 58 | F | 346 |
| 1099062 | 2356 | 2371 | N/A | N/A | CGGCTCTCCGGACCTC | 70 | F | 347 |
| 1099066 | 2360 | 2375 | N/A | N/A | GGGCCGGCTCTCCGGA | 96 | F | 348 |
| 1099070 | 2539 | 2554 | N/A | N/A | CCCACCCCGGAAGGGA | 90 | F | 349 |
| 1099074 | 2552 | 2567 | N/A | N/A | TGGGACAGCCCGCCCC | 95 | F | 350 |
| 1099078 | 2561 | 2576 | N/A | N/A | GAGCCCCCCTGGGACA | 60 | F | 351 |
| 1099082 | 2567 | 2582 | N/A | N/A | GGCGGTGAGCCCCCCT | 56 | F | 352 |
| 1099086 | 2610 | 2625 | N/A | N/A | TTGTAAAGGCCCACAG | 67 | F | 353 |
| 1099090 | 2614 | 2629 | N/A | N/A | GCCCTTGTAAAGGCCC | 80 | F | 354 |
| 1099094 | 2618 | 2633 | N/A | N/A | AGCCGCCCTTGTAAAG | 58 | F | 355 |
| 1099098 | 2649 | 2664 | N/A | N/A | CCTGCCCGGACAGCCA | 51 | F | 356 |
| 1099102 | 2653 | 2668 | N/A | N/A | GAGGCCTGCCCGGACA | 30 | F | 357 |
| 1099106 | 2696 | 2711 | N/A | N/A | CGTGCACCTCAGCCGG | 65 | F | 358 |
| 1099110 | 2705 | 2720 | N/A | N/A | GCGGGCTCCCGTGCAC | 34 | F | 359 |
| 1099114 | 2709 | 2724 | N/A | N/A | GCCGGCGGGCTCCCGT | 37 | F | 360 |
| 1099118 | 2715 | 2730 | N/A | N/A | AGAGAGGCCGGCGGGC | 39 | F | 361 |
| 1099122 | 2733 | 2748 | N/A | N/A | ACGGACGGACGCGGGC | 50 | F | 362 |
| 1099126 | 2753 | 2768 | N/A | N/A | TGAGCCCCGGCCGGAA | 34 | F | 363 |
| 1099130 | 2760 | 2775 | N/A | N/A | ATCGCGGTGAGCCCCG | 42 | F | 364 |
| 1099134 | 2765 | 2780 | N/A | N/A | GGGCCATCGCGGTGAG | 55 | F | 365 |
| 1099138 | N/A | N/A | 768 | 783 | CGGAGGCCAGCGAGGA | 29 | F | 366 |
| 1099150 | 4106 | 4121 | 1333 | 1348 | AGCCAGGTGTTCCCCG | 32 | F | 367 |
| 1099170 | 4650 | 4665 | 1515 | 1530 | GTGCGCACTGCGCGCA | 68 | F | 368 |
| 1099175 | 4682 | 4697 | 1547 | 1562 | AGGCCAGCGAGCTCCC | 18† | F | 369 |
| 1099179 | 4830 | 4845 | 1695 | 1710 | CTATAGGATCCACAGG | 46 | F | 370 |
| 1099183 | 4834 | 4849 | N/A | N/A | TCTTCTATAGGATCCA | 35 | F | 371 |
| 613910 | 2899 | 2914 | 126 | 141 | GGCGATGCCCGGGTAC | 32 | G | 248 |
| 1098876 | 4306 | 4321 | N/A | N/A | TGCCCGCAAACCCGCG | 78 | G | 372 |
| 1098880 | 4310 | 4325 | N/A | N/A | CGGCTGCCCGCAAACC | 88 | G | 373 |
| 1098884 | 4341 | 4356 | N/A | N/A | AGCTCTGCCCGGGCTG | 128 | G | 374 |
| 1098889 | 4465 | 4480 | N/A | N/A | GCCCGGTGTTTCGCGG | 140 | G | 375 |
| 1098894 | 4492 | 4507 | N/A | N/A | CGGTGTCAGGCCCGGA | 42 | G | 376 |
| 1098898 | 4498 | 4513 | N/A | N/A | CCGGAGCGGTGTCAGG | 89 | G | 377 |
| 1098902 | 4502 | 4517 | N/A | N/A | GCCGCCGGAGCGGTGT | 151 | G | 378 |
| 1098906 | 4506 | 4521 | N/A | N/A | GCGAGCCGCCGGAGCG | 66 | G | 379 |
| 1098910 | 4511 | 4526 | N/A | N/A | AGGAGGCGAGCCGCCG | 57 | G | 380 |
| 1098914 | 4518 | 4533 | N/A | N/A | GGCGCAGAGGAGGCGA | 74 | G | 381 |
| 1098918 | 4538 | 4553 | N/A | N/A | GCGGGCGACGGTGGCG | 103 | G | 382 |
| 1098923 | 4581 | 4596 | N/A | N/A | GCGCTCCGTGCTGGCA | 142 | G | 383 |
| 1099051 | 2174 | 2189 | N/A | N/A | GCCGTTGCCGGGACGG | 74 | G | 384 |
| 1099055 | 2178 | 2193 | N/A | N/A | CGTCGCCGTTGCCGGG | 88 | G | 385 |
| 1099059 | 2341 | 2356 | N/A | N/A | CTCCAGGGATCCCGCG | 147 | G | 386 |
| 1099063 | 2357 | 2372 | N/A | N/A | CCGGCTCTCCGGACCT | 88 | G | 387 |
| 1099067 | 2534 | 2549 | N/A | N/A | CCCGGAAGGGACCCAG | 84 | G | 388 |
| 1099071 | 2541 | 2556 | N/A | N/A | GCCCCACCCCGGAAGG | 168 | G | 389 |
| 1099075 | 2553 | 2568 | N/A | N/A | CTGGGACAGCCCGCCC | 94 | G | 390 |
| 1099079 | 2562 | 2577 | N/A | N/A | TGAGCCCCCCTGGGAC | 150 | G | 391 |
| 1099083 | 2568 | 2583 | N/A | N/A | TGGCGGTGAGCCCCCC | 98 | G | 392 |
| 1099087 | 2611 | 2626 | N/A | N/A | CTTGTAAAGGCCCACA | 106 | G | 393 |
| 1099091 | 2615 | 2630 | N/A | N/A | CGCCCTTGTAAAGGCC | 132 | G | 394 |
| 1099095 | 2619 | 2634 | N/A | N/A | CAGCCGCCCTTGTAAA | 96 | G | 395 |
| 1099099 | 2650 | 2665 | N/A | N/A | GCCTGCCCGGACAGCC | 65 | G | 396 |
| 1099103 | 2675 | 2690 | N/A | N/A | GCACTGCGGCAGGTGC | 87 | G | 397 |
| 1099107 | 2697 | 2712 | N/A | N/A | CCGTGCACCTCAGCCG | 33 | G | 398 |
| 1099111 | 2706 | 2721 | N/A | N/A | GGCGGGCTCCCGTGCA | 73 | G | 399 |
| 1099115 | 2711 | 2726 | N/A | N/A | AGGCCGGCGGGCTCCC | 63 | G | 400 |
| 1099119 | 2716 | 2731 | N/A | N/A | GAGAGAGGCCGGCGGG | 120 | G | 401 |
| 1099123 | 2750 | 2765 | N/A | N/A | GCCCCGGCCGGAATTT | 64 | G | 402 |
| 1099127 | 2755 | 2770 | N/A | N/A | GGTGAGCCCCGGCCGG | 71 | G | 403 |
| 1099131 | 2761 | 2776 | N/A | N/A | CATCGCGGTGAGCCCC | 75 | G | 404 |
| 1099135 | 2766 | 2781 | N/A | N/A | AGGGCCATCGCGGTGA | 76 | G | 405 |
| 1099139 | N/A | N/A | 770 | 785 | TGCGGAGGCCAGCGAG | 47 | G | 406 |
| 1099147 | 4103 | 4118 | 1330 | 1345 | CAGGTGTTCCCCGCGA | 49 | G | 407 |
| 1099151 | 4114 | 4129 | 1341 | 1356 | CCGTAGCCAGCCAGGT | 48 | G | 408 |
| 1099167 | 4646 | 4661 | 1511 | 1526 | GCACTGCGCGCAGGTC | 81 | G | 409 |
| 1099172 | 4652 | 4667 | 1517 | 1532 | GGGTGCGCACTGCGCG | 91† | G | 410 |
| 1099176 | 4683 | 4698 | 1548 | 1563 | GAGGCCAGCGAGCTCC | 57† | G | 411 |
| 1099180 | 4831 | 4846 | N/A | N/A | TCTATAGGATCCACAG | 56 | G | 412 |
| 1099184 | 4835 | 4850 | N/A | N/A | ATCTTCTATAGGATCC | 39 | G | 413 |
| 613910 | 2899 | 2914 | 126 | 141 | GGCGATGCCCGGGTAC | 37 | H | 248 |
| 1098877 | 4307 | 4322 | N/A | N/A | CTGCCCGCAAACCCGC | 37 | H | 414 |
| 1098881 | 4313 | 4328 | N/A | N/A | AGGCGGCTGCCCGCAA | 31 | H | 415 |
| 1098886 | 4344 | 4359 | N/A | N/A | GAGAGCTCTGCCCGGG | 78 | H | 416 |
| 1098890 | 4466 | 4481 | N/A | N/A | GGCCCGGTGTTTCGCG | 77 | H | 417 |
| 1098895 | 4494 | 4509 | N/A | N/A | AGCGGTGTCAGGCCCG | 30 | H | 418 |
| 1098899 | 4499 | 4514 | N/A | N/A | GCCGGAGCGGTGTCAG | 60 | H | 419 |
| 1098903 | 4503 | 4518 | N/A | N/A | AGCCGCCGGAGCGGTG | 56 | H | 420 |
| 1098907 | 4507 | 4522 | N/A | N/A | GGCGAGCCGCCGGAGC | 33 | H | 421 |
| 1098911 | 4512 | 4527 | N/A | N/A | GAGGAGGCGAGCCGCC | 37 | H | 422 |
| 1098915 | 4535 | 4550 | N/A | N/A | GGCGACGGTGGCGCGG | 39 | H | 423 |
| 1099052 | 2175 | 2190 | N/A | N/A | CGCCGTTGCCGGGACG | 56 | H | 424 |
| 1099056 | 2213 | 2228 | N/A | N/A | GGCCCTCTCTCCGTGA | 83 | H | 425 |
| 1099060 | 2354 | 2369 | N/A | N/A | GCTCTCCGGACCTCTC | 73 | H | 426 |
| 1099064 | 2358 | 2373 | N/A | N/A | GCCGGCTCTCCGGACC | 61 | H | 427 |
| 1099068 | 2535 | 2550 | N/A | N/A | CCCCGGAAGGGACCCA | 62 | H | 428 |
| 1099072 | 2542 | 2557 | N/A | N/A | CGCCCCACCCCGGAAG | 77 | H | 429 |
| 1099076 | 2554 | 2569 | N/A | N/A | CCTGGGACAGCCCGCC | 41 | H | 430 |
| 1099080 | 2565 | 2580 | N/A | N/A | CGGTGAGCCCCCCTGG | 36 | H | 431 |
| 1099084 | 2569 | 2584 | N/A | N/A | ATGGCGGTGAGCCCCC | 56 | H | 432 |
| 1099088 | 2612 | 2627 | N/A | N/A | CCTTGTAAAGGCCCAC | 62 | H | 433 |
| 1099092 | 2616 | 2631 | N/A | N/A | CCGCCCTTGTAAAGGC | 81 | H | 434 |
| 1099096 | 2621 | 2636 | N/A | N/A | GCCAGCCGCCCTTGTA | 44 | H | 435 |
| 1099100 | 2651 | 2666 | N/A | N/A | GGCCTGCCCGGACAGC | 52 | H | 436 |
| 1099104 | 2693 | 2708 | N/A | N/A | GCACCTCAGCCGGACT | 39 | H | 437 |
| 1099108 | 2698 | 2713 | N/A | N/A | CCCGTGCACCTCAGCC | 32 | H | 438 |
| 1099112 | 2707 | 2722 | N/A | N/A | CGGCGGGCTCCCGTGC | 48 | H | 439 |
| 1099116 | 2712 | 2727 | N/A | N/A | GAGGCCGGCGGGCTCC | 37 | H | 440 |
| 1099120 | 2731 | 2746 | N/A | N/A | GGACGGACGCGGGCAG | 49 | H | 441 |
| 1099124 | 2751 | 2766 | N/A | N/A | AGCCCCGGCCGGAATT | 44 | H | 442 |
| 1099128 | 2756 | 2771 | N/A | N/A | CGGTGAGCCCCGGCCG | 42 | H | 443 |
| 1099132 | 2762 | 2777 | N/A | N/A | CCATCGCGGTGAGCCC | 32 | H | 444 |
| 1099136 | 2767 | 2782 | N/A | N/A | GAGGGCCATCGCGGTG | 56 | H | 445 |
| 1099140 | N/A | N/A | 771 | 786 | GTGCGGAGGCCAGCGA | 26 | H | 446 |
| 1099148 | 4104 | 4119 | 1331 | 1346 | CCAGGTGTTCCCCGCG | 32 | H | 447 |
| 1099152 | 4191 | 4206 | 1418 | 1433 | CACCGGGCCTAGACCT | 59 | H | 448 |
| 1099168 | 4648 | 4663 | 1513 | 1528 | GCGCACTGCGCGCAGG | 68 | H | 449 |
| 1099173 | 4680 | 4695 | 1545 | 1560 | GCCAGCGAGCTCCCTT | 10† | H | 450 |
| 1099177 | 4828 | 4843 | 1693 | 1708 | ATAGGATCCACAGGGA | 55 | H | 451 |
| 1099181 | 4832 | 4847 | N/A | N/A | TTCTATAGGATCCACA | 45 | H | 452 |
| 1099185 | 4836 | 4851 | N/A | N/A | AATCTTCTATAGGATC | 85 | H | 453 |

TABLE 4
Reduction of DUX4 RNA by 3-10-3 cEt gapmers with uniform phosphorothioate internucleoside
linkages in differentiated 54-2 cells

| | SEQ | SEQ | | | | |
|---|---|---|---|---|---|---|
| | ID | ID | | | | |
| | No: 3 | No: 3 | | | | SEQ |
| Compound | Start | Stop | | DUX4 | Analysis | ID |
| Number | Site | Site | Sequence (5′ to 3′) | (% UTC) | ID | NO |
| 613806 | 1208 | 1223 | GGCACCTGGGCGGCTG | 90 | C | 454 |
| | 4504 | 4519 | | | | |
| 613807 | 1256 | 1271 | TGTGCGCCGGGCCTGG | 70 | C | 455 |
| | 4552 | 4567 | | | | |
| 613808 | 1332 | 1347 | CGCCCCTCTAGGTCTC | 82 | C | 456 |
| | 4628 | 4643 | | | | |
| 613809 | 1366 | 1381 | TCCCTCCCTCCTAACG | 93 | C | 457 |
| | 4662 | 4677 | | | | |
| 613810 | 1441 | 1456 | GGCTCCCTCCCGCCCG | 128 | C | 458 |
| | 4737 | 4752 | | | | |
| 613811 | 1533 | 1548 | CGCTGACCGTTTTCCC | 97 | C | 459 |
| | 4826 | 4841 | | | | |
| 613812 | 4887 | 4902 | ATGGGTGGTGCCCCGC | 110 | C | 460 |
| 613813 | 1630 | 1645 | AGGAAACCGCCCACTC | 117 | C | 461 |
| | 4926 | 4941 | | | | |
| 613814 | 1693 | 1708 | GCGGTCTGTGAACCGC | 115 | C | 462 |
| | 4989 | 5004 | | | | |
| 613815 | 1733 | 1748 | TCGGCCTCGCGCCGCG | 123 | C | 463 |
| | 5029 | 5044 | | | | |
| 613816 | 1796 | 1811 | CCACGCGGAAACCAAA | 96 | C | 464 |
| | 5092 | 5107 | | | | |
| 613817 | 1856 | 1871 | TGGCCAGCCTTTCGGG | 96 | C | 465 |
| | 5152 | 5167 | | | | |
| 613818 | 1921 | 1936 | CGGGCTTGCACCCTTC | 111 | C | 466 |
| | 5217 | 5232 | | | | |
| 613819 | 1969 | 1984 | GCAGTGTGGCCGGTTT | 95 | C | 467 |
| | 5265 | 5280 | | | | |
| 613820 | 2031 | 2046 | AGATGCCTCCCCGGCG | 122 | C | 468 |
| | 5327 | 5342 | | | | |
| 613821 | 2060 | 2075 | TAGAAGACCAGAGCGA | 118 | C | 469 |
| | 5356 | 5371 | | | | |
| 613852 | 7318 | 7333 | AGAGAGGCCACGGCCC | 95 | C | 470 |
| 613861 | 7808 | 7823 | CCGCGCTGGCAGCTGG | 110 | C | 471 |
| 613869 | 8156 | 8171 | TGATCACCGAAGTTAT | 115 | C | 472 |
| 613884 | 1232 | 1247 | TTCCGCCGCCAGGCGC | 100 | D | 473 |
| | 4528 | 4543 | | | | |
| 613885 | 1302 | 1317 | ATCTCTGCCCGCCTTC | 77 | D | 474 |
| | 4598 | 4613 | | | | |
| 613886 | 1347 | 1362 | CTCCGCCCGTCCTTCC | 58 | D | 475 |
| | 4643 | 4658 | | | | |
| 613887 | 1403 | 1418 | GTCTTTCCCTCCGTTC | 81 | D | 476 |
| | 4699 | 4714 | | | | |
| 613888 | 1494 | 1509 | CCGGAGGCCGAGGACC | 91 | D | 477 |
| | 4787 | 4802 | | | | |
| 613889 | 1569 | 1584 | CGGCGGCTGTGGGCCC | 145 | D | 478 |
| | 4862 | 4877 | | | | |
| 613890 | 4910 | 4925 | CCTGGGCCCCGGAACC | 147 | D | 479 |
| 613891 | 1661 | 1676 | CGGCAACCCGAGTCCC | 152 | D | 480 |
| | 4957 | 4972 | | | | |
| 613892 | 5013 | 5028 | TTGCAGGGCTGAGCCT | 148 | D | 481 |
| 613893 | 1753 | 1768 | CTCCTCCGTGGCCGGG | 125 | D | 482 |
| | 5049 | 5064 | | | | |
| 613894 | 1825 | 1840 | AGCAACAGGCCGCCTT | 106 | D | 483 |
| | 5121 | 5136 | | | | |
| 613895 | 1878 | 1893 | CTCCGGGAGCAAACAG | 77 | D | 484 |
| | 5174 | 5189 | | | | |
| 613896 | 1954 | 1969 | TGGAACCTGGCAAGGA | 109 | D | 485 |
| | 5250 | 5265 | | | | |
| 613897 | 2006 | 2021 | GACGATGGATTCCCGC | 132 | D | 486 |
| | 5302 | 5317 | | | | |
| 613898 | 2047 | 2062 | CGAGACCCCAGAGAGG | 133 | D | 487 |
| | 5343 | 5358 | | | | |
| 613945 | 8141 | 8156 | TGTAAACCAATTTCAG | 120 | D | 488 |
| 613954 | 8585 | 8600 | GTTTTGACATATCTCT | 105 | D | 489 |
| 1098920 | 1209 | 1224 | TGGCACCTGGGCGGCT | 52 | E | 490 |
| | 4505 | 4520 | | | | |
| 1098925 | 1234 | 1249 | CGTTCCGCCGCCAGGC | 61 | E | 491 |
| | 4530 | 4545 | | | | |
| 1098929 | 1260 | 1275 | CCGGTGTGCGCCGGGC | 54 | E | 492 |
| | 4556 | 4571 | | | | |
| 1098933 | 1264 | 1279 | GTCCCCGGTGTGCGCC | 46 | E | 493 |
| | 4560 | 4575 | | | | |
| 1098937 | 1305 | 1320 | TCCATCTCTGCCCGCC | 42 | E | 494 |
| | 4601 | 4616 | | | | |
| 1098941 | 1333 | 1348 | CCGCCCCTCTAGGTCT | 35 | E | 495 |
| | 4629 | 4644 | | | | |
| 1098945 | 1339 | 1354 | GTCCTTCCGCCCCTCT | 70 | E | 496 |
| | 4635 | 4650 | | | | |
| 1098950 | 1344 | 1359 | CGCCCGTCCTTCCGCC | 45 | E | 497 |
| | 4640 | 4655 | | | | |
| 1098954 | 1349 | 1364 | CCCTCCGCCCGTCCTT | 70 | E | 498 |
| | 4645 | 4660 | | | | |
| 1098958 | 4746 | 4761 | CCGTCCCCCGGCTCCC | 63 | E | 499 |
| 1098962 | 1491 | 1506 | GAGGCCGAGGACCGCT | 49 | E | 500 |
| | 4784 | 4799 | | | | |
| 1098966 | 1500 | 1515 | CTACTCCCGGAGGCCG | 39 | E | 501 |
| | 4793 | 4808 | | | | |
| 1098970 | 1505 | 1520 | TCCCGCTACTCCCGGA | 64 | E | 502 |
| | 4798 | 4813 | | | | |
| 1098974 | 1546 | 1561 | AGCCCGCGCCGGACGC | 48 | E | 503 |
| | 4839 | 4854 | | | | |
| 1098979 | 1552 | 1567 | GCCCTCAGCCCGCGCC | 129 | E | 504 |
| | 4845 | 4860 | | | | |
| 1098983 | 1571 | 1586 | CGCGGCGGCTGTGGGC | 90 | E | 505 |
| | 4864 | 4879 | | | | |
| 1098987 | 1575 | 1590 | CCGGCGCGGCGGCTGT | 53 | E | 506 |
| | 4868 | 4883 | | | | |
| 1098992 | 4885 | 4900 | GGGTGGTGCCCCGCCG | 64 | E | 507 |
| 1098996 | 4911 | 4926 | CCCTGGGCCCCGGAAC | 44 | E | 508 |
| 1099000 | 1656 | 1671 | ACCCGAGTCCCGGTCT | 69 | E | 509 |
| | 4952 | 4967 | | | | |
| 1099004 | 1667 | 1682 | ACCCGACGGCAACCCG | 62 | E | 510 |
| | 4963 | 4978 | | | | |
| 1099008 | 1735 | 1750 | TGTCGGCCTCGCGCCG | 49 | E | 511 |
| | 5031 | 5046 | | | | |
| 1099012 | 1739 | 1754 | GGGCTGTCGGCCTCGC | 63 | E | 512 |
| | 5035 | 5050 | | | | |
| 1099016 | 1815 | 1830 | CGCCTTGCGGAGGGCA | 48 | E | 513 |
| | 5111 | 5126 | | | | |
| 1099020 | 1819 | 1834 | AGGCCGCCTTGCGGAG | 46 | E | 514 |
| | 5115 | 5130 | | | | |
| 1099024 | 1884 | 1899 | GCAGAGCTCCGGGAGC | 80 | E | 515 |
| | 5180 | 5195 | | | | |
| 1099028 | 1889 | 1904 | TGCCCGCAGAGCTCCG | 56 | E | 516 |
| | 5185 | 5200 | | | | |
| 1099033 | 1894 | 1909 | CCGGGTGCCCGCAGAG | 63 | E | 517 |
| | 5190 | 5205 | | | | |
| 1099037 | 1898 | 1913 | GTTTCCGGGTGCCCGC | 42 | E | 518 |
| | 5194 | 5209 | | | | |
| 1099041 | 5222 | 5237 | CGTGCCGGGCTTGCAC | 89 | E | 519 |
| 1099045 | 5229 | 5244 | AAGGCACCGTGCCGGG | 61 | E | 520 |
| 1099145 | 7315 | 7330 | GAGGCCACGGCCCTGC | 45 | E | 521 |
| 1099157 | 7811 | 7826 | GCTCCGCGCTGGCAGC | 46 | E | 522 |
| 1099161 | 7815 | 7830 | AGGAGCTCCGCGCTGG | 68 | E | 523 |
| 1099165 | 7819 | 7834 | CGCCAGGAGCTCCGCG | 41 | E | 524 |
| 1098922 | 1219 | 1234 | CGCTCCGTGCTGGCAC | 45 | F | 525 |
| | 4515 | 4530 | | | | |
| 1098926 | 1257 | 1272 | GTGTGCGCCGGGCCTG | 60 | F | 526 |
| | 4553 | 4568 | | | | |
| 1098930 | 1261 | 1276 | CCCGGTGTGCGCCGGG | 58 | F | 527 |
| | 4557 | 4572 | | | | |
| 1098934 | 1265 | 1280 | CGTCCCCGGTGTGCGC | 51 | F | 528 |
| | 4561 | 4576 | | | | |
| 1098938 | 1329 | 1344 | CCCTCTAGGTCTCCCG | 55 | F | 529 |
| | 4625 | 4640 | | | | |
| 1098942 | 1334 | 1349 | TCCGCCCCTCTAGGTC | 75 | F | 530 |
| | 4630 | 4645 | | | | |
| 1098946 | 1340 | 1355 | CGTCCTTCCGCCCCTC | 63 | F | 531 |
| | 4636 | 4651 | | | | |
| 1098951 | 1345 | 1360 | CCGCCCGTCCTTCCGC | 58 | F | 532 |
| | 4641 | 4656 | | | | |
| 1098955 | 1351 | 1366 | GTCCCTCCGCCCGTCC | 60 | F | 533 |
| | 4647 | 4662 | | | | |
| 1098959 | 4747 | 4762 | CCCGTCCCCCGGCTCC | 89 | F | 534 |
| 1098963 | 1493 | 1508 | CGGAGGCCGAGGACCG | 44 | F | 535 |
| | 4786 | 4801 | | | | |
| 1098967 | 1501 | 1516 | GCTACTCCCGGAGGCC | 57 | F | 536 |
| | 4794 | 4809 | | | | |
| 1098971 | 1506 | 1521 | GTCCCGCTACTCCCGG | 52 | F | 537 |
| | 4799 | 4814 | | | | |
| 1098975 | 1548 | 1563 | TCAGCCCGCGCCGGAC | 66 | F | 538 |
| | 4841 | 4856 | | | | |
| 1098980 | 1553 | 1568 | AGCCCTCAGCCCGCGC | 91 | F | 539 |
| | 4846 | 4861 | | | | |
| 1098984 | 1572 | 1587 | GCGCGGCGGCTGTGGG | 105 | F | 540 |
| | 4865 | 4880 | | | | |
| 1098988 | 4881 | 4896 | GGTGCCCCGCCGGCCG | 89 | F | 541 |
| 1098993 | 4886 | 4901 | TGGGTGGTGCCCCGCC | 48 | F | 542 |
| 1098997 | 4912 | 4927 | TCCCTGGGCCCCGGAA | 53 | F | 543 |
| 1099001 | 1659 | 1674 | GCAACCCGAGTCCCGG | 77 | F | 544 |
| | 4955 | 4970 | | | | |
| 1099005 | 4964 | 4979 | GACCCGACGGCAACCC | 86 | F | 545 |
| 1099009 | 1736 | 1751 | CTGTCGGCCTCGCGCC | 32 | F | 546 |
| | 5032 | 5047 | | | | |
| 1099013 | 1754 | 1769 | GCTCCTCCGTGGCCGG | 73 | F | 547 |
| | 5050 | 5065 | | | | |
| 1099017 | 1816 | 1831 | CCGCCTTGCGGAGGGC | 73 | F | 548 |
| | 5112 | 5127 | | | | |
| 1099021 | 1820 | 1835 | CAGGCCGCCTTGCGGA | 59 | F | 549 |
| | 5116 | 5131 | | | | |
| 1099025 | 1886 | 1901 | CCGCAGAGCTCCGGGA | 80 | F | 550 |
| | 5182 | 5197 | | | | |
| 1099029 | 1890 | 1905 | GTGCCCGCAGAGCTCC | 65 | F | 551 |
| | 5186 | 5201 | | | | |
| 1099034 | 1895 | 1910 | TCCGGGTGCCCGCAGA | 57 | F | 552 |
| | 5191 | 5206 | | | | |
| 1099038 | 1923 | 1938 | GCCGGGCTTGCACCCT | 66 | F | 553 |
| | 5219 | 5234 | | | | |
| 1099042 | 5223 | 5238 | CCGTGCCGGGCTTGCA | 43 | F | 554 |
| 1099046 | 5230 | 5245 | GAAGGCACCGTGCCGG | 91 | F | 555 |
| 1099142 | 7307 | 7322 | GGCCCTGCCCCGAACC | 51 | F | 556 |
| 1099146 | 7316 | 7331 | AGAGGCCACGGCCCTG | 47 | F | 557 |
| 1099154 | 7807 | 7822 | CGCGCTGGCAGCTGGG | 79 | F | 558 |
| 1099158 | 7812 | 7827 | AGCTCCGCGCTGGCAG | 54 | F | 559 |
| 1099162 | 7816 | 7831 | CAGGAGCTCCGCGCTG | 68 | F | 560 |
| 1099166 | 7822 | 7837 | GACCGCCAGGAGCTCC | 49 | F | 561 |
| 1098927 | 1258 | 1273 | GGTGTGCGCCGGGCCT | 107 | G | 562 |
| | 4554 | 4569 | | | | |
| 1098931 | 1262 | 1277 | CCCCGGTGTGCGCCGG | 105 | G | 563 |
| | 4558 | 4573 | | | | |
| 1098935 | 1285 | 1300 | CTCCCGCCTGGAACGC | 89 | G | 564 |
| | 4581 | 4596 | | | | |
| 1098939 | 1330 | 1345 | CCCCTCTAGGTCTCCC | 69 | G | 565 |
| | 4626 | 4641 | | | | |
| 1098943 | 1335 | 1350 | TTCCGCCCCTCTAGGT | 80 | G | 566 |
| | 4631 | 4646 | | | | |
| 1098947 | 1341 | 1356 | CCGTCCTTCCGCCCCT | 63 | G | 567 |
| | 4637 | 4652 | | | | |
| 1098952 | 1346 | 1361 | TCCGCCCGTCCTTCCG | 76 | G | 568 |
| | 4642 | 4657 | | | | |
| 1098956 | 1352 | 1367 | CGTCCCTCCGCCCGTC | 100 | G | 569 |
| | 4648 | 4663 | | | | |
| 1098960 | 4748 | 4763 | CCCCGTCCCCCGGCTC | 70 | G | 570 |
| 1098964 | 1495 | 1510 | CCCGGAGGCCGAGGAC | 72 | G | 571 |
| | 4788 | 4803 | | | | |
| 1098968 | 1502 | 1517 | CGCTACTCCCGGAGGC | 73 | G | 572 |
| | 4795 | 4810 | | | | |
| 1098972 | 1507 | 1522 | GGTCCCGCTACTCCCG | 97 | G | 573 |
| | 4800 | 4815 | | | | |
| 1098976 | 1549 | 1564 | CTCAGCCCGCGCCGGA | 64 | G | 574 |
| | 4842 | 4857 | | | | |
| 1098981 | 1554 | 1569 | CAGCCCTCAGCCCGCG | 154 | G | 575 |
| | 4847 | 4862 | | | | |
| 1098985 | 1573 | 1588 | GGCGCGGCGGCTGTGG | 128 | G | 576 |
| | 4866 | 4881 | | | | |
| 1098989 | 4882 | 4897 | TGGTGCCCCGCCGGCC | 91 | G | 577 |
| 1098994 | 4908 | 4923 | TGGGCCCCGGAACCGG | 118 | G | 578 |
| 1098998 | 1635 | 1650 | CCCGGAGGAAACCGCC | 149 | G | 579 |
| | 4931 | 4946 | | | | |
| 1099002 | 1660 | 1675 | GGCAACCCGAGTCCCG | 64 | G | 580 |
| | 4956 | 4971 | | | | |
| 1099006 | 4975 | 4990 | GCGCGGGTGAAGACCC | 167 | G | 581 |
| 1099010 | 1737 | 1752 | GCTGTCGGCCTCGCGC | 63 | G | 582 |
| | 5033 | 5048 | | | | |
| 1099014 | 1755 | 1770 | GGCTCCTCCGTGGCCG | 112 | G | 583 |
| | 5051 | 5066 | | | | |
| 1099018 | 1817 | 1832 | GCCGCCTTGCGGAGGG | 101 | G | 584 |
| | 5113 | 5128 | | | | |
| 1099022 | 1821 | 1836 | ACAGGCCGCCTTGCGG | 64 | G | 585 |
| | 5117 | 5132 | | | | |
| 1099026 | 1887 | 1902 | CCCGCAGAGCTCCGGG | 183 | G | 586 |
| | 5183 | 5198 | | | | |
| 1099031 | 1892 | 1907 | GGGTGCCCGCAGAGCT | 110 | G | 587 |
| | 5188 | 5203 | | | | |
| 1099035 | 1896 | 1911 | TTCCGGGTGCCCGCAG | 108 | G | 588 |
| | 5192 | 5207 | | | | |
| 1099039 | 1924 | 1939 | TGCCGGGCTTGCACCC | 105 | G | 589 |
| | 5220 | 5235 | | | | |
| 1099043 | 5224 | 5239 | ACCGTGCCGGGCTTGC | 60 | G | 590 |
| 1099047 | 5232 | 5247 | GCGAAGGCACCGTGCC | 112 | G | 591 |
| 1099143 | 7308 | 7323 | CGGCCCTGCCCCGAAC | 120 | G | 592 |
| 1099155 | 7809 | 7824 | TCCGCGCTGGCAGCTG | 94 | G | 593 |
| 1099159 | 7813 | 7828 | GAGCTCCGCGCTGGCA | 103 | G | 594 |
| 1099163 | 7817 | 7832 | CCAGGAGCTCCGCGCT | 87 | G | 595 |
| 1098919 | 1207 | 1222 | GCACCTGGGCGGCTGC | 84 | H | 596 |
| | 4503 | 4518 | | | | |
| 1098924 | 1233 | 1248 | GTTCCGCCGCCAGGCG | 78 | H | 597 |
| | 4529 | 4544 | | | | |
| 1098928 | 1259 | 1274 | CGGTGTGCGCCGGGCC | 78 | H | 598 |
| | 4555 | 4570 | | | | |
| 1098932 | 1263 | 1278 | TCCCCGGTGTGCGCCG | 59 | H | 599 |
| | 4559 | 4574 | | | | |
| 1098936 | 1286 | 1301 | CCTCCCGCCTGGAACG | 77 | H | 600 |
| | 4582 | 4597 | | | | |
| 1098940 | 1331 | 1346 | GCCCCTCTAGGTCTCC | 68 | H | 601 |
| | 4627 | 4642 | | | | |
| 1098944 | 1338 | 1353 | TCCTTCCGCCCCTCTA | 63 | H | 602 |
| | 4634 | 4649 | | | | |
| 1098949 | 1343 | 1358 | GCCCGTCCTTCCGCCC | 46 | H | 603 |
| | 4639 | 4654 | | | | |
| 1098953 | 1348 | 1363 | CCTCCGCCCGTCCTTC | 68 | H | 604 |
| | 4644 | 4659 | | | | |
| 1098957 | 4745 | 4760 | CGTCCCCCGGCTCCCT | 69 | H | 605 |
| 1098961 | 4749 | 4764 | CCCCCGTCCCCCGGCT | 79 | H | 606 |
| 1098965 | 1499 | 1514 | TACTCCCGGAGGCCGA | 60 | H | 607 |
| | 4792 | 4807 | | | | |
| 1098969 | 1504 | 1519 | CCCGCTACTCCCGGAG | 58 | H | 608 |
| | 4797 | 4812 | | | | |
| 1098973 | 1508 | 1523 | GGGTCCCGCTACTCCC | 84 | H | 609 |
| | 4801 | 4816 | | | | |
| 1098977 | 1550 | 1565 | CCTCAGCCCGCGCCGG | 61 | H | 610 |
| | 4843 | 4858 | | | | |
| 1098982 | 1570 | 1585 | GCGGCGGCTGTGGGCC | 75 | H | 611 |
| | 4863 | 4878 | | | | |
| 1098986 | 1574 | 1589 | CGGCGCGGCGGCTGTG | 49 | H | 612 |
| | 4867 | 4882 | | | | |
| 1098990 | 4883 | 4898 | GTGGTGCCCCGCCGGC | 65 | H | 613 |
| 1098995 | 4909 | 4924 | CTGGGCCCCGGAACCG | 67 | H | 614 |
| 1098999 | 1655 | 1670 | CCCGAGTCCCGGTCTT | 61 | H | 615 |
| | 4951 | 4966 | | | | |
| 1099003 | 1666 | 1681 | CCCGACGGCAACCCGA | 51 | H | 616 |
| | 4962 | 4977 | | | | |
| 1099007 | 1734 | 1749 | GTCGGCCTCGCGCCGC | 76 | H | 617 |
| | 5030 | 5045 | | | | |
| 1099011 | 1738 | 1753 | GGCTGTCGGCCTCGCG | 90 | H | 618 |
| | 5034 | 5049 | | | | |
| 1099015 | 1760 | 1775 | CGTGTGGCTCCTCCGT | 24 | H | 619 |
| | 5056 | 5071 | | | | |
| 1099019 | 1818 | 1833 | GGCCGCCTTGCGGAGG | 73 | H | 620 |
| | 5114 | 5129 | | | | |
| 1099023 | 1863 | 1878 | GTCGGCATGGCCAGCC | 113 | H | 621 |
| | 5159 | 5174 | | | | |
| 1099027 | 1888 | 1903 | GCCCGCAGAGCTCCGG | 87 | H | 622 |
| | 5184 | 5199 | | | | |
| 1099032 | 1893 | 1908 | CGGGTGCCCGCAGAGC | 91 | H | 623 |
| | 5189 | 5204 | | | | |
| 1099036 | 1897 | 1912 | TTTCCGGGTGCCCGCA | 41 | H | 62 |
| | 5193 | 5208 | | | | |
| 1099040 | 5221 | 5236 | GTGCCGGGCTTGCACC | 81 | H | 625 |
| 1099044 | 5226 | 5241 | GCACCGTGCCGGGCTT | 60 | H | 626 |
| 1099048 | 2034 | 2049 | AGGAGATGCCTCCCCG | 46 | H | 627 |
| | 5330 | 5345 | | | | |
| 1099144 | 7309 | 7324 | ACGGCCCTGCCCCGAA | 93 | H | 628 |
| 1099156 | 7810 | 7825 | CTCCGCGCTGGCAGCT | 47 | H | 629 |
| 1099160 | 7814 | 7829 | GGAGCTCCGCGCTGGC | 82 | H | 630 |
| 1099164 | 7818 | 7833 | GCCAGGAGCTCCGCGC | 59 | H | 631 |

### Example 3: Dose-Dependent Inhibition of Human DUX4 in 54-2 Cells by Modified Oligonucleotides

Modified oligonucleotides selected from the examples above were tested at various doses in 54-2 cells (described herein above). 54-2 cells plated at a density of 6,000 cells per well were differentiated (as described herein above) and treated using cytofectin with various concentrations of modified oligonucleotide as specified in the table below. After a treatment period of approximately 24 hours, total RNA was isolated from the cells and DUX4 RNA levels were measured by quantitative real-time RTPCR. Human DUX4 primer-probe set RTS3502 (described herein above) was used to measure RNA levels as described above. DUX4 RNA levels were normalized to total RNA content, as measured by RIBOGREEN®. Reduction of DUX4 RNA is presented in the table below as percent DUX4 RNA, relative to the amount of DUX4 RNA in untreated control cells (% UTC).

The half maximal inhibitory concentration (IC₅₀) of each modified oligonucleotide was calculated using a linear regression on a log/linear plot of the data in Excel and is also presented in the table below.

TABLE 5
Dose-dependent reduction of human DUX4 RNA
in 54-2 cells by modified oligonucleotides

| | DUX4 RNA (% UTC) | | | | | |
|---|---|---|---|---|---|---|
| Compound | 18.75 | 37.5 | 75.0 | 150.0 | 300.0 | IC₅₀ |
| No. | nM | nM | nM | nM | nM | (μM) |
| 541109 | 104 | 103 | 88 | 63 | 36 | 0.23 |
| 541114 | 101 | 100 | 71 | 44 | 17 | 0.13 |
| 541117 | 89 | 85 | 77 | 62 | 28 | 0.18 |
| 541135 | 87 | 88 | 81 | 62 | 33 | 0.21 |
| 541146 | 114 | 96 | 90 | 60 | 35 | 0.21 |
| 541170 | 124 | 120 | 98 | 79 | 51 | >0.3 |
| 541176 | 104 | 100 | 83 | 40 | 20 | 0.14 |
| 541200 | 100 | 113 | 81 | 56 | 36 | 0.21 |
| 541204 | 88 | 76 | 62 | 43 | 30 | 0.12 |
| 541205 | 80 | 80 | 65 | 46 | 29 | 0.12 |
| 541222 | 90 | 95 | 63 | 39 | 29 | 0.13 |
| 541255 | 134 | 111 | 108 | 87 | 59 | >0.3 |
| 541259 | 116 | 111 | 89 | 44 | 29 | 0.17 |
| 541260 | 100 | 95 | 90 | 42 | 18 | 0.14 |

### Example 4: Dose-Dependent Inhibition of Human DUX4 in 54-2 Cells by Modified Oligonucleotides

Modified oligonucleotides selected from the examples above were tested at various doses in 54-2 cells (described herein above). 54-2 cells plated at a density of 10,000 cells per well, were differentiated (as described herein above) and treated using cytofectin with various concentrations of modified oligonucleotide as specified in the tables below. After a treatment period of approximately 24 hours, total RNA was isolated from the cells and DUX4 RNA levels were measured by quantitative real-time RTPCR. Human DUX4 primer-probe set RTS3502 (described herein above) was used to measure RNA levels as described above. DUX4 RNA levels were normalized to total RNA content, as measured by RIBOGREEN®. Reduction of DUX4 RNA is presented in the tables below as percent DUX4 RNA relative to the amount of DUX4 RNA in untreated control cells (% UTC). The modified oligonucleotides were tested in a series of experiments that had the same culture conditions, and the results for each experiment are presented in separate tables below.

The half maximal inhibitory concentration (IC₅₀) of each modified oligonucleotide was calculated using a linear regression on a log/linear plot of the data in Excel and is also presented in the tables below.

TABLE 6
Dose-dependent reduction of human DUX4 RNA
in 54-2 cells by modified oligonucleotides

| | DUX4 RNA (% UTC) | | | | | |
|---|---|---|---|---|---|---|
| Compound | 25.0 | 50.0 | 100.0 | 200.0 | 400.0 | IC₅₀ |
| No. | nM | nM | nM | nM | nM | (μM) |
| 541114 | 83 | 62 | 69 | 42 | 20 | 0.13 |
| 613801 | 98 | 98 | 74 | 52 | 28 | 0.21 |
| 613802 | 99 | 78 | 93 | 56 | 58 | >0.4 |
| 613804 | 70 | 59 | 51 | 33 | 23 | 0.08 |
| 613807 | 90 | 91 | 68 | 54 | 25 | 0.19 |
| 613823 | 102 | 102 | 93 | 75 | 43 | >0.4 |
| 613831 | 75 | 55 | 46 | 21 | 15 | 0.07 |
| 613834 | 89 | 69 | 67 | 50 | 31 | 0.18 |
| 613841 | 97 | 65 | 50 | 35 | 17 | 0.11 |
| 613844 | 96 | 80 | 50 | 49 | 26 | 0.15 |
| 613849 | 93 | 87 | 77 | 62 | 29 | 0.24 |
| 613851 | 72 | 59 | 53 | 46 | 31 | 0.12 |
| 613855 | 90 | 69 | 59 | 40 | 24 | 0.13 |
| 613867 | 76 | 74 | 59 | 48 | 23 | 0.14 |

TABLE 7
Dose-dependent reduction of human DUX4 RNA
in 54-2 cells by modified oligonucleotides

| | DUX4 RNA (% UTC) | | | | | |
|---|---|---|---|---|---|---|
| Compound | 25.0 | 50.0 | 100.0 | 200.0 | 400.0 | IC₅₀ |
| No. | nM | nM | nM | nM | nM | (μM) |
| 541114 | 82 | 64 | 77 | 46 | 31 | 0.18 |
| 613879 | 94 | 79 | 62 | 56 | 28 | 0.18 |
| 613882 | 90 | 86 | 96 | 68 | 30 | 0.34 |
| 613885 | 67 | 82 | 80 | 39 | 27 | 0.16 |
| 613886 | 96 | 67 | 58 | 61 | 33 | 0.20 |
| 613887 | 90 | 64 | 57 | 45 | 34 | 0.15 |
| 613895 | 78 | 85 | 82 | 66 | 51 | >0.4 |
| 613910 | 79 | 89 | 46 | 45 | 13 | 0.12 |
| 613913 | 85 | 94 | 75 | 61 | 36 | 0.29 |
| 613916 | 101 | 69 | 60 | 56 | 41 | 0.22 |
| 613922 | 90 | 66 | 69 | 49 | 45 | 0.25 |
| 613942 | 40 | 41 | 31 | 28 | 13 | <0.025 |
| 613949 | 118 | 77 | 92 | 79 | 52 | >0.4 |
| 613956 | 91 | 81 | 70 | 48 | 42 | 0.24 |

TABLE 8
Dose-dependent reduction of human DUX4 RNA
in 54-2 cells by modified oligonucleotides

| Compound | DUX4 RNA (% UTC) | | | | IC₅₀ |
|---|---|---|---|---|---|
| No. | 19 nM | 56 nM | 167 nM | 500 nM | (μM) |
| 613910 | 65 | 38 | 21 | 15 | 0.04 |
| 1098882 | 80 | 50 | 19 | 8 | 0.06 |
| 1098904 | 99 | 52 | 20 | 7 | 0.08 |
| 1098909 | 83 | 68 | 63 | 77 | >0.5 |
| 1098912 | 47 | 25 | 13 | 9 | <0.02 |
| 1098941 | 40 | 28 | 13 | 13 | <0.02 |
| 1099009 | 74 | 59 | 22 | 6 | 0.06 |
| 1099081 | 63 | 37 | 20 | 11 | 0.03 |
| 1099102 | 64 | 43 | 28 | 22 | 0.04 |
| 1099109 | 104 | 64 | 22 | 12 | 0.10 |
| 1099110 | 78 | 55 | 39 | 33 | 0.11 |
| 1099121 | 64 | 48 | 34 | 26 | 0.05 |
| 1099126 | 79 | 43 | 18 | 7 | 0.05 |
| 1099133 | 70 | 54 | 27 | 15 | 0.06 |
| 1099138 | 69 | 38 | 15 | 7 | 0.04 |
| 1099149 | 92 | 44 | 19 | 9 | 0.07 |
| 1099150 | 72 | 36 | 19 | 9 | 0.04 |
| 1099183 | 86 | 51 | 18 | 11 | 0.07 |

TABLE 9
Dose-dependent reduction of human DUX4 RNA
in 54-2 cells by modified oligonucleotides

| Compound | DUX4 RNA (% UTC) | | | | IC₅₀ |
|---|---|---|---|---|---|
| No. | 19 nM | 56 nM | 167 nM | 500 nM | (μM) |
| 1098881 | 80 | 71 | 31 | 10 | 0.09 |
| 1098894 | 111 | 81 | 19 | 6 | 0.11 |
| 1098895 | 65 | 45 | 23 | 13 | 0.04 |
| 1098907 | 82 | 58 | 21 | 7 | 0.07 |
| 1098910 | 61 | 40 | 17 | 8 | 0.03 |
| 1099015 | 68 | 45 | 16 | 6 | 0.04 |
| 1099043 | 40 | 24 | 8 | 4 | <0.02 |
| 1099080 | 118 | 82 | 25 | 13 | 0.13 |
| 1099107 | 42 | 24 | 10 | 4 | <0.02 |
| 1099108 | 67 | 62 | 26 | 10 | 0.06 |
| 1099132 | 69 | 61 | 31 | 9 | 0.07 |
| 1099139 | 152 | 90 | 15 | 5 | 0.13 |
| 1099140 | 57 | 33 | 15 | 8 | 0.02 |
| 1099147 | 151 | 64 | 18 | 8 | 0.13 |
| 1099148 | 60 | 39 | 31 | 18 | 0.03 |
| 1099151 | 163 | 92 | 20 | 5 | 0.14 |
| 1099180 | 95 | 38 | 16 | 9 | 0.06 |
| 1099184 | 76 | 54 | 20 | 11 | 0.06 |

### Example 5: Design of a Modified Oligonucleotides Complementary to a Human DUX4 Nucleic Acid

A modified oligonucleotide complementary to a human DUX4 nucleic acid was designed, as described in the table below.

The modified oligonucleotide in Table 10 is a 3-10-3 cEt gapmer conjugated to a 6-palmitamidohexyl conjugate moiety attached to the 5′-OH of the oligonucleotide through a phosphodiester linker. The structure for the conjugate group is:

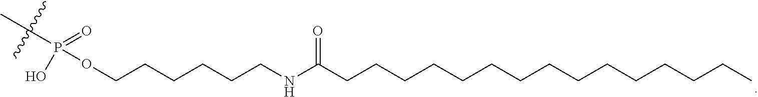 [SMILES: *P(=O)(O)OCCCCCCNC(=O)CCCCCCCCCCCCCCC]

The gapmer is 16 nucleosides in length, wherein the central gap segment consists of ten 2′-β-D-deoxynucleosides and the 5′ and 3′ wings each consists of three cEt nucleosides. The sugar motif for the gapmer is (from 5′ to 3′): kkkddddddddddkkk; wherein ‘d’ represents a 2′-β-D-deoxyribosyl sugar moiety; and ‘k’ represents a cEt sugar moiety. Each internucleoside linkages is a phosphorothioate internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. The modified oligonucleotide listed in the table below is 100% complementary to SEQ ID NO: 1 (described herein above), and to SEQ ID NO: 2 (described herein above).

TABLE 10
6-Palmitamidohexyl conjugated 3-10-3 cEt gapmer with PS internucleoside linkages complementary
to human DUX4

| | | SEQ | SEQ | SEQ | SEQ | | |
|---|---|---|---|---|---|---|---|
| | | ID | ID | ID | ID | | |
| | | NO: | NO: | NO: | NO: | | |
| | | 1 | 1 | 2 | 2 | | SEQ |
| Compound | Parent | Start | Stop | Start | Stop | | ID |
| ID | Compound | Site | Site | Site | Site | Sequence (5′ to 3′) | NO |
| 941806 | 613910 | 2899 | 2914 | 126 | 141 | GGCGATGCCCGGGTAC | 248 |

### Example 6: Activity of Modified Oligonucleotides Complementary to Human DUX4 in Transgenic Mice

Transgenic mice expressing human DUX4 were used to test activity of modified oligonucleotides described above. The transgenic mouse model was developed using random integration of a linearized modified human DUX4 gene. The clone was digested at AseI and XhoI restriction sites to produce a region containing Mck (muscle specific) promoter driven human DUX4 transgene with a single amino acid mutation of F67A. The gene fragment was introduced into fertilized eggs from C57BL6NJ strain mice by pronuclear injection to produce 7 founder lines. Line 9111 was used in the experiments described herein. Human DUX4 RNA expression is found in the muscle in this model.

### Treatment

Each animal in a group of 4 transgenic mice was administered 50 mg/kg of Compound No. 941806 by subcutaneous injection twice a week for a total of six injections. A group of 4 mice received PBS as a negative control.

### RNA Analysis

Mice were sacrificed 72 hours after the final dose, and RNA was extracted from the quadriceps muscle, transverse abdominal muscle (TA), and gastrocnemius muscle for RTPCR analysis to measure amount of DUX4 RNA using human primer probe set RTS3502 (described herein above). Results are presented as percent human DUX4 RNA relative to PBS control, normalized to mouse GAPDH (% control). Mouse GAPDH RNA was amplified using mouse prime probe set mGapdh_LTS00102 (forward sequence GGCAAATTCAACGGCACAGT, designated herein as SEQ ID NO: 14; reverse sequence GGGTCTCGCTCCTGGAAGAT, designated herein as SEQ ID NO: 15; probe sequence AAGGCCGAGAATGGGAAGCTTGTCATC, designated herein as SEQ ID NO: 16).

TABLE 11
Reduction of human DUX4 RNA in transgenic mice

| Compound | DUX4 RNA (% control) | | |
|---|---|---|---|
| ID | Quadriceps | TA | Gastrocnemius |
| PBS | 100 | 100 | 100 |
| 941806 | 17 | 32 | 25 |

### Example 7: Effect of 3-10-3 cEt Modified Oligonucleotides with Uniform Phosphorothioate Internucleoside Linkages on Human DUX4 RNA In Vitro, Single Dose

Modified oligonucleotides complementary to human DUX4 nucleic acid were designed and tested for their single dose effects on human DUX4 RNA in vitro. The modified oligonucleotides were tested in a series of experiments that had the same culture conditions.

The modified oligonucleotides in the table below are 3-10-3 cET modified oligonucleotides with uniform phosphorothioate internucleoside linkages. The modified oligonucleotides are 16 nucleosides in length. The sugar motif for the modified oligonucleotides is (from 5′ to 3′): kkkddddddddddkkk; wherein each “d” represents a 2′-β-D-deoxyribosyl sugar moiety, and each “k” represents a cEt modified sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methylcytosine. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to one or more of the following SEQ ID NOs: SEQ ID NO: 1 (described herein above), to SEQ ID NO: 2 (describe herein above), to SEQ ID NO: 3 (described herein above). “N/A” indicates that the modified oligonucleotide is not 100% complementary to that particular target nucleic acid sequence.

54-2 cells plated at a density of 10,000 cells per well, were differentiated as described herein above, and were treated with modified oligonucleotide at a concentration of 200 nM using cytofectin. After a treatment period of approximately 24 hours, total RNA was isolated from the cells and human DUX4 RNA levels were measured by quantitative real-time RTPCR. Human DUX4 RNA levels were measured by probe set RTS40199 (forward sequence CTCTCTGTGCCCTTGTTCTT, designated herein as SEQ ID NO: 17; reverse sequence CATCCAGGAGATGTAACTCTAATCC, designated herein as SEQ ID NO: 18; probe sequence CCTTCCGACGCTGTCTAGGCAAA, designated herein as SEQ ID NO: 19). Human DUX4 RNA levels were normalized to total RNA content, as measured by RIBOGREEN®. Reduction of human DUX4 RNA is presented in the tables below as percent DUX4 RNA relative to the amount of DUX4 RNA in untreated control cells (% UTC). The values marked with a “t” indicate that the modified oligonucleotide is complementary to the amplicon region of the primer probe set. Additional assays may be used to measure the potency and efficacy of the modified oligonucleotides complementary to the amplicon region. “N.D.” in the tables below refers to instances where the value was Not Defined.

Each separate experiment described in this example is identified by an Assay Identification letter in the table column labeled “Analysis ID”.

TABLE 12
Reduction of human DUX4 RNA by 3-10-3 cEt modified oligonucleotides with uniform phosphorothioate
internucleoside linkages in differentiated 54-2 cells

| | SEQ ID | SEQ ID | SEQ ID | SEQ ID | | | | |
|---|---|---|---|---|---|---|---|---|
| | NO: 1 | NO: 1 | NO: 2 | NO: 2 | | DUX4 | | SEQ |
| Compound | Start | Stop | Start | Stop | | (% | Analysis | ID |
| No. | Site | Site | Site | Site | Sequence (5′ to 3′) | UTC) | ID | NO |
| 1098912 | 4513 | 4528 | N/A | N/A | AGAGGAGGCGAGCCGC | 25 | I | 301 |
| 1582463 | 4841 | 4856 | N/A | N/A | ATGCAAATCTTCTATA | 55 | I | 632 |
| 1582480 | 3632 | 3647 | 859 | 874 | CCGCTTGAGCGGGCCC | 28 | I | 633 |
| 1582486 | 3639 | 3654 | 866 | 881 | TGCGGCCCCGCTTGAG | 39 | I | 634 |
| 1582493 | 3658 | 3673 | 885 | 900 | CGCAAGCACCCCTTGG | 22 | I | 635 |
| 1582496 | 2718 | 2733 | N/A | N/A | CAGAGAGAGGCCGGCG | 38 | I | 636 |
| 1582500 | 3684 | 3699 | 911 | 926 | CACGGACTCCCCTGGG | 39 | I | 637 |
| 1582506 | 3721 | 3736 | 948 | 963 | CGCCCCGGCGACCTGG | 66 | I | 638 |
| 1582513 | 3727 | 3742 | 954 | 969 | CCACGCCGCCCCGGCG | 66 | I | 639 |
| 1582519 | 2729 | 2744 | N/A | N/A | ACGGACGCGGGCAGAG | 79 | I | 640 |

| | | | | | | | | |
|---|---|---|---|---|---|---|---|---|
| 1582525 | 3793 | 3808 | 1020 | 1035 | CCGCGCGGAGGCGGAG | 57 | I | 641 |
| 1582531 | 3820 | 3835 | 1047 | 1062 | CGCCGGGATGCCTTGC | 29 | I | 642 |
| 1582539 | 3847 | 3862 | 1074 | 1089 | CGCCGGCTCCTGGAGC | 73 | I | 643 |
| 1582546 | 3857 | 3872 | 1084 | 1099 | CAGACCAGGGCGCCGG | 35 | I | 644 |
| 1582549 | 2740 | 2755 | N/A | N/A | GAATTTCACGGACGGA | 22 | I | 645 |
| 1582555 | 3898 | 3913 | 1125 | 1140 | GCTCGCCAGGAGCTCA | 20 | I | 646 |
| 1582562 | 3909 | 3924 | 1136 | 1151 | AGAAACTCCGGGCTCG | 16 | I | 647 |
| 1582570 | 3931 | 3946 | 1158 | 1173 | TAGGAGAGGTTGCGCC | 53 | I | 648 |
| 1582576 | 3938 | 3953 | 1165 | 1180 | CCGTTTCTAGGAGAGG | 25 | I | 649 |
| 1582579 | 2747 | 2762 | N/A | N/A | CCGGCCGGAATTTCAC | 46 | I | 650 |
| 1582586 | 3982 | 3997 | 1209 | 1224 | CAGCGAGGCGGCCTCT | 20 | I | 651 |
| 1582592 | 4012 | 4027 | 1239 | 1254 | CCGGTATTCTTCCTCG | 35 | I | 652 |
| 1582598 | 4021 | 4036 | 1248 | 1263 | CAGCAGAGCCCGGTAT | 17 | I | 653 |
| 1582610 | N/A | N/A | 1440 | 1455 | TTCTCCGCGGTGTGGA | 54 | I | 654 |
| 1582617 | 2809 | 2824 | 36 | 51 | TCCCCGGGCTTCCGCG | 59 | I | 655 |
| 1582628 | 4602 | 4617 | N/A | N/A | AGGTATGCTTTTGACC | 38 | I | 656 |
| 1582634 | 4622 | 4637 | N/A | N/A | AAGCGGGCAAAGACAG | 35 | I | 657 |
| 1582646 | 4671 | 4686 | 1536 | 1551 | CTCCCTTGCACGTCAG | 20 | I | 658 |
| 1582652 | 4703 | 4718 | 1568 | 1583 | CGGAAGAACAAGGGCA | 11† | I | 659 |
| 1582658 | 4710 | 4725 | 1575 | 1590 | AATTTCACGGAAGAAC | 19† | I | 660 |
| 1582664 | 4717 | 4732 | 1582 | 1597 | CAGCCAGAATTTCACG | 15 | I | 661 |
| 1582671 | 4748 | 4763 | 1613 | 1628 | TAGACAGCGTCGGAAG | 12† | I | 662 |
| 1582677 | 4764 | 4779 | 1629 | 1644 | CTAATCCAGGTTTGCC | 5† | I | 663 |
| 1582683 | 4771 | 4786 | 1636 | 1651 | TGTAACTCTAATCCAG | 10 | I | 664 |
| 1582689 | 4780 | 4795 | 1645 | 1660 | TCCAGGAGATGTAACT | 12 | I | 665 |
| 1582695 | 4788 | 4803 | 1653 | 1668 | ACTAATCATCCAGGAG | 11 | I | 666 |
| 1582701 | 4794 | 4809 | 1659 | 1674 | CTCTGAACTAATCATC | 21 | I | 667 |
| 1582704 | 2835 | 2850 | 62 | 77 | CAAACGAGTCTCCGTC | 18 | I | 668 |
| 1582710 | 2865 | 2880 | 92 | 107 | GCTCGCAGGGCCTCGC | 21 | I | 669 |
| 1582716 | 2877 | 2892 | 104 | 119 | CGCTCAAAGCAGGCTC | 47 | I | 670 |
| 1582722 | 2897 | 2912 | 124 | 139 | CGATGCCCGGGTACGG | 11 | I | 671 |
| 1582729 | 2934 | 2949 | 161 | 176 | GGAATGCCGATGGCCT | 22 | I | 672 |
| 1582735 | 2955 | 2970 | 182 | 197 | CAAATCTGGACCCTGG | 20 | I | 673 |
| 1582741 | 2980 | 2995 | 207 | 222 | CTGGCGTGACCTCTCA | 24 | I | 674 |
| 1582747 | 3008 | 3023 | 235 | 250 | GCCGAGATTCCCGCCG | 65 | I | 675 |
| 1582753 | 3033 | 3048 | 260 | 275 | GGCGGGCCGCGTCTCC | 65 | I | 676 |
| 1582759 | 3068 | 3083 | 295 | 310 | ATCCGGTGACGGCGGT | 24 | I | 677 |
| 1582765 | 3097 | 3112 | 324 | 339 | AAAGGCTCGGAGGAGC | 29 | I | 678 |
| 1582771 | 3104 | 3119 | 331 | 346 | CCTTCTCAAAGGCTCG | 13 | I | 679 |
| 1582777 | 3115 | 3130 | 342 | 357 | TGGAAAGCGATCCTTC | 20 | I | 680 |
| 1582783 | 3121 | 3136 | 348 | 363 | GATGCCTGGAAAGCGA | 20 | I | 681 |
| 1582789 | 3132 | 3147 | 359 | 374 | TCCCGGGCGGCGATGC | 69 | I | 682 |
| 1582795 | 3162 | 3177 | 389 | 404 | TCCGGGAGGCCCGTCT | 40 | I | 683 |
| 1582801 | 3176 | 3191 | 403 | 418 | TCTGAATCCTGGACTC | 11 | I | 684 |
| 1582807 | 3221 | 3236 | 448 | 463 | TGCCACCCTGTCCCGG | 43 | I | 685 |
| 1582813 | 3235 | 3250 | 462 | 477 | CTGCGCGGGCGCCCTG | 61 | I | 686 |
| 1582819 | 3260 | 3275 | 487 | 502 | GGGCCGCGCTGCACAG | 32 | I | 687 |
| 1582825 | 3294 | 3309 | 521 | 536 | GCGACCCACGAGGGAG | 47 | I | 688 |
| 1582831 | 3300 | 3315 | 527 | 542 | GCGAAGGCGACCCACG | 24 | I | 689 |
| 1582837 | 3313 | 3328 | 540 | 555 | CGCGCCGGTGTGGGCG | 50 | I | 690 |
| 1582843 | 3320 | 3335 | 547 | 562 | TTCCCCACGCGCCGGT | 24 | I | 691 |
| 1582850 | 3333 | 3348 | 560 | 575 | GCGGGAAGCCCCGTTC | 69 | I | 692 |
| 1582853 | 2686 | 2701 | N/A | N/A | AGCCGGACTGTGCACT | 45 | I | 693 |
| 1582858 | 3355 | 3370 | 582 | 597 | AGGCGCGCAGGGCACG | 20 | I | 694 |
| 1582864 | 3362 | 3377 | 589 | 604 | GAGCCCCAGGCGCGCA | 20 | I | 695 |
| 1582870 | 3379 | 3394 | 606 | 621 | GAAAGCCCCCTGTGGG | 44 | I | 696 |
| 1582877 | 3385 | 3400 | 612 | 627 | GCTCACGAAAGCCCCC | 16 | I | 697 |
| 1582883 | 3445 | 3460 | 672 | 687 | GATCCCCTCTGCCGGC | 88 | I | 698 |
| 1582890 | 3475 | 3490 | 702 | 717 | GAAATCCCCGCGCGCC | 33 | I | 699 |
| 1582896 | 3483 | 3498 | 710 | 725 | GCGTAGGCGAAATCCC | 26 | I | 700 |
| 1582902 | 3502 | 3517 | 729 | 744 | GTCCGGAGGAGCCGGG | 28 | I | 701 |
| 1582908 | 3509 | 3524 | 736 | 751 | GCGCCCCGTCCGGAGG | 55 | I | 702 |
| 1582914 | 3549 | 3564 | 776 | 791 | CCCGGGTGCGGAGGCC | 74 | I | 703 |
| 1582920 | 3577 | 3592 | 804 | 819 | CTGCGGGTCCCGGTCC | 38 | I | 704 |
| 1582927 | 3597 | 3612 | 824 | 839 | GGGCCCGGCAGGCCGT | 81 | I | 705 |
| 1098912 | 4513 | 4528 | N/A | N/A | AGAGGAGGCGAGCCGC | 27 | J | 301 |
| 1582462 | 4840 | 4855 | N/A | N/A | TGCAAATCTTCTATAG | 55 | J | 706 |
| 1582479 | 3631 | 3646 | 858 | 873 | CGCTTGAGCGGGCCCA | 78 | J | 707 |
| 1582484 | 3638 | 3653 | 865 | 880 | GCGGCCCCGCTTGAGC | 44 | J | 708 |
| 1582489 | 2717 | 2732 | N/A | N/A | AGAGAGAGGCCGGCGG | 43 | J | 709 |
| 1582492 | 3656 | 3671 | 883 | 898 | CAAGCACCCCTTGGCC | 37 | J | 710 |
| 1582499 | 3683 | 3698 | 910 | 925 | ACGGACTCCCCTGGGA | 52 | J | 711 |
| 1582505 | 3692 | 3707 | 919 | 934 | AGCCCCACCACGGACT | 20 | J | 712 |
| 1582512 | 3726 | 3741 | 953 | 968 | CACGCCGCCCCGGCGA | 58 | J | 713 |
| 1582518 | 2728 | 2743 | N/A | N/A | CGGACGCGGGCAGAGA | 37 | J | 714 |
| 1582524 | 3792 | 3807 | 1019 | 1034 | CGCGCGGAGGCGGAGG | 70 | J | 715 |
| 1582530 | 3818 | 3833 | 1045 | 1060 | CCGGGATGCCTTGCAT | 52 | J | 716 |
| 1582538 | 3842 | 3857 | 1069 | 1084 | GCTCCTGGAGCGCCTG | 35 | J | 717 |
| 1582545 | 3856 | 3871 | 1083 | 1098 | AGACCAGGGCGCCGGC | 48 | J | 718 |
| 1582548 | 2738 | 2753 | N/A | N/A | ATTTCACGGACGGACG | 12 | J | 719 |
| 1582554 | 3897 | 3912 | 1124 | 1139 | CTCGCCAGGAGCTCAT | 13 | J | 720 |
| 1582561 | 3908 | 3923 | 1135 | 1150 | GAAACTCCGGGCTCGC | 15 | J | 721 |
| 1582569 | 3929 | 3944 | 1156 | 1171 | GGAGAGGTTGCGCCTG | 18 | J | 722 |
| 1582575 | 3937 | 3952 | 1164 | 1179 | CGTTTCTAGGAGAGGT | 64 | J | 723 |
| 1582578 | 2746 | 2761 | N/A | N/A | CGGCCGGAATTTCACG | 63 | J | 724 |
| 1582585 | 3981 | 3996 | 1208 | 1223 | AGCGAGGCGGCCTCTT | 14 | J | 725 |
| 1582591 | 4011 | 4026 | 1238 | 1253 | CGGTATTCTTCCTCGC | 10 | J | 726 |
| 1582597 | 4019 | 4034 | 1246 | 1261 | GCAGAGCCCGGTATTC | 22 | J | 727 |
| 1582609 | N/A | N/A | 1439 | 1454 | TCTCCGCGGTGTGGAG | 57 | J | 728 |
| 1582616 | 2808 | 2823 | 35 | 50 | CCCCGGGCTTCCGCGG | 69 | J | 729 |
| 1582627 | 4598 | 4613 | N/A | N/A | ATGCTTTTGACCGCCA | 17 | J | 730 |
| 1582633 | 4621 | 4636 | N/A | N/A | AGCGGGCAAAGACAGA | 17 | J | 731 |
| 1582645 | 4666 | 4681 | 1531 | 1546 | TTGCACGTCAGCCGGG | 96 | J | 732 |
| 1582651 | 4688 | 4703 | 1553 | 1568 | ACAGAGAGGCCAGCGA | 17† | J | 733 |
| 1582657 | 4709 | 4724 | 1574 | 1589 | ATTTCACGGAAGAACA | 13† | J | 734 |
| 1582663 | 4716 | 4731 | 1581 | 1596 | AGCCAGAATTTCACGG | 7 | J | 735 |
| 1582670 | 4747 | 4762 | 1612 | 1627 | AGACAGCGTCGGAAGG | 8† | J | 736 |
| 1582676 | 4763 | 4778 | 1628 | 1643 | TAATCCAGGTTTGCCT | 8 | J | 737 |
| 1582682 | 4769 | 4784 | 1634 | 1649 | TAACTCTAATCCAGGT | 18 | J | 738 |
| 1582688 | 4776 | 4791 | 1641 | 1656 | GGAGATGTAACTCTAA | 11 | J | 739 |
| 1582694 | 4787 | 4802 | 1652 | 1667 | CTAATCATCCAGGAGA | 10† | J | 740 |
| 1582700 | 4793 | 4808 | 1658 | 1673 | TCTGAACTAATCATCC | 19† | J | 741 |
| 1582703 | 2834 | 2849 | 61 | 76 | AAACGAGTCTCCGTCG | 13 | J | 742 |
| 1582709 | 2864 | 2879 | 91 | 106 | CTCGCAGGGCCTCGCT | 31 | J | 743 |
| 1582715 | 2875 | 2890 | 102 | 117 | CTCAAAGCAGGCTCGC | 23 | J | 744 |
| 1582721 | 2887 | 2902 | 114 | 129 | GTACGGGTTCCGCTCA | 46 | J | 745 |
| 1582728 | 2933 | 2948 | 160 | 175 | GAATGCCGATGGCCTG | 16 | J | 746 |
| 1582734 | 2953 | 2968 | 180 | 195 | AATCTGGACCCTGGGC | 27 | J | 747 |
| 1582740 | 2960 | 2975 | 187 | 202 | GAAACCAAATCTGGAC | 12 | J | 748 |
| 1582746 | 3003 | 3018 | 230 | 245 | GATTCCCGCCGGTGCT | 14 | J | 749 |
| 1582752 | 3028 | 3043 | 255 | 270 | GCCGCGTCTCCCGGGC | 51 | J | 750 |
| 1582758 | 3061 | 3076 | 288 | 303 | GACGGCGGTCCGCTTT | 14 | J | 751 |
| 1582764 | 3093 | 3108 | 320 | 335 | GCTCGGAGGAGCAGGG | 29 | J | 752 |
| 1582770 | 3102 | 3117 | 329 | 344 | TTCTCAAAGGCTCGGA | 16 | J | 753 |
| 1582776 | 3114 | 3129 | 341 | 356 | GGAAAGCGATCCTTCT | 35 | J | 754 |
| 1582782 | 3120 | 3135 | 347 | 362 | ATGCCTGGAAAGCGAT | 28 | J | 755 |
| 1582788 | 3131 | 3146 | 358 | 373 | CCCGGGCGGCGATGCC | 90 | J | 756 |
| 1582794 | 3161 | 3176 | 388 | 403 | CCGGGAGGCCCGTCTC | 47 | J | 757 |
| 1582800 | 3175 | 3190 | 402 | 417 | CTGAATCCTGGACTCC | 6 | J | 758 |
| 1582806 | 3212 | 3227 | 439 | 454 | GTCCCGGGTGCCTGGC | 45 | J | 759 |
| 1582812 | 3234 | 3249 | 461 | 476 | TGCGCGGGCGCCCTGC | 74 | J | 760 |
| 1582818 | 3259 | 3274 | 486 | 501 | GGCCGCGCTGCACAGG | 56 | J | 761 |
| 1582824 | 3293 | 3308 | 520 | 535 | CGACCCACGAGGGAGC | 36 | J | 762 |
| 1582830 | 3299 | 3314 | 526 | 541 | CGAAGGCGACCCACGA | 28 | J | 763 |
| 1582836 | 3312 | 3327 | 539 | 554 | GCGCCGGTGTGGGCGA | 45 | J | 764 |
| 1582842 | 3318 | 3333 | 545 | 560 | CCCCACGCGCCGGTGT | 20 | J | 765 |
| 1582844 | 2684 | 2699 | N/A | N/A | CCGGACTGTGCACTGC | 61 | J | 766 |
| 1582849 | 3332 | 3347 | 559 | 574 | CGGGAAGCCCCGTTCC | 54 | J | 767 |
| 1582857 | 3354 | 3369 | 581 | 596 | GGCGCGCAGGGCACGT | 46 | J | 768 |
| 1582863 | 3361 | 3376 | 588 | 603 | AGCCCCAGGCGCGCAG | 23 | J | 769 |
| 1582869 | 3378 | 3393 | 605 | 620 | AAAGCCCCCTGTGGGA | 45 | J | 770 |
| 1582876 | 3384 | 3399 | 611 | 626 | CTCACGAAAGCCCCCT | 15 | J | 771 |
| 1582882 | 3436 | 3451 | 663 | 678 | TGCCGGCGCGGCCTGG | 50 | J | 772 |
| 1582889 | 3474 | 3489 | 701 | 716 | AAATCCCCGCGCGCCG | 35 | J | 773 |
| 1582895 | 3482 | 3497 | 709 | 724 | CGTAGGCGAAATCCCC | 17 | J | 774 |
| 1582901 | 3488 | 3503 | 715 | 730 | GGGCGGCGTAGGCGAA | 56 | J | 775 |
| 1582907 | 3508 | 3523 | 735 | 750 | CGCCCCGTCCGGAGGA | 19 | J | 776 |
| 1582913 | N/A | N/A | 769 | 784 | GCGGAGGCCAGCGAGG | 36 | J | 777 |
| 1582919 | 3562 | 3577 | 789 | 804 | CTCCCGGCTTTTGCCC | 31 | J | 778 |
| 1582926 | 3595 | 3610 | 822 | 837 | GCCCGGCAGGCCGTCG | 78 | J | 779 |
| 1098912 | 4513 | 4528 | N/A | N/A | AGAGGAGGCGAGCCGC | 20 | K | 301 |
| 1582461 | 4839 | 4854 | N/A | N/A | GCAAATCTTCTATAGG | 37 | K | 780 |
| 1582478 | 3629 | 3644 | 856 | 871 | CTTGAGCGGGCCCAGG | 20 | K | 781 |
| 1582485 | 3636 | 3651 | 863 | 878 | GGCCCCGCTTGAGCGG | 132 | K | 782 |
| 1582491 | 3654 | 3669 | 881 | 896 | AGCACCCCTTGGCCCT | 22 | K | 783 |
| 1582498 | 3680 | 3695 | 907 | 922 | GACTCCCCTGGGACGT | 22 | K | 784 |
| 1582504 | 3688 | 3703 | 915 | 930 | CCACCACGGACTCCCC | 29 | K | 785 |
| 1582511 | 3725 | 3740 | 952 | 967 | ACGCCGCCCCGGCGAC | 39 | K | 786 |
| 1582517 | 2727 | 2742 | N/A | N/A | GGACGCGGGCAGAGAG | 58 | K | 787 |
| 1582523 | 3787 | 3802 | 1014 | 1029 | GGAGGCGGAGGCGTCC | 32 | K | 788 |
| 1582529 | 3800 | 3815 | 1027 | 1042 | GCCCCTGCCGCGCGGA | 26 | K | 789 |
| 1582537 | 3841 | 3856 | 1068 | 1083 | CTCCTGGAGCGCCTGG | 19 | K | 790 |
| 1582543 | 2737 | 2752 | N/A | N/A | TTTCACGGACGGACGC | 17 | K | 791 |
| 1582544 | 3855 | 3870 | 1082 | 1097 | GACCAGGGCGCCGGCT | 21 | K | 792 |
| 1582553 | 3896 | 3911 | 1123 | 1138 | TCGCCAGGAGCTCATC | 21 | K | 793 |
| 1582560 | 3907 | 3922 | 1134 | 1149 | AAACTCCGGGCTCGCC | 20 | K | 794 |
| 1582567 | 3913 | 3928 | 1140 | 1155 | CTGCAGAAACTCCGGG | 39 | K | 795 |
| 1582568 | 2744 | 2759 | N/A | N/A | GCCGGAATTTCACGGA | 13 | K | 796 |
| 1582574 | 3935 | 3950 | 1162 | 1177 | TTTCTAGGAGAGGTTG | 20 | K | 797 |
| 1582583 | 3970 | 3985 | 1197 | 1212 | CTCTTCCGAGGCCTCC | 22 | K | 798 |
| 1582590 | 4008 | 4023 | 1235 | 1250 | TATTCTTCCTCGCTGA | 17 | K | 799 |
| 1582596 | 4017 | 4032 | 1244 | 1259 | AGAGCCCGGTATTCTT | 16 | K | 800 |
| 1582608 | N/A | N/A | 1438 | 1453 | CTCCGCGGTGTGGAGT | 60 | K | 801 |
| 1582612 | 2784 | 2799 | 11 | 26 | CTGTCCGAGGGTGTCG | 18 | K | 802 |
| 1582626 | 4597 | 4612 | N/A | N/A | TGCTTTTGACCGCCAG | 26 | K | 803 |
| 1582632 | 4620 | 4635 | N/A | N/A | GCGGGCAAAGACAGAC | 40 | K | 804 |
| 1582644 | 4643 | 4658 | N/A | N/A | CTGCGCGCAGGTCTAG | 74 | K | 805 |
| 1582650 | 4686 | 4701 | 1551 | 1566 | AGAGAGGCCAGCGAGC | 7† | K | 806 |
| 1582656 | 4708 | 4723 | 1573 | 1588 | TTTCACGGAAGAACAA | 12† | K | 807 |
| 1582662 | 4715 | 4730 | 1580 | 1595 | GCCAGAATTTCACGGA | 10† | K | 808 |
| 1582669 | 4745 | 4760 | 1610 | 1625 | ACAGCGTCGGAAGGTG | 8† | K | 809 |
| 1582675 | 4752 | 4767 | 1617 | 1632 | TGCCTAGACAGCGTCG | 5† | K | 810 |
| 1582681 | 4768 | 4783 | 1633 | 1648 | AACTCTAATCCAGGTT | 13 | K | 811 |
| 1582687 | 4775 | 4790 | 1640 | 1655 | GAGATGTAACTCTAAT | 19 | K | 812 |
| 1582693 | 4786 | 4801 | 1651 | 1666 | TAATCATCCAGGAGAT | 15 | K | 813 |
| 1582699 | 4792 | 4807 | 1657 | 1672 | CTGAACTAATCATCCA | 12 | K | 814 |
| 1582702 | 2833 | 2848 | 60 | 75 | AACGAGTCTCCGTCGC | 17 | K | 815 |
| 1582708 | 2863 | 2878 | 90 | 105 | TCGCAGGGCCTCGCTT | 37 | K | 816 |
| 1582714 | 2874 | 2889 | 101 | 116 | TCAAAGCAGGCTCGCA | 37 | K | 817 |
| 1582720 | 2886 | 2901 | 113 | 128 | TACGGGTTCCGCTCAA | 17 | K | 818 |
| 1582724 | 2644 | 2659 | N/A | N/A | CCGGACAGCCAGCCAG | 42 | K | 819 |
| 1582727 | 2932 | 2947 | 159 | 174 | AATGCCGATGGCCTGG | 18 | K | 820 |
| 1582733 | 2939 | 2954 | 166 | 181 | GCTCCGGAATGCCGAT | 19 | K | 821 |
| 1582739 | 2959 | 2974 | 186 | 201 | AAACCAAATCTGGACC | 26 | K | 822 |
| 1582745 | 3001 | 3016 | 228 | 243 | TTCCCGCCGGTGCTGC | 19 | K | 823 |
| 1582751 | 3013 | 3028 | 240 | 255 | CCAGGGCCGAGATTCC | 27 | K | 824 |
| 1582757 | 3060 | 3075 | 287 | 302 | ACGGCGGTCCGCTTTC | 22 | K | 825 |
| 1582763 | 3091 | 3106 | 318 | 333 | TCGGAGGAGCAGGGCG | 29 | K | 826 |
| 1582769 | 3101 | 3116 | 328 | 343 | TCTCAAAGGCTCGGAG | 21 | K | 827 |
| 1582775 | 3113 | 3128 | 340 | 355 | GAAAGCGATCCTTCTC | 18 | K | 828 |
| 1582781 | 3119 | 3134 | 346 | 361 | TGCCTGGAAAGCGATC | 18 | K | 829 |
| 1582787 | 3130 | 3145 | 357 | 372 | CCGGGCGGCGATGCCT | 22 | K | 830 |
| 1582793 | 3157 | 3172 | 384 | 399 | GAGGCCCGTCTCTCTG | 23 | K | 831 |
| 1582799 | 3173 | 3188 | 400 | 415 | GAATCCTGGACTCCGG | 21 | K | 832 |
| 1582805 | 3211 | 3226 | 438 | 453 | TCCCGGGTGCCTGGCC | 58 | K | 833 |
| 1582811 | 3233 | 3248 | 460 | 475 | GCGCGGGCGCCCTGCC | 20 | K | 834 |
| 1582817 | 3258 | 3273 | 485 | 500 | GCCGCGCTGCACAGGC | 22 | K | 835 |
| 1582823 | 3292 | 3307 | 519 | 534 | GACCCACGAGGGAGCA | 26 | K | 836 |
| 1582829 | 3298 | 3313 | 525 | 540 | GAAGGCGACCCACGAG | 29 | K | 837 |
| 1582835 | 3311 | 3326 | 538 | 553 | CGCCGGTGTGGGCGAA | 33 | K | 838 |
| 1582841 | 3317 | 3332 | 544 | 559 | CCCACGCGCCGGTGTG | 17 | K | 839 |
| 1582848 | 3329 | 3344 | 556 | 571 | GAAGCCCCGTTCCCCA | 38 | K | 840 |
| 1582856 | 3353 | 3368 | 580 | 595 | GCGCGCAGGGCACGTG | 47 | K | 841 |
| 1582862 | 3360 | 3375 | 587 | 602 | GCCCCAGGCGCGCAGG | 44 | K | 842 |
| 1582868 | 3377 | 3392 | 604 | 619 | AAGCCCCCTGTGGGAG | 22 | K | 843 |
| 1582875 | 3383 | 3398 | 610 | 625 | TCACGAAAGCCCCCTG | 16 | K | 844 |
| 1582881 | 3435 | 3450 | 662 | 677 | GCCGGCGCGGCCTGGC | 29 | K | 845 |
| 1582888 | 3473 | 3488 | 700 | 715 | AATCCCCGCGCGCCGG | 23 | K | 846 |
| 1582894 | 3479 | 3494 | 706 | 721 | AGGCGAAATCCCCGCG | 23 | K | 847 |
| 1582900 | 3487 | 3502 | 714 | 729 | GGCGGCGTAGGCGAAA | 27 | K | 848 |
| 1582906 | 3507 | 3522 | 734 | 749 | GCCCCGTCCGGAGGAG | 23 | K | 849 |
| 1582912 | 3516 | 3531 | 743 | 758 | TGGGAGAGCGCCCCGT | 26 | K | 850 |
| 1582918 | 3560 | 3575 | 787 | 802 | CCCGGCTTTTGCCCGG | 81 | K | 851 |
| 1582921 | 2710 | 2725 | N/A | N/A | GGCCGGCGGGCTCCCG | 48 | K | 852 |
| 1582925 | 3594 | 3609 | 821 | 836 | CCCGGCAGGCCGTCGC | 21 | K | 853 |
| 1098912 | 4513 | 4528 | N/A | N/A | AGAGGAGGCGAGCCGC | 29 | L | 301 |
| 1582460 | 4838 | 4853 | N/A | N/A | CAAATCTTCTATAGGA | 50 | L | 854 |
| 1582466 | 4517 | 4532 | N/A | N/A | GCGCAGAGGAGGCGAG | 95 | L | 855 |
| 1582469 | 2575 | 2590 | N/A | N/A | TCATGAATGGCGGTGA | 45 | L | 856 |
| 1582472 | 2699 | 2714 | N/A | N/A | TCCCGTGCACCTCAGC | 30 | L | 857 |
| 1582477 | 3626 | 3641 | 853 | 868 | GAGCGGGCCCAGGCTG | 47 | L | 858 |
| 1582483 | 3635 | 3650 | 862 | 877 | GCCCCGCTTGAGCGGG | 66 | L | 859 |
| 1582490 | 3653 | 3668 | 880 | 895 | GCACCCCTTGGCCCTG | 30 | L | 860 |
| 1582497 | 3678 | 3693 | 905 | 920 | CTCCCCTGGGACGTGG | 31 | L | 861 |
| 1582503 | 3687 | 3702 | 914 | 929 | CACCACGGACTCCCCT | 27 | L | 862 |
| 1582510 | 3724 | 3739 | 951 | 966 | CGCCGCCCCGGCGACC | 40 | L | 863 |
| 1582516 | 2726 | 2741 | N/A | N/A | GACGCGGGCAGAGAGA | 11 | L | 864 |
| 1582522 | 3786 | 3801 | 1013 | 1028 | GAGGCGGAGGCGTCCG | 40 | L | 865 |
| 1582528 | 3796 | 3811 | 1023 | 1038 | CTGCCGCGCGGAGGCG | 21 | L | 866 |
| 1582535 | 2735 | 2750 | N/A | N/A | TCACGGACGGACGCGG | 23 | L | 867 |
| 1582536 | 3838 | 3853 | 1065 | 1080 | CTGGAGCGCCTGGGAG | 52 | L | 868 |
| 1582542 | 3851 | 3866 | 1078 | 1093 | AGGGCGCCGGCTCCTG | 30 | L | 869 |
| 1582552 | 3895 | 3910 | 1122 | 1137 | CGCCAGGAGCTCATCC | 26 | L | 870 |
| 1582559 | 3906 | 3921 | 1133 | 1148 | AACTCCGGGCTCGCCA | 38 | L | 871 |
| 1582565 | 3912 | 3927 | 1139 | 1154 | TGCAGAAACTCCGGGC | 45 | L | 872 |
| 1582566 | 2743 | 2758 | N/A | N/A | CCGGAATTTCACGGAC | 20 | L | 873 |
| 1582573 | 3934 | 3949 | 1161 | 1176 | TTCTAGGAGAGGTTGC | 18 | L | 874 |
| 1582582 | 3967 | 3982 | 1194 | 1209 | TTCCGAGGCCTCCAGC | 39 | L | 875 |
| 1582589 | 4007 | 4022 | 1234 | 1249 | ATTCTTCCTCGCTGAG | 22 | L | 876 |
| 1582595 | 4016 | 4031 | 1243 | 1258 | GAGCCCGGTATTCTTC | 15 | L | 877 |
| 1582601 | 4033 | 4048 | 1260 | 1275 | CTAAAGCTCCTCCAGC | 31 | L | 878 |
| 1582607 | N/A | N/A | 1436 | 1451 | CCGCGGTGTGGAGTCT | 58 | L | 879 |
| 1582611 | 2783 | 2798 | 10 | 25 | TGTCCGAGGGTGTCGG | 17 | L | 880 |
| 1582625 | 4596 | 4611 | N/A | N/A | GCTTTTGACCGCCAGG | 17 | L | 881 |
| 1582631 | 4609 | 4624 | N/A | N/A | CAGACAGAGGTATGCT | 23 | L | 882 |
| 1582649 | 4679 | 4694 | 1544 | 1559 | CCAGCGAGCTCCCTTG | 23 | L | 883 |
| 1582655 | 4707 | 4722 | 1572 | 1587 | TTCACGGAAGAACAAG | 12 | L | 884 |
| 1582661 | 4714 | 4729 | 1579 | 1594 | CCAGAATTTCACGGAA | 15† | L | 885 |
| 1582666 | 2826 | 2841 | 53 | 68 | CTCCGTCGCCGTCCTC | 28 | L | 886 |
| 1582668 | 4744 | 4759 | 1609 | 1624 | CAGCGTCGGAAGGTGG | 10† | L | 887 |
| 1582674 | 4751 | 4766 | 1616 | 1631 | GCCTAGACAGCGTCGG | 2† | L | 888 |
| 1582680 | 4767 | 4782 | 1632 | 1647 | ACTCTAATCCAGGTTT | 9† | L | 889 |
| 1582686 | 4774 | 4789 | 1639 | 1654 | AGATGTAACTCTAATC | 13† | L | 890 |
| 1582692 | 4783 | 4798 | 1648 | 1663 | TCATCCAGGAGATGTA | 23† | L | 891 |
| 1582698 | 4791 | 4806 | 1656 | 1671 | TGAACTAATCATCCAG | 14† | L | 892 |
| 1582707 | 2838 | 2853 | 65 | 80 | GTCCAAACGAGTCTCC | 41 | L | 893 |
| 1582713 | 2873 | 2888 | 100 | 115 | CAAAGCAGGCTCGCAG | 43 | L | 894 |
| 1582719 | 2885 | 2900 | 112 | 127 | ACGGGTTCCGCTCAAA | 25 | L | 895 |
| 1582726 | 2931 | 2946 | 158 | 173 | ATGCCGATGGCCTGGG | 20 | L | 896 |
| 1582732 | 2938 | 2953 | 165 | 180 | CTCCGGAATGCCGATG | 20 | L | 897 |
| 1582738 | 2958 | 2973 | 185 | 200 | AACCAAATCTGGACCC | 26 | L | 898 |
| 1582744 | 3000 | 3015 | 227 | 242 | TCCCGCCGGTGCTGCC | 22 | L | 899 |
| 1582750 | 3012 | 3027 | 239 | 254 | CAGGGCCGAGATTCCC | 28 | L | 900 |
| 1582756 | 3051 | 3066 | 278 | 293 | CGCTTTCGCCGGCCTT | 18 | L | 901 |
| 1582762 | 3088 | 3103 | 315 | 330 | GAGGAGCAGGGCGGTC | 76 | L | 902 |
| 1582768 | 3100 | 3115 | 327 | 342 | CTCAAAGGCTCGGAGG | 27 | L | 903 |
| 1582774 | 3112 | 3127 | 339 | 354 | AAAGCGATCCTTCTCA | 25 | L | 904 |
| 1582780 | 3118 | 3133 | 345 | 360 | GCCTGGAAAGCGATCC | 28 | L | 905 |
| 1582786 | 3128 | 3143 | 355 | 370 | GGGCGGCGATGCCTGG | 25 | L | 906 |
| 1582792 | 3135 | 3150 | 362 | 377 | TCCTCCCGGGCGGCGA | 78 | L | 907 |
| 1582798 | 3171 | 3186 | 398 | 413 | ATCCTGGACTCCGGGA | 46 | L | 908 |
| 1582804 | 3198 | 3213 | 425 | 440 | GCCCTTCGATTCTGAA | 24 | L | 909 |
| 1582810 | 3232 | 3247 | 459 | 474 | CGCGGGCGCCCTGCCA | 37 | L | 910 |
| 1582816 | 3257 | 3272 | 484 | 499 | CCGCGCTGCACAGGCC | 29 | L | 911 |
| 1582822 | 3291 | 3306 | 518 | 533 | ACCCACGAGGGAGCAG | 34 | L | 912 |
| 1582828 | 3297 | 3312 | 524 | 539 | AAGGCGACCCACGAGG | 16 | L | 913 |
| 1582834 | 3304 | 3319 | 531 | 546 | GTGGGCGAAGGCGACC | 78 | L | 914 |
| 1582840 | 3316 | 3331 | 543 | 558 | CCACGCGCCGGTGTGG | 55 | L | 915 |
| 1582847 | 3328 | 3343 | 555 | 570 | AAGCCCCGTTCCCCAC | 33 | L | 916 |
| 1582854 | 3352 | 3367 | 579 | 594 | CGCGCAGGGCACGTGG | 67 | L | 917 |
| 1582861 | 3359 | 3374 | 586 | 601 | CCCCAGGCGCGCAGGG | 54 | L | 918 |
| 1582867 | 3376 | 3391 | 603 | 618 | AGCCCCCTGTGGGAGA | 39 | L | 919 |
| 1582873 | 3382 | 3397 | 609 | 624 | CACGAAAGCCCCCTGT | 24 | L | 920 |
| 1582880 | 3434 | 3449 | 661 | 676 | CCGGCGCGGCCTGGCT | 51 | L | 921 |
| 1582886 | 3472 | 3487 | 699 | 714 | ATCCCCGCGCGCCGGG | 50 | L | 922 |
| 1582893 | 3478 | 3493 | 705 | 720 | GGCGAAATCCCCGCGC | 47 | L | 923 |
| 1582899 | 3486 | 3501 | 713 | 728 | GCGGCGTAGGCGAAAT | 28 | L | 924 |
| 1582905 | 3506 | 3521 | 733 | 748 | CCCCGTCCGGAGGAGC | 21 | L | 925 |
| 1582911 | 3514 | 3529 | 741 | 756 | GGAGAGCGCCCCGTCC | 23 | L | 926 |
| 1582917 | 3556 | 3571 | 783 | 798 | GCTTTTGCCCGGGTGC | 34 | L | 927 |
| 1582924 | 3593 | 3608 | 820 | 835 | CCGGCAGGCCGTCGCG | 61 | L | 928 |
| 1098912 | 4513 | 4528 | N/A | N/A | AGAGGAGGCGAGCCGC | 20 | M | 301 |
| 1582465 | 4516 | 4531 | N/A | N/A | CGCAGAGGAGGCGAGC | 43 | M | 929 |
| 1582468 | 2574 | 2589 | N/A | N/A | CATGAATGGCGGTGAG | 68 | M | 930 |
| 1582471 | 2690 | 2705 | N/A | N/A | CCTCAGCCGGACTGTG | 35 | M | 931 |
| 1582474 | 4837 | 4852 | N/A | N/A | AAATCTTCTATAGGAT | 55 | M | 932 |
| 1582476 | 3625 | 3640 | 852 | 867 | AGCGGGCCCAGGCTGT | 56 | M | 933 |
| 1582482 | 3634 | 3649 | 861 | 876 | CCCCGCTTGAGCGGGC | 120 | M | 934 |
| 1582488 | 3650 | 3665 | 877 | 892 | CCCCTTGGCCCTGCGG | 27 | M | 935 |
| 1582495 | 3660 | 3675 | 887 | 902 | GGCGCAAGCACCCCTT | 17 | M | 936 |
| 1582502 | 3686 | 3701 | 913 | 928 | ACCACGGACTCCCCTG | 16 | M | 937 |
| 1582509 | 3723 | 3738 | 950 | 965 | GCCGCCCCGGCGACCT | 25 | M | 938 |
| 1582515 | 2725 | 2740 | N/A | N/A | ACGCGGGCAGAGAGAG | 33 | M | 939 |
| 1582521 | 3785 | 3800 | 1012 | 1027 | AGGCGGAGGCGTCCGG | 39 | M | 940 |
| 1582527 | 3795 | 3810 | 1022 | 1037 | TGCCGCGCGGAGGCGG | 20 | M | 941 |
| 1582533 | 3822 | 3837 | 1049 | 1064 | GGCGCCGGGATGCCTT | 45 | M | 942 |
| 1582534 | 2734 | 2749 | N/A | N/A | CACGGACGGACGCGGG | 33 | M | 943 |
| 1582541 | 3849 | 3864 | 1076 | 1091 | GGCGCCGGCTCCTGGA | 58 | M | 944 |
| 1582550 | 3883 | 3898 | 1110 | 1125 | ATCCAGCAGCAGGCCG | 20 | M | 945 |
| 1582557 | 2742 | 2757 | N/A | N/A | CGGAATTTCACGGACG | 13 | M | 946 |
| 1582558 | 3904 | 3919 | 1131 | 1146 | CTCCGGGCTCGCCAGG | 24 | M | 947 |
| 1582564 | 3911 | 3926 | 1138 | 1153 | GCAGAAACTCCGGGCT | 29 | M | 948 |
| 1582572 | 3933 | 3948 | 1160 | 1175 | TCTAGGAGAGGTTGCG | 27 | M | 949 |
| 1582581 | 3966 | 3981 | 1193 | 1208 | TCCGAGGCCTCCAGCT | 49 | M | 950 |
| 1582584 | 2749 | 2764 | N/A | N/A | CCCCGGCCGGAATTTC | 37 | M | 951 |
| 1582588 | 3985 | 4000 | 1212 | 1227 | TTCCAGCGAGGCGGCC | 26 | M | 952 |
| 1582594 | 4014 | 4029 | 1241 | 1256 | GCCCGGTATTCTTCCT | 21 | M | 953 |
| 1582600 | 4032 | 4047 | 1259 | 1274 | TAAAGCTCCTCCAGCA | 24 | M | 954 |
| 1582622 | 2811 | 2826 | 38 | 53 | CGTCCCCGGGCTTCCG | 48 | M | 955 |
| 1582630 | 4608 | 4623 | N/A | N/A | AGACAGAGGTATGCTT | 21 | M | 956 |
| 1582648 | 4678 | 4693 | 1543 | 1558 | CAGCGAGCTCCCTTGC | 25 | M | 957 |
| 1582654 | 4705 | 4720 | 1570 | 1585 | CACGGAAGAACAAGGG | 11 | M | 958 |
| 1582660 | 4713 | 4728 | 1578 | 1593 | CAGAATTTCACGGAAG | 16 | M | 959 |
| 1582667 | 4743 | 4758 | 1608 | 1623 | AGCGTCGGAAGGTGGG | 9† | M | 960 |
| 1582673 | 4750 | 4765 | 1615 | 1630 | CCTAGACAGCGTCGGA | 17† | M | 961 |
| 1582679 | 4766 | 4781 | 1631 | 1646 | CTCTAATCCAGGTTTG | 9 | M | 962 |
| 1582685 | 4773 | 4788 | 1638 | 1653 | GATGTAACTCTAATCC | 21† | M | 963 |
| 1582691 | 4782 | 4797 | 1647 | 1662 | CATCCAGGAGATGTAA | 25 | M | 964 |
| 1582697 | 4790 | 4805 | 1655 | 1670 | GAACTAATCATCCAGG | 12† | M | 965 |
| 1582706 | 2837 | 2852 | 64 | 79 | TCCAAACGAGTCTCCG | 25 | M | 966 |
| 1582712 | 2872 | 2887 | 99 | 114 | AAAGCAGGCTCGCAGG | 32 | M | 967 |
| 1582718 | 2881 | 2896 | 108 | 123 | GTTCCGCTCAAAGCAG | 26 | M | 968 |
| 1582725 | 2929 | 2944 | 156 | 171 | GCCGATGGCCTGGGCC | 34 | M | 969 |
| 1582731 | 2937 | 2952 | 164 | 179 | TCCGGAATGCCGATGG | 31 | M | 970 |
| 1582737 | 2957 | 2972 | 184 | 199 | ACCAAATCTGGACCCT | 23 | M | 971 |
| 1582743 | 2999 | 3014 | 226 | 241 | CCCGCCGGTGCTGCCT | 27 | M | 972 |
| 1582749 | 3011 | 3026 | 238 | 253 | AGGGCCGAGATTCCCG | 52 | M | 973 |
| 1582755 | 3049 | 3064 | 276 | 291 | CTTTCGCCGGCCTTCT | 31 | M | 974 |
| 1582761 | 3085 | 3100 | 312 | 327 | GAGCAGGGCGGTCTGG | 35 | M | 975 |
| 1582767 | 3099 | 3114 | 326 | 341 | TCAAAGGCTCGGAGGA | 18 | M | 976 |
| 1582773 | 3111 | 3126 | 338 | 353 | AAGCGATCCTTCTCAA | 33 | M | 977 |
| 1582779 | 3117 | 3132 | 344 | 359 | CCTGGAAAGCGATCCT | 14 | M | 978 |
| 1582785 | 3127 | 3142 | 354 | 369 | GGCGGCGATGCCTGGA | 17 | M | 979 |
| 1582791 | 3134 | 3149 | 361 | 376 | CCTCCCGGGCGGCGAT | 44 | M | 980 |
| 1582797 | 3165 | 3180 | 392 | 407 | GACTCCGGGAGGCCCG | 36 | M | 981 |
| 1582803 | 3196 | 3211 | 423 | 438 | CCTTCGATTCTGAAAC | 27 | M | 982 |
| 1582809 | 3230 | 3245 | 457 | 472 | CGGGCGCCCTGCCACC | 39 | M | 983 |
| 1582815 | 3252 | 3267 | 479 | 494 | CTGCACAGGCCGCCTG | 49 | M | 984 |
| 1582821 | 3290 | 3305 | 517 | 532 | CCCACGAGGGAGCAGG | 38 | M | 985 |
| 1582827 | 3296 | 3311 | 523 | 538 | AGGCGACCCACGAGGG | 30 | M | 986 |
| 1582833 | 3302 | 3317 | 529 | 544 | GGGCGAAGGCGACCCA | 41 | M | 987 |
| 1582839 | 3315 | 3330 | 542 | 557 | CACGCGCCGGTGTGGG | 23 | M | 988 |
| 1582846 | 3327 | 3342 | 554 | 569 | AGCCCCGTTCCCCACG | 40 | M | 989 |
| 1582852 | 3335 | 3350 | 562 | 577 | GTGCGGGAAGCCCCGT | 35 | M | 990 |
| 1582860 | 3357 | 3372 | 584 | 599 | CCAGGCGCGCAGGGCA | 27 | M | 991 |
| 1582866 | 3373 | 3388 | 600 | 615 | CCCCTGTGGGAGAGCC | 21 | M | 992 |
| 1582872 | 3381 | 3396 | 608 | 623 | ACGAAAGCCCCCTGTG | 29 | M | 993 |
| 1582879 | 3402 | 3417 | 629 | 644 | GCGGCCCTCGCTGCCT | 43 | M | 994 |
| 1582885 | 3455 | 3470 | 682 | 697 | CAGGTTGGGAGATCCC | 22 | M | 995 |
| 1582892 | 3477 | 3492 | 704 | 719 | GCGAAATCCCCGCGCG | 41 | M | 996 |
| 1582898 | 3485 | 3500 | 712 | 727 | CGGCGTAGGCGAAATC | 25 | M | 997 |
| 1582904 | 3504 | 3519 | 731 | 746 | CCGTCCGGAGGAGCCG | 37 | M | 998 |
| 1582910 | 3511 | 3526 | 738 | 753 | GAGCGCCCCGTCCGGA | 84 | M | 999 |
| 1582916 | 3552 | 3567 | 779 | 794 | TTGCCCGGGTGCGGAG | 76 | M | 1000 |
| 1582923 | 3589 | 3604 | 816 | 831 | CAGGCCGTCGCGCTGC | 32 | M | 1001 |
| 1098912 | 4513 | 4528 | N/A | N/A | AGAGGAGGCGAGCCGC | 18 | N | 301 |
| 1582464 | 4514 | 4529 | N/A | N/A | CAGAGGAGGCGAGCCG | 39 | N | 1002 |
| 1582467 | 2573 | 2588 | N/A | N/A | ATGAATGGCGGTGAGC | 56 | N | 1003 |
| 1582470 | 2687 | 2702 | N/A | N/A | CAGCCGGACTGTGCAC | 21 | N | 1004 |
| 1582473 | 4827 | 4842 | 1692 | 1707 | TAGGATCCACAGGGAG | 32 | N | 1005 |
| 1582475 | 3623 | 3638 | 850 | 865 | CGGGCCCAGGCTGTGC | 90 | N | 1006 |
| 1582481 | 3633 | 3648 | 860 | 875 | CCCGCTTGAGCGGGCC | 38 | N | 1007 |
| 1582487 | 3640 | 3655 | 867 | 882 | CTGCGGCCCCGCTTGA | 42 | N | 1008 |
| 1582494 | 3659 | 3674 | 886 | 901 | GCGCAAGCACCCCTTG | 31 | N | 1009 |
| 1582501 | 3685 | 3700 | 912 | 927 | CCACGGACTCCCCTGG | 37 | N | 1010 |
| 1582507 | 3722 | 3737 | 949 | 964 | CCGCCCCGGCGACCTG | 36 | N | 1011 |
| 1582508 | 2724 | 2739 | N/A | N/A | CGCGGGCAGAGAGAGG | 76 | N | 1012 |
| 1582514 | 3729 | 3744 | 956 | 971 | TCCCACGCCGCCCCGG | 24 | N | 1013 |
| 1582520 | 2730 | 2745 | N/A | N/A | GACGGACGCGGGCAGA | 42 | N | 1014 |
| 1582526 | 3794 | 3809 | 1021 | 1036 | GCCGCGCGGAGGCGGA | 38 | N | 1015 |
| 1582532 | 3821 | 3836 | 1048 | 1063 | GCGCCGGGATGCCTTG | 61 | N | 1016 |
| 1582540 | 3848 | 3863 | 1075 | 1090 | GCGCCGGCTCCTGGAG | 73 | N | 1017 |
| 1582547 | 3858 | 3873 | 1085 | 1100 | GCAGACCAGGGCGCCG | 43 | N | 1018 |
| 1582551 | 2741 | 2756 | N/A | N/A | GGAATTTCACGGACGG | 31 | N | 1019 |
| 1582556 | 3902 | 3917 | 1129 | 1144 | CCGGGCTCGCCAGGAG | 39 | N | 1020 |
| 1582563 | 3910 | 3925 | 1137 | 1152 | CAGAAACTCCGGGCTC | 29 | N | 1021 |
| 1582571 | 3932 | 3947 | 1159 | 1174 | CTAGGAGAGGTTGCGC | 80 | N | 1022 |
| 1582577 | 3940 | 3955 | 1167 | 1182 | CTCCGTTTCTAGGAGA | 60 | N | 1023 |
| 1582580 | 2748 | 2763 | N/A | N/A | CCCGGCCGGAATTTCA | 38 | N | 1024 |
| 1582587 | 3983 | 3998 | 1210 | 1225 | CCAGCGAGGCGGCCTC | 32 | N | 1025 |
| 1582593 | 4013 | 4028 | 1240 | 1255 | CCCGGTATTCTTCCTC | 19 | N | 1026 |
| 1582599 | 4022 | 4037 | 1249 | 1264 | CCAGCAGAGCCCGGTA | 31 | N | 1027 |
| 1582618 | 2810 | 2825 | 37 | 52 | GTCCCCGGGCTTCCGC | 27 | N | 1028 |
| 1582629 | 4605 | 4620 | N/A | N/A | CAGAGGTATGCTTTTG | 28 | N | 1029 |
| 1582635 | 4623 | 4638 | N/A | N/A | GAAGCGGGCAAAGACA | 33 | N | 1030 |
| 1582647 | 4677 | 4692 | 1542 | 1557 | AGCGAGCTCCCTTGCA | 37 | N | 1031 |
| 1582653 | 4704 | 4719 | 1569 | 1584 | ACGGAAGAACAAGGGC | 33 | N | 1032 |
| 1582659 | 4712 | 4727 | 1577 | 1592 | AGAATTTCACGGAAGA | 19 | N | 1033 |
| 1582665 | 4723 | 4738 | 1588 | 1603 | GACATTCAGCCAGAAT | 15† | N | 1034 |
| 1582672 | 4749 | 4764 | 1614 | 1629 | CTAGACAGCGTCGGAA | 9† | N | 1035 |
| 1582678 | 4765 | 4780 | 1630 | 1645 | TCTAATCCAGGTTTGC | 10† | N | 1036 |
| 1582684 | 4772 | 4787 | 1637 | 1652 | ATGTAACTCTAATCCA | 12† | N | 1037 |
| 1582690 | 4781 | 4796 | 1646 | 1661 | ATCCAGGAGATGTAAC | 11† | N | 1038 |
| 1582696 | 4789 | 4804 | 1654 | 1669 | AACTAATCATCCAGGA | 18† | N | 1039 |
| 1582705 | 2836 | 2851 | 63 | 78 | CCAAACGAGTCTCCGT | 14 | N | 1040 |
| 1582711 | 2871 | 2886 | 98 | 113 | AAGCAGGCTCGCAGGG | 71 | N | 1041 |
| 1582717 | 2880 | 2895 | 107 | 122 | TTCCGCTCAAAGCAGG | 25 | N | 1042 |
| 1582723 | 2917 | 2932 | 144 | 159 | GGCCAGCCGTTCTCTG | 51 | N | 1043 |
| 1582730 | 2936 | 2951 | 163 | 178 | CCGGAATGCCGATGGC | 49 | N | 1044 |
| 1582736 | 2956 | 2971 | 183 | 198 | CCAAATCTGGACCCTG | 18 | N | 1045 |
| 1582742 | 2986 | 3001 | 213 | 228 | CCTCAGCTGGCGTGAC | 21 | N | 1046 |
| 1582748 | 3010 | 3025 | 237 | 252 | GGGCCGAGATTCCCGC | 32 | N | 1047 |
| 1582754 | 3047 | 3062 | 274 | 289 | TTCGCCGGCCTTCTGG | 27 | N | 1048 |
| 1582760 | 3069 | 3084 | 296 | 311 | GATCCGGTGACGGCGG | 33 | N | 1049 |
| 1582766 | 3098 | 3113 | 325 | 340 | CAAAGGCTCGGAGGAG | 31 | N | 1050 |
| 1582772 | 3110 | 3125 | 337 | 352 | AGCGATCCTTCTCAAA | 19 | N | 1051 |
| 1582778 | 3116 | 3131 | 343 | 358 | CTGGAAAGCGATCCTT | 12 | N | 1052 |
| 1582784 | 3126 | 3141 | 353 | 368 | GCGGCGATGCCTGGAA | 32 | N | 1053 |
| 1582790 | 3133 | 3148 | 360 | 375 | CTCCCGGGCGGCGATG | 41 | N | 1054 |
| 1582796 | 3163 | 3178 | 390 | 405 | CTCCGGGAGGCCCGTC | 19 | N | 1055 |
| 1582802 | 3195 | 3210 | 422 | 437 | CTTCGATTCTGAAACC | 22 | N | 1056 |
| 1582808 | 3229 | 3244 | 456 | 471 | GGGCGCCCTGCCACCC | 51 | N | 1057 |
| 1582814 | 3236 | 3251 | 463 | 478 | CCTGCGCGGGCGCCCT | 38 | N | 1058 |
| 1582820 | 3281 | 3296 | 508 | 523 | GAGCAGGGTGACCCCC | 25 | N | 1059 |
| 1582826 | 3295 | 3310 | 522 | 537 | GGCGACCCACGAGGGA | 50 | N | 1060 |
| 1582832 | 3301 | 3316 | 528 | 543 | GGCGAAGGCGACCCAC | 22 | N | 1061 |
| 1582838 | 3314 | 3329 | 541 | 556 | ACGCGCCGGTGTGGGC | 19 | N | 1062 |
| 1582845 | 3326 | 3341 | 553 | 568 | GCCCCGTTCCCCACGC | 22 | N | 1063 |
| 1582851 | 3334 | 3349 | 561 | 576 | TGCGGGAAGCCCCGTT | 31 | N | 1064 |
| 1582859 | 3356 | 3371 | 583 | 598 | CAGGCGCGCAGGGCAC | 29 | N | 1065 |
| 1582865 | 3363 | 3378 | 590 | 605 | AGAGCCCCAGGCGCGC | 36 | N | 1066 |
| 1582871 | 3380 | 3395 | 607 | 622 | CGAAAGCCCCCTGTGG | 34 | N | 1067 |
| 1582878 | 3386 | 3401 | 613 | 628 | GGCTCACGAAAGCCCC | 33 | N | 1068 |
| 1582884 | 3450 | 3465 | 677 | 692 | TGGGAGATCCCCTCTG | 31 | N | 1069 |
| 1582891 | 3476 | 3491 | 703 | 718 | CGAAATCCCCGCGCGC | 31 | N | 1070 |
| 1582897 | 3484 | 3499 | 711 | 726 | GGCGTAGGCGAAATCC | 17 | N | 1071 |
| 1582903 | 3503 | 3518 | 730 | 745 | CGTCCGGAGGAGCCGG | 30 | N | 1072 |
| 1582909 | 3510 | 3525 | 737 | 752 | AGCGCCCCGTCCGGAG | 50 | N | 1073 |
| 1582915 | 3551 | 3566 | 778 | 793 | TGCCCGGGTGCGGAGG | 83 | N | 1074 |
| 1582922 | 3587 | 3602 | 814 | 829 | GGCCGTCGCGCTGCGG | 51 | N | 1075 |
| 1604077 | 3917 | 3932 | 1144 | 1159 | CCTGCTGCAGAAACTC | 24 | O | 1076 |
| 1604078 | 3926 | 3941 | 1153 | 1168 | GAGGTTGCGCCTGCTG | 12 | O | 1077 |
| 1604079 | 3927 | 3942 | 1154 | 1169 | AGAGGTTGCGCCTGCT | 9 | O | 1078 |
| 1604080 | 4010 | 4025 | 1237 | 1252 | GGTATTCTTCCTCGCT | 4 | O | 1079 |
| 1604081 | N/A | N/A | 1433 | 1448 | CGGTGTGGAGTCTCTC | 30 | O | 1080 |
| 1604082 | N/A | N/A | 1437 | 1452 | TCCGCGGTGTGGAGTC | 10 | O | 1081 |
| 1604083 | N/A | N/A | 1441 | 1456 | GTTCTCCGCGGTGTGG | 15 | O | 1082 |
| 1604084 | N/A | N/A | 1442 | 1457 | AGTTCTCCGCGGTGTG | 11 | O | 1083 |
| 1604085 | N/A | N/A | 1443 | 1458 | CAGTTCTCCGCGGTGT | 11 | O | 1084 |
| 1604086 | N/A | N/A | 1444 | 1459 | GCAGTTCTCCGCGGTG | 7 | O | 1085 |
| 1604087 | N/A | N/A | 1445 | 1460 | GGCAGTTCTCCGCGGT | 11 | O | 1086 |
| 1604088 | N/A | N/A | 1447 | 1462 | ATGGCAGTTCTCCGCG | 6 | O | 1087 |
| 1604089 | 2785 | 2800 | 12 | 27 | GCTGTCCGAGGGTGTC | 17 | O | 1088 |
| 1604090 | 2788 | 2803 | 15 | 30 | GGTGCTGTCCGAGGGT | 25 | O | 1089 |
| 1604091 | 2791 | 2806 | 18 | 33 | GAGGGTGCTGTCCGAG | 29 | O | 1090 |
| 1604092 | 2792 | 2807 | 19 | 34 | GGAGGGTGCTGTCCGA | 50 | O | 1091 |
| 1604093 | 4532 | 4547 | N/A | N/A | GACGGTGGCGCGGGGG | 61 | O | 1092 |
| 1604094 | 4559 | 4574 | N/A | N/A | AGGCTGCAGGGGCCCG | 27 | O | 1093 |
| 1604096 | 4599 | 4614 | N/A | N/A | TATGCTTTTGACCGCC | 7 | O | 1094 |
| 1604097 | 4601 | 4616 | N/A | N/A | GGTATGCTTTTGACCG | 30 | O | 1095 |
| 1604098 | 4624 | 4639 | N/A | N/A | GGAAGCGGGCAAAGAC | 142 | O | 1096 |
| 1604106 | 4644 | 4659 | N/A | N/A | ACTGCGCGCAGGTCTA | 22 | O | 1097 |
| 1604107 | 4645 | 4660 | 1510 | 1525 | CACTGCGCGCAGGTCT | 6 | O | 1098 |
| 1604108 | 4654 | 4669 | 1519 | 1534 | CGGGGTGCGCACTGCG | 36 | O | 1099 |
| 1604109 | 4655 | 4670 | 1520 | 1535 | CCGGGGTGCGCACTGC | 24 | O | 1100 |
| 1604110 | 4656 | 4671 | 1521 | 1536 | GCCGGGGTGCGCACTG | 70 | O | 1101 |
| 1604111 | 4662 | 4677 | 1527 | 1542 | ACGTCAGCCGGGGTGC | 52 | O | 1102 |
| 1604112 | 4663 | 4678 | 1528 | 1543 | CACGTCAGCCGGGGTG | 21 | O | 1103 |
| 1604113 | 4664 | 4679 | 1529 | 1544 | GCACGTCAGCCGGGGT | 49 | O | 1104 |
| 1604114 | 4667 | 4682 | 1532 | 1547 | CTTGCACGTCAGCCGG | 11 | O | 1105 |
| 1604115 | 4668 | 4683 | 1533 | 1548 | CCTTGCACGTCAGCCG | 4 | O | 1106 |
| 1604116 | 4669 | 4684 | 1534 | 1549 | CCCTTGCACGTCAGCC | 8 | O | 1107 |
| 1604117 | 4670 | 4685 | 1535 | 1550 | TCCCTTGCACGTCAGC | 12 | O | 1108 |
| 1604118 | 4672 | 4687 | 1537 | 1552 | GCTCCCTTGCACGTCA | 12 | O | 1109 |
| 1604119 | 4674 | 4689 | 1539 | 1554 | GAGCTCCCTTGCACGT | 15 | O | 1110 |
| 1604120 | 4675 | 4690 | 1540 | 1555 | CGAGCTCCCTTGCACG | 23 | O | 1111 |
| 1604121 | 4676 | 4691 | 1541 | 1556 | GCGAGCTCCCTTGCAC | 10 | O | 1112 |
| 1604122 | 4685 | 4700 | 1550 | 1565 | GAGAGGCCAGCGAGCT | 14† | O | 1113 |
| 1604123 | 4687 | 4702 | 1552 | 1567 | CAGAGAGGCCAGCGAG | 20† | O | 1114 |
| 1604124 | 4724 | 4739 | 1589 | 1604 | AGACATTCAGCCAGAA | N.D.† | O | 1115 |
| 1604125 | 4727 | 4742 | 1592 | 1607 | GGGAGACATTCAGCCA | 3† | O | 1116 |
| 1604126 | 4728 | 4743 | 1593 | 1608 | GGGGAGACATTCAGCC | 22† | O | 1117 |
| 1604127 | 2825 | 2840 | 52 | 67 | TCCGTCGCCGTCCTCG | 7 | O | 1118 |
| 1604128 | 4741 | 4756 | 1606 | 1621 | CGTCGGAAGGTGGGGG | 15† | O | 1119 |
| 1604129 | 4742 | 4757 | 1607 | 1622 | GCGTCGGAAGGTGGGG | 11† | O | 1120 |
| 1604130 | 4746 | 4761 | 1611 | 1626 | GACAGCGTCGGAAGGT | N.D.† | O | 1121 |
| 1604131 | 4756 | 4771 | 1621 | 1636 | GGTTTGCCTAGACAGC | 23† | O | 1122 |
| 1604132 | 4759 | 4774 | 1624 | 1639 | CCAGGTTTGCCTAGAC | 9† | O | 1123 |
| 1604133 | 4760 | 4775 | 1625 | 1640 | TCCAGGTTTGCCTAGA | N.D.† | O | 1124 |
| 1604134 | 4800 | 4815 | 1665 | 1680 | ATATATCTCTGAACTA | 27 | O | 1125 |
| 1604135 | 4801 | 4816 | 1666 | 1681 | AATATATCTCTGAACT | 19 | O | 1126 |
| 1604136 | 4813 | 4828 | 1678 | 1693 | AGGGGGCATTTTAATA | 87 | O | 1127 |
| 1604137 | 4814 | 4829 | 1679 | 1694 | GAGGGGGCATTTTAAT | 131 | O | 1128 |
| 1604138 | 2867 | 2882 | 94 | 109 | AGGCTCGCAGGGCCTC | 47 | O | 1129 |
| 1604139 | 2882 | 2897 | 109 | 124 | GGTTCCGCTCAAAGCA | 8 | O | 1130 |
| 1604140 | 2969 | 2984 | 196 | 211 | TCTCATTCTGAAACCA | 7 | O | 1131 |
| 1604141 | 2975 | 2990 | 202 | 217 | GTGACCTCTCATTCTG | 27 | O | 1132 |
| 1604142 | 2981 | 2996 | 208 | 223 | GCTGGCGTGACCTCTC | 5 | O | 1133 |
| 1604143 | 2993 | 3008 | 220 | 235 | GGTGCTGCCTCAGCTG | 4 | O | 1134 |
| 1604144 | 3103 | 3118 | 330 | 345 | CTTCTCAAAGGCTCGG | 23 | O | 1135 |
| 1604145 | 3105 | 3120 | 332 | 347 | TCCTTCTCAAAGGCTC | 7 | O | 1136 |
| 1604146 | 3137 | 3152 | 364 | 379 | GCTCCTCCCGGGCGGC | N.D. | O | 1137 |
| 1604147 | 3145 | 3160 | 372 | 387 | TCTGGCCAGCTCCTCC | 11 | O | 1138 |
| 1604148 | 3151 | 3166 | 378 | 393 | CGTCTCTCTGGCCAGC | 8 | O | 1139 |
| 1604149 | 3152 | 3167 | 379 | 394 | CCGTCTCTCTGGCCAG | 11 | O | 1140 |
| 1604150 | 3174 | 3189 | 401 | 416 | TGAATCCTGGACTCCG | 8 | O | 1141 |
| 1604151 | 3180 | 3195 | 407 | 422 | CAGATCTGAATCCTGG | 18 | O | 1142 |
| 1604152 | 3181 | 3196 | 408 | 423 | CCAGATCTGAATCCTG | 10 | O | 1143 |
| 1604153 | 3193 | 3208 | 420 | 435 | TCGATTCTGAAACCAG | 7 | O | 1144 |
| 1604154 | 3206 | 3221 | 433 | 448 | GGTGCCTGGCCCTTCG | N.D. | O | 1145 |

TABLE 13
Reduction of DUX4 RNA by 3-10-3 cEt modified oligonucleotides with uniform phosphorothioate
internucleoside linkages in differentiated 54-2 cells

| | SEQ ID | SEQ ID | | | | |
|---|---|---|---|---|---|---|
| | NO: 3 | NO: 3 | | | | SEQ |
| Compound | Start | Stop | | DUX4 | Analysis | ID |
| No. | Site | Site | Sequence (5′ to 3′) | (% UTC) | ID | NO |
| 1582604 | 7320 | 7335 | AAAGAGAGGCCACGGC | 25 | I | 1146 |
| 1582621 | 7823 | 7838 | TGACCGCCAGGAGCTC | 27 | I | 1147 |
| 1582603 | 7319 | 7334 | AAGAGAGGCCACGGCC | 25 | J | 1148 |
| 1582620 | 7821 | 7836 | ACCGCCAGGAGCTCCG | 32 | J | 1149 |
| 1582602 | 7310 | 7325 | CACGGCCCTGCCCCGA | 48 | K | 1150 |
| 1582619 | 7820 | 7835 | CCGCCAGGAGCTCCGC | 12 | K | 1151 |
| 1582606 | 7322 | 7337 | CGAAAGAGAGGCCACG | 19 | M | 1152 |
| 1582624 | 7827 | 7842 | CTTTTGACCGCCAGGA | 20 | M | 1153 |
| 1582605 | 7321 | 7336 | GAAAGAGAGGCCACGG | 35 | N | 1154 |
| 1582623 | 7825 | 7840 | TTTGACCGCCAGGAGC | 27 | N | 1155 |
| 1604095 | 7824 | 7839 | TTGACCGCCAGGAGCT | 6 | o | 1156 |

The compounds in the table below have a single mismatch to SEQ ID NO: 1 (described herein above) located at the position indicated in the column labeled “Position of mismatch on Compound (5′ to 3′)”. Additionally, the mismatched nucleobase is marked as bold and underlined in the column labeled “Sequence (5′ to 3′).”

TABLE 14
Reduction of DUX4 RNA by 3-10-3 cEt modified oligonucleotides with uniform phosphorothioate
internucleoside linkages in differentiated 54-2 cells

| | Position of | SEQ | | | | | |
|---|---|---|---|---|---|---|---|
| | mismatch | ID | SEQ ID | | | | |
| | on | NO: 1 | NO: 1 | | DUX4 | | |
| Compound | Compound | Start | Stop | | (% | Analysis | SEQ ID |
| No. | (5′ to 3′) | Site | Site | Sequence (5′ to 3′) | UTC) | ID | NO |
| 1582640 | 10 | 4636 | 4651 | CAGGTCTAGTCAGGAA | 31 | I | 1157 |
| 1582639 | 7 | 4633 | 4648 | GTCTAGTCAGGAAGCG | 35 | J | 1158 |
| 1582638 | 5 | 4631 | 4646 | CTAGTCAGGAAGCGGG | 65 | K | 1159 |
| 1582637 | 4 | 4630 | 4645 | TAGTCAGGAAGCGGGC | 38 | L | 1160 |
| 1582643 | 16 | 4642 | 4657 | TGCGCGCAGGTCTAGT | 77 | L | 1161 |
| 1582636 | 3 | 4629 | 4644 | AGTCAGGAAGCGGGCA | 54 | M | 1162 |
| 1582642 | 14 | 4640 | 4655 | CGCGCAGGTCTAGTCA | 81 | M | 1163 |
| 1582641 | 13 | 4639 | 4654 | GCGCAGGTCTAGTCAG | 46 | N | 1164 |
| 1604099 | 2 | 4628 | 4643 | GTCAGGAAGCGGGCAA | 25 | O | 1165 |
| 1604100 | 6 | 4632 | 4647 | TCTAGTCAGGAAGCGG | 9 | O | 1166 |
| 1604101 | 8 | 4634 | 4649 | GGTCTAGTCAGGAAGC | 32 | O | 1167 |
| 1604102 | 9 | 4635 | 4650 | AGGTCTAGTCAGGAAG | 26 | O | 1168 |
| 1604103 | 11 | 4637 | 4652 | GCAGGTCTAGTCAGGA | 21 | O | 1169 |
| 1604104 | 12 | 4638 | 4653 | CGCAGGTCTAGTCAGG | 8 | O | 1170 |
| 1604105 | 15 | 4641 | 4656 | GCGCGCAGGTCTAGTC | 58 | O | 1171 |

### Example 8: Dose-Dependent Inhibition of Human DUX4 in 54-2 Cells by Modified Oligonucleotides

Modified oligonucleotides selected from the example above were tested at various doses in 54-2 cells (described herein above). 54-2 cells plated at a density of 20,000 cells per well were differentiated (as described herein above) and treated using cytofectin with various concentrations of modified oligonucleotide as specified in the table below. After a treatment period of approximately 24 hours, total RNA was isolated from the cells and DUX4 RNA levels were measured by quantitative real-time RTPCR. Human DUX4 primer-probe set RTS40199 (described herein above) was used to measure RNA levels as described above. DUX4 RNA levels were normalized to total RNA content, as measured by RIBOGREEN®. Reduction of DUX4 RNA is presented in the table below as percent DUX4 RNA relative to the amount of DUX4 RNA in untreated control cells (% UTC).

The half maximal inhibitory concentration (IC₅₀) of each modified oligonucleotide was calculated using a linear regression on a log/linear plot of the data in Excel and is also presented in the table below. “N.D.” in the table below refers to instances where the value was Not Defined.

TABLE 15
Dose-dependent reduction of human DUX4 RNA
in 54-2 cells by modified oligonucleotides

| Compound | DMPK RNA (% UTC) | | | | IC₅₀ |
|---|---|---|---|---|---|
| No. | 15 nM | 44 nM | 133 nM | 400 nM | (μM) |
| 1582502 | 72 | 49 | 4 | 4 | 0.04 |
| 1582516 | 104 | 27 | 4 | 2 | 0.05 |
| 1582548 | 80 | 27 | 2 | 1 | 0.03 |
| 1582554 | 132 | 40 | 7 | 2 | 0.07 |
| 1582557 | 83 | 32 | 3 | 3 | 0.03 |
| 1582561 | 88 | 39 | 5 | 2 | 0.04 |
| 1582562 | 96 | 49 | 5 | 2 | 0.05 |
| 1582568 | 82 | 37 | 5 | 3 | 0.04 |
| 1582585 | 63 | 29 | 3 | 8 | 0.02 |
| 1582590 | 102 | 99 | 17 | 5 | 0.09 |
| 1582591 | 107 | 42 | 8 | 5 | 0.06 |
| 1582595 | 86 | 47 | 7 | 1 | 0.04 |
| 1582596 | 106 | 59 | 14 | 3 | 0.07 |
| 1582598 | 89 | 40 | 5 | 3 | 0.04 |
| 1582619 | 84 | 44 | 10 | 8 | 0.04 |
| 1582654 | 56 | 9 | 3 | 1 | <0.02 |

TABLE 16
Dose-dependent reduction of human DUX4 RNA
in 54-2 cells by modified oligonucleotides

| Compound | DMPK RNA (% UTC) | | | | IC₅₀ |
|---|---|---|---|---|---|
| No. | 15 nM | 44 nM | 133 nM | 400 nM | (μM) |
| 1582728 | 75 | 46 | 11 | 2 | 0.04 |
| 1582740 | 131 | 71 | 15 | 3 | 0.09 |
| 1582746 | 84 | 42 | 8 | 7 | 0.04 |
| 1582758 | 105 | 56 | 20 | 14 | 0.07 |
| 1582770 | 86 | 56 | 9 | 4 | 0.05 |
| 1582771 | 85 | 53 | 7 | 3 | 0.05 |
| 1582778 | 82 | 51 | 14 | 3 | 0.05 |
| 1582779 | 74 | 37 | 6 | 2 | 0.03 |
| 1582785 | 26 | 27 | 11 | 13 | <0.02 |
| 1582800 | 99 | 36 | 10 | 3 | 0.05 |
| 1582801 | 84 | 45 | 5 | 4 | 0.04 |
| 1582828 | 92 | 53 | 23 | 8 | 0.06 |
| 1582875 | 84 | 56 | 16 | 2 | 0.05 |
| 1582876 | 92 | 83 | 18 | 4 | 0.08 |
| 1582877 | 79 | 47 | 11 | 1 | 0.04 |

TABLE 17
Dose-dependent reduction of human DUX4 RNA
in 54-2 cells by modified oligonucleotides

| | | |
|---|---|---|
| Compound | DMPK RNA (% UTC) | IC₅₀ |

| No. | 6 nM | 25 nM | 100 nM | 400 nM | (μM) |
|---|---|---|---|---|---|
| 1604078 | 28 | 15 | 4 | 10 | <0.01 |
| 1604079 | 36 | 20 | 7 | 4 | <0.01 |
| 1604080 | 34 | 10 | 5 | 16 | <0.01 |
| 1604082 | 47 | 17 | 10 | 7 | <0.01 |
| 1604084 | 29 | 7 | 8 | 14 | <0.01 |
| 1604085 | 23 | 5 | 6 | 8 | <0.01 |
| 1604086 | 13 | 2 | 3 | 4 | <0.01 |
| 1604087 | 22 | 4 | 5 | 11 | <0.01 |
| 1604088 | 28 | 29 | 6 | 9 | <0.01 |
| 1604095 | 108 | 49 | 20 | 26 | 0.05 |
| 1604096 | 57 | 27 | 8 | 17 | 0.01 |
| 1604100 | 70 | 26 | 28 | 14 | 0.01 |
| 1604104 | 57 | 23 | 25 | 27 | <0.01 |
| 1604107 | 45 | 17 | 2 | 1 | <0.01 |
| 1604114 | 28 | 19 | 12 | 25 | <0.01 |

TABLE 18
Dose-dependent reduction of human DUX4 RNA
in 54-2 cells by modified oligonucleotides

| | | |
|---|---|---|
| Compound | DMPK RNA (% UTC) | IC₅₀ |

| No. | 6 nM | 25 nM | 100 nM | 400 nM | (μM) |
|---|---|---|---|---|---|
| 1604115 | 38 | 6 | 2 | 15 | <0.01 |
| 1604116 | 35 | 10 | 5 | 11 | <0.01 |
| 1604121 | 65 | 16 | 4 | 80 | <0.01 |
| 1604127 | 19 | 6 | 0 | 28 | >0.4 |
| 1604139 | 18 | 3 | 8 | 8 | <0.01 |
| 1604140 | 48 | 6 | 1 | 2 | <0.01 |
| 1604142 | 5 | 2 | 3 | 13 | >0.4 |
| 1604143 | 12 | 9 | 1 | 4 | <0.01 |
| 1604145 | 38 | 25 | 2 | 3 | <0.01 |
| 1604147 | 27 | 3 | 15 | 11 | <0.01 |
| 1604148 | 9 | 12 | 3 | 10 | <0.01 |
| 1604149 | 18 | 3 | 5 | 6 | <0.01 |
| 1604150 | 53 | 15 | 8 | 33 | <0.01 |
| 1604152 | 19 | 2 | 4 | 2 | <0.01 |
| 1604153 | 2 | 1 | 3 | 2 | >0.4 |

TABLE 19
Dose-dependent reduction of human DUX4 RNA
in 54-2 cells by modified oligonucleotides

| Compound | DMPK RNA (% UTC) | | | | IC₅₀ |
|---|---|---|---|---|---|
| No. | 15 nM | 44 nM | 133 nM | 400 nM | (μM) |
| 1582655 | 55 | 26 | 4 | 1 | 0.02 |
| 1582660 | 61 | 45 | 6 | 3 | 0.03 |
| 1582663 | 29 | 11 | 2 | N.D. | <0.02 |
| 1582664 | 70 | 14 | 2 | 2 | 0.02 |
| 1582676 | 85 | 31 | 16 | 1 | 0.04 |
| 1582679 | 57 | 30 | 3 | 2 | 0.02 |
| 1582681 | 43 | 24 | 7 | 4 | <0.02 |
| 1582683 | 48 | 22 | 5 | N.D. | <0.02 |
| 1582688 | 102 | 27 | 5 | 2 | 0.05 |
| 1582689 | 43 | 15 | 5 | 10 | <0.02 |
| 1582693 | 84 | 36 | 10 | 2 | 0.04 |
| 1582695 | 55 | 25 | 4 | 2 | <0.02 |
| 1582699 | 108 | 26 | 5 | N.D. | 0.04 |
| 1582703 | 74 | 22 | 2 | 4 | 0.02 |
| 1582705 | 92 | 28 | 6 | 2 | 0.04 |
| 1582722 | 49 | 15 | 5 | 6 | <0.02 |

### Example 9: Design of an RNAi Compound that Targets a Human DUX4 Nucleic Acid

RNAi compounds comprising antisense RNAi oligonucleotides complementary to a human DUX4 nucleic acid, and sense RNAi oligonucleotides complementary to the antisense RNAi oligonucleotides were designed as follows.

“Start site” indicates the 5′-most nucleoside to which the antisense RNAi oligonucleotide is complementary in the human gene sequence. “Stop site” indicates the 3′-most nucleoside to which the antisense RNAi oligonucleotide is complementary in the human gene sequence. Each antisense RNAi oligonucleotide listed in the table below is 100% complementary to SEQ ID NO: 1 (described herein above), and to SEQ ID NO: 2 (described herein above).

The antisense RNAi oligonucleotide in the table below is 25 nucleosides in length; wherein the sugar motif for the antisense RNAi oligonucleotide is (from 5′ to 3′): rrrrrrrrrrrrrrrrrrrrrrrrr, wherein each “r” represents a ribosyl sugar moiety. The internucleoside linkage motif for the antisense RNAi oligonucleotides is (from 5′ to 3′): oooooooooooooooooooooooo, wherein each “o” represents a phosphodiester internucleoside linkage.

The sense RNAi oligonucleotide in the table below is 25 nucleosides in length; wherein the sugar motif for the antisense RNAi oligonucleotide is (from 5′ to 3′): rrrrrrrrrrrrrrrrrrrrrrrrr, wherein each “r” represents a ribosyl sugar moiety. The internucleoside linkage motif for the antisense RNAi oligonucleotides is (from 5′ to 3′): oooooooooooooooooooooooo, wherein each “o” represents a phosphodiester internucleoside linkage.

The sense RNAi oligonucleotide is 100% complementary to the antisense RNAi oligonucleotide (from 5′ to 3′).

TABLE 20
Design of an RNAi compound targeted to human DUX4

| | | | | SEQ | SEQ | SEQ | SEQ | | | |
|---|---|---|---|---|---|---|---|---|---|---|
| | | | | ID NO: | ID NO: | ID NO: | ID NO: | Sense | | |
| | Antisense | Antisense | | 1 Anti- | 1 Anti- | 2 Anti- | 2 Anti- | Strand | | |
| Duplex | Strand | strand | SEQ | sense | sense | sense | sense | Com- | Sense strand | |
| Compound | Compound | Sequence | ID | Start | Stop | Start | Stop | pound | Sequence | SEQ |
| Number | Number | (5′ to 3′) | NO | Site | Site | Site | Site | Number | (5′ to 3′) | ID NO |
| 1586340 | 1586145 | AAAGCGAU | 1172 | 3103 | 3127 | 330 | 354 | 1586338 | CCGAGCC | 1173 |
| | | CCUUCUCA | | | | | | | UUUGAGA | |
| | | AAGGCUCG | | | | | | | AGGAUCG | |
| | | G | | | | | | | CUUU | |

### Example 10: Design of an RNAi Compound that Targets a Human DUX4 Nucleic Acid

RNAi compounds comprising antisense RNAi oligonucleotides complementary to a human DUX4 nucleic acid, and sense RNAi oligonucleotides complementary to the antisense RNAi oligonucleotides were designed as follows.

“Start site” indicates the 5′-most nucleoside to which the antisense RNAi oligonucleotide is complementary in the human gene sequence. “Stop site” indicates the 3′-most nucleoside to which the antisense RNAi oligonucleotide is complementary in the human gene sequence. Each antisense RNAi oligonucleotide listed in the table below is 100% complementary to SEQ ID NO: 1 (described herein above), and to SEQ ID NO: 2 (described herein above).

The antisense RNAi oligonucleotide in the table below is 19 nucleosides in length; wherein the sugar motif for the antisense RNAi oligonucleotide is (from 5′ to 3′): rrrrrrrrrrrrrrrrrrrrrrrrr, wherein each “r” represents a ribosyl sugar moiety. The internucleoside linkage motif for the antisense RNAi oligonucleotides is (from 5′ to 3′): oooooooooooooooooo, wherein each “o” represents a phosphodiester internucleoside linkage.

The sense RNAi oligonucleotide in the table below is 19 nucleosides in length; wherein the sugar motif for the antisense RNAi oligonucleotide is (from 5′ to 3′): rrrrrrrrrrrrrrrrrrrrrrrrr, wherein each “r” represents a ribosyl sugar moiety. The internucleoside linkage motif for the antisense RNAi oligonucleotides is (from 5′ to 3′): oooooooooooooooooo, wherein each “o” represents a phosphodiester internucleoside linkage.

The sense RNAi oligonucleotide is 100% complementary to the antisense RNAi oligonucleotide (from 5′ to 3′).

TABLE 21
Design of an RNAi compound targeted to human DUX4

| | | | | SEQ | SEQ | SEQ | SEQ | | | |
|---|---|---|---|---|---|---|---|---|---|---|
| | | | | ID NO: | ID NO: | ID NO: | ID NO: | Sense | | |
| | Antisense | Antisense | | 1 Anti- | 1 Anti- | 2 Anti- | 2 Anti- | Strand | | |
| Duplex | Strand | strand | SEQ | sense | sense | sense | sense | Com- | Sense strand | SEQ |
| Compound | Compound | Sequence | ID | Start | Stop | Start | Stop | pound | Sequence | ID |
| Number | Number | (5′ to 3′) | NO | Site | Site | Site | Site | Number | (5′ to 3′) | NO |
| 1586341 | 1586147 | UUCCGCUC | 1174 | 2877 | 2895 | 104 | 122 | 1586339 | GAGCCUG | 1175 |
| | | AAAGCAG | | | | | | | CUUUGAG | |
| | | GCUC | | | | | | | CGGAA | |

### Example 11: Design of an RNAi Compound that Targets a Human DUX4 Nucleic Acid

RNAi compounds comprising antisense RNAi oligonucleotides complementary to a human DUX4 nucleic acid, and sense RNAi oligonucleotides complementary to the antisense RNAi oligonucleotides were designed as follows.

“Start site” indicates the 5′-most nucleoside to which the antisense RNAi oligonucleotide is complementary in the human gene sequence. “Stop site” indicates the 3′-most nucleoside to which the antisense RNAi oligonucleotide is complementary in the human gene sequence. Each antisense RNAi oligonucleotide listed in the table below is 100% complementary to SEQ ID NO: 1 (described herein above), to SEQ ID NO: 2 (described herein above), or to both. “N/A” indicates that the antisense RNAi oligonucleotide is not 100% complementary to that particular target nucleic acid sequence

The antisense RNAi oligonucleotide in the table below is 23 nucleosides in length; wherein the sugar motif for the antisense RNAi oligonucleotide is (from 5′ to 3′ yfyfyfyfyfyfyfyfyfyfyyy, wherein each “y” represents a 2′-O-methylribosyl sugar moiety, and each “f” represents a 2′-fluororibosyl sugar moiety. The internucleoside linkage motif for the antisense RNAi oligonucleotides is (from 5′ to 3′ ssooooooooooooooooooss, wherein each “o” represents a phosphodiester internucleoside linkage, and each “s” represents a phosphorothioate internucleoside linkage.

The sense RNAi oligonucleotide in the table below is 21 nucleosides in length; wherein the sugar motif for the antisense RNAi oligonucleotide is (from 5′ to 3′): fyfyfyfyfyfyfyfyfyfyf, wherein each “y” represents a 2′-O-methylribosyl sugar moiety, and each “f” represents a 2′-fluororibosyl sugar moiety. The internucleoside linkage motif for the antisense RNAi oligonucleotides is (from 5′ to 3′): ssooooooooooooooooss, wherein each “o” represents a phosphodiester internucleoside linkage, and each “s” represents a phosphorothioate internucleoside linkage.

Each sense RNAi oligonucleotide is complementary to the first of the 21 nucleosides of the antisense RNAi oligonucleotide (from 5′ to 3′) wherein the last two 3′-nucleosides of the antisense RNAi oligonucleotides are unpaired overhanging nucleosides.

TABLE 22
Design of RNAi compounds targeted to human DUX4, SEQ ID NO: 1 and/or SEQ ID NO: 2

| | | | | SEQ | SEQ | SEQ | SEQ | | | |
|---|---|---|---|---|---|---|---|---|---|---|
| | | | | ID NO: | ID NO: | ID NO: | ID NO: | Sense | | |
| | Antisense | Antisense | | 1 Anti- | 1 Anti- | 2 Anti- | 2 Anti- | Strand | | |
| Duplex | Strand | strand | SEQ | sense | sense | sense | sense | Com- | Sense strand | SEQ |
| Compound | Compound | Sequence | ID | Start | Stop | Start | Stop | pound | Sequence | ID |
| Number | Number | (5′ to 3′) | NO | Site | Site | Site | Site | Number | (5′ to 3′) | NO |
| 1588121 | 1588119 | UUUAAUA | 1176 | 4797 | 4819 | 1662 | 1684 | 1588120 | UUAGUUC | 1242 |
| | | UAUCUCU | | | | | | | AGAGAUA | |
| | | GAACUAA | | | | | | | UAUUAAA | |
| | | UC | | | | | | | | |
| 1588124 | 1588122 | AUCUCUG | 1177 | 4789 | 4811 | 1654 | 1676 | 1588123 | CUGGAUG | 1243 |
| | | AACUAAU | | | | | | | AUUAGUU | |
| | | CAUCCAG | | | | | | | CAGAGAU | |
| | | GA | | | | | | | | |
| 1588127 | 1588125 | ACUAAUC | 1178 | 4781 | 4803 | 1646 | 1668 | 1588126 | UACAUCU | 1244 |
| | | AUCCAGG | | | | | | | CCUGGAU | |
| | | AGAUGUA | | | | | | | GAUUAGU | |
| | | AC | | | | | | | | |
| 1588130 | 1588128 | UCCAGGA | 1179 | 4773 | 4795 | 1638 | 1660 | 1588129 | AUUAGAG | 1245 |
| | | GAUGUAA | | | | | | | UUACAUC | |
| | | CUCUAAU | | | | | | | UCCUGGA | |
| | | CC | | | | | | | | |
| 1588133 | 1588131 | AUGUAAC | 1180 | 4765 | 4787 | 1630 | 1652 | 1588132 | AAACCUG | 1246 |
| | | UCUAAUC | | | | | | | GAUUAGA | |
| | | CAGGUUU | | | | | | | GUUACAU | |
| | | GC | | | | | | | | |
| 1588142 | 1588140 | UAGACAG | 1181 | 4741 | 4763 | 1606 | 1628 | 1588141 | CCCACCU | 1247 |
| | | CGUCGGA | | | | | | | UCCGACG | |
| | | AGGUGGG | | | | | | | CUGUCUA | |
| | | GG | | | | | | | | |
| 1588151 | 1588149 | AGACAUU | 1182 | 4717 | 4739 | 1582 | 1604 | 1588150 | UGAAAUU | 1248 |
| | | CAGCCAG | | | | | | | CUGGCUG | |
| | | AAUUUCA | | | | | | | AAUGUCU | |
| | | CG | | | | | | | | |
| 1588154 | 1588152 | AGCCAGA | 1183 | 4709 | 4731 | 1574 | 1596 | 1588153 | UUCUUCC | 1249 |
| | | AUUUCAC | | | | | | | GUGAAAU | |
| | | GGAAGAA | | | | | | | UCUGGCU | |
| | | CA | | | | | | | | |
| 1588157 | 1588155 | UUUCACG | 1184 | 4701 | 4723 | 1566 | 1588 | 1588156 | UGCCCUU | 1250 |
| | | GAAGAAC | | | | | | | GUUCUUC | |
| | | AAGGGCA | | | | | | | CGUGAAA | |
| | | CA | | | | | | | | |
| 1588160 | 1588158 | AAGAACA | 1185 | 4693 | 4715 | 1558 | 1580 | 1588159 | CCUCUCU | 1251 |
| | | AGGGCAC | | | | | | | GUGCCCU | |
| | | AGAGAGG | | | | | | | UGUUCUU | |
| | | CC | | | | | | | | |
| 1588166 | 1588164 | AGAGGCC | 1186 | 4677 | 4699 | 1542 | 1564 | 1588165 | CAAGGGA | 1252 |
| | | AGCGAGC | | | | | | | GCUCGCU | |
| | | UCCCUUG | | | | | | | GGCCUCU | |
| | | CA | | | | | | | | |
| 1588190 | 1588188 | UGGGCCG | 1187 | 4251 | 4273 | 1478 | 1500 | 1588189 | GGGGAUC | 1253 |
| | | GCUCUGG | | | | | | | CCAGAGC | |
| | | GAUCCCC | | | | | | | CGGCCCA | |
| | | GG | | | | | | | | |
| 1588196 | 1588194 | UCCCCGG | 1188 | 4235 | 4257 | 1462 | 1484 | 1588195 | CUUUCCU | 1254 |
| | | GAUGCCC | | | | | | | GGGCAUC | |
| | | AGGAAAG | | | | | | | CCGGGGA | |
| | | AA | | | | | | | | |
| 1588226 | 1588224 | UCCUCCA | 1189 | 4019 | 4041 | 1246 | 1268 | 1588225 | AUACCGG | 1255 |
| | | GCAGAGC | | | | | | | GCUCUGC | |
| | | CCGGUAU | | | | | | | UGGAGGA | |
| | | UC | | | | | | | | |
| 1588235 | 1588233 | UCCUCGC | 1190 | 3995 | 4017 | 1222 | 1244 | 1588234 | GGAAGCA | 1256 |
| | | UGAGGGG | | | | | | | CCCCUCA | |
| | | UGCUUCC | | | | | | | GCGAGGA | |
| | | AG | | | | | | | | |
| 1588250 | 1588248 | AGGCCUC | 1191 | 3955 | 3977 | 1182 | 1204 | 1588249 | CCCCGGG | 1257 |
| | | CAGCUCC | | | | | | | GGAGCUG | |
| | | CCCGGGG | | | | | | | GAGGCCU | |
| | | CC | | | | | | | | |
| 1588253 | 1588251 | AGCUCCC | 1192 | 3947 | 3969 | 1174 | 1196 | 1588252 | AACGGAG | 1258 |
| | | CCGGGGC | | | | | | | GCCCCGG | |
| | | CUCCGUU | | | | | | | GGGAGCU | |
| | | UC | | | | | | | | |
| 1588262 | 1588260 | AGGAGAG | 1193 | 3923 | 3945 | 1150 | 1172 | 1588261 | GCAGCAG | 1259 |
| | | GUUGCGC | | | | | | | GCGCAAC | |
| | | CUGCUGC | | | | | | | CUCUCCU | |
| | | AG | | | | | | | | |
| 1588265 | 1588263 | UUGCGCC | 1194 | 3915 | 3937 | 1142 | 1164 | 1588264 | GAGUUUC | 1260 |
| | | UGCUGCA | | | | | | | UGCAGCA | |
| | | GAAACUC | | | | | | | GGCGCAA | |
| | | CG | | | | | | | | |
| 1588271 | 1588269 | AACUCCG | 1195 | 3899 | 3921 | 1126 | 1148 | 1588270 | GCUCCUG | 1261 |
| | | GGCUCGC | | | | | | | GCGAGCC | |
| | | CAGGAGC | | | | | | | CGGAGUU | |
| | | UC | | | | | | | | |
| 1588280 | 1588278 | UCCAGCA | 1196 | 3875 | 3897 | 1102 | 1124 | 1588279 | CCCCUGC | 1262 |
| | | GCAGGCC | | | | | | | GGCCUGC | |
| | | GCAGGGG | | | | | | | UGCUGGA | |
| | | AG | | | | | | | | |
| 1588286 | 1588284 | AGGGGAG | 1197 | 3859 | 3881 | 1086 | 1108 | 1588285 | CGCCCUG | 1263 |
| | | UGCAGAC | | | | | | | GUCUGCA | |
| | | CAGGGCG | | | | | | | CUCCCCU | |
| | | CC | | | | | | | | |
| 1588298 | 1588296 | AGCGCCU | 1198 | 3827 | 3849 | 1054 | 1076 | 1588297 | CCCGGCG | 1264 |
| | | GGGAGGG | | | | | | | CCCUCCC | |
| | | CGCCGGG | | | | | | | AGGCGCU | |
| | | AU | | | | | | | | |
| 1588328 | 1588326 | AGGUGGA | 1199 | 3747 | 3769 | 974 | 996 | 1588327 | CAAGCCG | 1265 |
| | | GCUGCCC | | | | | | | GGGCAGC | |
| | | CGGCUUG | | | | | | | UCCACCU | |
| | | GG | | | | | | | | |
| 1588338 | 1588336 | UUCCCAC | 1200 | 3723 | 3745 | 950 | 972 | 1588337 | GUCGCCG | 1266 |
| | | GCCGCCC | | | | | | | GGGCGGC | |
| | | CGGCGAC | | | | | | | GUGGGAA | |
| | | CU | | | | | | | | |
| 1588383 | 1588381 | UGCCACC | 1201 | 3603 | 3625 | 830 | 852 | 1588382 | CCGGGCC | 1267 |
| | | GCGCAGG | | | | | | | CCUGCGC | |
| | | GGCCCGG | | | | | | | GGUGGCA | |
| | | CA | | | | | | | | |
| 1588398 | 1588396 | UCCCGGU | 1202 | 3563 | 3585 | 790 | 812 | 1588397 | CAAAAGC | 1268 |
| | | CCUCCCG | | | | | | | CGGGAGG | |
| | | GCUUUUG | | | | | | | ACCGGGA | |
| | | CC | | | | | | | | |
| 1588416 | 1588414 | UGAGGGU | 1203 | 3515 | 3537 | 742 | 764 | 1588415 | CGGGGCG | 1269 |
| | | GGGAGAG | | | | | | | CUCUCCC | |
| | | CGCCCCG | | | | | | | ACCCUCA | |
| | | UC | | | | | | | | |
| 1588434 | 1588432 | AAAUCCC | 1204 | 3467 | 3489 | 694 | 716 | 1588433 | UGCCCCG | 1270 |
| | | CGCGCGC | | | | | | | GCGCGCG | |
| | | CGGGGCA | | | | | | | GGGAUUU | |
| | | GG | | | | | | | | |
| 1588443 | 1588441 | UGGGAGA | 1205 | 3443 | 3465 | 670 | 692 | 1588442 | GCCGGCA | 1271 |
| | | UCCCCUC | | | | | | | GAGGGGA | |
| | | UGCCGGC | | | | | | | UCUCCCA | |
| | | GC | | | | | | | | |
| 1588470 | 1588468 | AAAGCCC | 1206 | 3371 | 3393 | 598 | 620 | 1588469 | GGCUCUC | 1272 |
| | | CCUGUGG | | | | | | | CCACAGG | |
| | | GAGAGCC | | | | | | | GGGCUUU | |
| | | CC | | | | | | | | |
| 1588494 | 1588492 | ACGCGCC | 1207 | 3307 | 3329 | 534 | 556 | 1588493 | CCUUCGC | 1273 |
| | | GGUGUGG | | | | | | | CCACACC | |
| | | GCGAAGG | | | | | | | GGCGCGU | |
| | | CG | | | | | | | | |
| 1588524 | 1588522 | UGCGCGG | 1208 | 3227 | 3249 | 454 | 476 | 1588523 | GGGUGGC | 1274 |
| | | GCGCCCU | | | | | | | AGGGCGC | |
| | | GCCACCC | | | | | | | CCGCGCA | |
| | | UG | | | | | | | | |
| 1588539 | 1588537 | UUCGAUU | 1209 | 3187 | 3209 | 414 | 436 | 1588538 | AGAUCUG | 1275 |
| | | CUGAAAC | | | | | | | GUUUCAG | |
| | | CAGAUCU | | | | | | | AAUCGAA | |
| | | GA | | | | | | | | |
| 1588542 | 1588540 | UGAAACC | 1210 | 3179 | 3201 | 406 | 428 | 1588541 | CAGGAUU | 1276 |
| | | AGAUCUG | | | | | | | CAGAUCU | |
| | | AAUCCUG | | | | | | | GGUUUCA | |
| | | GA | | | | | | | | |
| 1588548 | 1588546 | UCCUGGA | 1211 | 3163 | 3185 | 390 | 412 | 1588547 | CGGGCCU | 1277 |
| | | CUCCGGG | | | | | | | CCCGGAG | |
| | | AGGCCCG | | | | | | | UCCAGGA | |
| | | UC | | | | | | | | |
| 1588551 | 1588549 | UCCGGGA | 1212 | 3155 | 3177 | 382 | 404 | 1588550 | CAGAGAG | 1278 |
| | | GGCCCGU | | | | | | | ACGGGCC | |
| | | CUCUCUG | | | | | | | UCCCGGA | |
| | | GC | | | | | | | | |
| 1588560 | 1588558 | AGCUCCU | 1213 | 3131 | 3153 | 358 | 380 | 1588559 | CAUCGCC | 1279 |
| | | CCCGGGC | | | | | | | GCCCGGG | |
| | | GGCGAUG | | | | | | | AGGAGCU | |
| | | CC | | | | | | | | |
| 1588572 | 1588570 | AUCCUUC | 1214 | 3099 | 3121 | 326 | 348 | 1588571 | CUCCGAG | 1280 |
| | | UCAAAGG | | | | | | | CCUUUGA | |
| | | CUCGGAG | | | | | | | GAAGGAU | |
| | | GA | | | | | | | | |
| 1588584 | 1588582 | UCUGGGA | 1215 | 3067 | 3089 | 294 | 316 | 1588583 | CCGCCGU | 1281 |
| | | UCCGGUG | | | | | | | CACCGGA | |
| | | ACGGCGG | | | | | | | UCCCAGA | |
| | | UC | | | | | | | | |
| 1588599 | 1588597 | UGGCGGG | 1216 | 3027 | 3049 | 254 | 276 | 1588598 | CCCGGGA | 1282 |
| | | CCGCGUC | | | | | | | GACGCGG | |
| | | UCCCGGG | | | | | | | CCCGCCA | |
| | | CC | | | | | | | | |
| 1588611 | 1588609 | AUUCCCG | 1217 | 2995 | 3017 | 222 | 244 | 1588610 | UGAGGCA | 1283 |
| | | CCGGUGC | | | | | | | GCACCGG | |
| | | UGCCUCA | | | | | | | CGGGAAU | |
| | | GC | | | | | | | | |
| 1588629 | 1588627 | AAAUCUG | 1218 | 2947 | 2969 | 174 | 196 | 1588628 | CGGAGCC | 1284 |
| | | GACCCUG | | | | | | | CAGGGUC | |
| | | GGCUCCG | | | | | | | CAGAUUU | |
| | | GA | | | | | | | | |
| 1588632 | 1588630 | ACCCUGG | 1219 | 2939 | 2961 | 166 | 188 | 1588631 | CGGCAUU | 1285 |
| | | GCUCCGG | | | | | | | CCGGAGC | |
| | | AAUGCCG | | | | | | | CCAGGGU | |
| | | AU | | | | | | | | |
| 1588638 | 1588636 | UGCCGAU | 1220 | 2923 | 2945 | 150 | 172 | 1588637 | GGCUGGC | 1286 |
| | | GGCCUGG | | | | | | | CCAGGCC | |
| | | GCCAGCC | | | | | | | AUCGGCA | |
| | | GU | | | | | | | | |
| 1588659 | 1588657 | UCAAAGC | 1221 | 2867 | 2889 | 94 | 116 | 1588658 | GGCCCUG | 1287 |
| | | AGGCUCG | | | | | | | CGAGCCU | |
| | | CAGGGCC | | | | | | | GCUUUGA | |
| | | UC | | | | | | | | |
| 1588674 | 1588672 | AAACGAG | 1222 | 2827 | 2849 | 54 | 76 | 1588673 | GACGGCG | 1288 |
| | | UCUCCGU | | | | | | | ACGGAGA | |
| | | CGCCGUC | | | | | | | CUCGUUU | |
| | | CU | | | | | | | | |
| 1588692 | 1588690 | UGCUGUC | 1223 | 2779 | 2801 | 6 | 28 | 1588691 | UCCCGAC | 1289 |
| | | CGAGGGU | | | | | | | ACCCUCG | |
| | | GUCGGGA | | | | | | | GACAGCA | |
| | | GG | | | | | | | | |
| 1588710 | 1588708 | AUUUCAC | 1224 | 2731 | 2753 | N/A | N/A | 1588709 | GCCCGCG | 1290 |
| | | GGACGGA | | | | | | | UCCGUCC | |
| | | CGCGGGC | | | | | | | GUGAAAU | |
| | | AG | | | | | | | | |
| 1588749 | 1588747 | AGCCAGC | 1225 | 2623 | 2645 | N/A | N/A | 1588748 | AGGGCGG | 1291 |
| | | CAGCCAG | | | | | | | CUGGCUG | |
| | | CCGCCCU | | | | | | | GCUGGCU | |
| | | UG | | | | | | | | |
| 1588752 | 1588750 | AGCCAGC | 1226 | 2615 | 2637 | N/A | N/A | 1588751 | CCUUUAC | 1292 |
| | | CGCCCUU | | | | | | | AAGGGCG | |
| | | GUAAAGG | | | | | | | GCUGGCU | |
| | | CC | | | | | | | | |
| 1588758 | 1588756 | AGGGGGC | 1227 | 4806 | 4828 | 1671 | 1693 | 1588757 | AGAUAUA | 1293 |
| | | AUUUUAA | | | | | | | UUAAAAU | |
| | | UAUAUCU | | | | | | | GCCCCCU | |
| | | CU | | | | | | | | |
| 1588764 | 1588762 | AAUCCAG | 1228 | 4755 | 4777 | 1620 | 1642 | 1588763 | CUGUCUA | 1294 |
| | | GUUUGCC | | | | | | | GGCAAAC | |
| | | UAGACAG | | | | | | | CUGGAUU | |
| | | CG | | | | | | | | |
| 1588767 | 1588765 | ACAGCGU | 1229 | 4738 | 4760 | 1603 | 1625 | 1588766 | CCCCCCA | 1295 |
| | | CGGAAGG | | | | | | | CCUUCCG | |
| | | UGGGGGG | | | | | | | ACGCUGU | |
| | | AG | | | | | | | | |
| 1588776 | 1588774 | AAGGGCA | 1230 | 4687 | 4709 | 1552 | 1574 | 1588775 | CGCUGGC | 1296 |
| | | CAGAGAG | | | | | | | CUCUCUG | |
| | | GCCAGCG | | | | | | | UGCCCUU | |
| | | AG | | | | | | | | |
| 1588779 | 1588777 | AGCGAGC | 1231 | 4670 | 4692 | 1535 | 1557 | 1588778 | UGACGUG | 1297 |
| | | UCCCUUG | | | | | | | CAAGGGA | |
| | | CACGUCA | | | | | | | GCUCGCU | |
| | | GC | | | | | | | | |
| 1588788 | 1588786 | UGGGAUC | 1232 | 4240 | 4262 | 1467 | 1489 | 1588787 | CUGGGCA | 1298 |
| | | CCCGGGA | | | | | | | UCCCGGG | |
| | | UGCCCAG | | | | | | | GAUCCCA | |
| | | GA | | | | | | | | |
| 1588794 | 1588792 | UUCUCCG | 1233 | 4206 | 4228 | N/A | N/A | 1588793 | GAGACUC | 1299 |
| | | CGGAGUG | | | | | | | CACUCCG | |
| | | GAGUCUC | | | | | | | CGGAGAA | |
| | | UC | | | | | | | | |
| 1588797 | 1588795 | UCUCUCA | 1234 | 4189 | 4211 | 1416 | 1438 | 1588796 | AGGUCUA | 1300 |
| | | CCGGGCC | | | | | | | GGCCCGG | |
| | | UAGACCU | | | | | | | UGAGAGA | |
| | | AG | | | | | | | | |
| 1588800 | 1588798 | ACCUAGA | 1235 | 4172 | 4194 | 1399 | 1421 | 1588799 | UGGGAUU | 1301 |
| | | AGGCAGG | | | | | | | CCUGCCU | |
| | | AAUCCCA | | | | | | | UCUAGGU | |
| | | GG | | | | | | | | |
| 1588812 | 1588810 | UAGCCAG | 1236 | 4104 | 4126 | 1331 | 1353 | 1588811 | CGGGGAA | 1302 |
| | | CCAGGUG | | | | | | | CACCUGG | |
| | | UUCCCCG | | | | | | | CUGGCUA | |
| | | CG | | | | | | | | |
| 1588181 | 1588179 | ACUGCGC | 1237 | N/A | N/A | 1502 | 1524 | 1588180 | ACCAGCA | 1303 |
| | | GCAGGUC | | | | | | | GACCUGC | |
| | | UGCUGGU | | | | | | | GCGCAGU | |
| | | AC | | | | | | | | |
| 1588199 | 1588197 | AUGCCCA | 1238 | N/A | N/A | 1454 | 1476 | 1588198 | CUGCCAU | 1304 |
| | | GGAAAGA | | | | | | | UCUUUCC | |
| | | AUGGCAG | | | | | | | UGGGCAU | |
| | | UU | | | | | | | | |
| 1588208 | 1588206 | UCCGCGG | 1239 | N/A | N/A | 1430 | 1452 | 1588207 | UGAGAGA | 1305 |
| | | UGUGGAG | | | | | | | CUCCACA | |
| | | UCUCUCA | | | | | | | CCGCGGA | |
| | | CC | | | | | | | | |
| 1588404 | 1588402 | UUUUGCC | 1240 | N/A | N/A | 774 | 796 | 1588403 | GGCCUCC | 1306 |
| | | CGGGUGC | | | | | | | GCACCCG | |
| | | GGAGGCC | | | | | | | GGCAAAA | |
| | | AG | | | | | | | | |
| 1588410 | 1588408 | AGGCCAG | 1241 | N/A | N/A | 758 | 780 | 1588409 | CCUCAGG | 1307 |
| | | CGAGGAG | | | | | | | CUCCUCG | |
| | | CCUGAGG | | | | | | | CUGGCCU | |
| | | GU | | | | | | | | |

“Start site” indicates the 5′-most nucleoside to which the antisense RNAi oligonucleotide is complementary in the human gene sequence. “Stop site” indicates the 3′-most nucleoside to which the antisense RNAi oligonucleotide is complementary in the human gene sequence. The antisense RNAi oligonucleotide listed in the table below is 100% complementary to SEQ ID NO: 4 (GENBANK Accession No. NM_001293798.2).

TABLE 23
Design of RNAi compounds targeted to human DUX4, SEQ ID NO: 4

| | Antisense | Antisense | | SEQ ID | SEQ ID | Sense | Sense | |
|---|---|---|---|---|---|---|---|---|
| Duplex | Strand | Strand | | NO: 4 | NO: 4 | Strand | Strand | SEQ |
| Compound | Compound | Sequence | SEQ | Antisense | Antisense | Compound | Sequence | ID |
| Number | Number | (5′ to 3′) | ID NO | Start Site | Stop Site | Number | (5′ to 3′) | NO |
| 1588214 | 1588212 | UCUCACCG | 1308 | 1278 | 1300 | 1588213 | CGGGGUC | 1309 |
| | | GGCCUAGA | | | | | UAGGCCC | |
| | | CCCCGCG | | | | | GGUGAGA | |

### Example 12: Design of an RNAi Compound that Targets a Human DUX4 Nucleic Acid

RNAi compounds comprising antisense RNAi oligonucleotides complementary to a human DUX4 nucleic acid, and sense RNAi oligonucleotides complementary to the antisense RNAi oligonucleotides were designed as follows.

“Start site” indicates the 5′-most nucleoside to which the antisense RNAi oligonucleotide is complementary in the human gene sequence. “Stop site” indicates the 3′-most nucleoside to which the antisense RNAi oligonucleotide is complementary in the human gene sequence. Each antisense RNAi oligonucleotide listed in the table below is complementary to SEQ ID NO: 1 (described herein above), to SEQ ID NO: 2 (described herein above), or to both, with the exception of a single mismatch at position 1 (from 5′ to 3′) of the antisense RNAi oligonucleotide. ‘N/A’ indicates that the modified oligonucleotide has two or more mismatches to that particular target nucleic acid sequence in the table below.

The antisense RNAi oligonucleotide in the table below is 23 nucleosides in length; wherein the sugar motif for the antisense RNAi oligonucleotide is (from 5′ to 3′ yfyfyfyfyfyfyfyfyfyfyyy, wherein each “y” represents a 2′-O-methylribosyl sugar moiety, and each “f” represents a 2′-fluororibosyl sugar moiety. The internucleoside linkage motif for the antisense RNAi oligonucleotides is (from 5′ to 3′ ssooooooooooooooooooss, wherein each “o” represents a phosphodiester internucleoside linkage, and each “s” represents a phosphorothioate internucleoside linkage.

The sense RNAi oligonucleotide in the table below is 21 nucleosides in length; wherein the sugar motif for the antisense RNAi oligonucleotide is (from 5′ to 3′): 2fyfyfyfyfyfyfyfyfyf, wherein each “y” represents a methylribosyl sugar moiety, and each “f” represents a 2′-fluororibosyl sugar moiety. The internucleoside linkage motif for the antisense RNAi oligonucleotides is (from 5′ to 3′): ssooooooooooooooooss, wherein each “o” represents a phosphodiester internucleoside linkage, and each “s” represents a phosphorothioate internucleoside linkage.

Each sense RNAi oligonucleotide is complementary to the first of the 21 nucleosides of the antisense RNAi oligonucleotide (from 5′ to 3′) wherein the last two 3′-nucleosides of the antisense RNAi oligonucleotides are unpaired overhanging nucleosides.

TABLE 24
Design of RNAi compounds targeted to human DUX4, SEQ ID NO: 1 and/or SEQ ID NO: 2

| | | | | SEQ | SEQ | SEQ | SEQ | | | |
|---|---|---|---|---|---|---|---|---|---|---|
| | | | | ID NO: | ID NO: | ID NO: | ID NO: | Sense | | |
| | Antisense | Antisense | | 1 Anti- | 1 Anti- | 2 Anti- | 2 Anti- | Strand | | |
| Duplex | Strand | strand | SEQ | sense | sense | sense | sense | Com- | Sense strand | SEQ |
| Compound | Compound | Sequence | ID | Start | Stop | Start | Stop | pound | Sequence | ID |
| Number | Number | (5′ to 3′) | NO | Site | Site | Site | Site | Number | (5′ to 3′) | NO |
| 1588136 | 1588134 | AUAAUCC | 1310 | 4757 | 4778 | 1622 | 1643 | 1588135 | GUCUAGG | 1474 |
| | | AGGUUUG | | | | | | | CAAACCU | |
| | | CCUAGAC | | | | | | | GGAUUAU | |
| | | AG | | | | | | | | |
| 1588139 | 1588137 | AGUUUGC | 1311 | 4749 | 4770 | 1614 | 1635 | 1588138 | CCGACGC | 1475 |
| | | CUAGACA | | | | | | | UGUCUAG | |
| | | GCGUCGG | | | | | | | GCAAACU | |
| | | AA | | | | | | | | |
| 1588145 | 1588143 | AUCGGAA | 1312 | 4733 | 4754 | 1598 | 1619 | 1588144 | UGUCUCC | 1476 |
| | | GGUGGGG | | | | | | | CCCCACC | |
| | | GGAGACA | | | | | | | UUCCGAU | |
| | | UU | | | | | | | | |
| 1588148 | 1588146 | AUGGGGG | 1313 | 4725 | 4746 | 1590 | 1611 | 1588147 | UGGCUGA | 1477 |
| | | GAGACAU | | | | | | | AUGUCUC | |
| | | UCAGCCA | | | | | | | CCCCCAU | |
| | | GA | | | | | | | | |
| 1588163 | 1588161 | AGGCACA | 1314 | 4685 | 4706 | 1550 | 1571 | 1588162 | CUCGCUG | 1478 |
| | | GAGAGGC | | | | | | | GCCUCUC | |
| | | CAGCGAG | | | | | | | UGUGCCU | |
| | | CU | | | | | | | | |
| 1588169 | 1588167 | ACGAGCU | 1315 | 4669 | 4690 | 1534 | 1555 | 1588168 | CUGACGU | 1479 |
| | | CCCUUGCA | | | | | | | GCAAGGG | |
| | | CGUCAGCC | | | | | | | AGCUCGU | |
| 1588172 | 1588170 | ACUUGCA | 1316 | 4661 | 4682 | 1526 | 1547 | 1588171 | CACCCCG | 1480 |
| | | CGUCAGCC | | | | | | | GCUGACG | |
| | | GGGGUGC | | | | | | | UGCAAGU | |
| | | G | | | | | | | | |
| 1588175 | 1588173 | AUCAGCC | 1317 | 4653 | 4674 | 1518 | 1539 | 1588174 | GCAGUGC | 1481 |
| | | GGGGUGC | | | | | | | GCACCCC | |
| | | GCACUGC | | | | | | | GGCUGAU | |
| | | GC | | | | | | | | |
| 1588178 | 1588176 | AGGUGCG | 1318 | 4645 | 4666 | 1510 | 1531 | 1588177 | ACCUGCG | 1482 |
| | | CACUGCGC | | | | | | | CGCAGUG | |
| | | GCAGGUC | | | | | | | CGCACCU | |
| | | U | | | | | | | | |
| 1588187 | 1588185 | AUGGUAC | 1319 | 4259 | 4280 | 1486 | 1507 | 1588186 | CAGAGCC | 1483 |
| | | CUGGGCC | | | | | | | GGCCCAG | |
| | | GGCUCUG | | | | | | | GUACCAU | |
| | | GG | | | | | | | | |
| 1588193 | 1588191 | AUCUGGG | 1320 | 4243 | 4264 | 1470 | 1491 | 1588192 | GGCAUCC | 1484 |
| | | AUCCCCGG | | | | | | | CGGGGAU | |
| | | GAUGCCC | | | | | | | CCCAGAU | |
| | | A | | | | | | | | |
| 1588211 | 1588209 | AUGGAGU | 1321 | 4195 | 4216 | 1422 | 1443 | 1588210 | AGGCCCG | 1485 |
| | | CUCUCACC | | | | | | | GUGAGAG | |
| | | GGGCCUA | | | | | | | ACUCCAU | |
| | | G | | | | | | | | |
| 1588220 | 1588218 | ACCCGCGU | 1322 | 4035 | 4056 | 1262 | 1283 | 1588219 | GAGGAGC | 1486 |
| | | CCUAAAG | | | | | | | UUUAGGA | |
| | | CUCCUCCA | | | | | | | CGCGGGU | |
| 1588223 | 1588221 | ACUAAAG | 1323 | 4027 | 4048 | 1254 | 1275 | 1588222 | CUCUGCU | 1487 |
| | | CUCCUCCA | | | | | | | GGAGGAG | |
| | | GCAGAGC | | | | | | | CUUUAGU | |
| | | C | | | | | | | | |
| 1588229 | 1588227 | AAGAGCC | 1324 | 4011 | 4032 | 1238 | 1259 | 1588228 | GAGGAAG | 1488 |
| | | CGGUAUU | | | | | | | AAUACCG | |
| | | CUUCCUCG | | | | | | | GGCUCUU | |
| | | C | | | | | | | | |
| 1588232 | 1588230 | AGUAUUC | 1325 | 4003 | 4024 | 1230 | 1251 | 1588231 | CCCUCAG | 1489 |
| | | UUCCUCGC | | | | | | | CGAGGAA | |
| | | UGAGGGG | | | | | | | GAAUACU | |
| | | U | | | | | | | | |
| 1588238 | 1588236 | AAGGGGU | 1326 | 3987 | 4008 | 1214 | 1235 | 1588237 | GCCUCGC | 1490 |
| | | GCUUCCA | | | | | | | UGGAAGC | |
| | | GCGAGGC | | | | | | | ACCCCUU | |
| | | GG | | | | | | | | |
| 1588241 | 1588239 | AUUCCAG | 1327 | 3979 | 4000 | 1206 | 1227 | 1588240 | AAGAGGC | 1491 |
| | | CGAGGCG | | | | | | | CGCCUCG | |
| | | GCCUCUUC | | | | | | | CUGGAAU | |
| | | C | | | | | | | | |
| 1588244 | 1588242 | AAGGCGG | 1328 | 3971 | 3992 | 1198 | 1219 | 1588243 | GGCCUCG | 1492 |
| | | CCUCUUCC | | | | | | | GAAGAGG | |
| | | GAGGCCU | | | | | | | CCGCCUU | |
| | | C | | | | | | | | |
| 1588247 | 1588245 | AUCUUCC | 1329 | 3963 | 3984 | 1190 | 1211 | 1588246 | GAGCUGG | 1493 |
| | | GAGGCCU | | | | | | | AGGCCUC | |
| | | CCAGCUCC | | | | | | | GGAAGAU | |
| | | C | | | | | | | | |
| 1588256 | 1588254 | AGGGGCC | 1330 | 3939 | 3960 | 1166 | 1187 | 1588255 | CUCCUAG | 1494 |
| | | UCCGUUU | | | | | | | AAACGGA | |
| | | CUAGGAG | | | | | | | GGCCCCU | |
| | | AG | | | | | | | | |
| 1588259 | 1588257 | ACGUUUC | 1331 | 3931 | 3952 | 1158 | 1179 | 1588258 | CGCAACC | 1495 |
| | | UAGGAGA | | | | | | | UCUCCUA | |
| | | GGUUGCG | | | | | | | GAAACGU | |
| | | CC | | | | | | | | |
| 1588268 | 1588266 | ACUGCAG | 1332 | 3907 | 3928 | 1134 | 1155 | 1588267 | CGAGCCC | 1496 |
| | | AAACUCC | | | | | | | GGAGUUU | |
| | | GGGCUCG | | | | | | | CUGCAGU | |
| | | CC | | | | | | | | |
| 1588274 | 1588272 | ACUCGCCA | 1333 | 3891 | 3912 | 1118 | 1139 | 1588273 | CUGGAUG | 1497 |
| | | GGAGCUC | | | | | | | AGCUCCU | |
| | | AUCCAGC | | | | | | | GGCGAGU | |
| | | A | | | | | | | | |
| 1588277 | 1588275 | AGAGCUC | 1334 | 3883 | 3904 | 1110 | 1131 | 1588276 | GCCUGCU | 1498 |
| | | AUCCAGC | | | | | | | GCUGGAU | |
| | | AGCAGGC | | | | | | | GAGCUCU | |
| | | CG | | | | | | | | |
| 1588283 | 1588281 | AAGGCCG | 1335 | 3867 | 3888 | 1094 | 1115 | 1588282 | UCUGCAC | 1499 |
| | | CAGGGGA | | | | | | | UCCCCUG | |
| | | GUGCAGA | | | | | | | CGGCCUU | |
| | | CC | | | | | | | | |
| 1588289 | 1588287 | ACAGACC | 1336 | 3851 | 3872 | 1078 | 1099 | 1588288 | GGAGCCG | 1500 |
| | | AGGGCGC | | | | | | | GCGCCCU | |
| | | CGGCUCCU | | | | | | | GGUCUGU | |
| | | G | | | | | | | | |
| 1588292 | 1588290 | AGGCGCC | 1337 | 3843 | 3864 | 1070 | 1091 | 1588291 | GCGCUCC | 1501 |
| | | GGCUCCU | | | | | | | AGGAGCC | |
| | | GGAGCGC | | | | | | | GGCGCCU | |
| | | CU | | | | | | | | |
| 1588295 | 1588293 | ACUCCUG | 1338 | 3835 | 3856 | 1062 | 1083 | 1588294 | CCUCCCA | 1502 |
| | | GAGCGCC | | | | | | | GGCGCUC | |
| | | UGGGAGG | | | | | | | CAGGAGU | |
| | | GC | | | | | | | | |
| 1588301 | 1588299 | AGAGGGC | 1339 | 3819 | 3840 | 1046 | 1067 | 1588300 | CAAGGCA | 1503 |
| | | GCCGGGA | | | | | | | UCCCGGC | |
| | | UGCCUUG | | | | | | | GCCCUCU | |
| | | CA | | | | | | | | |
| 1588304 | 1588302 | ACGGGAU | 1340 | 3811 | 3832 | 1038 | 1059 | 1588303 | GGCAGAU | 1504 |
| | | GCCUUGC | | | | | | | GCAAGGC | |
| | | AUCUGCCC | | | | | | | AUCCCGU | |
| | | C | | | | | | | | |
| 1588307 | 1588305 | ACUUGCA | 1341 | 3803 | 3824 | 1030 | 1051 | 1588306 | GCGGCAG | 1505 |
| | | UCUGCCCC | | | | | | | GGGCAGA | |
| | | UGCCGCGC | | | | | | | UGCAAGU | |
| 1588310 | 1588308 | AUGCCCCU | 1342 | 3795 | 3816 | 1022 | 1043 | 1588309 | GCCUCCG | 1506 |
| | | GCCGCGCG | | | | | | | CGCGGCA | |
| | | GAGGCGG | | | | | | | GGGGCAU | |
| 1588313 | 1588311 | ACCGCGCG | 1343 | 3787 | 3808 | 1014 | 1035 | 1588312 | ACGCCUC | 1507 |
| | | GAGGCGG | | | | | | | CGCCUCC | |
| | | AGGCGUC | | | | | | | GCGCGGU | |
| | | C | | | | | | | | |
| 1588316 | 1588314 | AAGGCGG | 1344 | 3779 | 3800 | 1006 | 1027 | 1588315 | GCCCCCG | 1508 |
| | | AGGCGUC | | | | | | | GACGCCU | |
| | | CGGGGGC | | | | | | | CCGCCUU | |
| | | GC | | | | | | | | |
| 1588319 | 1588317 | AGCGUCC | 1345 | 3771 | 3792 | 998 | 1019 | 1588318 | CAGCCCG | 1509 |
| | | GGGGGCG | | | | | | | CGCCCCC | |
| | | CGGGCUG | | | | | | | GGACGCU | |
| | | GG | | | | | | | | |
| 1588322 | 1588320 | AGGGCGC | 1346 | 3763 | 3784 | 990 | 1011 | 1588321 | CACCUCC | 1510 |
| | | GGGCUGG | | | | | | | CCAGCCC | |
| | | GGAGGUG | | | | | | | GCGCCCU | |
| | | GA | | | | | | | | |
| 1588325 | 1588323 | AGCUGGG | 1347 | 3755 | 3776 | 982 | 1003 | 1588324 | GGCAGCU | 1511 |
| | | GAGGUGG | | | | | | | CCACCUC | |
| | | AGCUGCCC | | | | | | | CCCAGCU | |
| | | C | | | | | | | | |
| 1588332 | 1588329 | AUGCCCCG | 1348 | 3739 | 3760 | 966 | 987 | 1588330 | GGGAACC | 1512 |
| | | GCUUGGG | | | | | | | CCAAGCC | |
| | | GUUCCCAC | | | | | | | GGGGCAU | |
| 1588335 | 1588333 | ACUUGGG | 1349 | 3731 | 3752 | 958 | 979 | 1588334 | GGCGGCG | 1513 |
| | | GUUCCCAC | | | | | | | UGGGAAC | |
| | | GCCGCCCC | | | | | | | CCCAAGU | |
| 1588341 | 1588339 | ACGCCCCG | 1350 | 3715 | 3736 | 942 | 963 | 1588340 | GUCCCCA | 1514 |
| | | GCGACCU | | | | | | | GGUCGCC | |
| | | GGGGACC | | | | | | | GGGGCGU | |
| | | C | | | | | | | | |
| 1588344 | 1588342 | ACGACCU | 1351 | 3707 | 3728 | 934 | 955 | 1588343 | GGGCCGG | 1515 |
| | | GGGGACC | | | | | | | GGUCCCC | |
| | | CCGGCCCC | | | | | | | AGGUCGU | |
| | | A | | | | | | | | |
| 1588347 | 1588345 | AGGACCCC | 1352 | 3699 | 3720 | 926 | 947 | 1588346 | UGGGGCU | 1516 |
| | | GGCCCCAG | | | | | | | GGGGCCG | |
| | | CCCCACC | | | | | | | GGGUCCU | |
| 1588350 | 1588348 | AGCCCCAG | 1353 | 3691 | 3712 | 918 | 939 | 1588349 | GUCCGUG | 1517 |
| | | CCCCACCA | | | | | | | GUGGGGC | |
| | | CGGACUC | | | | | | | UGGGGCU | |
| 1588353 | 1588351 | ACCCACCA | 1354 | 3683 | 3704 | 910 | 931 | 1588352 | CCAGGGG | 1518 |
| | | CGGACUCC | | | | | | | AGUCCGU | |
| | | CCUGGGA | | | | | | | GGUGGGU | |
| 1588356 | 1588354 | AGGACUC | 1355 | 3675 | 3696 | 902 | 923 | 1588355 | CCCACGU | 1519 |
| | | CCCUGGG | | | | | | | CCCAGGG | |
| | | ACGUGGG | | | | | | | GAGUCCU | |
| | | UG | | | | | | | | |
| 1588359 | 1588357 | ACUGGGA | 1356 | 3667 | 3688 | 894 | 915 | 1588358 | UUGCGCC | 1520 |
| | | CGUGGGU | | | | | | | ACCCACG | |
| | | GGCGCAA | | | | | | | UCCCAGU | |
| | | GC | | | | | | | | |
| 1588362 | 1588360 | AUGGGUG | 1357 | 3659 | 3680 | 886 | 907 | 1588361 | AGGGGUG | 1521 |
| | | GCGCAAG | | | | | | | CUUGCGC | |
| | | CACCCCUU | | | | | | | CACCCAU | |
| | | G | | | | | | | | |
| 1588365 | 1588363 | AGCAAGC | 1358 | 3651 | 3672 | 878 | 899 | 1588364 | CAGGGCC | 1522 |
| | | ACCCCUUG | | | | | | | AAGGGGU | |
| | | GCCCUGCG | | | | | | | GCUUGCU | |
| 1588368 | 1588366 | ACCCUUG | 1359 | 3643 | 3664 | 870 | 891 | 1588367 | CGGGGCC | 1523 |
| | | GCCCUGCG | | | | | | | GCAGGGC | |
| | | GCCCCGCU | | | | | | | CAAGGGU | |
| 1588371 | 1588369 | ACCUGCG | 1360 | 3635 | 3656 | 862 | 883 | 1588370 | CGCUCAA | 1524 |
| | | GCCCCGCU | | | | | | | GCGGGGC | |
| | | UGAGCGG | | | | | | | CGCAGGU | |
| | | G | | | | | | | | |
| 1588374 | 1588372 | ACCCGCUU | 1361 | 3627 | 3648 | 854 | 875 | 1588373 | CCUGGGC | 1525 |
| | | GAGCGGG | | | | | | | CCGCUCA | |
| | | CCCAGGCU | | | | | | | AGCGGGU | |
| 1588377 | 1588375 | AAGCGGG | 1362 | 3619 | 3640 | 846 | 867 | 1588376 | UGGCACA | 1526 |
| | | CCCAGGCU | | | | | | | GCCUGGG | |
| | | GUGCCACC | | | | | | | CCCGCUU | |
| 1588380 | 1588378 | ACAGGCU | 1363 | 3611 | 3632 | 838 | 859 | 1588379 | CUGCGCG | 1527 |
| | | GUGCCACC | | | | | | | GUGGCAC | |
| | | GCGCAGG | | | | | | | AGCCUGU | |
| | | G | | | | | | | | |
| 1588386 | 1588384 | AGCAGGG | 1364 | 3595 | 3616 | 822 | 843 | 1588385 | ACGGCCU | 1528 |
| | | GCCCGGCA | | | | | | | GCCGGGC | |
| | | GGCCGUC | | | | | | | CCCUGCU | |
| | | G | | | | | | | | |
| 1588389 | 1588387 | ACCGGCA | 1365 | 3587 | 3608 | 814 | 835 | 1588388 | GCAGCGC | 1529 |
| | | GGCCGUC | | | | | | | GACGGCC | |
| | | GCGCUGC | | | | | | | UGCCGGU | |
| | | GG | | | | | | | | |
| 1588392 | 1588390 | ACCGUCGC | 1366 | 3579 | 3600 | 806 | 827 | 1588391 | CGGGACC | 1530 |
| | | GCUGCGG | | | | | | | CGCAGCG | |
| | | GUCCCGG | | | | | | | CGACGGU | |
| | | U | | | | | | | | |
| 1588395 | 1588393 | ACUGCGG | 1367 | 3571 | 3592 | 798 | 819 | 1588394 | GGGAGGA | 1531 |
| | | GUCCCGG | | | | | | | CCGGGAC | |
| | | UCCUCCCG | | | | | | | CCGCAGU | |
| | | G | | | | | | | | |
| 1588401 | 1588399 | AUCCCGGC | 1368 | 3555 | 3576 | 782 | 803 | 1588400 | CACCCGG | 1532 |
| | | UUUUGCC | | | | | | | GCAAAAG | |
| | | CGGGUGC | | | | | | | CCGGGAU | |
| | | G | | | | | | | | |
| 1588413 | 1588411 | AAGGAGC | 1369 | 3523 | 3544 | 750 | 771 | 1588412 | UCUCCCA | 1533 |
| | | CUGAGGG | | | | | | | CCCUCAG | |
| | | UGGGAGA | | | | | | | GCUCCUU | |
| | | GC | | | | | | | | |
| 1588419 | 1588417 | AGAGAGC | 1370 | 3507 | 3528 | 734 | 755 | 1588418 | CCUCCGG | 1534 |
| | | GCCCCGUC | | | | | | | ACGGGGC | |
| | | CGGAGGA | | | | | | | GCUCUCU | |
| | | G | | | | | | | | |
| 1588422 | 1588420 | ACCCGUCC | 1371 | 3499 | 3520 | 726 | 747 | 1588421 | CCCCGGC | 1535 |
| | | GGAGGAG | | | | | | | UCCUCCG | |
| | | CCGGGGC | | | | | | | GACGGGU | |
| | | G | | | | | | | | |
| 1588425 | 1588423 | AGAGGAG | 1372 | 3491 | 3512 | 718 | 739 | 1588424 | CUACGCC | 1536 |
| | | CCGGGGC | | | | | | | GCCCCGG | |
| | | GGCGUAG | | | | | | | CUCCUCU | |
| | | GC | | | | | | | | |
| 1588428 | 1588426 | AGGGGCG | 1373 | 3483 | 3504 | 710 | 731 | 1588427 | GAUUUCG | 1537 |
| | | GCGUAGG | | | | | | | CCUACGC | |
| | | CGAAAUC | | | | | | | CGCCCCU | |
| | | CC | | | | | | | | |
| 1588431 | 1588429 | AGUAGGC | 1374 | 3475 | 3496 | 702 | 723 | 1588430 | CGCGCGG | 1538 |
| | | GAAAUCC | | | | | | | GGAUUUC | |
| | | CCGCGCGC | | | | | | | GCCUACU | |
| | | C | | | | | | | | |
| 1588437 | 1588435 | ACGCGCCG | 1375 | 3459 | 3480 | 686 | 707 | 1588436 | UCCCAAC | 1539 |
| | | GGGCAGG | | | | | | | CUGCCCC | |
| | | UUGGGAG | | | | | | | GGCGCGU | |
| | | A | | | | | | | | |
| 1588440 | 1588438 | AGGCAGG | 1376 | 3451 | 3472 | 678 | 699 | 1588439 | AGGGGAU | 1540 |
| | | UUGGGAG | | | | | | | CUCCCAA | |
| | | AUCCCCUC | | | | | | | CCUGCCU | |
| | | U | | | | | | | | |
| 1588446 | 1588444 | ACCCUCUG | 1377 | 3435 | 3456 | 662 | 683 | 1588445 | CAGGCCG | 1541 |
| | | CCGGCGCG | | | | | | | CGCCGGC | |
| | | GCCUGGC | | | | | | | AGAGGGU | |
| 1588449 | 1588447 | ACGGCGC | 1378 | 3427 | 3448 | 654 | 675 | 1588448 | AGCCCAG | 1542 |
| | | GGCCUGG | | | | | | | CCAGGCC | |
| | | CUGGGCU | | | | | | | GCGCCGU | |
| | | GC | | | | | | | | |
| 1588452 | 1588450 | ACCUGGC | 1379 | 3419 | 3440 | 646 | 667 | 1588451 | CGCGCUG | 1543 |
| | | UGGGCUG | | | | | | | CAGCCCA | |
| | | CAGCGCG | | | | | | | GCCAGGU | |
| | | GG | | | | | | | | |
| 1588455 | 1588453 | AGGCUGC | 1380 | 3411 | 3432 | 638 | 659 | 1588454 | GCCGCCC | 1544 |
| | | AGCGCGG | | | | | | | CCGCGCU | |
| | | GGGCGGC | | | | | | | GCAGCCU | |
| | | CC | | | | | | | | |
| 1588458 | 1588456 | ACGCGGG | 1381 | 3403 | 3424 | 630 | 651 | 1588457 | CAGCGAG | 1545 |
| | | GGCGGCCC | | | | | | | GGCCGCC | |
| | | UCGCUGCC | | | | | | | CCCGCGU | |
| 1588461 | 1588459 | ACGGCCCU | 1382 | 3395 | 3416 | 622 | 643 | 1588460 | GAGCCAG | 1546 |
| | | CGCUGCCU | | | | | | | GCAGCGA\| | |
| | | GGCUCAC | | | | | | | GGGCCGU | |
| 1588464 | 1588462 | AGCUGCC | 1383 | 3387 | 3408 | 614 | 635 | 1588463 | GCUUUCG | 1547 |
| | | UGGCUCA | | | | | | | UGAGCCA | |
| | | CGAAAGC | | | | | | | GGCAGCU | |
| | | CC | | | | | | | | |
| 1588467 | 1588465 | AGCUCAC | 1384 | 3379 | 3400 | 606 | 627 | 1588466 | CACAGGG | 1548 |
| | | GAAAGCC | | | | | | | GGCUUUC | |
| | | CCCUGUG | | | | | | | GUGAGCU | |
| | | GG | | | | | | | | |
| 1588473 | 1588471 | AUGUGGG | 1385 | 3363 | 3384 | 590 | 611 | 1588472 | GCGCCUG | 1549 |
| | | AGAGCCCC | | | | | | | GGGCUCU | |
| | | AGGCGCG | | | | | | | CCCACAU | |
| | | C | | | | | | | | |
| 1588476 | 1588474 | AAGCCCCA | 1386 | 3355 | 3376 | 582 | 603 | 1588475 | UGCCCUG | 1550 |
| | | GGCGCGC | | | | | | | CGCGCCU | |
| | | AGGGCAC | | | | | | | GGGGCUU | |
| | | G | | | | | | | | |
| 1588479 | 1588477 | AGCGCGC | 1387 | 3347 | 3368 | 574 | 595 | 1588478 | ACCCCAC | 1551 |
| | | AGGGCAC | | | | | | | GUGCCCU | |
| | | GUGGGGU | | | | | | | GCGCGCU | |
| | | GC | | | | | | | | |
| 1588482 | 1588480 | AGGCACG | 1388 | 3339 | 3360 | 566 | 587 | 1588481 | CUUCCCG | 1552 |
| | | UGGGGUG | | | | | | | CACCCCA | |
| | | CGGGAAG | | | | | | | CGUGCCU | |
| | | CC | | | | | | | | |
| 1588485 | 1588483 | AGGGUGC | 1389 | 3331 | 3352 | 558 | 579 | 1588484 | GAACGGG | 1553 |
| | | GGGAAGC | | | | | | | GCUUCCC | |
| | | CCCGUUCC | | | | | | | GCACCCU | |
| | | C | | | | | | | | |
| 1588488 | 1588486 | AGAAGCC | 1390 | 3323 | 3344 | 550 | 571 | 1588487 | CGCGUGG | 1554 |
| | | CCGUUCCC | | | | | | | GGAACGG | |
| | | CACGCGCC | | | | | | | GGCUUCU | |
| 1588491 | 1588489 | AGUUCCCC | 1391 | 3315 | 3336 | 542 | 563 | 1588490 | CACACCG | 1555 |
| | | ACGCGCCG | | | | | | | GCGCGUG | |
| | | GUGUGGG | | | | | | | GGGAACU | |
| 1588497 | 1588495 | AUGUGGG | 1392 | 3299 | 3320 | 526 | 547 | 1588496 | GUGGGUC | 1556 |
| | | CGAAGGC | | | | | | | GCCUUCG | |
| | | GACCCACG | | | | | | | CCCACAU | |
| | | A | | | | | | | | |
| 1588500 | 1588498 | AAAGGCG | 1393 | 3291 | 3312 | 518 | 539 | 1588499 | GCUCCCU | 1557 |
| | | ACCCACGA | | | | | | | CGUGGGU | |
| | | GGGAGCA | | | | | | | CGCCUUU | |
| | | G | | | | | | | | |
| 1588503 | 1588501 | ACCACGA | 1394 | 3283 | 3304 | 510 | 531 | 1588502 | GUCACCC | 1558 |
| | | GGGAGCA | | | | | | | UGCUCCC | |
| | | GGGUGAC | | | | | | | UCGUGGU | |
| | | CC | | | | | | | | |
| 1588506 | 1588504 | AGAGCAG | 1395 | 3275 | 3296 | 502 | 523 | 1588505 | CGGCGGG | 1559 |
| | | GGUGACC | | | | | | | GGUCACC | |
| | | CCCGCCGG | | | | | | | CUGCUCU | |
| | | G | | | | | | | | |
| 1588509 | 1588507 | AUGACCCC | 1396 | 3267 | 3288 | 494 | 515 | 1588508 | GCGGCCC | 1560 |
| | | CGCCGGG | | | | | | | CCGGCGG | |
| | | GGCCGCGC | | | | | | | GGGUCAU | |
| 1588512 | 1588510 | AGCCGGG | 1397 | 3259 | 3280 | 486 | 507 | 1588511 | UGUGCAG | 1561 |
| | | GGCCGCGC | | | | | | | CGCGGCC | |
| | | UGCACAG | | | | | | | CCCGGCU | |
| | | G | | | | | | | | |
| 1588515 | 1588513 | ACCGCGCU | 1398 | 3251 | 3272 | 478 | 499 | 1588514 | AGGCGGC | 1562 |
| | | GCACAGG | | | | | | | CUGUGCA | |
| | | CCGCCUGC | | | | | | | GCGCGGU | |
| 1588518 | 1588516 | ACACAGG | 1399 | 3243 | 3264 | 470 | 491 | 1588517 | GCGCAGG | 1563 |
| | | CCGCCUGC | | | | | | | CAGGCGG | |
| | | CUGCGCG | | | | | | | CCUGUGU | |
| | | G | | | | | | | | |
| 1588521 | 1588519 | AGCCUGCC | 1400 | 3235 | 3256 | 462 | 483 | 1588520 | GGGCGCC | 1564 |
| | | UGCGCGG | | | | | | | CGCGCAG | |
| | | GCGCCCUG | | | | | | | GCAGGCU | |
| 1588527 | 1588525 | AGCCCUGC | 1401 | 3219 | 3240 | 446 | 467 | 1588526 | CCGGGAC | 1565 |
| | | CACCCUGU | | | | | | | AGGGUGG | |
| | | CCCGGGU | | | | | | | CAGGGCU | |
| 1588530 | 1588528 | AACCCUG | 1402 | 3211 | 3232 | 438 | 459 | 1588529 | CCAGGCA | 1566 |
| | | UCCCGGG | | | | | | | CCCGGGA | |
| | | UGCCUGG | | | | | | | CAGGGUU | |
| | | CC | | | | | | | | |
| 1588533 | 1588531 | ACCGGGU | 1403 | 3203 | 3224 | 430 | 451 | 1588532 | UCGAAGG | 1567 |
| | | GCCUGGCC | | | | | | | GCCAGGC | |
| | | CUUCGAU | | | | | | | ACCCGGU | |
| | | U | | | | | | | | |
| 1588536 | 1588534 | ACUGGCCC | 1404 | 3195 | 3216 | 422 | 443 | 1588535 | UUUCAGA | 1568 |
| | | UUCGAUU | | | | | | | AUCGAAG | |
| | | CUGAAAC | | | | | | | GGCCAGU | |
| | | C | | | | | | | | |
| 1588545 | 1588543 | AAUCUGA | 1405 | 3171 | 3192 | 398 | 419 | 1588544 | CCGGAGU | 1569 |
| | | AUCCUGG | | | | | | | CCAGGAU | |
| | | ACUCCGG | | | | | | | UCAGAUU | |
| | | GA | | | | | | | | |
| 1588554 | 1588552 | ACCCGUCU | 1406 | 3147 | 3168 | 374 | 395 | 1588553 | GAGCUGG | 1570 |
| | | CUCUGGCC | | | | | | | CCAGAGA | |
| | | AGCUCCU | | | | | | | GACGGGU | |
| 1588557 | 1588555 | AUCUGGC | 1407 | 3139 | 3160 | 366 | 387 | 1588556 | CCCGGGA | 1571 |
| | | CAGCUCCU | | | | | | | GGAGCUG | |
| | | CCCGGGCG | | | | | | | GCCAGAU | |
| 1588563 | 1588561 | ACGGGCG | 1408 | 3123 | 3144 | 350 | 371 | 1588562 | UUUCCAG | 1572 |
| | | GCGAUGC | | | | | | | GCAUCGC | |
| | | CUGGAAA | | | | | | | CGCCCGU | |
| | | GC | | | | | | | | |
| 1588566 | 1588564 | AGAUGCC | 1409 | 3115 | 3136 | 342 | 363 | 1588565 | AGGAUCG | 1573 |
| | | UGGAAAG | | | | | | | CUUUCCA | |
| | | CGAUCCU | | | | | | | GGCAUCU | |
| | | UC | | | | | | | | |
| 1588569 | 1588567 | AGAAAGC | 1410 | 3107 | 3128 | 334 | 355 | 1588568 | CUUUGAG | 1574 |
| | | GAUCCUU | | | | | | | AAGGAUC | |
| | | CUCAAAG | | | | | | | GCUUUCU | |
| | | GC | | | | | | | | |
| 1588575 | 1588573 | AAAAGGC | 1411 | 3091 | 3112 | 318 | 339 | 1588574 | CCCUGCU | 1575 |
| | | UCGGAGG | | | | | | | CCUCCGA | |
| | | AGCAGGG | | | | | | | GCCUUUU | |
| | | CG | | | | | | | | |
| 1588578 | 1588576 | AGGAGGA | 1412 | 3083 | 3104 | 310 | 331 | 1588577 | CCAGACC | 1576 |
| | | GCAGGGC | | | | | | | GCCCUGC | |
| | | GGUCUGG | | | | | | | UCCUCCU | |
| | | GA | | | | | | | | |
| 1588581 | 1588579 | AAGGGCG | 1413 | 3075 | 3096 | 302 | 323 | 1588580 | ACCGGAU | 1577 |
| | | GUCUGGG | | | | | | | CCCAGAC | |
| | | AUCCGGU | | | | | | | CGCCCUU | |
| | | GA | | | | | | | | |
| 1588587 | 1588585 | ACGGUGA | 1414 | 3059 | 3080 | 286 | 307 | 1588586 | AAAGCGG | 1578 |
| | | CGGCGGU | | | | | | | ACCGCCG | |
| | | CCGCUUUC | | | | | | | UCACCGU | |
| | | G | | | | | | | | |
| 1588590 | 1588588 | AGCGGUC | 1415 | 3051 | 3072 | 278 | 299 | 1588589 | GGCCGGC | 1579 |
| | | CGCUUUC | | | | | | | GAAAGCG | |
| | | GCCGGCCU | | | | | | | GACCGCU | |
| | | U | | | | | | | | |
| 1588593 | 1588591 | ACUUUCG | 1416 | 3043 | 3064 | 270 | 291 | 1588592 | CGCCAGA | 1580 |
| | | CCGGCCUU | | | | | | | AGGCCGG | |
| | | CUGGCGG | | | | | | | CGAAAGU | |
| | | G | | | | | | | | |
| 1588596 | 1588594 | AGGCCUU | 1417 | 3035 | 3056 | 262 | 283 | 1588595 | ACGCGGC | 1581 |
| | | CUGGCGG | | | | | | | CCGCCAG | |
| | | GCCGCGUC | | | | | | | AAGGCCU | |
| | | U | | | | | | | | |
| 1588602 | 1588600 | AGCGUCU | 1418 | 3019 | 3040 | 246 | 267 | 1588601 | GGCCCUG | 1582 |
| | | CCCGGGCC | | | | | | | GCCCGGG | |
| | | AGGGCCG | | | | | | | AGACGCU | |
| | | A | | | | | | | | |
| 1588605 | 1588603 | ACGGGCC | 1419 | 3011 | 3032 | 238 | 259 | 1588604 | GGAAUCU | 1583 |
| | | AGGGCCG | | | | | | | CGGCCCU | |
| | | AGAUUCC | | | | | | | GGCCCGU | |
| | | CG | | | | | | | | |
| 1588608 | 1588606 | AGGCCGA | 1420 | 3003 | 3024 | 230 | 251 | 1588607 | CACCGGC | 1584 |
| | | GAUUCCC | | | | | | | GGGAAUC | |
| | | GCCGGUG | | | | | | | UCGGCCU | |
| | | CU | | | | | | | | |
| 1588614 | 1588612 | AGGUGCU | 1421 | 2987 | 3008 | 214 | 235 | 1588613 | ACGCCAG | 1585 |
| | | GCCUCAGC | | | | | | | CUGAGGC | |
| | | UGGCGUG | | | | | | | AGCACCU | |
| | | A | | | | | | | | |
| 1588617 | 1588615 | ACUCAGC | 1422 | 2979 | 3000 | 206 | 227 | 1588616 | GAGAGGU | 1586 |
| | | UGGCGUG | | | | | | | CACGCCA | |
| | | ACCUCUCA | | | | | | | GCUGAGU | |
| | | U | | | | | | | | |
| 1588620 | 1588618 | AGCGUGA | 1423 | 2971 | 2992 | 198 | 219 | 1588619 | UUCAGAA | 1587 |
| | | CCUCUCAU | | | | | | | UGAGAGG | |
| | | UCUGAAA | | | | | | | UCACGCU | |
| | | C | | | | | | | | |
| 1588623 | 1588621 | AUCUCAU | 1424 | 2963 | 2984 | 190 | 211 | 1588622 | GAUUUGG | 1588 |
| | | UCUGAAA | | | | | | | UUUCAGA | |
| | | CCAAAUC | | | | | | | AUGAGAU | |
| | | UG | | | | | | | | |
| 1588626 | 1588624 | AUGAAAC | 1425 | 2955 | 2976 | 182 | 203 | 1588625 | AGGGUCC | 1589 |
| | | CAAAUCU | | | | | | | AGAUUUG | |
| | | GGACCCU | | | | | | | GUUUCAU | |
| | | GG | | | | | | | | |
| 1588635 | 1588633 | AUCCGGA | 1426 | 2931 | 2952 | 158 | 179 | 1588634 | CAGGCCA | 1590 |
| | | AUGCCGA | | | | | | | UCGGCAU | |
| | | UGGCCUG | | | | | | | UCCGGAU | |
| | | GG | | | | | | | | |
| 1588641 | 1588639 | ACCUGGG | 1427 | 2915 | 2936 | 142 | 163 | 1588640 | CAGAGAA | 1591 |
| | | CCAGCCGU | | | | | | | CGGCUGG | |
| | | UCUCUGG | | | | | | | CCCAGGU | |
| | | U | | | | | | | | |
| 1588644 | 1588642 | AAGCCGU | 1428 | 2907 | 2928 | 134 | 155 | 1588643 | AUCGCCA | 1592 |
| | | UCUCUGG | | | | | | | CCAGAGA | |
| | | UGGCGAU | | | | | | | ACGGCUU | |
| | | GC | | | | | | | | |
| 1588647 | 1588645 | AUCUGGU | 1429 | 2899 | 2920 | 126 | 147 | 1588646 | ACCCGGG | 1593 |
| | | GGCGAUG | | | | | | | CAUCGCC | |
| | | CCCGGGU | | | | | | | ACCAGAU | |
| | | AC | | | | | | | | |
| 1588650 | 1588648 | ACGAUGC | 1430 | 2891 | 2912 | 118 | 139 | 1588649 | GAACCCG | 1594 |
| | | CCGGGUA | | | | | | | UACCCGG | |
| | | CGGGUUC | | | | | | | GCAUCGU | |
| | | CG | | | | | | | | |
| 1588653 | 1588651 | AGGGUAC | 1431 | 2883 | 2904 | 110 | 131 | 1588652 | UUUGAGC | 1595 |
| | | GGGUUCC | | | | | | | GGAACCC | |
| | | GCUCAAA | | | | | | | GUACCCU | |
| | | GC | | | | | | | | |
| 1588656 | 1588654 | AGUUCCG | 1432 | 2875 | 2896 | 102 | 123 | 1588655 | GAGCCUG | 1596 |
| | | CUCAAAG | | | | | | | CUUUGAG | |
| | | CAGGCUC | | | | | | | CGGAACU | |
| | | GC | | | | | | | | |
| 1588662 | 1588660 | AGCUCGC | 1433 | 2859 | 2880 | 86 | 107 | 1588661 | CAAAGCG | 1597 |
| | | AGGGCCU | | | | | | | AGGCCCU | |
| | | CGCUUUG | | | | | | | GCGAGCU | |
| | | GC | | | | | | | | |
| 1588665 | 1588663 | AGGCCUC | 1434 | 2851 | 2872 | 78 | 99 | 1588664 | CCCCGAG | 1598 |
| | | GCUUUGG | | | | | | | CCAAAGC | |
| | | CUCGGGG | | | | | | | GAGGCCU | |
| | | UC | | | | | | | | |
| 1588668 | 1588666 | AUUUGGC | 1435 | 2843 | 2864 | 70 | 91 | 1588667 | CGUUUGG | \|1599 |
| | | UCGGGGU | | | | | | | ACCCCGA | |
| | | CCAAACG | | | | | | | GCCAAAU | |
| | | AG | | | | | | | | |
| 1588671 | 1588669 | AGGGGUC | 1436 | 2835 | 2856 | 62 | 83 | 1588670 | CGGAGAC | 1600 |
| | | CAAACGA | | | | | | | UCGUUUG | |
| | | GUCUCCG | | | | | | | GACCCCU | |
| | | UC | | | | | | | | |
| 1588677 | 1588675 | AUCCGUC | 1437 | 2819 | 2840 | 46 | 67 | 1588676 | GGGACGA | 1601 |
| | | GCCGUCCU | | | | | | | GGACGGC | |
| | | CGUCCCCG | | | | | | | GACGGAU | |
| 1588680 | 1588678 | ACGUCCUC | 1438 | 2811 | 2832 | 38 | 59 | 1588679 | GAAGCCC | 1602 |
| | | GUCCCCGG | | | | | | | GGGGACG | |
| | | GCUUCCG | | | | | | | AGGACGU | |
| 1588683 | 1588681 | AUCCCCGG | 1439 | 2803 | 2824 | 30 | 51 | 1588682 | UCCCCGC | 1603 |
| | | GCUUCCGC | | | | | | | GGAAGCC | |
| | | GGGGAGG | | | | | | | CGGGGAU | |
| 1588686 | 1588684 | ACUUCCGC | 1440 | 2795 | 2816 | 22 | 43 | 1588685 | CAGCACC | 1604 |
| | | GGGGAGG | | | | | | | CUCCCCG | |
| | | GUGCUGU | | | | | | | CGGAAGU | |
| | | C | | | | | | | | |
| 1588689 | 1588687 | AGGGAGG | 1441 | 2787 | 2808 | 14 | 35 | 1588688 | CCCUCGG | 1605 |
| | | GUGCUGU | | | | | | | ACAGCAC | |
| | | CCGAGGG | | | | | | | CCUCCCU | |
| | | UG | | | | | | | | |
| 1588695 | 1588693 | AAGGGUG | 1442 | 2771 | 2792 | N/A | N/A | 1588694 | GAUGGCC | 1606 |
| | | UCGGGAG | | | | | | | CUCCCGA | |
| | | GGCCAUC | | | | | | | CACCCUU | |
| | | GC | | | | | | | | |
| 1588698 | 1588696 | AGGGAGG | 1443 | 2763 | 2784 | N/A | N/A | 1588697 | CUCACCG | 1607 |
| | | GCCAUCGC | | | | | | | CGAUGGC | |
| | | GGUGAGC | | | | | | | CCUCCCU | |
| | | C | | | | | | | | |
| 1588701 | 1588699 | ACAUCGC | 1444 | 2755 | 2776 | N/A | N/A | 1588700 | GGCCGGG | 1608 |
| | | GGUGAGC | | | | | | | GCUCACC | |
| | | CCCGGCCG | | | | | | | GCGAUGU | |
| | | G | | | | | | | | |
| 1588704 | 1588702 | AUGAGCC | 1445 | 2747 | 2768 | N/A | N/A | 1588703 | GAAAUUC | 1609 |
| | | CCGGCCGG | | | | | | | CGGCCGG | |
| | | AAUUUCA | | | | | | | GGCUCAU | |
| | | C | | | | | | | | |
| 1588707 | 1588705 | AGGCCGG | 1446 | 2739 | 2760 | N/A | N/A | 1588706 | CCGUCCG | 1610 |
| | | AAUUUCA | | | | | | | UGAAAUU | |
| | | CGGACGG | | | | | | | CCGGCCU | |
| | | AC | | | | | | | | |
| 1588713 | 1588711 | AACGGAC | 1447 | 2723 | 2744 | N/A | N/A | 1588712 | CUCUCUC | 1611 |
| | | GCGGGCA | | | | | | | UGCCCGC | |
| | | GAGAGAG | | | | | | | GUCCGUU | |
| | | GC | | | | | | | | |
| 1588716 | 1588714 | AGGGCAG | 1448 | 2715 | 2736 | N/A | N/A | 1588715 | CCGCCGG | 1612 |
| | | AGAGAGG | | | | | | | CCUCUCU | |
| | | CCGGCGG | | | | | | | CUGCCCU | |
| | | GC | | | | | | | | |
| 1588719 | 1588717 | AAGAGGC | 1449 | 2707 | 2728 | N/A | N/A | 1588718 | ACGGGAG | 1613 |
| | | CGGCGGG | | | | | | | CCCGCCG | |
| | | CUCCCGUG | | | | | | | GCCUCUU | |
| | | C | | | | | | | | |
| 1588722 | 1588720 | AGCGGGC | 1450 | 2699 | 2720 | N/A | N/A | 1588721 | UGAGGUG | 1614 |
| | | UCCCGUGC | | | | | | | CACGGGA | |
| | | ACCUCAGC | | | | | | | GCCCGCU | |
| 1588725 | 1588723 | ACCGUGC | 1451 | 2691 | 2712 | N/A | N/A | 1588724 | AGUCCGG | 1615 |
| | | ACCUCAGC | | | | | | | CUGAGGU | |
| | | CGGACUG | | | | | | | GCACGGU | |
| | | U | | | | | | | | |
| 1588728 | 1588726 | ACUCAGCC | 1452 | 2683 | 2704 | N/A | N/A | 1588727 | CAGUGCA | 1616 |
| | | GGACUGU | | | | | | | CAGUCCG | |
| | | GCACUGC | | | | | | | GCUGAGU | |
| | | G | | | | | | | | |
| 1588731 | 1588729 | AGACUGU | 1453 | 2675 | 2696 | N/A | N/A | 1588730 | ACCUGCC | 1617 |
| | | GCACUGC | | | | | | | GCAGUGC | |
| | | GGCAGGU | | | | | | | ACAGUCU | |
| | | GC | | | | | | | | |
| 1588734 | 1588732 | AACUGCG | 1454 | 2667 | 2688 | N/A | N/A | 1588733 | CUGGCUG | 1618 |
| | | GCAGGUG | | | | | | | CACCUGC | |
| | | CAGCCAG | | | | | | | CGCAGUU | |
| | | GA | | | | | | | | |
| 1588737 | 1588735 | AAGGUGC | 1455 | 2659 | 2680 | N/A | N/A | 1588736 | CAGGCCU | 1619 |
| | | AGCCAGG | | | | | | | CCUGGCU | |
| | | AGGCCUG | | | | | | | GCACCUU | |
| | | CC | | | | | | | | |
| 1588740 | 1588738 | ACCAGGA | 1456 | 2651 | 2672 | N/A | N/A | 1588739 | UGUCCGG | 1620 |
| | | GGCCUGCC | | | | | | | GCAGGCC | |
| | | CGGACAG | | | | | | | UCCUGGU | |
| | | C | | | | | | | | |
| 1588743 | 1588741 | ACCUGCCC | 1457 | 2643 | 2664 | N/A | N/A | 1588742 | UGGCUGG | 1621 |
| | | GGACAGC | | | | | | | CUGUCCG | |
| | | CAGCCAGC | | | | | | | GGCAGGU | |
| 1588746 | 1588744 | AGACAGC | 1458 | 2635 | 2656 | N/A | N/A | 1588745 | UGGCUGG | 1622 |
| | | CAGCCAGC | | | | | | | CUGGCUG | |
| | | CAGCCAGC | | | | | | | GCUGUCU | |
| 1588755 | 1588753 | AUAUAGG | 1459 | 4823 | 4844 | 1688 | 1709 | 1588754 | CCCUCCC | 1623 |
| | | AUCCACA | | | | | | | UGUGGAU | |
| | | GGGAGGG | | | | | | | CCUAUAU | |
| | | GG | | | | | | | | |
| 1588761 | 1588759 | ACAGGAG | 1460 | 4772 | 4793 | 1637 | 1658 | 1588760 | GAUUAGA | 1624 |
| | | AUGUAAC | | | | | | | GUUACAU | |
| | | UCUAAUC | | | | | | | CUCCUGU | |
| | | CA | | | | | | | | |
| 1588770 | 1588768 | AGGGAGA | 1461 | 4721 | 4742 | 1586 | 1607 | 1588769 | AUUCUGG | 1625 |
| | | CAUUCAG | | | | | | | CUGAAUG | |
| | | CCAGAAU | | | | | | | UCUCCCU | |
| | | UU | | | | | | | | |
| 1588773 | 1588771 | AAAUUUC | 1462 | 4704 | 4725 | 1569 | 1590 | 1588772 | CCUUGUU | 1626 |
| | | ACGGAAG | | | | | | | CUUCCGU | |
| | | AACAAGG | | | | | | | GAAAUUU | |
| | | GC | | | | | | | | |
| 1588785 | 1588783 | AGUACCU | 1463 | 4257 | 4278 | 1484 | 1505 | 1588784 | CCCAGAG | 1627 |
| | | GGGCCGG | | | | | | | CCGGCCC | |
| | | CUCUGGG | | | | | | | AGGUACU | |
| | | AU | | | | | | | | |
| 1588791 | 1588789 | ACAGGAA | 1464 | 4223 | 4244 | N/A | N/A | 1588790 | AGAACUG | 1628 |
| | | AGAAAGG | | | | | | | CCUUUCU | |
| | | CAGUUCU | | | | | | | UUCCUGU | |
| | | CC | | | | | | | | |
| 1588803 | 1588801 | ACCAGGCC | 1465 | 4155 | 4176 | 1382 | 1403 | 1588802 | CACCGGG | 1629 |
| | | GGUCAGC | | | | | | | CUGACCG | |
| | | CCGGUGG | | | | | | | GCCUGGU | |
| | | A | | | | | | | | |
| 1588806 | 1588804 | AGUGGAG | 1466 | 4138 | 4159 | 1365 | 1386 | 1588805 | CUCCGCC | 1630 |
| | | GGGGCGG | | | | | | | CCGCCCC | |
| | | GGCGGAG | | | | | | | CUCCACU | |
| | | AC | | | | | | | | |
| 1588809 | 1588807 | AGAGACA | 1467 | 4121 | 4142 | 1348 | 1369 | 1588808 | GCUACGG | 1631 |
| | | CGCCCCUC | | | | | | | AGGGGCG | |
| | | CGUAGCC | | | | | | | UGUCUCU | |
| | | A | | | | | | | | |
| 1588815 | 1588813 | ACCGCGA | 1468 | 4087 | 4108 | 1314 | 1335 | 1588814 | CGGUGGC | 1632 |
| | | AAGAGAG | | | | | | | CUCUCUU | |
| | | GCCACCGC | | | | | | | UCGCGGU | |
| | | C | | | | | | | | |
| 1588184 | 1588182 | AAGGUCU | 1469 | N/A | N/A | 1494 | 1515 | 1588183 | GCCCAGG | 1633 |
| | | GCUGGUA | | | | | | | UACCAGC | |
| | | CCUGGGCC | | | | | | | AGACCUU | |
| | | G | | | | | | | | |
| 1588202 | 1588200 | AAAAGAA | 1470 | N/A | N/A | 1446 | 1467 | 1588201 | GCGGAGA | 1634 |
| | | UGGCAGU | | | | | | | ACUGCCA | |
| | | UCUCCGCG | | | | | | | UUCUUUU | |
| | | G | | | | | | | | |
| 1588205 | 1588203 | AGCAGUU | 1471 | N/A | N/A | 1438 | 1459 | 1588204 | UCCACAC | 1635 |
| | | CUCCGCGG | | | | | | | CGCGGAG | |
| | | UGUGGAG | | | | | | | AACUGCU | |
| | | U | | | | | | | | |
| 1588407 | 1588405 | AGGUGCG | 1472 | N/A | N/A | 766 | 787 | 1588406 | UCCUCGC | 1636 |
| | | GAGGCCA | | | | | | | UGGCCUC | |
| | | GCGAGGA | | | | | | | CGCACCU | |
| | | GC | | | | | | | | |
| 1588782 | 1588780 | AUGCGCG | 1473 | N/A | N/A | 1501 | 1522 | 1588781 | UACCAGC | 1637 |
| | | CAGGUCU | | | | | | | AGACCUG | |
| | | GCUGGUA | | | | | | | CGCGCAU | |
| | | CC | | | | | | | | |

“Start site” indicates the 5′-most nucleoside to which the antisense RNAi oligonucleotide is complementary in the human gene sequence. “Stop site” indicates the 3′-most nucleoside to which the antisense RNAi oligonucleotide is complementary in the human gene sequence. The antisense RNAi oligonucleotide listed in the table below is complementary to SEQ ID NO: 4 (GENBANK Accession No. NM_001293798.2), with the exception of a single mismatch at position 1 (from 5′ to 3′) of the antisense RNAi oligonucleotide.

TABLE 25
Design of RNAi compounds targeted to human DUX4, SEQ ID NO: 4

| | | Antisense | | SEQ ID | SEQ ID | | Sense | |
|---|---|---|---|---|---|---|---|---|
| Duplex | Antisense | Strand | | NO: 4 | NO: 4 | Sense | Strand | SEQ |
| Compound | Strand | Sequence | SEQ | Antisense | Antisense | Strand | Sequence | ID |
| Number | Oligo ID | (5′ to 3′) | ID NO | Start Site | Stop Site | Oligo ID | (5′ to 3′) | NO |
| 1588217 | 1588215 | AGCCUAGA | 1638 | 1270 | 1291 | 1588216 | UUAGGAC | 1639 |
| | | CCCCGCGUC | | | | | GCGGGGU | |
| | | CUAAAG | | | | | CUAGGCU | |

### Example 13: Effect of RNAi Compounds on Human DUX4 RNA In Vitro, Single Dose

RNAi compounds described herein above were tested in 54-2 cells (Resnick et al., 2019, Cell Reports 29, 1812-1820). The RNAi compounds were tested in a series of experiments that had the same culture conditions.

54-2 cells were differentiated as described herein above and transfected with RNAi compounds at a concentration of 200 nM using Lipofectin. After a treatment period of approximately 24 hours, total RNA was isolated from the cells and DUX4 RNA levels were measured by quantitative real-time RTPCR.

DUX4 RNA was measured by primer probe set RTS40199 (described herein above). DUX4 RNA levels were normalized to total RNA content, as measured by RIBOGREEN®. The level of DUX4 RNA is presented in the tables below as percent DUX4 RNA relative to the amount of DUX4 RNA in untreated control cells (% UTC). Each table represents results from an individual assay plate. The values marked with a “?” indicate that the RNAi compound is complementary to the amplicon region of the primer probe set.

TABLE 26
Reduction of human DUX4 RNA by RNAi compounds
in differentiated 54-2 cells

| Compound No. | DUX4 (% UTC) |
|---|---|
| 1586340 | 13 |
| 1586341 | 17 |
| 1588142 | 15† |
| 1588166 | 114† |
| 1588190 | 140 |
| 1588196 | 66 |
| 1588199 | 76 |
| 1588226 | 107 |
| 1588235 | 85 |
| 1588250 | 110 |
| 1588253 | 109 |
| 1588617 | 83 |
| 1588620 | 45 |
| 1588623 | 8 |
| 1588626 | 13 |
| 1588629 | 259 |
| 1588632 | 64 |
| 1588635 | 137 |
| 1588638 | 97 |
| 1588641 | 130 |
| 1588644 | 60 |
| 1588647 | 110 |
| 1588650 | 72 |
| 1588653 | 74 |
| 1588656 | 43 |
| 1588659 | 96 |
| 1588662 | 56 |
| 1588665 | 57 |
| 1588668 | 78 |
| 1588671 | 68 |
| 1588674 | 21 |
| 1588677 | 100 |
| 1588680 | 92 |
| 1588683 | 93 |
| 1588686 | 73 |
| 1588689 | 111 |
| 1588692 | 52 |
| 1588695 | 93 |
| 1588698 | 107 |
| 1588701 | 154 |
| 1588704 | 63 |
| 1588707 | 55 |
| 1588710 | 20 |
| 1588713 | 105 |
| 1588716 | 72 |
| 1588719 | 116 |
| 1588722 | 71 |
| 1588725 | 145 |
| 1588728 | 107 |
| 1588731 | 81 |
| 1588734 | 50 |
| 1588737 | 117 |
| 1588740 | 88 |
| 1588743 | 96 |
| 1588746 | 81 |
| 1588749 | 184 |
| 1588752 | 86 |
| 1588755 | 79 |
| 1588758 | 23 |
| 1588761 | 11† |
| 1588764 | 26† |
| 1588767 | 56† |
| 1588770 | 48† |
| 1588773 | 12† |
| 1588776 | 68† |
| 1588779 | 221 |
| 1588782 | 96 |
| 1588785 | 83 |
| 1588788 | 102 |
| 1588791 | 133 |
| 1588794 | 90 |
| 1588797 | 107 |
| 1588800 | 116 |
| 1588803 | 130 |
| 1588806 | 49 |
| 1588809 | 80 |
| 1588812 | 108 |
| 1588815 | 81 |

TABLE 27
Reduction of human DUX4 RNA by RNAi compounds
in differentiated 54-2 cells

| Compound No. | DUX4 (% UTC) |
|---|---|
| 1588383 | 216 |
| 1588386 | 86 |
| 1588389 | 108 |
| 1588392 | 170 |
| 1588395 | 150 |
| 1588398 | 129 |
| 1588401 | 175 |
| 1588404 | 85 |
| 1588407 | 53 |
| 1588410 | 120 |
| 1588413 | 62 |
| 1588416 | 92 |
| 1588419 | 50 |
| 1588422 | 80 |
| 1588425 | 75 |
| 1588428 | 93 |
| 1588431 | 29 |
| 1588434 | 45 |
| 1588437 | 50 |
| 1588440 | 93 |
| 1588443 | 51 |
| 1588446 | 110 |
| 1588449 | 79 |
| 1588452 | 111 |
| 1588455 | 88 |
| 1588458 | 145 |
| 1588461 | 59 |
| 1588464 | 86 |
| 1588467 | 16 |
| 1588470 | 72 |
| 1588473 | 108 |
| 1588476 | 110 |
| 1588479 | 40 |
| 1588482 | 112 |
| 1588485 | 90 |
| 1588488 | 56 |
| 1588491 | 44 |
| 1588494 | 101 |
| 1588497 | 71 |
| 1588500 | 98 |
| 1588503 | 72 |
| 1588506 | 80 |
| 1588509 | 46 |
| 1588512 | 98 |
| 1588515 | 71 |
| 1588518 | 230 |
| 1588521 | 78 |
| 1588524 | 88 |
| 1588527 | 128 |
| 1588530 | 218 |
| 1588533 | 114 |
| 1588536 | 58 |
| 1588539 | 7 |
| 1588542 | 7 |
| 1588545 | 36 |
| 1588548 | 107 |
| 1588551 | 162 |
| 1588554 | 42 |
| 1588557 | 77 |
| 1588560 | 85 |
| 1588563 | 127 |
| 1588566 | 73 |
| 1588569 | 9 |
| 1588572 | 37 |
| 1588575 | 105 |
| 1588578 | 42 |
| 1588581 | 96 |
| 1588584 | 83 |
| 1588587 | 112 |
| 1588590 | 80 |
| 1588593 | 75 |
| 1588596 | 79 |
| 1588599 | 81 |
| 1588602 | 75 |
| 1588605 | 104 |
| 1588608 | 98 |
| 1588611 | 69 |
| 1588614 | 50 |

TABLE 28
Reduction of human DUX4 RNA by RNAi compounds
in differentiated 54-2 cells

| Compound No. | DUX4 (% UTC) |
|---|---|
| 1588121 | 7† |
| 1588124 | 23† |
| 1588127 | 5† |
| 1588130 | 22† |
| 1588133 | 4† |
| 1588136 | 25† |
| 1588139 | 10† |
| 1588145 | 12† |
| 1588148 | 39† |
| 1588151 | 16† |
| 1588154 | 2† |
| 1588157 | 12† |
| 1588160 | 17† |
| 1588163 | 36† |
| 1588169 | 76 |
| 1588172 | 44 |
| 1588175 | 92 |
| 1588178 | 58 |
| 1588181 | 77 |
| 1588184 | 115 |
| 1588187 | 95 |
| 1588193 | 75 |
| 1588202 | 94 |
| 1588205 | 48 |
| 1588208 | 87 |
| 1588211 | 99 |
| 1588214 | 112 |
| 1588217 | 79 |
| 1588220 | 90 |
| 1588223 | 75 |
| 1588229 | 34 |
| 1588232 | 36 |
| 1588238 | 41 |
| 1588241 | 50 |
| 1588244 | 131 |
| 1588247 | 32 |
| 1588256 | 63 |
| 1588259 | 49 |
| 1588262 | 51 |
| 1588265 | 78 |
| 1588268 | 55 |
| 1588271 | 82 |
| 1588274 | 62 |
| 1588277 | 83 |
| 1588280 | 71 |
| 1588283 | 40 |
| 1588286 | 20 |
| 1588289 | 109 |
| 1588292 | 102 |
| 1588295 | 146 |
| 1588298 | 60 |
| 1588301 | 85 |
| 1588304 | 89 |
| 1588307 | 29 |
| 1588310 | 84 |
| 1588313 | 33 |
| 1588316 | 53 |
| 1588319 | 152 |
| 1588322 | 11 |
| 1588325 | 236 |
| 1588328 | 91 |
| 1588332 | 105 |
| 1588335 | 109 |
| 1588338 | 109 |
| 1588341 | 193 |
| 1588344 | 91 |
| 1588347 | 43 |
| 1588350 | 76 |
| 1588353 | 123 |
| 1588356 | 57 |
| 1588359 | 88 |
| 1588362 | 83 |
| 1588365 | 126 |
| 1588368 | 129 |
| 1588371 | 31 |
| 1588374 | 117 |
| 1588377 | 59 |
| 1588380 | 80 |

1.-4. (canceled)

5. An oligomeric compound comprising a modified oligonucleotide consisting of 12 to 50 linked nucleosides and having a nucleobase sequence comprising at least 8, at least 9, at least 10, at least 11, at least 12, at least 13, at least 14, at least 15, at least 16, at least 17, at least 18, at least 19, at least 20, at least 21, at least 22, or 23 nucleobases complementary to:
wherein the modified oligonucleotide comprises at least one modification selected from a modified sugar moiety and a modified internucleoside linkage.

6. An oligomeric compound comprising a modified oligonucleotide consisting of 12 to 50 linked nucleosides and having a nucleobase sequence comprising at least 8, at least 9, at least 10, at least 11, at least 12, at least 13, at least 14, at least 15, at least 16, at least 17, at least 18, at least 19, at least 20, at least 21, at least 22, or 23 nucleobases of a sequence selected from:
wherein the modified oligonucleotide comprises at least one modification selected from a modified sugar moiety and a modified internucleoside linkage.

7. The oligomeric compound of claim 5, wherein the modified oligonucleotide has a nucleobase sequence that is at least 8500, at least 900%, at least 9500, or 100% complementary to any of the nucleobase sequences of SEQ ID NOs: 1-4 when measured across the entire nucleobase sequence of the modified oligonucleotide.

8. The oligomeric compound of claim 5, wherein the modified oligonucleotide consists of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

9. The oligomeric compound of claim 5, wherein the modified oligonucleotide comprises at least one modified nucleoside.

10. The oligomeric compound of claim 9, wherein the modified oligonucleotide comprises at least one modified nucleoside comprising a modified sugar moiety.

11. The oligomeric compound of claim 10, wherein the modified sugar moiety is a bicyclic sugar moiety.

12. The oligomeric compound of claim 11, wherein the bicyclic sugar moiety comprises a 2′-4′ bridge selected from —O—CH₂—; and —O—CH(CH₃)—.

13. The oligomeric compound of claim 10, wherein the modified sugar moiety is a non-bicyclic modified sugar moiety.

14. The oligomeric compound of claim 13, wherein the non-bicyclic modified sugar moiety is a 2′-O(CH₂)₂OCH₃ ribosyl sugar moiety, a cEt sugar moiety, a 2′-OMe sugar moiety, or a 2′-F sugar moiety.

15. The oligomeric compound of claim 10, wherein the modified sugar moiety is a sugar surrogate.

16. The oligomeric compound of claim 15, wherein the sugar surrogate is any of morpholino, modified morpholino, PNA, THP, and F-HNA.

17. The oligomeric compound of claim 5, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides; wherein
each of the 5′-region nucleosides and each of the 3′-region nucleosides comprises a modified sugar moiety and each of the central region nucleosides comprises a 2′-β-D-deoxyribosyl sugar moiety.

18.-21. (canceled)

22. The oligomeric compound of claim 5, wherein the modified oligonucleotide comprises at least one modified internucleoside linkage.

23. (canceled)

24. The oligomeric compound of claim 22, wherein at least one internucleoside linkage is a phosphorothioate internucleoside linkage.

25. (canceled)

26. The oligomeric compound of claim 22, wherein each internucleoside linkage is independently selected from a phosphodiester internucleoside linkage and a phosphorothioate internucleoside linkage.

27. (canceled)

28. The oligomeric compound of claim 5, wherein the modified oligonucleotide comprises a modified nucleobase.

29. The oligomeric compound of claim 28, wherein the modified nucleobase is a 5-methylcytosine.

30. The oligomeric compound of claim 5, wherein the modified oligonucleotide consists of 12-30, 12-22, 12-20,14-18, 14-20, 15-17, 15-25, 16-20, 18-22 or 18-20 linked nucleosides, or a pharmaceutically acceptable salt thereof.

31. The oligomeric compound of claim 30, which is a pharmaceutically acceptable salt comprising one or more cations selected from sodium, potassium, calcium, and magnesium.

32.-35. (canceled)

36. The oligomeric compound of claim 5, consisting of the modified oligonucleotide.

37. The oligomeric compound of claim 5, consisting of the modified oligonucleotide and a conjugate group.

38.-113. (canceled)

114. A pharmaceutical composition comprising an oligomeric compound of claim 5 and a pharmaceutically acceptable diluent.

115. The pharmaceutical composition of claim 114, wherein the pharmaceutically acceptable diluent is phosphate buffered saline (PBS).

116. The pharmaceutical composition of claim 115, wherein the pharmaceutical composition consists essentially of the oligomeric compound and PBS.

117.-133. (canceled)

134. The oligomeric compound of claim 6, wherein the modified sugar moiety is a bicyclic modified sugar moiety.

135. The oligomeric compound of claim 6, wherein the modified sugar moiety is a non-bicyclic modified sugar moiety.

136. The oligomeric compound of claim 6, wherein the modified internucleoside linkage is a phosphorothioate internucleoside linkage.

137. A pharmaceutical composition comprising an oligomeric compound of claim 6 and a pharmaceutically acceptable diluent.

138. The pharmaceutical composition of claim 137, wherein the pharmaceutically acceptable diluent is phosphate buffered saline (PBS).


SEQUENCE LISTING (1639 sequences)

SEQ ID NO: 1 - DNA, 16000 bp, Homo sapiens
  misc_feature (1)..(2121): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctcccccttcctcttcgtctctccggccccaccaccaccaccgccaccacgccctcccccaccacccccccccccaccaccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctggctggctggctggctgtccgggcaggcctcctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccactccgcggagaactgcctttctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagcctcccagctgccagcacggagcgcctggcggtcaaaagcatacctctgtctgtctttgcccgcttcctggctagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatagaagatttgcatcttttgtgtgatgagtgcagagatatgtcacaatatcccctgtagaaaaagcctgaaattgatttacagaacttcggtgatcagtgcagatgtgtttcagaactccatagtagactgaacctagagaatggttacatcacttaggtgatcagtgtagagatatgttaaaattctcgtgtagacagagcctagacaattgttacatcacctagtgatcagtgcagggataagtcataaagcctcctgtaggcagagtgtaggcaagtgttccctccctgggctgatcagtgcagagatatctcacaaagcccctataagccaaaccttgacaagggttacatcacctgtttgagcagtggaaatatatatcacaaagccccctgtagacaaagcccagacaatttttacatctcctgagtgagcattggagagatctgtcacaatgcccctgtaggcagagcttagacaagtgttacatcacctgggtgatcagtgcagagatgtgtcaaaacgctcctgtagtctgaacctagacaggagttacatcaccttggggatcagtgcagagatacgtgagaattcccttgtaggcagggcctagacaagtgttacatcacctaggttatcagtgcagagatatgtgagaattcccgtgtaggcagagcctagacaagtgttacatcacctagtttatcagtgtaattattagtcataaagcctcctgtaggcagaacgtagacaagagttccctcctcagggtgatcagtgcagagatgtgtcacaaagcccctgtaggcagagcctagacaagagtttcatcacttggttgatcagttcagagatgtgtcagaatgcccatgtaggcagatctaagacaagcgtccatcacctgggtgatcagtgcagagatatgtaccagtgtcccctgtaggcagtgcctagacaagagttgaatcacctcagagatcagtgcatagatatgtcacaaagccttctgtaggcaaagcccatacaaggtttagatcacctaggtgatcagtgcagtgatatgtcacaaaaatccctgtagacagagcctagacaagagttacttcacctgggtgatcagtgcagatatttgacacaatgcccccatagacagagcctaggcaagacttccatcacctgggtgatcagtgcagagatatgtcacaaatccccctctaggcagagtatagagaagagtcccatcacctgggtgatcagtgcagagatatttcacaatgtcccctgtaggcagagagtggacaagagttacatcacctagatgatctgtgcagagctatgtcaaaacgcccctgtaggcagagcctagatgagtgttacatcacctgggtgatcattgcagagatacgtcacaataccccctgtaggtggggcctagacaagagttacatcacctgggtgatcagtacagaagtatgtcacaaagcccctgtaggcagagcctagacaagagttacatcacctgggttatcagtgcagaaatatgtcacaaagcccctgtaggtcgagtctagacaagagttacatctcctgggtgatcagtgcaaagatatgtcacaaagccccctgtagacaaatcccagaaaattgttacatcacctgggtgatcagtggagatatgtgtcacaattcccctttaggcacagcttagacaagcgttacatcacatgagtgatcattgcagagttatgtcactatgcccccataggcagatccaagacaagagtccatcacctgggtgatcagtgcagaaatatgccacaatgccgccagtaggcagatatagacaagagttacatcacctgcgtgatcactgcagagatatatcacaatgcccctgtaggcagagcctagacaagagtcccatcacctgggtgatcagtgcagagttatgtcacaatgcccccttttggcagagcctagacaagggttacatcacctgggtgatcagtgcagagatatgtcacaatgtccctgtagccatatccttgacaaaagtgacatcacctgggtgatcagtgtggagatatgtcacaatgtctccagtaggcagagcctagacaagagttacatcacctgggtgatcagggcagagatatgtcacaatgccccctgtaagcagatcccagacaagagttgcatcacctcggtgatcagtgcagagatacgtcacaatgcccctgtaggcagagcctagacaagagttacataacccaggtgatccgtacagagtgatgtcacaacgccctctgtaggcagagactagaaaagagttacattacctgggtgatcagtgcagagatatgtcacaatgccccctgtaggcagagcatagagaagagttgcatcacctgggtgatcagtgtagagatatgtcacaatgtcccctgtaggcagagcacagagaagagttgcaccacctgggtgatcagtgcagagatatgtcacaatgtcccctgtaggcaaagcctaggcaagagttacatcacctttgtcatcagttcagggatatgtgaaaacgcccctgtaggcagagcttagacaagagttacatcacctagttgatcagtgcagagatatttcacaataccccctgtaggcagatcctagacaagagttgcatcacctgggtgatcagtgcagagatatttcacaacgccccctgtaggcagagggtagacaagagttacatcacttaggtgatcagtgcagagatttgtcgaaattccctgtaggcagtgcttataaaagtgttacatcacctaagtgatcagtgcagagatatgtcacaaagctcctgtaggcagagcttagatgagttacatcacctgggtgatcagagcaaaggtatgtcacaaagccccctgtaggccaaagcctagacaatagttacatcacttgggtgatcagtggcgagatctctcacaattcccctgtaggcagagcttatacaacagttacatcacctgggtgatcagtgcagatctatgtcacaatgcccccataggcagatccaagacaagagtccgtctcctgggtgatcagtgcagaaatacgtcacaatgcccccttaggcagagcctagacaaaagccccatcacctggatgattagtgcagagttatgtcacaaagtccctttaggcagatcctagaaaagagtcccattacttgggtgatcagtgcagagatatgtcacaatgccactgtaggcagagcctagacaagagttatatgacctaggtgatcagtgcagagatacattgcaatgcccctgtaggcagagccctgacaagtggtacatcacctgggtgatcattgcagggatatgtcacaaagcaccctgtaggcagatcctagacaagagttacatgacctgggtgatcagtgcagagatatgtcacaatgccactgtaggcagagcctagacaagagttacatgacctaggtgatcagtgcagagatacatcgcaatgcccctgtaggcagagccttgacaagtggtacatcacctgggtgatcattgcagggatatgtcacaaagcaccctgtaagcagatcctagagaagagttatatcacctgggtgatcagtgcagagatatgtcacaagccccctgtaggcagagcctagacaagagttatatcacctgggtgatcagtgcagtgatatgtcacaatgctgtgtagccagagcctagacaaaagttacagcacctgggagatcagtgcagagatatgtcacaatgtccccagtaggcagagaccaggcaagagttggatcacctcgggatcagtgcagagatatgtctcaatccccctgtgggcacagcctagacaagagttacatcacctcggttaacagtgcagagatatgtcaaaatgcccctgtaggcaagcctacacaagtattacatcacttaggtgatcagtgcagagatatgtcacaataccccctgtaagcagagcctagacaagagttacatcacctgggtgatcagtgcagagatatgtgacaaggcccctttaagcagagcctagacaatagttacatcacctgagtgatcagtgcagagatctgtcacaatgcccctttaggcagagcttaaactagagttacatcacctgtgtgatcagtgcagagatatgtcacattgcccccataggcgaatccaagacaagagtcagtcacctgggtgatcagtgcagaaatatgtgacaatgccgccagtaggcagagcctagagaagagtcccatcacctgggtgatcagtgcagagttatgccacaatgccctctgtaggcagagcctagacaagagttacatcatctgggtgatcagtaaagagatatttcacaatgcccctgcaggcagagcgtaagcaaaaggagtatgtgatcaaacaaatttagacccactcgattaaacgtgtgtcctttctgcagagtttcttagaacttctcatacactggtgggcattgtggctgtctaagtcagacgggtggcctgcaggtttcacaaagatcttggaccactgaattctgttattgcttgagaatattttaaaactgttttgcaaaggagtacaccttggaaaacatgagataacagttctatacctctgtcattttcattattaaagtattttttggcatgaaaaacttctttttaaagaaaacatgtggtttgatgtttcatcatcttggaaccttctgtcactttccacatccaccaccttgtgctaaactgtccacctgttcactgcaaaactcctcacctttttgccatttgttcactctgcccagtatttttactttcaaagttttacctcctttcacaagaggttatgtcaggaaatatacaatcgaagtgagaaggagaaagaaattcattacataagtcttaaaggaaaaaacagaaaatccaaagcatattgaaagaaagccttgcaagagaggaggctcaagagctttatgtcatagcaggacaccaagaaagttctaagaaggccatatcagaatgccctctgccgactttcaaccccaggatcaacacttcacatgcattagcagagagagataaatcaaaagtgatcagtgcagagatatgtcacaataccccctgtaagcagagcctagacaagagttacatcacctgggtgatcagtgcagagatatgtgacaaggcccctttaagcagagcctagacaatagttacatcacctgagtgatcagtgcagagatctgtcacaatgcccctttaggcagagcttagactagagttacatcacctgggtgatcagtgcagagatatgtcacattgcccccataggcaaatccaagacaagagtctgtcacctgggtgatcagtgcagaaatatgtgacaatgccgccagtaggcagagcctaaagaagagtcccatcccctgggtgatctctgcaaagatatgccacaatgccctctgtaggcagagcctaaacaagagttacatcatctggttgatcagtaagagatatttcacaatgcccctgcaggcagagcgtaagcaagagttacatcacctagatgatcagtgcagagatacgtcacaaggccccctataggcagagcctggacaagagttacatcacctcggtgatcaatgcagcgatatgtcactatgccccgtaggcagagcctagtcaagcgttacatcacctgggtgatcagtgcagagatatgtcagaaagccccctgtaggcagagcctagacaagagtcccatcacctgggtgatcagtgcagaaatatgtcacaatgcccccataggcagatccaacacaagagttacatcacctgggtgatcagtgcagagatatgtaacaatgcccccagtaggcagagcctagaggagagttacatcatctgggtgatctttgcagagatatgtcacaatcccccaagtaagcagagcctagacaaaagttacatcatctgggcgatcagtgcagagagaagtcacaaaacccacataggaaaagactagacaagagttacatcatctgggtcatcagtgcagacatatgtcaaagctgccgtagacagagtgtagacaattattacatcacttgggtgatcagtgcagagatctatcacagtgcccccataggcagagcctagacaagagttccatcacctgtgtgatcagtgtagagatatgtcacaatgcctcctgtaggcagaggctaaacaagagttacatcacctggattttgtttcctgcaatatgtcacaatggcgagggtgagggttagggtgagggtgagggttagggtgagggtcagggtgagggtgagggttagggttagggtgagcattaggtttagggttagggttagggttaggggttaggcttaggcttagggttaggcttaggcttagggtaaggcttagggttcaggttcaagtttggattcgggttcaggttaagagttagggttagggttactggttagggttaggggttagggttaggggttagggctgggttaggtttagggttagggttagggttatgggttacagttggggttagggttaggttttagggttaaggttagggttaggattggggttaggtttagggttagggtagtgtacctggtttcacattattactaataataaattattatttatacctaactgtaacccataaccctaaccctaacgggaacctaacccagccctaacccctaaccctatttgtttccctaaccagtaaccctaaccctaactcttaacctgaacctagaccagagttccatcacgtgtgtgatcagtgtagagatatgtcactagtgcctcccgtaggcagaggctaaacagagttacatcaccgggattttgtttcctgcaatatgtcacaatagcgaggatgagggttagggtgaggatgagggttagggtgagggtcacggtgagggtgagggttagggttagggtgagcattaggtttaggattagggttagggttaggggttaggcttaggcttagggttaggcttaggcttagggtaaggcttagggttcaggttcaagtttggattcgggttcaggttaagagttagggttagggttactggttagggttaggggttagggttaggggttagggctgggttaggtttagggttagggttagggttatgggttacagttagggttagggttaggttttagggttaaggttagggttaggattggggttaggtttagggttagggtagtgtaaataatttcacattattactaataataaattattatttatattacactattacttaatatataggctattaagacatgtttgtcttcaaagaatggccttggtttctgtggacagtttctcctcatggaaaggtagtgtgttcctgctaaatcatggacaaaacgggtccccaggagctacaggctgcagaagcagcttctcctctatgttcttcactgcctcatactgttgttgaccttgaaaccttcttttggtctagttttatcaacagagctagtatttacatgaggttctactacataccaggttccagaaagctaaatgctttttgtttgtttttattcactaaatacaaatcacaactctcttctcattacacacacaacaaaatttagctgagggagattgagtgactttcccagggtcacacagctactaatagcagagtagtgtttagattcatatgggaatactgaacacagaaatgaaccaatggaaacatcctacgttccaaaagcctactcaagccatttgttcttattttaaggaaaatatgctaattttaaactccaaatacttatgaatggcagagatctacagatttgattctgatgtaagaaatgatgctcaccagctggttactgctaccaccccacaacccgcagcatactggacaaatgtctaagcctcgtggttagtggggacattgctggtggagtctgaaattgtcatgcagtgactcactcaagcttaggcagatttggtgatatatgacacagagatgcaaagaaatgttgtagctgacacacacaggctggctctgggagatgcagaaggagcacgtcccccaaaatgaagccagacagacatccttaaggaaggagcaaaggggcttcatcttaaagaatgaagaagggatttgtcatgagagatgtggcaggaatttcttgagaggcagagggagagcatgagaatgttaggaaggcaggagagactctcacacatctgggaagctgacaatccatcagcatgtccaggaggaaaataaggaggaggagcagaaatagatgaggctggatatagaagcagggctgaagctgtgttgtttgtggtaaagagttgtgattctatccagaaggcaataggtagcattctaaacagagatcctttttttttttgagatggagtcttgctctgttgcccgggctggaatgcagtgacacgatcccgggtcactgcaagctcctcctcccaggttcacaccattctcctgcctcagcctctggagtagctgggactacagtcgcccgccaccacacccggctaatttttttgtatttttagtagagacgaggtttcactgggttagccaggatggtcttgatctcctggcctcgtgatccgctagcctcggcgtcccaaagtgctgggattacagacgtgagccaccacgcctggccacagagatcttttaaaacaagagtcagcaaatattttctgcaaagggctaaatgttaaatattttaaattttccaagccatatggtctctctctcaatgactcagctcttccattataccatgaaagtagccagagacgttatataacacatgtacgtggctgtgtcccattacaactttacttacaaacgcagactgtgtcagacatagtccatgtatggtagtttgccacaccttgttttagaaagctcaggtttatgatgtgatggagaattcctacaagagttcttgttttaaatggtagagtgaacatacactggaattctatactgcttgacccaagcttttgatagcaaaaggtagaaaagacagatagtaaacagatagatagatgataggaaggtagatagatagatgatagataaagaaagtacatagctgttccagaaaacagaaactgaaaatttcatgaaccaaaagcagagtaatatactttagaaaggaagcaggctggaaaacccacagttgcaaagcaaatggaatttccaactgcctcttgtagccccttccttgaagtagtcatagctcagggtgtttgacttcttcctctgttttttgtttgtttgttgtttgcttttctgtggggtttttgttgttgttgtttgctttttttaaaaaaaattccctttcactgcttttttgtcacagcagcctttgtcgcttcaaacaccgcaagtgttcttttaaaagaattatatcaacctttcaagtgaaatgcaacatgtctgaaacgtggtatctggagaggtgagatggacaaaggagcccttgttactgcacgttttcattctccaaacttcaccttgcacacagtaacagacaatgcacaaatccacttccttatggacggaaattctgaaatccttttatgcctggcctttccatccttcaacttcccctttcccatgctgtgaatgattgtattggacatttttgttttaatctcagtgacaggggaacacaggtagctctaatatagctgtgactgagatgcttctgtttctagcgtgtatttattttgcagcaaacatttacatccatgattttcactgtcttttgaaaataattaagcaatatctcatctgaggtagaatgtttctagtggttgtgttctgagggagaaaaactaatcttttctctttccactgcattctaggaacagtaagaggaccttgtgagtgaataatttgtttccacattacagagtgggtaataagcagattagtaaaaacaattctgcttcacttcaataacagcctcctccaactcattttttctcaacaaacttatttttccagcagaagaatcccagacttcttagagaacccagtgactttttgcaccttaaatctgtgaaatcctcatgttttcttctgctgtatctatagttcaaacaaagatgaggcaaaactagacgcattcctgaaggaacccaagaaattcctctttctgtctcggaatgaaatgaattctccagaccactagttctaaccttcaaaaaccaaacctgtttgtgagatctccttcaaatactactgtagaccctagtatttattcattaaatttttaaaatatttgttttatttggaatcaaagtatttgtaattttagtatttgtattaatataagggagaaatgtttaaatctgcctatgccatatgtgcctctggcttattgcccaattaattgtagtctcaggctaaactttcgtttctgtcttcaatttttgtcagaagaaatataactgatctcaaaacatctgcttttatgtagggacttgtgctgccatctccattcctctctctctttttgcaatctgggtggaagttctttaatatgaacatttcaaccagcttcattctaccacatccactatgagcacattcaaacgtatccagccaagactgtcatcttaggccagggatcttttaggaatctattttgctgtgatgtggctggcacccctttgattcactgtatcaccccagggttcttttcattttataagcccaagagggcagaaaatgaagtagatgagcaattaaacactgtgagtcaggagcgtctccccttgtgttaggcaatgttgtagaacatcgtatttagtaagctcctagcagatgagccatgtggcttctgagcacacatgcttgcttgctgctgtgaggtcagacaccatcatgtcttttccatctctggagggaattgtaagggccacttaataacctgtaaatcagagagataaaggtgcttccccaaaacactgatgacagaatgaaaggtgaggagtgttagccacaggtcaaaa

SEQ ID NO: 2 - DNA, 1710 bp, Homo sapiens
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 3 - DNA, 8671 bp, Homo sapiens
gggatgcgcgcgcctggggctctcccacagggggctttcgtgagccaggcagcgagggccgcccccgcgctgcagcccagccaggccgcgacggcagagggggtctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacgcctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagccgcccaggtgccagcacggagcgcctggcggcggaacgcagaccccaggcccggcgcacaccggggacgctgagcgttccaggcgggagggaaggcgggcagagatggagagaggaacgggagacctagaggggcggaaggacgggcggagggacgttaggagggagggagggaggcagggaggcagggaggaacggagggaaagacagagcgacgcagggactgggggcgggcgggagggagccggggacggacggggggaggaaggcagggaggaaaagcggtcctcggcctccgggagtagcgggacccccgccctccgggaaaacggtcagcgtccggcgcgggctgagggctgggcccacagccgccgcgccggccggcgcggcacccattcgccccggttccgtggcccagggagtgggcggtttcctccgggacaaaagaccgggactcgggttgccgtcgggttttcacccgcgcggttcacagaccgcacatccccaggctgagccctgcaacgcggcgcgaggccgacagccccggccacggaggagccacacgcaggacgacggaggcgtgattttggtttccgcgtggctttgccctccgcaaggcggcctgttgctcacgtctctccggcccccgaaaggctggccatgccgactgtttgctcccggagctctgcgggcacccggaaacatgcagggaagggtgcaagcccggcatggtgccttcgctctccttgccaggttccaaaccggccacactgcagactccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctccccgttcctcttcgtctctccggccccaccaccaccaccgccaccacgccctcccccaccaccccccccccccccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctgggtggctggctgtccgggcaggccccctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttggcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagccgcccaggtgccagcacggagcgcctggcggcggaacgcagaccccaggcccggcgcacaccggggacgctgagcgttccaggcgggagggaaggcgggcagagatggagagaggaacgggagacctagaggggcggaaggacgggcggagggacgttaggagggagggagggaggcagggaggcagggaggaacggagggaaagacagagcgacgcagggactgggggcgggcgggagggagccgggggacggggggaggaaggcagggaggaaaagcggtcctcggcctccgggagtagcgggacccccgccctccgggaaaacggtcagcgtccggcgcgggctgagggctgggcccacagccgccgcgccggccggcggggcaccacccattcgccccggttccggggcccagggagtgggcggtttcctccgggacaaaagaccgggactcgggttgccgtcgggtcttcacccgcgcggttcacagaccgcacatccccaggctcagccctgcaacgcggcgcgaggccgacagccccggccacggaggagccacacgcaggacgacggaggcgtgattttggtttccgcgtggctttgccctccgcaaggcggcctgttgctcacgtctctccggcccccgaaaggctggccatgccgactgtttgctcccggagctctgcgggcacccggaaacatgcagggaagggtgcaagcccggcacggtgccttcgctctccttgccaggttccaaaccggccacactgcagactccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctcccccttcctcttcgtctctccggccccaccaccaccaccgccaccacgccctccccccccaccccccccccccaccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctggctggctggctgtccgggcaggcctcctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggccgtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagcctcccagctgccagcgcggagctcctggcggtcaaaagcatacctctgtctgtctttgcccgcttcctggctagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatagaagatttgcatcttttgtgtgatgagtgcagagatatgtcacaatatcccctgtagaaaaagcctgaaattggtttacataacttcggtgatcagtgcagatgtgtttcagaactccatagtagactgaacctagagaatggttacatcacttaggtgatcagtgtagagatatgttaaaattctcgtgtagacagagcctagacaattgttacatcacctagtgatcagtgcagggataagtcataaagcctcctgtaggcagagtgtaggcaagtgttccctccctgggctgatcagtgcagagatatctcacaaagcccctataagccaaaccttgacaagggttacatcacctgtttgagcagtggaaatatatatcacaaagccccctgtagacaaagcccagacaatttttacatctcctgagtgagcattggagagatctgtcacaatgcccctgtaggcagagcttagacaagtgttacatcacctgggtgatcagtgcagagatatgtcaaaacgctcctgtagtctgaacctagacaggagttacatcaccttggggatcagtgcagagatacgtgagaattcc

SEQ ID NO: 4 - DNA, 1574 bp, Homo sapiens
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggtctaggcccggtgagagactccacaccgcggagaactgccattctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 5-10 - skipped (6 entries)

SEQ ID NO: 11 - DNA, 16 bp, Artificial sequence
  Primer
cccggctgacgtgcaa

SEQ ID NO: 12 - DNA, 22 bp, Artificial sequence
  Primer
agccagaatttcacggaagaac

SEQ ID NO: 13 - DNA, 22 bp, Artificial sequence
  Probe
agctcgctggcctctctgtgcc

SEQ ID NO: 14 - DNA, 20 bp, Artificial sequence
  Primer
ggcaaattcaacggcacagt

SEQ ID NO: 15 - DNA, 20 bp, Artificial sequence
  Primer
gggtctcgctcctggaagat

SEQ ID NO: 16 - DNA, 27 bp, Artificial sequence
  Probe
aaggccgagaatgggaagcttgtcatc

SEQ ID NO: 17 - DNA, 20 bp, Artificial sequence
  Primer
ctctctgtgcccttgttctt

SEQ ID NO: 18 - DNA, 25 bp, Artificial sequence
  Primer
catccaggagatgtaactctaatcc

SEQ ID NO: 19 - DNA, 23 bp, Artificial sequence
  Probe
ccttccgacgctgtctaggcaaa

SEQ ID NO: 20 - DNA, 20 bp, Artificial sequence
  Synthetic oligonucleotide
ccagccgcccttgtaaaggc

SEQ ID NO: 21 - DNA, 20 bp, Artificial sequence
  Synthetic oligonucleotide
gcccggacagccagccagcc

SEQ ID NO: 22 - DNA, 20 bp, Artificial sequence
  Synthetic oligonucleotide
ggcaggtgcagccaggaggc

SEQ ID NO: 23 - DNA, 20 bp, Artificial sequence
  Synthetic oligonucleotide
cctcagccggactgtgcact

SEQ ID NO: 24 - DNA, 20 bp, Artificial sequence
  Synthetic oligonucleotide
gaggccggcgggctcccgtg

SEQ ID NO: 25 - DNA, 20 bp, Artificial sequence
  Synthetic oligonucleotide
cacggacggacgcgggcaga

SEQ ID NO: 26 - DNA, 20 bp, Artificial sequence
  Synthetic oligonucleotide
cggtgagccccggccggaat

SEQ ID NO: 27 - DNA, 20 bp, Artificial sequence
  Synthetic oligonucleotide
tcgtccccgggcttccgcgg

SEQ ID NO: 28 - DNA, 20 bp, Artificial sequence
  Synthetic oligonucleotide
aaacgagtctccgtcgccgt

SEQ ID NO: 29 - DNA, 20 bp, Artificial sequence
  Synthetic oligonucleotide
agcaggctcgcagggcctcg

SEQ ID NO: 30 - DNA, 20 bp, Artificial sequence
  Synthetic oligonucleotide
gtggcgatgcccgggtacgg

SEQ ID NO: 31 - DNA, 20 bp, Artificial sequence
  Synthetic oligonucleotide
ggcctgggccagccgttctc

SEQ ID NO: 32 - DNA, 20 bp, Artificial sequence
  Synthetic oligonucleotide
ccctgggctccggaatgccg

SEQ ID NO: 33 - DNA, 20 bp, Artificial sequence
  Synthetic oligonucleotide
tcattctgaaaccaaatctg

SEQ ID NO: 34 - DNA, 20 bp, Artificial sequence
  Synthetic oligonucleotide
ctgcctcagctggcgtgacc

SEQ ID NO: 35 - DNA, 20 bp, Artificial sequence
  Synthetic oligonucleotide
agggccgagattcccgccgg

SEQ ID NO: 36 - DNA, 20 bp, Artificial sequence
  Synthetic oligonucleotide
ccttctggcgggccgcgtct

SEQ ID NO: 37 - DNA, 20 bp, Artificial sequence
  Synthetic oligonucleotide
gacggcggtccgctttcgcc

SEQ ID NO: 38 - DNA, 20 bp, Artificial sequence
  Synthetic oligonucleotide
ggaggagcagggcggtctgg

SEQ ID NO: 39 - DNA, 20 bp, Artificial sequence
  Synthetic oligonucleotide
aagcgatccttctcaaaggc

SEQ ID NO: 40 - DNA, 20 bp, Artificial sequence
  Synthetic oligonucleotide
ctcccgggcggcgatgcctg

SEQ ID NO: 41 - DNA, 20 bp, Artificial sequence
  Synthetic oligonucleotide
ggcccgtctctctggccagc

SEQ ID NO: 42 - DNA, 20 bp, Artificial sequence
  Synthetic oligonucleotide
atctgaatcctggactccgg

SEQ ID NO: 43 - DNA, 20 bp, Artificial sequence
  Synthetic oligonucleotide
ggcccttcgattctgaaacc

SEQ ID NO: 44 - DNA, 20 bp, Artificial sequence
  Synthetic oligonucleotide
tgccaccctgtcccgggtgc

SEQ ID NO: 45 - DNA, 20 bp, Artificial sequence
  Synthetic oligonucleotide
ccgcctgcctgcgcgggcgc

SEQ ID NO: 46 - DNA, 20 bp, Artificial sequence
  Synthetic oligonucleotide
gcgacccacgagggagcagg

SEQ ID NO: 47 - DNA, 20 bp, Artificial sequence
  Synthetic oligonucleotide
ccacgcgccggtgtgggcga

SEQ ID NO: 48 - DNA, 20 bp, Artificial sequence
  Synthetic oligonucleotide
caggcgcgcagggcacgtgg

SEQ ID NO: 49 - DNA, 20 bp, Artificial sequence
  Synthetic oligonucleotide
cctggctcacgaaagccccc

SEQ ID NO: 50 - DNA, 20 bp, Artificial sequence
  Synthetic oligonucleotide
ctggctgggctgcagcgcgg

SEQ ID NO: 51 - DNA, 20 bp, Artificial sequence
  Synthetic oligonucleotide
agatcccctctgccggcgcg

SEQ ID NO: 52 - DNA, 20 bp, Artificial sequence
  Synthetic oligonucleotide
cgaaatccccgcgcgccggg

SEQ ID NO: 53 - DNA, 20 bp, Artificial sequence
  Synthetic oligonucleotide
ccccgtccggaggagccggg

SEQ ID NO: 54 - DNA, 20 bp, Artificial sequence
  Synthetic oligonucleotide
agcgaggagcctgagggtgg

SEQ ID NO: 55 - DNA, 20 bp, Artificial sequence
  Synthetic oligonucleotide
cttttgcccgggtgcggagg

SEQ ID NO: 56 - DNA, 20 bp, Artificial sequence
  Synthetic oligonucleotide
ctgcgggtcccggtcctccc

SEQ ID NO: 57 - DNA, 20 bp, Artificial sequence
  Synthetic oligonucleotide
ggctgtgccaccgcgcaggg

SEQ ID NO: 58 - DNA, 20 bp, Artificial sequence
  Synthetic oligonucleotide
cggccccgcttgagcgggcc

SEQ ID NO: 59 - DNA, 20 bp, Artificial sequence
  Synthetic oligonucleotide
gcgcaagcaccccttggccc

SEQ ID NO: 60 - DNA, 20 bp, Artificial sequence
  Synthetic oligonucleotide
cggactcccctgggacgtgg

SEQ ID NO: 61 - DNA, 20 bp, Artificial sequence
  Synthetic oligonucleotide
gaccccggccccagccccac

SEQ ID NO: 62 - DNA, 20 bp, Artificial sequence
  Synthetic oligonucleotide
tggagctgccccggcttggg

SEQ ID NO: 63 - DNA, 20 bp, Artificial sequence
  Synthetic oligonucleotide
cccctgccgcgcggaggcgg

SEQ ID NO: 64 - DNA, 20 bp, Artificial sequence
  Synthetic oligonucleotide
gcgccgggatgccttgcatc

SEQ ID NO: 65 - DNA, 20 bp, Artificial sequence
  Synthetic oligonucleotide
cagggcgccggctcctggag

SEQ ID NO: 66 - DNA, 20 bp, Artificial sequence
  Synthetic oligonucleotide
tccagcagcaggccgcaggg

SEQ ID NO: 67 - DNA, 20 bp, Artificial sequence
  Synthetic oligonucleotide
ctccgggctcgccaggagct

SEQ ID NO: 68 - DNA, 20 bp, Artificial sequence
  Synthetic oligonucleotide
gaggttgcgcctgctgcaga

SEQ ID NO: 69 - DNA, 20 bp, Artificial sequence
  Synthetic oligonucleotide
aggcctccagctcccccggg

SEQ ID NO: 70 - DNA, 20 bp, Artificial sequence
  Synthetic oligonucleotide
agcagagcccggtattcttc

SEQ ID NO: 71 - DNA, 20 bp, Artificial sequence
  Synthetic oligonucleotide
ccccgcgtcctaaagctcct

SEQ ID NO: 72 - DNA, 20 bp, Artificial sequence
  Synthetic oligonucleotide
gaaccacccgaccccgtccc

SEQ ID NO: 73 - DNA, 20 bp, Artificial sequence
  Synthetic oligonucleotide
ccagccaggtgttccccgcg

SEQ ID NO: 74 - DNA, 20 bp, Artificial sequence
  Synthetic oligonucleotide
gcggagacacgcccctccgt

SEQ ID NO: 75 - DNA, 20 bp, Artificial sequence
  Synthetic oligonucleotide
ggaatcccaggccggtcagc

SEQ ID NO: 76 - DNA, 20 bp, Artificial sequence
  Synthetic oligonucleotide
ccgggcctagacctagaagg

SEQ ID NO: 77 - DNA, 20 bp, Artificial sequence
  Synthetic oligonucleotide
ctccgcggtgtggagtctct

SEQ ID NO: 78 - DNA, 20 bp, Artificial sequence
  Synthetic oligonucleotide
tgcccaggaaagaatggcag

SEQ ID NO: 79 - DNA, 20 bp, Artificial sequence
  Synthetic oligonucleotide
gccggctctgggatccccgg

SEQ ID NO: 80 - DNA, 20 bp, Artificial sequence
  Synthetic oligonucleotide
tgcgcagtaggcggcccacc

SEQ ID NO: 81 - DNA, 20 bp, Artificial sequence
  Synthetic oligonucleotide
cggctgcccgcaaacccgcg

SEQ ID NO: 82 - DNA, 20 bp, Artificial sequence
  Synthetic oligonucleotide
cgggctgctcccacagccca

SEQ ID NO: 83 - DNA, 20 bp, Artificial sequence
  Synthetic oligonucleotide
ggagaggcaggagagctctg

SEQ ID NO: 84 - DNA, 20 bp, Artificial sequence
  Synthetic oligonucleotide
ggtctccacccagcccaggg

SEQ ID NO: 85 - DNA, 20 bp, Artificial sequence
  Synthetic oligonucleotide
aggcccggacgctgcgcggg

SEQ ID NO: 86 - DNA, 20 bp, Artificial sequence
  Synthetic oligonucleotide
ggcgggcgacggtggcgcgg

SEQ ID NO: 87 - DNA, 20 bp, Artificial sequence
  Synthetic oligonucleotide
aggtatgcttttgaccgcca

SEQ ID NO: 88 - DNA, 20 bp, Artificial sequence
  Synthetic oligonucleotide
ggaagcgggcaaagacagac

SEQ ID NO: 89 - DNA, 20 bp, Artificial sequence
  Synthetic oligonucleotide
gcactgcgcgcaggtctagc

SEQ ID NO: 90 - DNA, 20 bp, Artificial sequence
  Synthetic oligonucleotide
ggccagcgagctcccttgca

SEQ ID NO: 91 - DNA, 20 bp, Artificial sequence
  Synthetic oligonucleotide
cggaagaacaagggcacaga

SEQ ID NO: 92 - DNA, 20 bp, Artificial sequence
  Synthetic oligonucleotide
agacagcgtcggaaggtggg

SEQ ID NO: 93 - DNA, 20 bp, Artificial sequence
  Synthetic oligonucleotide
taactctaatccaggtttgc

SEQ ID NO: 94 - DNA, 20 bp, Artificial sequence
  Synthetic oligonucleotide
tgaactaatcatccaggaga

SEQ ID NO: 95 - DNA, 20 bp, Artificial sequence
  Synthetic oligonucleotide
cagccagccagccagccgcc

SEQ ID NO: 96 - DNA, 20 bp, Artificial sequence
  Synthetic oligonucleotide
ccaggaggcctgcccggaca

SEQ ID NO: 97 - DNA, 20 bp, Artificial sequence
  Synthetic oligonucleotide
ctgtgcactgcggcaggtgc

SEQ ID NO: 98 - DNA, 20 bp, Artificial sequence
  Synthetic oligonucleotide
gctcccgtgcacctcagccg

SEQ ID NO: 99 - DNA, 20 bp, Artificial sequence
  Synthetic oligonucleotide
gcgggcagagagaggccggc

SEQ ID NO: 100 - DNA, 20 bp, Artificial sequence
  Synthetic oligonucleotide
ggccggaatttcacggacgg

SEQ ID NO: 101 - DNA, 20 bp, Artificial sequence
  Synthetic oligonucleotide
gagggccatcgcggtgagcc

SEQ ID NO: 102 - DNA, 20 bp, Artificial sequence
  Synthetic oligonucleotide
cgtcgccgtcctcgtccccg

SEQ ID NO: 103 - DNA, 20 bp, Artificial sequence
  Synthetic oligonucleotide
agggcctcgctttggctcgg

SEQ ID NO: 104 - DNA, 20 bp, Artificial sequence
  Synthetic oligonucleotide
gttccgctcaaagcaggctc

SEQ ID NO: 105 - DNA, 20 bp, Artificial sequence
  Synthetic oligonucleotide
gccgttctctggtggcgatg

SEQ ID NO: 106 - DNA, 20 bp, Artificial sequence
  Synthetic oligonucleotide
ggaatgccgatggcctgggc

SEQ ID NO: 107 - DNA, 20 bp, Artificial sequence
  Synthetic oligonucleotide
ccaaatctggaccctgggct

SEQ ID NO: 108 - DNA, 20 bp, Artificial sequence
  Synthetic oligonucleotide
ggcgtgacctctcattctga

SEQ ID NO: 109 - DNA, 20 bp, Artificial sequence
  Synthetic oligonucleotide
tcccgccggtgctgcctcag

SEQ ID NO: 110 - DNA, 20 bp, Artificial sequence
  Synthetic oligonucleotide
gccgcgtctcccgggccagg

SEQ ID NO: 111 - DNA, 20 bp, Artificial sequence
  Synthetic oligonucleotide
gctttcgccggccttctggc

SEQ ID NO: 112 - DNA, 20 bp, Artificial sequence
  Synthetic oligonucleotide
tgggatccggtgacggcggt

SEQ ID NO: 113 - DNA, 20 bp, Artificial sequence
  Synthetic oligonucleotide
ctcaaaggctcggaggagca

SEQ ID NO: 114 - DNA, 20 bp, Artificial sequence
  Synthetic oligonucleotide
cgatgcctggaaagcgatcc

SEQ ID NO: 115 - DNA, 20 bp, Artificial sequence
  Synthetic oligonucleotide
ctggccagctcctcccgggc

SEQ ID NO: 116 - DNA, 20 bp, Artificial sequence
  Synthetic oligonucleotide
ggactccgggaggcccgtct

SEQ ID NO: 117 - DNA, 20 bp, Artificial sequence
  Synthetic oligonucleotide
tctgaaaccagatctgaatc

SEQ ID NO: 118 - DNA, 20 bp, Artificial sequence
  Synthetic oligonucleotide
cccgggtgcctggcccttcg

SEQ ID NO: 119 - DNA, 20 bp, Artificial sequence
  Synthetic oligonucleotide
cgcgggcgccctgccaccct

SEQ ID NO: 120 - DNA, 20 bp, Artificial sequence
  Synthetic oligonucleotide
agcagggtgacccccgccgg

SEQ ID NO: 121 - DNA, 20 bp, Artificial sequence
  Synthetic oligonucleotide
tgtgggcgaaggcgacccac

SEQ ID NO: 122 - DNA, 20 bp, Artificial sequence
  Synthetic oligonucleotide
agccccgttccccacgcgcc

SEQ ID NO: 123 - DNA, 20 bp, Artificial sequence
  Synthetic oligonucleotide
aaagccccctgtgggagagc

SEQ ID NO: 124 - DNA, 20 bp, Artificial sequence
  Synthetic oligonucleotide
ggccctcgctgcctggctca

SEQ ID NO: 125 - DNA, 20 bp, Artificial sequence
  Synthetic oligonucleotide
gccggcgcggcctggctggg

SEQ ID NO: 126 - DNA, 20 bp, Artificial sequence
  Synthetic oligonucleotide
ggcaggttgggagatcccct

SEQ ID NO: 127 - DNA, 20 bp, Artificial sequence
  Synthetic oligonucleotide
ggcggcgtaggcgaaatccc

SEQ ID NO: 128 - DNA, 20 bp, Artificial sequence
  Synthetic oligonucleotide
gtgggagagcgccccgtccg

SEQ ID NO: 129 - DNA, 20 bp, Artificial sequence
  Synthetic oligonucleotide
gtgcggaggccagcgaggag

SEQ ID NO: 130 - DNA, 20 bp, Artificial sequence
  Synthetic oligonucleotide
ggtcctcccggcttttgccc

SEQ ID NO: 131 - DNA, 20 bp, Artificial sequence
  Synthetic oligonucleotide
aggccgtcgcgctgcgggtc

SEQ ID NO: 132 - DNA, 20 bp, Artificial sequence
  Synthetic oligonucleotide
gagcgggcccaggctgtgcc

SEQ ID NO: 133 - DNA, 20 bp, Artificial sequence
  Synthetic oligonucleotide
ccttggccctgcggccccgc

SEQ ID NO: 134 - DNA, 20 bp, Artificial sequence
  Synthetic oligonucleotide
ggacgtgggtggcgcaagca

SEQ ID NO: 135 - DNA, 20 bp, Artificial sequence
  Synthetic oligonucleotide
cagccccaccacggactccc

SEQ ID NO: 136 - DNA, 20 bp, Artificial sequence
  Synthetic oligonucleotide
cgccgccccggcgacctggg

SEQ ID NO: 137 - DNA, 20 bp, Artificial sequence
  Synthetic oligonucleotide
cggaggcggaggcgtccggg

SEQ ID NO: 138 - DNA, 20 bp, Artificial sequence
  Synthetic oligonucleotide
ccttgcatctgcccctgccg

SEQ ID NO: 139 - DNA, 20 bp, Artificial sequence
  Synthetic oligonucleotide
ctcctggagcgcctgggagg

SEQ ID NO: 140 - DNA, 20 bp, Artificial sequence
  Synthetic oligonucleotide
ggagtgcagaccagggcgcc

SEQ ID NO: 141 - DNA, 20 bp, Artificial sequence
  Synthetic oligonucleotide
ccaggagctcatccagcagc

SEQ ID NO: 142 - DNA, 20 bp, Artificial sequence
  Synthetic oligonucleotide
tgctgcagaaactccgggct

SEQ ID NO: 143 - DNA, 20 bp, Artificial sequence
  Synthetic oligonucleotide
cgtttctaggagaggttgcg

SEQ ID NO: 144 - DNA, 20 bp, Artificial sequence
  Synthetic oligonucleotide
gtattcttcctcgctgaggg

SEQ ID NO: 145 - DNA, 20 bp, Artificial sequence
  Synthetic oligonucleotide
aaagctcctccagcagagcc

SEQ ID NO: 146 - DNA, 20 bp, Artificial sequence
  Synthetic oligonucleotide
ccccgtcccaaccccgcgtc

SEQ ID NO: 147 - DNA, 20 bp, Artificial sequence
  Synthetic oligonucleotide
ttccccgcgaaagagaggcc

SEQ ID NO: 148 - DNA, 20 bp, Artificial sequence
  Synthetic oligonucleotide
cccctccgtagccagccagg

SEQ ID NO: 149 - DNA, 20 bp, Artificial sequence
  Synthetic oligonucleotide
ccggtcagcccggtggaggg

SEQ ID NO: 150 - DNA, 20 bp, Artificial sequence
  Synthetic oligonucleotide
cctagaaggcaggaatccca

SEQ ID NO: 151 - DNA, 20 bp, Artificial sequence
  Synthetic oligonucleotide
ggagtctctcaccgggccta

SEQ ID NO: 152 - DNA, 20 bp, Artificial sequence
  Synthetic oligonucleotide
gaatggcagttctccgcggt

SEQ ID NO: 153 - DNA, 20 bp, Artificial sequence
  Synthetic oligonucleotide
gatccccgggatgcccagga

SEQ ID NO: 154 - DNA, 20 bp, Artificial sequence
  Synthetic oligonucleotide
cggcccacctgctggtacct

SEQ ID NO: 155 - DNA, 20 bp, Artificial sequence
  Synthetic oligonucleotide
aaacccgcgcgtgcgcagta

SEQ ID NO: 156 - DNA, 20 bp, Artificial sequence
  Synthetic oligonucleotide
cacagcccaggcggctgccc

SEQ ID NO: 157 - DNA, 20 bp, Artificial sequence
  Synthetic oligonucleotide
agagctctgcccgggctgct

SEQ ID NO: 158 - DNA, 20 bp, Artificial sequence
  Synthetic oligonucleotide
ggtgggctggtggagaggca

SEQ ID NO: 159 - DNA, 20 bp, Artificial sequence
  Synthetic oligonucleotide
cccggtgtttcgcgggacgg

SEQ ID NO: 160 - DNA, 20 bp, Artificial sequence
  Synthetic oligonucleotide
ggcagctgggaggctgcagg

SEQ ID NO: 161 - DNA, 20 bp, Artificial sequence
  Synthetic oligonucleotide
aagacagacagaggtatgct

SEQ ID NO: 162 - DNA, 20 bp, Artificial sequence
  Synthetic oligonucleotide
aggtctagccaggaagcggg

SEQ ID NO: 163 - DNA, 20 bp, Artificial sequence
  Synthetic oligonucleotide
tcccttgcacgtcagccggg

SEQ ID NO: 164 - DNA, 20 bp, Artificial sequence
  Synthetic oligonucleotide
gggcacagagaggccagcga

SEQ ID NO: 165 - DNA, 20 bp, Artificial sequence
  Synthetic oligonucleotide
ccagaatttcacggaagaac

SEQ ID NO: 166 - DNA, 20 bp, Artificial sequence
  Synthetic oligonucleotide
caggtttgcctagacagcgt

SEQ ID NO: 167 - DNA, 20 bp, Artificial sequence
  Synthetic oligonucleotide
tccaggagatgtaactctaa

SEQ ID NO: 168 - DNA, 20 bp, Artificial sequence
  Synthetic oligonucleotide
taatatatctctgaactaat

SEQ ID NO: 169 - DNA, 20 bp, Artificial sequence
  Synthetic oligonucleotide
agagaggccacggccctgcc

SEQ ID NO: 170 - DNA, 20 bp, Artificial sequence
  Synthetic oligonucleotide
agctccgcgctggcagctgg

SEQ ID NO: 171 - DNA, 20 bp, Artificial sequence
  Synthetic oligonucleotide
ggccctgccccgaaccaccc

SEQ ID NO: 172 - DNA, 20 bp, Artificial sequence
  Synthetic oligonucleotide
tgaccgccaggagctccgcg

SEQ ID NO: 173 - DNA, 16 bp, Artificial sequence
  Synthetic oligonucleotide
ggcccacctgctggta

SEQ ID NO: 174 - DNA, 16 bp, Artificial sequence
  Synthetic oligonucleotide
cacagcccaggcggct

SEQ ID NO: 175 - DNA, 16 bp, Artificial sequence
  Synthetic oligonucleotide
ggtgggctggtggaga

SEQ ID NO: 176 - DNA, 16 bp, Artificial sequence
  Synthetic oligonucleotide
gtgtttcgcgggacgg

SEQ ID NO: 177 - DNA, 16 bp, Artificial sequence
  Synthetic oligonucleotide
aggcgagccgccggag

SEQ ID NO: 178 - DNA, 16 bp, Artificial sequence
  Synthetic oligonucleotide
cgttgccgggacggtc

SEQ ID NO: 179 - DNA, 16 bp, Artificial sequence
  Synthetic oligonucleotide
gggagtcttgagtgtg

SEQ ID NO: 180 - DNA, 16 bp, Artificial sequence
  Synthetic oligonucleotide
tcggcagcagggagaa

SEQ ID NO: 181 - DNA, 16 bp, Artificial sequence
  Synthetic oligonucleotide
acgggaagccgctctc

SEQ ID NO: 182 - DNA, 16 bp, Artificial sequence
  Synthetic oligonucleotide
cggagagacgaagagg

SEQ ID NO: 183 - DNA, 16 bp, Artificial sequence
  Synthetic oligonucleotide
aatggcggtgagcccc

SEQ ID NO: 184 - DNA, 16 bp, Artificial sequence
  Synthetic oligonucleotide
agccagccgcccttgt

SEQ ID NO: 185 - DNA, 16 bp, Artificial sequence
  Synthetic oligonucleotide
cggcaggtgcagccag

SEQ ID NO: 186 - DNA, 16 bp, Artificial sequence
  Synthetic oligonucleotide
aatttcacggacggac

SEQ ID NO: 187 - DNA, 16 bp, Artificial sequence
  Synthetic oligonucleotide
acgagtctccgtcgcc

SEQ ID NO: 188 - DNA, 16 bp, Artificial sequence
  Synthetic oligonucleotide
gctcaaagcaggctcg

SEQ ID NO: 189 - DNA, 16 bp, Artificial sequence
  Synthetic oligonucleotide
cagccgttctctggtg

SEQ ID NO: 190 - DNA, 16 bp, Artificial sequence
  Synthetic oligonucleotide
cggaatgccgatggcc

SEQ ID NO: 191 - DNA, 16 bp, Artificial sequence
  Synthetic oligonucleotide
cggccttctggcgggc

SEQ ID NO: 192 - DNA, 16 bp, Artificial sequence
  Synthetic oligonucleotide
aaggctcggaggagca

SEQ ID NO: 193 - DNA, 16 bp, Artificial sequence
  Synthetic oligonucleotide
ggccagctcctcccgg

SEQ ID NO: 194 - DNA, 16 bp, Artificial sequence
  Synthetic oligonucleotide
actccgggaggcccgt

SEQ ID NO: 195 - DNA, 16 bp, Artificial sequence
  Synthetic oligonucleotide
aggccgcctgcctgcg

SEQ ID NO: 196 - DNA, 16 bp, Artificial sequence
  Synthetic oligonucleotide
gggaagccccgttccc

SEQ ID NO: 197 - DNA, 16 bp, Artificial sequence
  Synthetic oligonucleotide
agatcccctctgccgg

SEQ ID NO: 198 - DNA, 16 bp, Artificial sequence
  Synthetic oligonucleotide
cccgtccggaggagcc

SEQ ID NO: 199 - DNA, 16 bp, Artificial sequence
  Synthetic oligonucleotide
tttgcccgggtgcgga

SEQ ID NO: 200 - DNA, 16 bp, Artificial sequence
  Synthetic oligonucleotide
gtgccaccgcgcaggg

SEQ ID NO: 201 - DNA, 16 bp, Artificial sequence
  Synthetic oligonucleotide
ttggccctgcggcccc

SEQ ID NO: 202 - DNA, 16 bp, Artificial sequence
  Synthetic oligonucleotide
gccccggcgacctggg

SEQ ID NO: 203 - DNA, 16 bp, Artificial sequence
  Synthetic oligonucleotide
gggatgccttgcatct

SEQ ID NO: 204 - DNA, 16 bp, Artificial sequence
  Synthetic oligonucleotide
tccagcagcaggccgc

SEQ ID NO: 205 - DNA, 16 bp, Artificial sequence
  Synthetic oligonucleotide
gtttctaggagaggtt

SEQ ID NO: 206 - DNA, 16 bp, Artificial sequence
  Synthetic oligonucleotide
tgcttccagcgaggcg

SEQ ID NO: 207 - DNA, 16 bp, Artificial sequence
  Synthetic oligonucleotide
ccgtcccaaccccgcg

SEQ ID NO: 208 - DNA, 16 bp, Artificial sequence
  Synthetic oligonucleotide
ctccgtagccagccag

SEQ ID NO: 209 - DNA, 16 bp, Artificial sequence
  Synthetic oligonucleotide
cggtcagcccggtgga

SEQ ID NO: 210 - DNA, 16 bp, Artificial sequence
  Synthetic oligonucleotide
ctctcaccgggcctag

SEQ ID NO: 211 - DNA, 16 bp, Artificial sequence
  Synthetic oligonucleotide
ccgggatgcccaggaa

SEQ ID NO: 212 - DNA, 16 bp, Artificial sequence
  Synthetic oligonucleotide
gcccacctgctggtac

SEQ ID NO: 213 - DNA, 16 bp, Artificial sequence
  Synthetic oligonucleotide
gcggctgcccgcaaac

SEQ ID NO: 214 - DNA, 16 bp, Artificial sequence
  Synthetic oligonucleotide
cgggctgctcccacag

SEQ ID NO: 215 - DNA, 16 bp, Artificial sequence
  Synthetic oligonucleotide
ccggtgtttcgcggga

SEQ ID NO: 216 - DNA, 16 bp, Artificial sequence
  Synthetic oligonucleotide
acagaggtatgctttt

SEQ ID NO: 217 - DNA, 16 bp, Artificial sequence
  Synthetic oligonucleotide
cgcactgcgcgcaggt

SEQ ID NO: 218 - DNA, 16 bp, Artificial sequence
  Synthetic oligonucleotide
tcacggaagaacaagg

SEQ ID NO: 219 - DNA, 16 bp, Artificial sequence
  Synthetic oligonucleotide
gtttgcctagacagcg

SEQ ID NO: 220 - DNA, 16 bp, Artificial sequence
  Synthetic oligonucleotide
caggagatgtaactct

SEQ ID NO: 221 - DNA, 16 bp, Artificial sequence
  Synthetic oligonucleotide
aggatccacagggagg

SEQ ID NO: 222 - DNA, 16 bp, Artificial sequence
  Synthetic oligonucleotide
tattgtgacatatctc

SEQ ID NO: 223 - DNA, 16 bp, Artificial sequence
  Synthetic oligonucleotide
taaccattctctaggt

SEQ ID NO: 224 - DNA, 16 bp, Artificial sequence
  Synthetic oligonucleotide
ctgtctacacgagaat

SEQ ID NO: 225 - DNA, 16 bp, Artificial sequence
  Synthetic oligonucleotide
ctctgcctacaggagg

SEQ ID NO: 226 - DNA, 16 bp, Artificial sequence
  Synthetic oligonucleotide
tttgtgagatatctct

SEQ ID NO: 227 - DNA, 16 bp, Artificial sequence
  Synthetic oligonucleotide
tgtaacccttgtcaag

SEQ ID NO: 228 - DNA, 16 bp, Artificial sequence
  Synthetic oligonucleotide
ggctttgtgatatata

SEQ ID NO: 229 - DNA, 16 bp, Artificial sequence
  Synthetic oligonucleotide
ctctccaatgctcact

SEQ ID NO: 230 - DNA, 16 bp, Artificial sequence
  Synthetic oligonucleotide
taacacttgtctaagc

SEQ ID NO: 231 - DNA, 16 bp, Artificial sequence
  Synthetic oligonucleotide
ctgtctaggttcagac

SEQ ID NO: 232 - DNA, 16 bp, Artificial sequence
  Synthetic oligonucleotide
gtgcgcagtaggcggc

SEQ ID NO: 233 - DNA, 16 bp, Artificial sequence
  Synthetic oligonucleotide
agagctctgcccgggc

SEQ ID NO: 234 - DNA, 16 bp, Artificial sequence
  Synthetic oligonucleotide
gcggtcaggcggcggg

SEQ ID NO: 235 - DNA, 16 bp, Artificial sequence
  Synthetic oligonucleotide
gcggtgtcaggcccgg

SEQ ID NO: 236 - DNA, 16 bp, Artificial sequence
  Synthetic oligonucleotide
gcgacggtggcgcggg

SEQ ID NO: 237 - DNA, 16 bp, Artificial sequence
  Synthetic oligonucleotide
tctcgcacacgcaggc

SEQ ID NO: 238 - DNA, 16 bp, Artificial sequence
  Synthetic oligonucleotide
ctctccgtgaaggagg

SEQ ID NO: 239 - DNA, 16 bp, Artificial sequence
  Synthetic oligonucleotide
tgtggaactgaacctc

SEQ ID NO: 240 - DNA, 16 bp, Artificial sequence
  Synthetic oligonucleotide
tctgggctcccacgcg

SEQ ID NO: 241 - DNA, 16 bp, Artificial sequence
  Synthetic oligonucleotide
ccggacctctccaggg

SEQ ID NO: 242 - DNA, 16 bp, Artificial sequence
  Synthetic oligonucleotide
ccggaagggacccagg

SEQ ID NO: 243 - DNA, 16 bp, Artificial sequence
  Synthetic oligonucleotide
taaaggcccacaggca

SEQ ID NO: 244 - DNA, 16 bp, Artificial sequence
  Synthetic oligonucleotide
tgcccggacagccagc

SEQ ID NO: 245 - DNA, 16 bp, Artificial sequence
  Synthetic oligonucleotide
cacctcagccggactg

SEQ ID NO: 246 - DNA, 16 bp, Artificial sequence
  Synthetic oligonucleotide
gtgagccccggccgga

SEQ ID NO: 247 - DNA, 16 bp, Artificial sequence
  Synthetic oligonucleotide
agggcctcgctttggc

SEQ ID NO: 248 - DNA, 16 bp, Artificial sequence
  Synthetic oligonucleotide
ggcgatgcccgggtac

SEQ ID NO: 249 - DNA, 16 bp, Artificial sequence
  Synthetic oligonucleotide
cctgggccagccgttc

SEQ ID NO: 250 - DNA, 16 bp, Artificial sequence
  Synthetic oligonucleotide
cgccggtgctgcctca

SEQ ID NO: 251 - DNA, 16 bp, Artificial sequence
  Synthetic oligonucleotide
ctgggatccggtgacg

SEQ ID NO: 252 - DNA, 16 bp, Artificial sequence
  Synthetic oligonucleotide
cgatgcctggaaagcg

SEQ ID NO: 253 - DNA, 16 bp, Artificial sequence
  Synthetic oligonucleotide
cgggaggcccgtctct

SEQ ID NO: 254 - DNA, 16 bp, Artificial sequence
  Synthetic oligonucleotide
ggcccttcgattctga

SEQ ID NO: 255 - DNA, 16 bp, Artificial sequence
  Synthetic oligonucleotide
tgggcgaaggcgaccc

SEQ ID NO: 256 - DNA, 16 bp, Artificial sequence
  Synthetic oligonucleotide
ctggctcacgaaagcc

SEQ ID NO: 257 - DNA, 16 bp, Artificial sequence
  Synthetic oligonucleotide
taggcgaaatccccgc

SEQ ID NO: 258 - DNA, 16 bp, Artificial sequence
  Synthetic oligonucleotide
cagcgaggagcctgag

SEQ ID NO: 259 - DNA, 16 bp, Artificial sequence
  Synthetic oligonucleotide
ggcccggcaggccgtc

SEQ ID NO: 260 - DNA, 16 bp, Artificial sequence
  Synthetic oligonucleotide
ttgagcgggcccaggc

SEQ ID NO: 261 - DNA, 16 bp, Artificial sequence
  Synthetic oligonucleotide
gcaagcaccccttggc

SEQ ID NO: 262 - DNA, 16 bp, Artificial sequence
  Synthetic oligonucleotide
agctgccccggcttgg

SEQ ID NO: 263 - DNA, 16 bp, Artificial sequence
  Synthetic oligonucleotide
gtgcagaccagggcgc

SEQ ID NO: 264 - DNA, 16 bp, Artificial sequence
  Synthetic oligonucleotide
tgcgcctgctgcagaa

SEQ ID NO: 265 - DNA, 16 bp, Artificial sequence
  Synthetic oligonucleotide
ggcctcttccgaggcc

SEQ ID NO: 266 - DNA, 16 bp, Artificial sequence
  Synthetic oligonucleotide
agcccggtattcttcc

SEQ ID NO: 267 - DNA, 16 bp, Artificial sequence
  Synthetic oligonucleotide
ccgaaccacccgaccc

SEQ ID NO: 268 - DNA, 16 bp, Artificial sequence
  Synthetic oligonucleotide
gttccccgcgaaagag

SEQ ID NO: 269 - DNA, 16 bp, Artificial sequence
  Synthetic oligonucleotide
ggcggagacacgcccc

SEQ ID NO: 270 - DNA, 16 bp, Artificial sequence
  Synthetic oligonucleotide
tagaaggcaggaatcc

SEQ ID NO: 271 - DNA, 16 bp, Artificial sequence
  Synthetic oligonucleotide
aagaatggcagttctc

SEQ ID NO: 272 - DNA, 16 bp, Artificial sequence
  Synthetic oligonucleotide
cctgggccggctctgg

SEQ ID NO: 273 - DNA, 16 bp, Artificial sequence
  Synthetic oligonucleotide
tgcgcagtaggcggcc

SEQ ID NO: 274 - DNA, 16 bp, Artificial sequence
  Synthetic oligonucleotide
ggcggctgcccgcaaa

SEQ ID NO: 275 - DNA, 16 bp, Artificial sequence
  Synthetic oligonucleotide
gtgtcaggcccggacg

SEQ ID NO: 276 - DNA, 16 bp, Artificial sequence
  Synthetic oligonucleotide
gtatgcttttgaccgc

SEQ ID NO: 277 - DNA, 16 bp, Artificial sequence
  Synthetic oligonucleotide
agaggccagcgagctc

SEQ ID NO: 278 - DNA, 16 bp, Artificial sequence
  Synthetic oligonucleotide
cattcagccagaattt

SEQ ID NO: 279 - DNA, 16 bp, Artificial sequence
  Synthetic oligonucleotide
gtaactctaatccagg

SEQ ID NO: 280 - DNA, 16 bp, Artificial sequence
  Synthetic oligonucleotide
taatatatctctgaac

SEQ ID NO: 281 - DNA, 16 bp, Artificial sequence
  Synthetic oligonucleotide
gatgcaaatcttctat

SEQ ID NO: 282 - DNA, 16 bp, Artificial sequence
  Synthetic oligonucleotide
ttcagtctactatgga

SEQ ID NO: 283 - DNA, 16 bp, Artificial sequence
  Synthetic oligonucleotide
ctacactgatcaccta

SEQ ID NO: 284 - DNA, 16 bp, Artificial sequence
  Synthetic oligonucleotide
acaattgtctaggctc

SEQ ID NO: 285 - DNA, 16 bp, Artificial sequence
  Synthetic oligonucleotide
gaacacttgcctacac

SEQ ID NO: 286 - DNA, 16 bp, Artificial sequence
  Synthetic oligonucleotide
ggtttggcttataggg

SEQ ID NO: 287 - DNA, 16 bp, Artificial sequence
  Synthetic oligonucleotide
tatatttccactgctc

SEQ ID NO: 288 - DNA, 16 bp, Artificial sequence
  Synthetic oligonucleotide
ctgggctttgtctaca

SEQ ID NO: 289 - DNA, 16 bp, Artificial sequence
  Synthetic oligonucleotide
ttgtgacagatctctc

SEQ ID NO: 290 - DNA, 16 bp, Artificial sequence
  Synthetic oligonucleotide
caaggtgatgtaactc

SEQ ID NO: 291 - DNA, 16 bp, Artificial sequence
  Synthetic oligonucleotide
ggccatcgcggtgagc

SEQ ID NO: 292 - DNA, 16 bp, Artificial sequence
  Synthetic oligonucleotide
cccgcaaacccgcgcg

SEQ ID NO: 293 - DNA, 16 bp, Artificial sequence
  Synthetic oligonucleotide
gctgcccgcaaacccg

SEQ ID NO: 294 - DNA, 16 bp, Artificial sequence
  Synthetic oligonucleotide
caggcggctgcccgca

SEQ ID NO: 295 - DNA, 16 bp, Artificial sequence
  Synthetic oligonucleotide
gggcggtcaggcggcg

SEQ ID NO: 296 - DNA, 16 bp, Artificial sequence
  Synthetic oligonucleotide
gggcccggtgtttcgc

SEQ ID NO: 297 - DNA, 16 bp, Artificial sequence
  Synthetic oligonucleotide
gagcggtgtcaggccc

SEQ ID NO: 298 - DNA, 16 bp, Artificial sequence
  Synthetic oligonucleotide
cgccggagcggtgtca

SEQ ID NO: 299 - DNA, 16 bp, Artificial sequence
  Synthetic oligonucleotide
gagccgccggagcggt

SEQ ID NO: 300 - DNA, 16 bp, Artificial sequence
  Synthetic oligonucleotide
gaggcgagccgccgga

SEQ ID NO: 301 - DNA, 16 bp, Artificial sequence
  Synthetic oligonucleotide
agaggaggcgagccgc

SEQ ID NO: 302 - DNA, 16 bp, Artificial sequence
  Synthetic oligonucleotide
gggcgacggtggcgcg

SEQ ID NO: 303 - DNA, 16 bp, Artificial sequence
  Synthetic oligonucleotide
ccgggacggtctcgca

SEQ ID NO: 304 - DNA, 16 bp, Artificial sequence
  Synthetic oligonucleotide
tcgccgttgccgggac

SEQ ID NO: 305 - DNA, 16 bp, Artificial sequence
  Synthetic oligonucleotide
aggccctctctccgtg

SEQ ID NO: 306 - DNA, 16 bp, Artificial sequence
  Synthetic oligonucleotide
ggctctccggacctct

SEQ ID NO: 307 - DNA, 16 bp, Artificial sequence
  Synthetic oligonucleotide
ggccggctctccggac

SEQ ID NO: 308 - DNA, 16 bp, Artificial sequence
  Synthetic oligonucleotide
ccaccccggaagggac

SEQ ID NO: 309 - DNA, 16 bp, Artificial sequence
  Synthetic oligonucleotide
ccgccccaccccggaa

SEQ ID NO: 310 - DNA, 16 bp, Artificial sequence
  Synthetic oligonucleotide
agcccccctgggacag

SEQ ID NO: 311 - DNA, 16 bp, Artificial sequence
  Synthetic oligonucleotide
gcggtgagcccccctg

SEQ ID NO: 312 - DNA, 16 bp, Artificial sequence
  Synthetic oligonucleotide
tgtaaaggcccacagg

SEQ ID NO: 313 - DNA, 16 bp, Artificial sequence
  Synthetic oligonucleotide
cccttgtaaaggccca

SEQ ID NO: 314 - DNA, 16 bp, Artificial sequence
  Synthetic oligonucleotide
gccgcccttgtaaagg

SEQ ID NO: 315 - DNA, 16 bp, Artificial sequence
  Synthetic oligonucleotide
gcccggacagccagcc

SEQ ID NO: 316 - DNA, 16 bp, Artificial sequence
  Synthetic oligonucleotide
aggcctgcccggacag

SEQ ID NO: 317 - DNA, 16 bp, Artificial sequence
  Synthetic oligonucleotide
gtgcacctcagccgga

SEQ ID NO: 318 - DNA, 16 bp, Artificial sequence
  Synthetic oligonucleotide
cgggctcccgtgcacc

SEQ ID NO: 319 - DNA, 16 bp, Artificial sequence
  Synthetic oligonucleotide
ccggcgggctcccgtg

SEQ ID NO: 320 - DNA, 16 bp, Artificial sequence
  Synthetic oligonucleotide
agaggccggcgggctc

SEQ ID NO: 321 - DNA, 16 bp, Artificial sequence
  Synthetic oligonucleotide
cggacggacgcgggca

SEQ ID NO: 322 - DNA, 16 bp, Artificial sequence
  Synthetic oligonucleotide
gagccccggccggaat

SEQ ID NO: 323 - DNA, 16 bp, Artificial sequence
  Synthetic oligonucleotide
tcgcggtgagccccgg

SEQ ID NO: 324 - DNA, 16 bp, Artificial sequence
  Synthetic oligonucleotide
gccatcgcggtgagcc

SEQ ID NO: 325 - DNA, 16 bp, Artificial sequence
  Synthetic oligonucleotide
ggagggccatcgcggt

SEQ ID NO: 326 - DNA, 16 bp, Artificial sequence
  Synthetic oligonucleotide
ggtgcggaggccagcg

SEQ ID NO: 327 - DNA, 16 bp, Artificial sequence
  Synthetic oligonucleotide
gccaggtgttccccgc

SEQ ID NO: 328 - DNA, 16 bp, Artificial sequence
  Synthetic oligonucleotide
tcaccgggcctagacc

SEQ ID NO: 329 - DNA, 16 bp, Artificial sequence
  Synthetic oligonucleotide
tgcgcactgcgcgcag

SEQ ID NO: 330 - DNA, 16 bp, Artificial sequence
  Synthetic oligonucleotide
ggccagcgagctccct

SEQ ID NO: 331 - DNA, 16 bp, Artificial sequence
  Synthetic oligonucleotide
tataggatccacaggg

SEQ ID NO: 332 - DNA, 16 bp, Artificial sequence
  Synthetic oligonucleotide
cttctataggatccac

SEQ ID NO: 333 - DNA, 16 bp, Artificial sequence
  Synthetic oligonucleotide
gcccgcaaacccgcgc

SEQ ID NO: 334 - DNA, 16 bp, Artificial sequence
  Synthetic oligonucleotide
ggctgcccgcaaaccc

SEQ ID NO: 335 - DNA, 16 bp, Artificial sequence
  Synthetic oligonucleotide
gctctgcccgggctgc

SEQ ID NO: 336 - DNA, 16 bp, Artificial sequence
  Synthetic oligonucleotide
cccggtgtttcgcggg

SEQ ID NO: 337 - DNA, 16 bp, Artificial sequence
  Synthetic oligonucleotide
tgtcaggcccggacgc

SEQ ID NO: 338 - DNA, 16 bp, Artificial sequence
  Synthetic oligonucleotide
ggagcggtgtcaggcc

SEQ ID NO: 339 - DNA, 16 bp, Artificial sequence
  Synthetic oligonucleotide
ccgccggagcggtgtc

SEQ ID NO: 340 - DNA, 16 bp, Artificial sequence
  Synthetic oligonucleotide
cgagccgccggagcgg

SEQ ID NO: 341 - DNA, 16 bp, Artificial sequence
  Synthetic oligonucleotide
ggaggcgagccgccgg

SEQ ID NO: 342 - DNA, 16 bp, Artificial sequence
  Synthetic oligonucleotide
gcagaggaggcgagcc

SEQ ID NO: 343 - DNA, 16 bp, Artificial sequence
  Synthetic oligonucleotide
cgggcgacggtggcgc

SEQ ID NO: 344 - DNA, 16 bp, Artificial sequence
  Synthetic oligonucleotide
ccgttgccgggacggt

SEQ ID NO: 345 - DNA, 16 bp, Artificial sequence
  Synthetic oligonucleotide
gtcgccgttgccggga

SEQ ID NO: 346 - DNA, 16 bp, Artificial sequence
  Synthetic oligonucleotide
gccgctctctgggctc

SEQ ID NO: 347 - DNA, 16 bp, Artificial sequence
  Synthetic oligonucleotide
cggctctccggacctc

SEQ ID NO: 348 - DNA, 16 bp, Artificial sequence
  Synthetic oligonucleotide
gggccggctctccgga

SEQ ID NO: 349 - DNA, 16 bp, Artificial sequence
  Synthetic oligonucleotide
cccaccccggaaggga

SEQ ID NO: 350 - DNA, 16 bp, Artificial sequence
  Synthetic oligonucleotide
tgggacagcccgcccc

SEQ ID NO: 351 - DNA, 16 bp, Artificial sequence
  Synthetic oligonucleotide
gagcccccctgggaca

SEQ ID NO: 352 - DNA, 16 bp, Artificial sequence
  Synthetic oligonucleotide
ggcggtgagcccccct

SEQ ID NO: 353 - DNA, 16 bp, Artificial sequence
  Synthetic oligonucleotide
ttgtaaaggcccacag

SEQ ID NO: 354 - DNA, 16 bp, Artificial sequence
  Synthetic oligonucleotide
gcccttgtaaaggccc

SEQ ID NO: 355 - DNA, 16 bp, Artificial sequence
  Synthetic oligonucleotide
agccgcccttgtaaag

SEQ ID NO: 356 - DNA, 16 bp, Artificial sequence
  Synthetic oligonucleotide
cctgcccggacagcca

SEQ ID NO: 357 - DNA, 16 bp, Artificial sequence
  Synthetic oligonucleotide
gaggcctgcccggaca

SEQ ID NO: 358 - DNA, 16 bp, Artificial sequence
  Synthetic oligonucleotide
cgtgcacctcagccgg

SEQ ID NO: 359 - DNA, 16 bp, Artificial sequence
  Synthetic oligonucleotide
gcgggctcccgtgcac

SEQ ID NO: 360 - DNA, 16 bp, Artificial sequence
  Synthetic oligonucleotide
gccggcgggctcccgt

SEQ ID NO: 361 - DNA, 16 bp, Artificial sequence
  Synthetic oligonucleotide
agagaggccggcgggc

SEQ ID NO: 362 - DNA, 16 bp, Artificial sequence
  Synthetic oligonucleotide
acggacggacgcgggc

SEQ ID NO: 363 - DNA, 16 bp, Artificial sequence
  Synthetic oligonucleotide
tgagccccggccggaa

SEQ ID NO: 364 - DNA, 16 bp, Artificial sequence
  Synthetic oligonucleotide
atcgcggtgagccccg

SEQ ID NO: 365 - DNA, 16 bp, Artificial sequence
  Synthetic oligonucleotide
gggccatcgcggtgag

SEQ ID NO: 366 - DNA, 16 bp, Artificial sequence
  Synthetic oligonucleotide
cggaggccagcgagga

SEQ ID NO: 367 - DNA, 16 bp, Artificial sequence
  Synthetic oligonucleotide
agccaggtgttccccg

SEQ ID NO: 368 - DNA, 16 bp, Artificial sequence
  Synthetic oligonucleotide
gtgcgcactgcgcgca

SEQ ID NO: 369 - DNA, 16 bp, Artificial sequence
  Synthetic oligonucleotide
aggccagcgagctccc

SEQ ID NO: 370 - DNA, 16 bp, Artificial sequence
  Synthetic oligonucleotide
ctataggatccacagg

SEQ ID NO: 371 - DNA, 16 bp, Artificial sequence
  Synthetic oligonucleotide
tcttctataggatcca

SEQ ID NO: 372 - DNA, 16 bp, Artificial sequence
  Synthetic oligonucleotide
tgcccgcaaacccgcg

SEQ ID NO: 373 - DNA, 16 bp, Artificial sequence
  Synthetic oligonucleotide
cggctgcccgcaaacc

SEQ ID NO: 374 - DNA, 16 bp, Artificial sequence
  Synthetic oligonucleotide
agctctgcccgggctg

SEQ ID NO: 375 - DNA, 16 bp, Artificial sequence
  Synthetic oligonucleotide
gcccggtgtttcgcgg

SEQ ID NO: 376 - DNA, 16 bp, Artificial sequence
  Synthetic oligonucleotide
cggtgtcaggcccgga

SEQ ID NO: 377 - DNA, 16 bp, Artificial sequence
  Synthetic oligonucleotide
ccggagcggtgtcagg

SEQ ID NO: 378 - DNA, 16 bp, Artificial sequence
  Synthetic oligonucleotide
gccgccggagcggtgt

SEQ ID NO: 379 - DNA, 16 bp, Artificial sequence
  Synthetic oligonucleotide
gcgagccgccggagcg

SEQ ID NO: 380 - DNA, 16 bp, Artificial sequence
  Synthetic oligonucleotide
aggaggcgagccgccg

SEQ ID NO: 381 - DNA, 16 bp, Artificial sequence
  Synthetic oligonucleotide
ggcgcagaggaggcga

SEQ ID NO: 382 - DNA, 16 bp, Artificial sequence
  Synthetic oligonucleotide
gcgggcgacggtggcg

SEQ ID NO: 383 - DNA, 16 bp, Artificial sequence
  Synthetic oligonucleotide
gcgctccgtgctggca

SEQ ID NO: 384 - DNA, 16 bp, Artificial sequence
  Synthetic oligonucleotide
gccgttgccgggacgg

SEQ ID NO: 385 - DNA, 16 bp, Artificial sequence
  Synthetic oligonucleotide
cgtcgccgttgccggg

SEQ ID NO: 386 - DNA, 16 bp, Artificial sequence
  Synthetic oligonucleotide
ctccagggatcccgcg

SEQ ID NO: 387 - DNA, 16 bp, Artificial sequence
  Synthetic oligonucleotide
ccggctctccggacct

SEQ ID NO: 388 - DNA, 16 bp, Artificial sequence
  Synthetic oligonucleotide
cccggaagggacccag

SEQ ID NO: 389 - DNA, 16 bp, Artificial sequence
  Synthetic oligonucleotide
gccccaccccggaagg

SEQ ID NO: 390 - DNA, 16 bp, Artificial sequence
  Synthetic oligonucleotide
ctgggacagcccgccc

SEQ ID NO: 391 - DNA, 16 bp, Artificial sequence
  Synthetic oligonucleotide
tgagcccccctgggac

SEQ ID NO: 392 - DNA, 16 bp, Artificial sequence
  Synthetic oligonucleotide
tggcggtgagcccccc

SEQ ID NO: 393 - DNA, 16 bp, Artificial sequence
  Synthetic oligonucleotide
cttgtaaaggcccaca

SEQ ID NO: 394 - DNA, 16 bp, Artificial sequence
  Synthetic oligonucleotide
cgcccttgtaaaggcc

SEQ ID NO: 395 - DNA, 16 bp, Artificial sequence
  Synthetic oligonucleotide
cagccgcccttgtaaa

SEQ ID NO: 396 - DNA, 16 bp, Artificial sequence
  Synthetic oligonucleotide
gcctgcccggacagcc

SEQ ID NO: 397 - DNA, 16 bp, Artificial sequence
  Synthetic oligonucleotide
gcactgcggcaggtgc

SEQ ID NO: 398 - DNA, 16 bp, Artificial sequence
  Synthetic oligonucleotide
ccgtgcacctcagccg

SEQ ID NO: 399 - DNA, 16 bp, Artificial sequence
  Synthetic oligonucleotide
ggcgggctcccgtgca

SEQ ID NO: 400 - DNA, 16 bp, Artificial sequence
  Synthetic oligonucleotide
aggccggcgggctccc

SEQ ID NO: 401 - DNA, 16 bp, Artificial sequence
  Synthetic oligonucleotide
gagagaggccggcggg

SEQ ID NO: 402 - DNA, 16 bp, Artificial sequence
  Synthetic oligonucleotide
gccccggccggaattt

SEQ ID NO: 403 - DNA, 16 bp, Artificial sequence
  Synthetic oligonucleotide
ggtgagccccggccgg

SEQ ID NO: 404 - DNA, 16 bp, Artificial sequence
  Synthetic oligonucleotide
catcgcggtgagcccc

SEQ ID NO: 405 - DNA, 16 bp, Artificial sequence
  Synthetic oligonucleotide
agggccatcgcggtga

SEQ ID NO: 406 - DNA, 16 bp, Artificial sequence
  Synthetic oligonucleotide
tgcggaggccagcgag

SEQ ID NO: 407 - DNA, 16 bp, Artificial sequence
  Synthetic oligonucleotide
caggtgttccccgcga

SEQ ID NO: 408 - DNA, 16 bp, Artificial sequence
  Synthetic oligonucleotide
ccgtagccagccaggt

SEQ ID NO: 409 - DNA, 16 bp, Artificial sequence
  Synthetic oligonucleotide
gcactgcgcgcaggtc

SEQ ID NO: 410 - DNA, 16 bp, Artificial sequence
  Synthetic oligonucleotide
gggtgcgcactgcgcg

SEQ ID NO: 411 - DNA, 16 bp, Artificial sequence
  Synthetic oligonucleotide
gaggccagcgagctcc

SEQ ID NO: 412 - DNA, 16 bp, Artificial sequence
  Synthetic oligonucleotide
tctataggatccacag

SEQ ID NO: 413 - DNA, 16 bp, Artificial sequence
  Synthetic oligonucleotide
atcttctataggatcc

SEQ ID NO: 414 - DNA, 16 bp, Artificial sequence
  Synthetic oligonucleotide
ctgcccgcaaacccgc

SEQ ID NO: 415 - DNA, 16 bp, Artificial sequence
  Synthetic oligonucleotide
aggcggctgcccgcaa

SEQ ID NO: 416 - DNA, 16 bp, Artificial sequence
  Synthetic oligonucleotide
gagagctctgcccggg

SEQ ID NO: 417 - DNA, 16 bp, Artificial sequence
  Synthetic oligonucleotide
ggcccggtgtttcgcg

SEQ ID NO: 418 - DNA, 16 bp, Artificial sequence
  Synthetic oligonucleotide
agcggtgtcaggcccg

SEQ ID NO: 419 - DNA, 16 bp, Artificial sequence
  Synthetic oligonucleotide
gccggagcggtgtcag

SEQ ID NO: 420 - DNA, 16 bp, Artificial sequence
  Synthetic oligonucleotide
agccgccggagcggtg

SEQ ID NO: 421 - DNA, 16 bp, Artificial sequence
  Synthetic oligonucleotide
ggcgagccgccggagc

SEQ ID NO: 422 - DNA, 16 bp, Artificial sequence
  Synthetic oligonucleotide
gaggaggcgagccgcc

SEQ ID NO: 423 - DNA, 16 bp, Artificial sequence
  Synthetic oligonucleotide
ggcgacggtggcgcgg

SEQ ID NO: 424 - DNA, 16 bp, Artificial sequence
  Synthetic oligonucleotide
cgccgttgccgggacg

SEQ ID NO: 425 - DNA, 16 bp, Artificial sequence
  Synthetic oligonucleotide
ggccctctctccgtga

SEQ ID NO: 426 - DNA, 16 bp, Artificial sequence
  Synthetic oligonucleotide
gctctccggacctctc

SEQ ID NO: 427 - DNA, 16 bp, Artificial sequence
  Synthetic oligonucleotide
gccggctctccggacc

SEQ ID NO: 428 - DNA, 16 bp, Artificial sequence
  Synthetic oligonucleotide
ccccggaagggaccca

SEQ ID NO: 429 - DNA, 16 bp, Artificial sequence
  Synthetic oligonucleotide
cgccccaccccggaag

SEQ ID NO: 430 - DNA, 16 bp, Artificial sequence
  Synthetic oligonucleotide
cctgggacagcccgcc

SEQ ID NO: 431 - DNA, 16 bp, Artificial sequence
  Synthetic oligonucleotide
cggtgagcccccctgg

SEQ ID NO: 432 - DNA, 16 bp, Artificial sequence
  Synthetic oligonucleotide
atggcggtgagccccc

SEQ ID NO: 433 - DNA, 16 bp, Artificial sequence
  Synthetic oligonucleotide
ccttgtaaaggcccac

SEQ ID NO: 434 - DNA, 16 bp, Artificial sequence
  Synthetic oligonucleotide
ccgcccttgtaaaggc

SEQ ID NO: 435 - DNA, 16 bp, Artificial sequence
  Synthetic oligonucleotide
gccagccgcccttgta

SEQ ID NO: 436 - DNA, 16 bp, Artificial sequence
  Synthetic oligonucleotide
ggcctgcccggacagc

SEQ ID NO: 437 - DNA, 16 bp, Artificial sequence
  Synthetic oligonucleotide
gcacctcagccggact

SEQ ID NO: 438 - DNA, 16 bp, Artificial sequence
  Synthetic oligonucleotide
cccgtgcacctcagcc

SEQ ID NO: 439 - DNA, 16 bp, Artificial sequence
  Synthetic oligonucleotide
cggcgggctcccgtgc

SEQ ID NO: 440 - DNA, 16 bp, Artificial sequence
  Synthetic oligonucleotide
gaggccggcgggctcc

SEQ ID NO: 441 - DNA, 16 bp, Artificial sequence
  Synthetic oligonucleotide
ggacggacgcgggcag

SEQ ID NO: 442 - DNA, 16 bp, Artificial sequence
  Synthetic oligonucleotide
agccccggccggaatt

SEQ ID NO: 443 - DNA, 16 bp, Artificial sequence
  Synthetic oligonucleotide
cggtgagccccggccg

SEQ ID NO: 444 - DNA, 16 bp, Artificial sequence
  Synthetic oligonucleotide
ccatcgcggtgagccc

SEQ ID NO: 445 - DNA, 16 bp, Artificial sequence
  Synthetic oligonucleotide
gagggccatcgcggtg

SEQ ID NO: 446 - DNA, 16 bp, Artificial sequence
  Synthetic oligonucleotide
gtgcggaggccagcga

SEQ ID NO: 447 - DNA, 16 bp, Artificial sequence
  Synthetic oligonucleotide
ccaggtgttccccgcg

SEQ ID NO: 448 - DNA, 16 bp, Artificial sequence
  Synthetic oligonucleotide
caccgggcctagacct

SEQ ID NO: 449 - DNA, 16 bp, Artificial sequence
  Synthetic oligonucleotide
gcgcactgcgcgcagg

SEQ ID NO: 450 - DNA, 16 bp, Artificial sequence
  Synthetic oligonucleotide
gccagcgagctccctt

SEQ ID NO: 451 - DNA, 16 bp, Artificial sequence
  Synthetic oligonucleotide
ataggatccacaggga

SEQ ID NO: 452 - DNA, 16 bp, Artificial sequence
  Synthetic oligonucleotide
ttctataggatccaca

SEQ ID NO: 453 - DNA, 16 bp, Artificial sequence
  Synthetic oligonucleotide
aatcttctataggatc

SEQ ID NO: 454 - DNA, 16 bp, Artificial sequence
  Synthetic oligonucleotide
ggcacctgggcggctg

SEQ ID NO: 455 - DNA, 16 bp, Artificial sequence
  Synthetic oligonucleotide
tgtgcgccgggcctgg

SEQ ID NO: 456 - DNA, 16 bp, Artificial sequence
  Synthetic oligonucleotide
cgcccctctaggtctc

SEQ ID NO: 457 - DNA, 16 bp, Artificial sequence
  Synthetic oligonucleotide
tccctccctcctaacg

SEQ ID NO: 458 - DNA, 16 bp, Artificial sequence
  Synthetic oligonucleotide
ggctccctcccgcccg

SEQ ID NO: 459 - DNA, 16 bp, Artificial sequence
  Synthetic oligonucleotide
cgctgaccgttttccc

SEQ ID NO: 460 - DNA, 16 bp, Artificial sequence
  Synthetic oligonucleotide
atgggtggtgccccgc

SEQ ID NO: 461 - DNA, 16 bp, Artificial sequence
  Synthetic oligonucleotide
aggaaaccgcccactc

SEQ ID NO: 462 - DNA, 16 bp, Artificial sequence
  Synthetic oligonucleotide
gcggtctgtgaaccgc

SEQ ID NO: 463 - DNA, 16 bp, Artificial sequence
  Synthetic oligonucleotide
tcggcctcgcgccgcg

SEQ ID NO: 464 - DNA, 16 bp, Artificial sequence
  Synthetic oligonucleotide
ccacgcggaaaccaaa

SEQ ID NO: 465 - DNA, 16 bp, Artificial sequence
  Synthetic oligonucleotide
tggccagcctttcggg

SEQ ID NO: 466 - DNA, 16 bp, Artificial sequence
  Synthetic oligonucleotide
cgggcttgcacccttc

SEQ ID NO: 467 - DNA, 16 bp, Artificial sequence
  Synthetic oligonucleotide
gcagtgtggccggttt

SEQ ID NO: 468 - DNA, 16 bp, Artificial sequence
  Synthetic oligonucleotide
agatgcctccccggcg

SEQ ID NO: 469 - DNA, 16 bp, Artificial sequence
  Synthetic oligonucleotide
tagaagaccagagcga

SEQ ID NO: 470 - DNA, 16 bp, Artificial sequence
  Synthetic oligonucleotide
agagaggccacggccc

SEQ ID NO: 471 - DNA, 16 bp, Artificial sequence
  Synthetic oligonucleotide
ccgcgctggcagctgg

SEQ ID NO: 472 - DNA, 16 bp, Artificial sequence
  Synthetic oligonucleotide
tgatcaccgaagttat

SEQ ID NO: 473 - DNA, 16 bp, Artificial sequence
  Synthetic oligonucleotide
ttccgccgccaggcgc

SEQ ID NO: 474 - DNA, 16 bp, Artificial sequence
  Synthetic oligonucleotide
atctctgcccgccttc

SEQ ID NO: 475 - DNA, 16 bp, Artificial sequence
  Synthetic oligonucleotide
ctccgcccgtccttcc

SEQ ID NO: 476 - DNA, 16 bp, Artificial sequence
  Synthetic oligonucleotide
gtctttccctccgttc

SEQ ID NO: 477 - DNA, 16 bp, Artificial sequence
  Synthetic oligonucleotide
ccggaggccgaggacc

SEQ ID NO: 478 - DNA, 16 bp, Artificial sequence
  Synthetic oligonucleotide
cggcggctgtgggccc

SEQ ID NO: 479 - DNA, 16 bp, Artificial sequence
  Synthetic oligonucleotide
cctgggccccggaacc

SEQ ID NO: 480 - DNA, 16 bp, Artificial sequence
  Synthetic oligonucleotide
cggcaacccgagtccc

SEQ ID NO: 481 - DNA, 16 bp, Artificial sequence
  Synthetic oligonucleotide
ttgcagggctgagcct

SEQ ID NO: 482 - DNA, 16 bp, Artificial sequence
  Synthetic oligonucleotide
ctcctccgtggccggg

SEQ ID NO: 483 - DNA, 16 bp, Artificial sequence
  Synthetic oligonucleotide
agcaacaggccgcctt

SEQ ID NO: 484 - DNA, 16 bp, Artificial sequence
  Synthetic oligonucleotide
ctccgggagcaaacag

SEQ ID NO: 485 - DNA, 16 bp, Artificial sequence
  Synthetic oligonucleotide
tggaacctggcaagga

SEQ ID NO: 486 - DNA, 16 bp, Artificial sequence
  Synthetic oligonucleotide
gacgatggattcccgc

SEQ ID NO: 487 - DNA, 16 bp, Artificial sequence
  Synthetic oligonucleotide
cgagaccccagagagg

SEQ ID NO: 488 - DNA, 16 bp, Artificial sequence
  Synthetic oligonucleotide
tgtaaaccaatttcag

SEQ ID NO: 489 - DNA, 16 bp, Artificial sequence
  Synthetic oligonucleotide
gttttgacatatctct

SEQ ID NO: 490 - DNA, 16 bp, Artificial sequence
  Synthetic oligonucleotide
tggcacctgggcggct

SEQ ID NO: 491 - DNA, 16 bp, Artificial sequence
  Synthetic oligonucleotide
cgttccgccgccaggc

SEQ ID NO: 492 - DNA, 16 bp, Artificial sequence
  Synthetic oligonucleotide
ccggtgtgcgccgggc

SEQ ID NO: 493 - DNA, 16 bp, Artificial sequence
  Synthetic oligonucleotide
gtccccggtgtgcgcc

SEQ ID NO: 494 - DNA, 16 bp, Artificial sequence
  Synthetic oligonucleotide
tccatctctgcccgcc

SEQ ID NO: 495 - DNA, 16 bp, Artificial sequence
  Synthetic oligonucleotide
ccgcccctctaggtct

SEQ ID NO: 496 - DNA, 16 bp, Artificial sequence
  Synthetic oligonucleotide
gtccttccgcccctct

SEQ ID NO: 497 - DNA, 16 bp, Artificial sequence
  Synthetic oligonucleotide
cgcccgtccttccgcc

SEQ ID NO: 498 - DNA, 16 bp, Artificial sequence
  Synthetic oligonucleotide
ccctccgcccgtcctt

SEQ ID NO: 499 - DNA, 16 bp, Artificial sequence
  Synthetic oligonucleotide
ccgtcccccggctccc

SEQ ID NO: 500 - DNA, 16 bp, Artificial sequence
  Synthetic oligonucleotide
gaggccgaggaccgct

SEQ ID NO: 501 - DNA, 16 bp, Artificial sequence
  Synthetic oligonucleotide
ctactcccggaggccg

SEQ ID NO: 502 - DNA, 16 bp, Artificial sequence
  Synthetic oligonucleotide
tcccgctactcccgga

SEQ ID NO: 503 - DNA, 16 bp, Artificial sequence
  Synthetic oligonucleotide
agcccgcgccggacgc

SEQ ID NO: 504 - DNA, 16 bp, Artificial sequence
  Synthetic oligonucleotide
gccctcagcccgcgcc

SEQ ID NO: 505 - DNA, 16 bp, Artificial sequence
  Synthetic oligonucleotide
cgcggcggctgtgggc

SEQ ID NO: 506 - DNA, 16 bp, Artificial sequence
  Synthetic oligonucleotide
ccggcgcggcggctgt

SEQ ID NO: 507 - DNA, 16 bp, Artificial sequence
  Synthetic oligonucleotide
gggtggtgccccgccg

SEQ ID NO: 508 - DNA, 16 bp, Artificial sequence
  Synthetic oligonucleotide
ccctgggccccggaac

SEQ ID NO: 509 - DNA, 16 bp, Artificial sequence
  Synthetic oligonucleotide
acccgagtcccggtct

SEQ ID NO: 510 - DNA, 16 bp, Artificial sequence
  Synthetic oligonucleotide
acccgacggcaacccg

SEQ ID NO: 511 - DNA, 16 bp, Artificial sequence
  Synthetic oligonucleotide
tgtcggcctcgcgccg

SEQ ID NO: 512 - DNA, 16 bp, Artificial sequence
  Synthetic oligonucleotide
gggctgtcggcctcgc

SEQ ID NO: 513 - DNA, 16 bp, Artificial sequence
  Synthetic oligonucleotide
cgccttgcggagggca

SEQ ID NO: 514 - DNA, 16 bp, Artificial sequence
  Synthetic oligonucleotide
aggccgccttgcggag

SEQ ID NO: 515 - DNA, 16 bp, Artificial sequence
  Synthetic oligonucleotide
gcagagctccgggagc

SEQ ID NO: 516 - DNA, 16 bp, Artificial sequence
  Synthetic oligonucleotide
tgcccgcagagctccg

SEQ ID NO: 517 - DNA, 16 bp, Artificial sequence
  Synthetic oligonucleotide
ccgggtgcccgcagag

SEQ ID NO: 518 - DNA, 16 bp, Artificial sequence
  Synthetic oligonucleotide
gtttccgggtgcccgc

SEQ ID NO: 519 - DNA, 16 bp, Artificial sequence
  Synthetic oligonucleotide
cgtgccgggcttgcac

SEQ ID NO: 520 - DNA, 16 bp, Artificial sequence
  Synthetic oligonucleotide
aaggcaccgtgccggg

SEQ ID NO: 521 - DNA, 16 bp, Artificial sequence
  Synthetic oligonucleotide
gaggccacggccctgc

SEQ ID NO: 522 - DNA, 16 bp, Artificial sequence
  Synthetic oligonucleotide
gctccgcgctggcagc

SEQ ID NO: 523 - DNA, 16 bp, Artificial sequence
  Synthetic oligonucleotide
aggagctccgcgctgg

SEQ ID NO: 524 - DNA, 16 bp, Artificial sequence
  Synthetic oligonucleotide
cgccaggagctccgcg

SEQ ID NO: 525 - DNA, 16 bp, Artificial sequence
  Synthetic oligonucleotide
cgctccgtgctggcac

SEQ ID NO: 526 - DNA, 16 bp, Artificial sequence
  Synthetic oligonucleotide
gtgtgcgccgggcctg

SEQ ID NO: 527 - DNA, 16 bp, Artificial sequence
  Synthetic oligonucleotide
cccggtgtgcgccggg

SEQ ID NO: 528 - DNA, 16 bp, Artificial sequence
  Synthetic oligonucleotide
cgtccccggtgtgcgc

SEQ ID NO: 529 - DNA, 16 bp, Artificial sequence
  Synthetic oligonucleotide
ccctctaggtctcccg

SEQ ID NO: 530 - DNA, 16 bp, Artificial sequence
  Synthetic oligonucleotide
tccgcccctctaggtc

SEQ ID NO: 531 - DNA, 16 bp, Artificial sequence
  Synthetic oligonucleotide
cgtccttccgcccctc

SEQ ID NO: 532 - DNA, 16 bp, Artificial sequence
  Synthetic oligonucleotide
ccgcccgtccttccgc

SEQ ID NO: 533 - DNA, 16 bp, Artificial sequence
  Synthetic oligonucleotide
gtccctccgcccgtcc

SEQ ID NO: 534 - DNA, 16 bp, Artificial sequence
  Synthetic oligonucleotide
cccgtcccccggctcc

SEQ ID NO: 535 - DNA, 16 bp, Artificial sequence
  Synthetic oligonucleotide
cggaggccgaggaccg

SEQ ID NO: 536 - DNA, 16 bp, Artificial sequence
  Synthetic oligonucleotide
gctactcccggaggcc

SEQ ID NO: 537 - DNA, 16 bp, Artificial sequence
  Synthetic oligonucleotide
gtcccgctactcccgg

SEQ ID NO: 538 - DNA, 16 bp, Artificial sequence
  Synthetic oligonucleotide
tcagcccgcgccggac

SEQ ID NO: 539 - DNA, 16 bp, Artificial sequence
  Synthetic oligonucleotide
agccctcagcccgcgc

SEQ ID NO: 540 - DNA, 16 bp, Artificial sequence
  Synthetic oligonucleotide
gcgcggcggctgtggg

SEQ ID NO: 541 - DNA, 16 bp, Artificial sequence
  Synthetic oligonucleotide
ggtgccccgccggccg

SEQ ID NO: 542 - DNA, 16 bp, Artificial sequence
  Synthetic oligonucleotide
tgggtggtgccccgcc

SEQ ID NO: 543 - DNA, 16 bp, Artificial sequence
  Synthetic oligonucleotide
tccctgggccccggaa

SEQ ID NO: 544 - DNA, 16 bp, Artificial sequence
  Synthetic oligonucleotide
gcaacccgagtcccgg

SEQ ID NO: 545 - DNA, 16 bp, Artificial sequence
  Synthetic oligonucleotide
gacccgacggcaaccc

SEQ ID NO: 546 - DNA, 16 bp, Artificial sequence
  Synthetic oligonucleotide
ctgtcggcctcgcgcc

SEQ ID NO: 547 - DNA, 16 bp, Artificial sequence
  Synthetic oligonucleotide
gctcctccgtggccgg

SEQ ID NO: 548 - DNA, 16 bp, Artificial sequence
  Synthetic oligonucleotide
ccgccttgcggagggc

SEQ ID NO: 549 - DNA, 16 bp, Artificial sequence
  Synthetic oligonucleotide
caggccgccttgcgga

SEQ ID NO: 550 - DNA, 16 bp, Artificial sequence
  Synthetic oligonucleotide
ccgcagagctccggga

SEQ ID NO: 551 - DNA, 16 bp, Artificial sequence
  Synthetic oligonucleotide
gtgcccgcagagctcc

SEQ ID NO: 552 - DNA, 16 bp, Artificial sequence
  Synthetic oligonucleotide
tccgggtgcccgcaga

SEQ ID NO: 553 - DNA, 16 bp, Artificial sequence
  Synthetic oligonucleotide
gccgggcttgcaccct

SEQ ID NO: 554 - DNA, 16 bp, Artificial sequence
  Synthetic oligonucleotide
ccgtgccgggcttgca

SEQ ID NO: 555 - DNA, 16 bp, Artificial sequence
  Synthetic oligonucleotide
gaaggcaccgtgccgg

SEQ ID NO: 556 - DNA, 16 bp, Artificial sequence
  Synthetic oligonucleotide
ggccctgccccgaacc

SEQ ID NO: 557 - DNA, 16 bp, Artificial sequence
  Synthetic oligonucleotide
agaggccacggccctg

SEQ ID NO: 558 - DNA, 16 bp, Artificial sequence
  Synthetic oligonucleotide
cgcgctggcagctggg

SEQ ID NO: 559 - DNA, 16 bp, Artificial sequence
  Synthetic oligonucleotide
agctccgcgctggcag

SEQ ID NO: 560 - DNA, 16 bp, Artificial sequence
  Synthetic oligonucleotide
caggagctccgcgctg

SEQ ID NO: 561 - DNA, 16 bp, Artificial sequence
  Synthetic oligonucleotide
gaccgccaggagctcc

SEQ ID NO: 562 - DNA, 16 bp, Artificial sequence
  Synthetic oligonucleotide
ggtgtgcgccgggcct

SEQ ID NO: 563 - DNA, 16 bp, Artificial sequence
  Synthetic oligonucleotide
ccccggtgtgcgccgg

SEQ ID NO: 564 - DNA, 16 bp, Artificial sequence
  Synthetic oligonucleotide
ctcccgcctggaacgc

SEQ ID NO: 565 - DNA, 16 bp, Artificial sequence
  Synthetic oligonucleotide
cccctctaggtctccc

SEQ ID NO: 566 - DNA, 16 bp, Artificial sequence
  Synthetic oligonucleotide
ttccgcccctctaggt

SEQ ID NO: 567 - DNA, 16 bp, Artificial sequence
  Synthetic oligonucleotide
ccgtccttccgcccct

SEQ ID NO: 568 - DNA, 16 bp, Artificial sequence
  Synthetic oligonucleotide
tccgcccgtccttccg

SEQ ID NO: 569 - DNA, 16 bp, Artificial sequence
  Synthetic oligonucleotide
cgtccctccgcccgtc

SEQ ID NO: 570 - DNA, 16 bp, Artificial sequence
  Synthetic oligonucleotide
ccccgtcccccggctc

SEQ ID NO: 571 - DNA, 16 bp, Artificial sequence
  Synthetic oligonucleotide
cccggaggccgaggac

SEQ ID NO: 572 - DNA, 16 bp, Artificial sequence
  Synthetic oligonucleotide
cgctactcccggaggc

SEQ ID NO: 573 - DNA, 16 bp, Artificial sequence
  Synthetic oligonucleotide
ggtcccgctactcccg

SEQ ID NO: 574 - DNA, 16 bp, Artificial sequence
  Synthetic oligonucleotide
ctcagcccgcgccgga

SEQ ID NO: 575 - DNA, 16 bp, Artificial sequence
  Synthetic oligonucleotide
cagccctcagcccgcg

SEQ ID NO: 576 - DNA, 16 bp, Artificial sequence
  Synthetic oligonucleotide
ggcgcggcggctgtgg

SEQ ID NO: 577 - DNA, 16 bp, Artificial sequence
  Synthetic oligonucleotide
tggtgccccgccggcc

SEQ ID NO: 578 - DNA, 16 bp, Artificial sequence
  Synthetic oligonucleotide
tgggccccggaaccgg

SEQ ID NO: 579 - DNA, 16 bp, Artificial sequence
  Synthetic oligonucleotide
cccggaggaaaccgcc

SEQ ID NO: 580 - DNA, 16 bp, Artificial sequence
  Synthetic oligonucleotide
ggcaacccgagtcccg

SEQ ID NO: 581 - DNA, 16 bp, Artificial sequence
  Synthetic oligonucleotide
gcgcgggtgaagaccc

SEQ ID NO: 582 - DNA, 16 bp, Artificial sequence
  Synthetic oligonucleotide
gctgtcggcctcgcgc

SEQ ID NO: 583 - DNA, 16 bp, Artificial sequence
  Synthetic oligonucleotide
ggctcctccgtggccg

SEQ ID NO: 584 - DNA, 16 bp, Artificial sequence
  Synthetic oligonucleotide
gccgccttgcggaggg

SEQ ID NO: 585 - DNA, 16 bp, Artificial sequence
  Synthetic oligonucleotide
acaggccgccttgcgg

SEQ ID NO: 586 - DNA, 16 bp, Artificial sequence
  Synthetic oligonucleotide
cccgcagagctccggg

SEQ ID NO: 587 - DNA, 16 bp, Artificial sequence
  Synthetic oligonucleotide
gggtgcccgcagagct

SEQ ID NO: 588 - DNA, 16 bp, Artificial sequence
  Synthetic oligonucleotide
ttccgggtgcccgcag

SEQ ID NO: 589 - DNA, 16 bp, Artificial sequence
  Synthetic oligonucleotide
tgccgggcttgcaccc

SEQ ID NO: 590 - DNA, 16 bp, Artificial sequence
  Synthetic oligonucleotide
accgtgccgggcttgc

SEQ ID NO: 591 - DNA, 16 bp, Artificial sequence
  Synthetic oligonucleotide
gcgaaggcaccgtgcc

SEQ ID NO: 592 - DNA, 16 bp, Artificial sequence
  Synthetic oligonucleotide
cggccctgccccgaac

SEQ ID NO: 593 - DNA, 16 bp, Artificial sequence
  Synthetic oligonucleotide
tccgcgctggcagctg

SEQ ID NO: 594 - DNA, 16 bp, Artificial sequence
  Synthetic oligonucleotide
gagctccgcgctggca

SEQ ID NO: 595 - DNA, 16 bp, Artificial sequence
  Synthetic oligonucleotide
ccaggagctccgcgct

SEQ ID NO: 596 - DNA, 16 bp, Artificial sequence
  Synthetic oligonucleotide
gcacctgggcggctgc

SEQ ID NO: 597 - DNA, 16 bp, Artificial sequence
  Synthetic oligonucleotide
gttccgccgccaggcg

SEQ ID NO: 598 - DNA, 16 bp, Artificial sequence
  Synthetic oligonucleotide
cggtgtgcgccgggcc

SEQ ID NO: 599 - DNA, 16 bp, Artificial sequence
  Synthetic oligonucleotide
tccccggtgtgcgccg

SEQ ID NO: 600 - DNA, 16 bp, Artificial sequence
  Synthetic oligonucleotide
cctcccgcctggaacg

SEQ ID NO: 601 - DNA, 16 bp, Artificial sequence
  Synthetic oligonucleotide
gcccctctaggtctcc

SEQ ID NO: 602 - DNA, 16 bp, Artificial sequence
  Synthetic oligonucleotide
tccttccgcccctcta

SEQ ID NO: 603 - DNA, 16 bp, Artificial sequence
  Synthetic oligonucleotide
gcccgtccttccgccc

SEQ ID NO: 604 - DNA, 16 bp, Artificial sequence
  Synthetic oligonucleotide
cctccgcccgtccttc

SEQ ID NO: 605 - DNA, 16 bp, Artificial sequence
  Synthetic oligonucleotide
cgtcccccggctccct

SEQ ID NO: 606 - DNA, 16 bp, Artificial sequence
  Synthetic oligonucleotide
cccccgtcccccggct

SEQ ID NO: 607 - DNA, 16 bp, Artificial sequence
  Synthetic oligonucleotide
tactcccggaggccga

SEQ ID NO: 608 - DNA, 16 bp, Artificial sequence
  Synthetic oligonucleotide
cccgctactcccggag

SEQ ID NO: 609 - DNA, 16 bp, Artificial sequence
  Synthetic oligonucleotide
gggtcccgctactccc

SEQ ID NO: 610 - DNA, 16 bp, Artificial sequence
  Synthetic oligonucleotide
cctcagcccgcgccgg

SEQ ID NO: 611 - DNA, 16 bp, Artificial sequence
  Synthetic oligonucleotide
gcggcggctgtgggcc

SEQ ID NO: 612 - DNA, 16 bp, Artificial sequence
  Synthetic oligonucleotide
cggcgcggcggctgtg

SEQ ID NO: 613 - DNA, 16 bp, Artificial sequence
  Synthetic oligonucleotide
gtggtgccccgccggc

SEQ ID NO: 614 - DNA, 16 bp, Artificial sequence
  Synthetic oligonucleotide
ctgggccccggaaccg

SEQ ID NO: 615 - DNA, 16 bp, Artificial sequence
  Synthetic oligonucleotide
cccgagtcccggtctt

SEQ ID NO: 616 - DNA, 16 bp, Artificial sequence
  Synthetic oligonucleotide
cccgacggcaacccga

SEQ ID NO: 617 - DNA, 16 bp, Artificial sequence
  Synthetic oligonucleotide
gtcggcctcgcgccgc

SEQ ID NO: 618 - DNA, 16 bp, Artificial sequence
  Synthetic oligonucleotide
ggctgtcggcctcgcg

SEQ ID NO: 619 - DNA, 16 bp, Artificial sequence
  Synthetic oligonucleotide
cgtgtggctcctccgt

SEQ ID NO: 620 - DNA, 16 bp, Artificial sequence
  Synthetic oligonucleotide
ggccgccttgcggagg

SEQ ID NO: 621 - DNA, 16 bp, Artificial sequence
  Synthetic oligonucleotide
gtcggcatggccagcc

SEQ ID NO: 622 - DNA, 16 bp, Artificial sequence
  Synthetic oligonucleotide
gcccgcagagctccgg

SEQ ID NO: 623 - DNA, 16 bp, Artificial sequence
  Synthetic oligonucleotide
cgggtgcccgcagagc

SEQ ID NO: 624 - DNA, 16 bp, Artificial sequence
  Synthetic oligonucleotide
tttccgggtgcccgca

SEQ ID NO: 625 - DNA, 16 bp, Artificial sequence
  Synthetic oligonucleotide
gtgccgggcttgcacc

SEQ ID NO: 626 - DNA, 16 bp, Artificial sequence
  Synthetic oligonucleotide
gcaccgtgccgggctt

SEQ ID NO: 627 - DNA, 16 bp, Artificial sequence
  Synthetic oligonucleotide
aggagatgcctccccg

SEQ ID NO: 628 - DNA, 16 bp, Artificial sequence
  Synthetic oligonucleotide
acggccctgccccgaa

SEQ ID NO: 629 - DNA, 16 bp, Artificial sequence
  Synthetic oligonucleotide
ctccgcgctggcagct

SEQ ID NO: 630 - DNA, 16 bp, Artificial sequence
  Synthetic oligonucleotide
ggagctccgcgctggc

SEQ ID NO: 631 - DNA, 16 bp, Artificial sequence
  Synthetic oligonucleotide
gccaggagctccgcgc

SEQ ID NO: 632 - DNA, 16 bp, Artificial sequence
  Synthetic oligonucleotide
atgcaaatcttctata

SEQ ID NO: 633 - DNA, 16 bp, Artificial sequence
  Synthetic oligonucleotide
ccgcttgagcgggccc

SEQ ID NO: 634 - DNA, 16 bp, Artificial sequence
  Synthetic oligonucleotide
tgcggccccgcttgag

SEQ ID NO: 635 - DNA, 16 bp, Artificial sequence
  Synthetic oligonucleotide
cgcaagcaccccttgg

SEQ ID NO: 636 - DNA, 16 bp, Artificial sequence
  Synthetic oligonucleotide
cagagagaggccggcg

SEQ ID NO: 637 - DNA, 16 bp, Artificial sequence
  Synthetic oligonucleotide
cacggactcccctggg

SEQ ID NO: 638 - DNA, 16 bp, Artificial sequence
  Synthetic oligonucleotide
cgccccggcgacctgg

SEQ ID NO: 639 - DNA, 16 bp, Artificial sequence
  Synthetic oligonucleotide
ccacgccgccccggcg

SEQ ID NO: 640 - DNA, 16 bp, Artificial sequence
  Synthetic oligonucleotide
acggacgcgggcagag

SEQ ID NO: 641 - DNA, 16 bp, Artificial sequence
  Synthetic oligonucleotide
ccgcgcggaggcggag

SEQ ID NO: 642 - DNA, 16 bp, Artificial sequence
  Synthetic oligonucleotide
cgccgggatgccttgc

SEQ ID NO: 643 - DNA, 16 bp, Artificial sequence
  Synthetic oligonucleotide
cgccggctcctggagc

SEQ ID NO: 644 - DNA, 16 bp, Artificial sequence
  Synthetic oligonucleotide
cagaccagggcgccgg

SEQ ID NO: 645 - DNA, 16 bp, Artificial sequence
  Synthetic oligonucleotide
gaatttcacggacgga

SEQ ID NO: 646 - DNA, 16 bp, Artificial sequence
  Synthetic oligonucleotide
gctcgccaggagctca

SEQ ID NO: 647 - DNA, 16 bp, Artificial sequence
  Synthetic oligonucleotide
agaaactccgggctcg

SEQ ID NO: 648 - DNA, 16 bp, Artificial sequence
  Synthetic oligonucleotide
taggagaggttgcgcc

SEQ ID NO: 649 - DNA, 16 bp, Artificial sequence
  Synthetic oligonucleotide
ccgtttctaggagagg

SEQ ID NO: 650 - DNA, 16 bp, Artificial sequence
  Synthetic oligonucleotide
ccggccggaatttcac

SEQ ID NO: 651 - DNA, 16 bp, Artificial sequence
  Synthetic oligonucleotide
cagcgaggcggcctct

SEQ ID NO: 652 - DNA, 16 bp, Artificial sequence
  Synthetic oligonucleotide
ccggtattcttcctcg

SEQ ID NO: 653 - DNA, 16 bp, Artificial sequence
  Synthetic oligonucleotide
cagcagagcccggtat

SEQ ID NO: 654 - DNA, 16 bp, Artificial sequence
  Synthetic oligonucleotide
ttctccgcggtgtgga

SEQ ID NO: 655 - DNA, 16 bp, Artificial sequence
  Synthetic oligonucleotide
tccccgggcttccgcg

SEQ ID NO: 656 - DNA, 16 bp, Artificial sequence
  Synthetic oligonucleotide
aggtatgcttttgacc

SEQ ID NO: 657 - DNA, 16 bp, Artificial sequence
  Synthetic oligonucleotide
aagcgggcaaagacag

SEQ ID NO: 658 - DNA, 16 bp, Artificial sequence
  Synthetic oligonucleotide
ctcccttgcacgtcag

SEQ ID NO: 659 - DNA, 16 bp, Artificial sequence
  Synthetic oligonucleotide
cggaagaacaagggca

SEQ ID NO: 660 - DNA, 16 bp, Artificial sequence
  Synthetic oligonucleotide
aatttcacggaagaac

SEQ ID NO: 661 - DNA, 16 bp, Artificial sequence
  Synthetic oligonucleotide
cagccagaatttcacg

SEQ ID NO: 662 - DNA, 16 bp, Artificial sequence
  Synthetic oligonucleotide
tagacagcgtcggaag

SEQ ID NO: 663 - DNA, 16 bp, Artificial sequence
  Synthetic oligonucleotide
ctaatccaggtttgcc

SEQ ID NO: 664 - DNA, 16 bp, Artificial sequence
  Synthetic oligonucleotide
tgtaactctaatccag

SEQ ID NO: 665 - DNA, 16 bp, Artificial sequence
  Synthetic oligonucleotide
tccaggagatgtaact

SEQ ID NO: 666 - DNA, 16 bp, Artificial sequence
  Synthetic oligonucleotide
actaatcatccaggag

SEQ ID NO: 667 - DNA, 16 bp, Artificial sequence
  Synthetic oligonucleotide
ctctgaactaatcatc

SEQ ID NO: 668 - DNA, 16 bp, Artificial sequence
  Synthetic oligonucleotide
caaacgagtctccgtc

SEQ ID NO: 669 - DNA, 16 bp, Artificial sequence
  Synthetic oligonucleotide
gctcgcagggcctcgc

SEQ ID NO: 670 - DNA, 16 bp, Artificial sequence
  Synthetic oligonucleotide
cgctcaaagcaggctc

SEQ ID NO: 671 - DNA, 16 bp, Artificial sequence
  Synthetic oligonucleotide
cgatgcccgggtacgg

SEQ ID NO: 672 - DNA, 16 bp, Artificial sequence
  Synthetic oligonucleotide
ggaatgccgatggcct

SEQ ID NO: 673 - DNA, 16 bp, Artificial sequence
  Synthetic oligonucleotide
caaatctggaccctgg

SEQ ID NO: 674 - DNA, 16 bp, Artificial sequence
  Synthetic oligonucleotide
ctggcgtgacctctca

SEQ ID NO: 675 - DNA, 16 bp, Artificial sequence
  Synthetic oligonucleotide
gccgagattcccgccg

SEQ ID NO: 676 - DNA, 16 bp, Artificial sequence
  Synthetic oligonucleotide
ggcgggccgcgtctcc

SEQ ID NO: 677 - DNA, 16 bp, Artificial sequence
  Synthetic oligonucleotide
atccggtgacggcggt

SEQ ID NO: 678 - DNA, 16 bp, Artificial sequence
  Synthetic oligonucleotide
aaaggctcggaggagc

SEQ ID NO: 679 - DNA, 16 bp, Artificial sequence
  Synthetic oligonucleotide
ccttctcaaaggctcg

SEQ ID NO: 680 - DNA, 16 bp, Artificial sequence
  Synthetic oligonucleotide
tggaaagcgatccttc

SEQ ID NO: 681 - DNA, 16 bp, Artificial sequence
  Synthetic oligonucleotide
gatgcctggaaagcga

SEQ ID NO: 682 - DNA, 16 bp, Artificial sequence
  Synthetic oligonucleotide
tcccgggcggcgatgc

SEQ ID NO: 683 - DNA, 16 bp, Artificial sequence
  Synthetic oligonucleotide
tccgggaggcccgtct

SEQ ID NO: 684 - DNA, 16 bp, Artificial sequence
  Synthetic oligonucleotide
tctgaatcctggactc

SEQ ID NO: 685 - DNA, 16 bp, Artificial sequence
  Synthetic oligonucleotide
tgccaccctgtcccgg

SEQ ID NO: 686 - DNA, 16 bp, Artificial sequence
  Synthetic oligonucleotide
ctgcgcgggcgccctg

SEQ ID NO: 687 - DNA, 16 bp, Artificial sequence
  Synthetic oligonucleotide
gggccgcgctgcacag

SEQ ID NO: 688 - DNA, 16 bp, Artificial sequence
  Synthetic oligonucleotide
gcgacccacgagggag

SEQ ID NO: 689 - DNA, 16 bp, Artificial sequence
  Synthetic oligonucleotide
gcgaaggcgacccacg

SEQ ID NO: 690 - DNA, 16 bp, Artificial sequence
  Synthetic oligonucleotide
cgcgccggtgtgggcg

SEQ ID NO: 691 - DNA, 16 bp, Artificial sequence
  Synthetic oligonucleotide
ttccccacgcgccggt

SEQ ID NO: 692 - DNA, 16 bp, Artificial sequence
  Synthetic oligonucleotide
gcgggaagccccgttc

SEQ ID NO: 693 - DNA, 16 bp, Artificial sequence
  Synthetic oligonucleotide
agccggactgtgcact

SEQ ID NO: 694 - DNA, 16 bp, Artificial sequence
  Synthetic oligonucleotide
aggcgcgcagggcacg

SEQ ID NO: 695 - DNA, 16 bp, Artificial sequence
  Synthetic oligonucleotide
gagccccaggcgcgca

SEQ ID NO: 696 - DNA, 16 bp, Artificial sequence
  Synthetic oligonucleotide
gaaagccccctgtggg

SEQ ID NO: 697 - DNA, 16 bp, Artificial sequence
  Synthetic oligonucleotide
gctcacgaaagccccc

SEQ ID NO: 698 - DNA, 16 bp, Artificial sequence
  Synthetic oligonucleotide
gatcccctctgccggc

SEQ ID NO: 699 - DNA, 16 bp, Artificial sequence
  Synthetic oligonucleotide
gaaatccccgcgcgcc

SEQ ID NO: 700 - DNA, 16 bp, Artificial sequence
  Synthetic oligonucleotide
gcgtaggcgaaatccc

SEQ ID NO: 701 - DNA, 16 bp, Artificial sequence
  Synthetic oligonucleotide
gtccggaggagccggg

SEQ ID NO: 702 - DNA, 16 bp, Artificial sequence
  Synthetic oligonucleotide
gcgccccgtccggagg

SEQ ID NO: 703 - DNA, 16 bp, Artificial sequence
  Synthetic oligonucleotide
cccgggtgcggaggcc

SEQ ID NO: 704 - DNA, 16 bp, Artificial sequence
  Synthetic oligonucleotide
ctgcgggtcccggtcc

SEQ ID NO: 705 - DNA, 16 bp, Artificial sequence
  Synthetic oligonucleotide
gggcccggcaggccgt

SEQ ID NO: 706 - DNA, 16 bp, Artificial sequence
  Synthetic oligonucleotide
tgcaaatcttctatag

SEQ ID NO: 707 - DNA, 16 bp, Artificial sequence
  Synthetic oligonucleotide
cgcttgagcgggccca

SEQ ID NO: 708 - DNA, 16 bp, Artificial sequence
  Synthetic oligonucleotide
gcggccccgcttgagc

SEQ ID NO: 709 - DNA, 16 bp, Artificial sequence
  Synthetic oligonucleotide
agagagaggccggcgg

SEQ ID NO: 710 - DNA, 16 bp, Artificial sequence
  Synthetic oligonucleotide
caagcaccccttggcc

SEQ ID NO: 711 - DNA, 16 bp, Artificial sequence
  Synthetic oligonucleotide
acggactcccctggga

SEQ ID NO: 712 - DNA, 16 bp, Artificial sequence
  Synthetic oligonucleotide
agccccaccacggact

SEQ ID NO: 713 - DNA, 16 bp, Artificial sequence
  Synthetic oligonucleotide
cacgccgccccggcga

SEQ ID NO: 714 - DNA, 16 bp, Artificial sequence
  Synthetic oligonucleotide
cggacgcgggcagaga

SEQ ID NO: 715 - DNA, 16 bp, Artificial sequence
  Synthetic oligonucleotide
cgcgcggaggcggagg

SEQ ID NO: 716 - DNA, 16 bp, Artificial sequence
  Synthetic oligonucleotide
ccgggatgccttgcat

SEQ ID NO: 717 - DNA, 16 bp, Artificial sequence
  Synthetic oligonucleotide
gctcctggagcgcctg

SEQ ID NO: 718 - DNA, 16 bp, Artificial sequence
  Synthetic oligonucleotide
agaccagggcgccggc

SEQ ID NO: 719 - DNA, 16 bp, Artificial sequence
  Synthetic oligonucleotide
atttcacggacggacg

SEQ ID NO: 720 - DNA, 16 bp, Artificial sequence
  Synthetic oligonucleotide
ctcgccaggagctcat

SEQ ID NO: 721 - DNA, 16 bp, Artificial sequence
  Synthetic oligonucleotide
gaaactccgggctcgc

SEQ ID NO: 722 - DNA, 16 bp, Artificial sequence
  Synthetic oligonucleotide
ggagaggttgcgcctg

SEQ ID NO: 723 - DNA, 16 bp, Artificial sequence
  Synthetic oligonucleotide
cgtttctaggagaggt

SEQ ID NO: 724 - DNA, 16 bp, Artificial sequence
  Synthetic oligonucleotide
cggccggaatttcacg

SEQ ID NO: 725 - DNA, 16 bp, Artificial sequence
  Synthetic oligonucleotide
agcgaggcggcctctt

SEQ ID NO: 726 - DNA, 16 bp, Artificial sequence
  Synthetic oligonucleotide
cggtattcttcctcgc

SEQ ID NO: 727 - DNA, 16 bp, Artificial sequence
  Synthetic oligonucleotide
gcagagcccggtattc

SEQ ID NO: 728 - DNA, 16 bp, Artificial sequence
  Synthetic oligonucleotide
tctccgcggtgtggag

SEQ ID NO: 729 - DNA, 16 bp, Artificial sequence
  Synthetic oligonucleotide
ccccgggcttccgcgg

SEQ ID NO: 730 - DNA, 16 bp, Artificial sequence
  Synthetic oligonucleotide
atgcttttgaccgcca

SEQ ID NO: 731 - DNA, 16 bp, Artificial sequence
  Synthetic oligonucleotide
agcgggcaaagacaga

SEQ ID NO: 732 - DNA, 16 bp, Artificial sequence
  Synthetic oligonucleotide
ttgcacgtcagccggg

SEQ ID NO: 733 - DNA, 16 bp, Artificial sequence
  Synthetic oligonucleotide
acagagaggccagcga

SEQ ID NO: 734 - DNA, 16 bp, Artificial sequence
  Synthetic oligonucleotide
atttcacggaagaaca

SEQ ID NO: 735 - DNA, 16 bp, Artificial sequence
  Synthetic oligonucleotide
agccagaatttcacgg

SEQ ID NO: 736 - DNA, 16 bp, Artificial sequence
  Synthetic oligonucleotide
agacagcgtcggaagg

SEQ ID NO: 737 - DNA, 16 bp, Artificial sequence
  Synthetic oligonucleotide
taatccaggtttgcct

SEQ ID NO: 738 - DNA, 16 bp, Artificial sequence
  Synthetic oligonucleotide
taactctaatccaggt

SEQ ID NO: 739 - DNA, 16 bp, Artificial sequence
  Synthetic oligonucleotide
ggagatgtaactctaa

SEQ ID NO: 740 - DNA, 16 bp, Artificial sequence
  Synthetic oligonucleotide
ctaatcatccaggaga

SEQ ID NO: 741 - DNA, 16 bp, Artificial sequence
  Synthetic oligonucleotide
tctgaactaatcatcc

SEQ ID NO: 742 - DNA, 16 bp, Artificial sequence
  Synthetic oligonucleotide
aaacgagtctccgtcg

SEQ ID NO: 743 - DNA, 16 bp, Artificial sequence
  Synthetic oligonucleotide
ctcgcagggcctcgct

SEQ ID NO: 744 - DNA, 16 bp, Artificial sequence
  Synthetic oligonucleotide
ctcaaagcaggctcgc

SEQ ID NO: 745 - DNA, 16 bp, Artificial sequence
  Synthetic oligonucleotide
gtacgggttccgctca

SEQ ID NO: 746 - DNA, 16 bp, Artificial sequence
  Synthetic oligonucleotide
gaatgccgatggcctg

SEQ ID NO: 747 - DNA, 16 bp, Artificial sequence
  Synthetic oligonucleotide
aatctggaccctgggc

SEQ ID NO: 748 - DNA, 16 bp, Artificial sequence
  Synthetic oligonucleotide
gaaaccaaatctggac

SEQ ID NO: 749 - DNA, 16 bp, Artificial sequence
  Synthetic oligonucleotide
gattcccgccggtgct

SEQ ID NO: 750 - DNA, 16 bp, Artificial sequence
  Synthetic oligonucleotide
gccgcgtctcccgggc

SEQ ID NO: 751 - DNA, 16 bp, Artificial sequence
  Synthetic oligonucleotide
gacggcggtccgcttt

SEQ ID NO: 752 - DNA, 16 bp, Artificial sequence
  Synthetic oligonucleotide
gctcggaggagcaggg

SEQ ID NO: 753 - DNA, 16 bp, Artificial sequence
  Synthetic oligonucleotide
ttctcaaaggctcgga

SEQ ID NO: 754 - DNA, 16 bp, Artificial sequence
  Synthetic oligonucleotide
ggaaagcgatccttct

SEQ ID NO: 755 - DNA, 16 bp, Artificial sequence
  Synthetic oligonucleotide
atgcctggaaagcgat

SEQ ID NO: 756 - DNA, 16 bp, Artificial sequence
  Synthetic oligonucleotide
cccgggcggcgatgcc

SEQ ID NO: 757 - DNA, 16 bp, Artificial sequence
  Synthetic oligonucleotide
ccgggaggcccgtctc

SEQ ID NO: 758 - DNA, 16 bp, Artificial sequence
  Synthetic oligonucleotide
ctgaatcctggactcc

SEQ ID NO: 759 - DNA, 16 bp, Artificial sequence
  Synthetic oligonucleotide
gtcccgggtgcctggc

SEQ ID NO: 760 - DNA, 16 bp, Artificial sequence
  Synthetic oligonucleotide
tgcgcgggcgccctgc

SEQ ID NO: 761 - DNA, 16 bp, Artificial sequence
  Synthetic oligonucleotide
ggccgcgctgcacagg

SEQ ID NO: 762 - DNA, 16 bp, Artificial sequence
  Synthetic oligonucleotide
cgacccacgagggagc

SEQ ID NO: 763 - DNA, 16 bp, Artificial sequence
  Synthetic oligonucleotide
cgaaggcgacccacga

SEQ ID NO: 764 - DNA, 16 bp, Artificial sequence
  Synthetic oligonucleotide
gcgccggtgtgggcga

SEQ ID NO: 765 - DNA, 16 bp, Artificial sequence
  Synthetic oligonucleotide
ccccacgcgccggtgt

SEQ ID NO: 766 - DNA, 16 bp, Artificial sequence
  Synthetic oligonucleotide
ccggactgtgcactgc

SEQ ID NO: 767 - DNA, 16 bp, Artificial sequence
  Synthetic oligonucleotide
cgggaagccccgttcc

SEQ ID NO: 768 - DNA, 16 bp, Artificial sequence
  Synthetic oligonucleotide
ggcgcgcagggcacgt

SEQ ID NO: 769 - DNA, 16 bp, Artificial sequence
  Synthetic oligonucleotide
agccccaggcgcgcag

SEQ ID NO: 770 - DNA, 16 bp, Artificial sequence
  Synthetic oligonucleotide
aaagccccctgtggga

SEQ ID NO: 771 - DNA, 16 bp, Artificial sequence
  Synthetic oligonucleotide
ctcacgaaagccccct

SEQ ID NO: 772 - DNA, 16 bp, Artificial sequence
  Synthetic oligonucleotide
tgccggcgcggcctgg

SEQ ID NO: 773 - DNA, 16 bp, Artificial sequence
  Synthetic oligonucleotide
aaatccccgcgcgccg

SEQ ID NO: 774 - DNA, 16 bp, Artificial sequence
  Synthetic oligonucleotide
cgtaggcgaaatcccc

SEQ ID NO: 775 - DNA, 16 bp, Artificial sequence
  Synthetic oligonucleotide
gggcggcgtaggcgaa

SEQ ID NO: 776 - DNA, 16 bp, Artificial sequence
  Synthetic oligonucleotide
cgccccgtccggagga

SEQ ID NO: 777 - DNA, 16 bp, Artificial sequence
  Synthetic oligonucleotide
gcggaggccagcgagg

SEQ ID NO: 778 - DNA, 16 bp, Artificial sequence
  Synthetic oligonucleotide
ctcccggcttttgccc

SEQ ID NO: 779 - DNA, 16 bp, Artificial sequence
  Synthetic oligonucleotide
gcccggcaggccgtcg

SEQ ID NO: 780 - DNA, 16 bp, Artificial sequence
  Synthetic oligonucleotide
gcaaatcttctatagg

SEQ ID NO: 781 - DNA, 16 bp, Artificial sequence
  Synthetic oligonucleotide
cttgagcgggcccagg

SEQ ID NO: 782 - DNA, 16 bp, Artificial sequence
  Synthetic oligonucleotide
ggccccgcttgagcgg

SEQ ID NO: 783 - DNA, 16 bp, Artificial sequence
  Synthetic oligonucleotide
agcaccccttggccct

SEQ ID NO: 784 - DNA, 16 bp, Artificial sequence
  Synthetic oligonucleotide
gactcccctgggacgt

SEQ ID NO: 785 - DNA, 16 bp, Artificial sequence
  Synthetic oligonucleotide
ccaccacggactcccc

SEQ ID NO: 786 - DNA, 16 bp, Artificial sequence
  Synthetic oligonucleotide
acgccgccccggcgac

SEQ ID NO: 787 - DNA, 16 bp, Artificial sequence
  Synthetic oligonucleotide
ggacgcgggcagagag

SEQ ID NO: 788 - DNA, 16 bp, Artificial sequence
  Synthetic oligonucleotide
ggaggcggaggcgtcc

SEQ ID NO: 789 - DNA, 16 bp, Artificial sequence
  Synthetic oligonucleotide
gcccctgccgcgcgga

SEQ ID NO: 790 - DNA, 16 bp, Artificial sequence
  Synthetic oligonucleotide
ctcctggagcgcctgg

SEQ ID NO: 791 - DNA, 16 bp, Artificial sequence
  Synthetic oligonucleotide
tttcacggacggacgc

SEQ ID NO: 792 - DNA, 16 bp, Artificial sequence
  Synthetic oligonucleotide
gaccagggcgccggct

SEQ ID NO: 793 - DNA, 16 bp, Artificial sequence
  Synthetic oligonucleotide
tcgccaggagctcatc

SEQ ID NO: 794 - DNA, 16 bp, Artificial sequence
  Synthetic oligonucleotide
aaactccgggctcgcc

SEQ ID NO: 795 - DNA, 16 bp, Artificial sequence
  Synthetic oligonucleotide
ctgcagaaactccggg

SEQ ID NO: 796 - DNA, 16 bp, Artificial sequence
  Synthetic oligonucleotide
gccggaatttcacgga

SEQ ID NO: 797 - DNA, 16 bp, Artificial sequence
  Synthetic oligonucleotide
tttctaggagaggttg

SEQ ID NO: 798 - DNA, 16 bp, Artificial sequence
  Synthetic oligonucleotide
ctcttccgaggcctcc

SEQ ID NO: 799 - DNA, 16 bp, Artificial sequence
  Synthetic oligonucleotide
tattcttcctcgctga

SEQ ID NO: 800 - DNA, 16 bp, Artificial sequence
  Synthetic oligonucleotide
agagcccggtattctt

SEQ ID NO: 801 - DNA, 16 bp, Artificial sequence
  Synthetic oligonucleotide
ctccgcggtgtggagt

SEQ ID NO: 802 - DNA, 16 bp, Artificial sequence
  Synthetic oligonucleotide
ctgtccgagggtgtcg

SEQ ID NO: 803 - DNA, 16 bp, Artificial sequence
  Synthetic oligonucleotide
tgcttttgaccgccag

SEQ ID NO: 804 - DNA, 16 bp, Artificial sequence
  Synthetic oligonucleotide
gcgggcaaagacagac

SEQ ID NO: 805 - DNA, 16 bp, Artificial sequence
  Synthetic oligonucleotide
ctgcgcgcaggtctag

SEQ ID NO: 806 - DNA, 16 bp, Artificial sequence
  Synthetic oligonucleotide
agagaggccagcgagc

SEQ ID NO: 807 - DNA, 16 bp, Artificial sequence
  Synthetic oligonucleotide
tttcacggaagaacaa

SEQ ID NO: 808 - DNA, 16 bp, Artificial sequence
  Synthetic oligonucleotide
gccagaatttcacgga

SEQ ID NO: 809 - DNA, 16 bp, Artificial sequence
  Synthetic oligonucleotide
acagcgtcggaaggtg

SEQ ID NO: 810 - DNA, 16 bp, Artificial sequence
  Synthetic oligonucleotide
tgcctagacagcgtcg

SEQ ID NO: 811 - DNA, 16 bp, Artificial sequence
  Synthetic oligonucleotide
aactctaatccaggtt

SEQ ID NO: 812 - DNA, 16 bp, Artificial sequence
  Synthetic oligonucleotide
gagatgtaactctaat

SEQ ID NO: 813 - DNA, 16 bp, Artificial sequence
  Synthetic oligonucleotide
taatcatccaggagat

SEQ ID NO: 814 - DNA, 16 bp, Artificial sequence
  Synthetic oligonucleotide
ctgaactaatcatcca

SEQ ID NO: 815 - DNA, 16 bp, Artificial sequence
  Synthetic oligonucleotide
aacgagtctccgtcgc

SEQ ID NO: 816 - DNA, 16 bp, Artificial sequence
  Synthetic oligonucleotide
tcgcagggcctcgctt

SEQ ID NO: 817 - DNA, 16 bp, Artificial sequence
  Synthetic oligonucleotide
tcaaagcaggctcgca

SEQ ID NO: 818 - DNA, 16 bp, Artificial sequence
  Synthetic oligonucleotide
tacgggttccgctcaa

SEQ ID NO: 819 - DNA, 16 bp, Artificial sequence
  Synthetic oligonucleotide
ccggacagccagccag

SEQ ID NO: 820 - DNA, 16 bp, Artificial sequence
  Synthetic oligonucleotide
aatgccgatggcctgg

SEQ ID NO: 821 - DNA, 16 bp, Artificial sequence
  Synthetic oligonucleotide
gctccggaatgccgat

SEQ ID NO: 822 - DNA, 16 bp, Artificial sequence
  Synthetic oligonucleotide
aaaccaaatctggacc

SEQ ID NO: 823 - DNA, 16 bp, Artificial sequence
  Synthetic oligonucleotide
ttcccgccggtgctgc

SEQ ID NO: 824 - DNA, 16 bp, Artificial sequence
  Synthetic oligonucleotide
ccagggccgagattcc

SEQ ID NO: 825 - DNA, 16 bp, Artificial sequence
  Synthetic oligonucleotide
acggcggtccgctttc

SEQ ID NO: 826 - DNA, 16 bp, Artificial sequence
  Synthetic oligonucleotide
tcggaggagcagggcg

SEQ ID NO: 827 - DNA, 16 bp, Artificial sequence
  Synthetic oligonucleotide
tctcaaaggctcggag

SEQ ID NO: 828 - DNA, 16 bp, Artificial sequence
  Synthetic oligonucleotide
gaaagcgatccttctc

SEQ ID NO: 829 - DNA, 16 bp, Artificial sequence
  Synthetic oligonucleotide
tgcctggaaagcgatc

SEQ ID NO: 830 - DNA, 16 bp, Artificial sequence
  Synthetic oligonucleotide
ccgggcggcgatgcct

SEQ ID NO: 831 - DNA, 16 bp, Artificial sequence
  Synthetic oligonucleotide
gaggcccgtctctctg

SEQ ID NO: 832 - DNA, 16 bp, Artificial sequence
  Synthetic oligonucleotide
gaatcctggactccgg

SEQ ID NO: 833 - DNA, 16 bp, Artificial sequence
  Synthetic oligonucleotide
tcccgggtgcctggcc

SEQ ID NO: 834 - DNA, 16 bp, Artificial sequence
  Synthetic oligonucleotide
gcgcgggcgccctgcc

SEQ ID NO: 835 - DNA, 16 bp, Artificial sequence
  Synthetic oligonucleotide
gccgcgctgcacaggc

SEQ ID NO: 836 - DNA, 16 bp, Artificial sequence
  Synthetic oligonucleotide
gacccacgagggagca

SEQ ID NO: 837 - DNA, 16 bp, Artificial sequence
  Synthetic oligonucleotide
gaaggcgacccacgag

SEQ ID NO: 838 - DNA, 16 bp, Artificial sequence
  Synthetic oligonucleotide
cgccggtgtgggcgaa

SEQ ID NO: 839 - DNA, 16 bp, Artificial sequence
  Synthetic oligonucleotide
cccacgcgccggtgtg

SEQ ID NO: 840 - DNA, 16 bp, Artificial sequence
  Synthetic oligonucleotide
gaagccccgttcccca

SEQ ID NO: 841 - DNA, 16 bp, Artificial sequence
  Synthetic oligonucleotide
gcgcgcagggcacgtg

SEQ ID NO: 842 - DNA, 16 bp, Artificial sequence
  Synthetic oligonucleotide
gccccaggcgcgcagg

SEQ ID NO: 843 - DNA, 16 bp, Artificial sequence
  Synthetic oligonucleotide
aagccccctgtgggag

SEQ ID NO: 844 - DNA, 16 bp, Artificial sequence
  Synthetic oligonucleotide
tcacgaaagccccctg

SEQ ID NO: 845 - DNA, 16 bp, Artificial sequence
  Synthetic oligonucleotide
gccggcgcggcctggc

SEQ ID NO: 846 - DNA, 16 bp, Artificial sequence
  Synthetic oligonucleotide
aatccccgcgcgccgg

SEQ ID NO: 847 - DNA, 16 bp, Artificial sequence
  Synthetic oligonucleotide
aggcgaaatccccgcg

SEQ ID NO: 848 - DNA, 16 bp, Artificial sequence
  Synthetic oligonucleotide
ggcggcgtaggcgaaa

SEQ ID NO: 849 - DNA, 16 bp, Artificial sequence
  Synthetic oligonucleotide
gccccgtccggaggag

SEQ ID NO: 850 - DNA, 16 bp, Artificial sequence
  Synthetic oligonucleotide
tgggagagcgccccgt

SEQ ID NO: 851 - DNA, 16 bp, Artificial sequence
  Synthetic oligonucleotide
cccggcttttgcccgg

SEQ ID NO: 852 - DNA, 16 bp, Artificial sequence
  Synthetic oligonucleotide
ggccggcgggctcccg

SEQ ID NO: 853 - DNA, 16 bp, Artificial sequence
  Synthetic oligonucleotide
cccggcaggccgtcgc

SEQ ID NO: 854 - DNA, 16 bp, Artificial sequence
  Synthetic oligonucleotide
caaatcttctatagga

SEQ ID NO: 855 - DNA, 16 bp, Artificial sequence
  Synthetic oligonucleotide
gcgcagaggaggcgag

SEQ ID NO: 856 - DNA, 16 bp, Artificial sequence
  Synthetic oligonucleotide
tcatgaatggcggtga

SEQ ID NO: 857 - DNA, 16 bp, Artificial sequence
  Synthetic oligonucleotide
tcccgtgcacctcagc

SEQ ID NO: 858 - DNA, 16 bp, Artificial sequence
  Synthetic oligonucleotide
gagcgggcccaggctg

SEQ ID NO: 859 - DNA, 16 bp, Artificial sequence
  Synthetic oligonucleotide
gccccgcttgagcggg

SEQ ID NO: 860 - DNA, 16 bp, Artificial sequence
  Synthetic oligonucleotide
gcaccccttggccctg

SEQ ID NO: 861 - DNA, 16 bp, Artificial sequence
  Synthetic oligonucleotide
ctcccctgggacgtgg

SEQ ID NO: 862 - DNA, 16 bp, Artificial sequence
  Synthetic oligonucleotide
caccacggactcccct

SEQ ID NO: 863 - DNA, 16 bp, Artificial sequence
  Synthetic oligonucleotide
cgccgccccggcgacc

SEQ ID NO: 864 - DNA, 16 bp, Artificial sequence
  Synthetic oligonucleotide
gacgcgggcagagaga

SEQ ID NO: 865 - DNA, 16 bp, Artificial sequence
  Synthetic oligonucleotide
gaggcggaggcgtccg

SEQ ID NO: 866 - DNA, 16 bp, Artificial sequence
  Synthetic oligonucleotide
ctgccgcgcggaggcg

SEQ ID NO: 867 - DNA, 16 bp, Artificial sequence
  Synthetic oligonucleotide
tcacggacggacgcgg

SEQ ID NO: 868 - DNA, 16 bp, Artificial sequence
  Synthetic oligonucleotide
ctggagcgcctgggag

SEQ ID NO: 869 - DNA, 16 bp, Artificial sequence
  Synthetic oligonucleotide
agggcgccggctcctg

SEQ ID NO: 870 - DNA, 16 bp, Artificial sequence
  Synthetic oligonucleotide
cgccaggagctcatcc

SEQ ID NO: 871 - DNA, 16 bp, Artificial sequence
  Synthetic oligonucleotide
aactccgggctcgcca

SEQ ID NO: 872 - DNA, 16 bp, Artificial sequence
  Synthetic oligonucleotide
tgcagaaactccgggc

SEQ ID NO: 873 - DNA, 16 bp, Artificial sequence
  Synthetic oligonucleotide
ccggaatttcacggac

SEQ ID NO: 874 - DNA, 16 bp, Artificial sequence
  Synthetic oligonucleotide
ttctaggagaggttgc

SEQ ID NO: 875 - DNA, 16 bp, Artificial sequence
  Synthetic oligonucleotide
ttccgaggcctccagc

SEQ ID NO: 876 - DNA, 16 bp, Artificial sequence
  Synthetic oligonucleotide
attcttcctcgctgag

SEQ ID NO: 877 - DNA, 16 bp, Artificial sequence
  Synthetic oligonucleotide
gagcccggtattcttc

SEQ ID NO: 878 - DNA, 16 bp, Artificial sequence
  Synthetic oligonucleotide
ctaaagctcctccagc

SEQ ID NO: 879 - DNA, 16 bp, Artificial sequence
  Synthetic oligonucleotide
ccgcggtgtggagtct

SEQ ID NO: 880 - DNA, 16 bp, Artificial sequence
  Synthetic oligonucleotide
tgtccgagggtgtcgg

SEQ ID NO: 881 - DNA, 16 bp, Artificial sequence
  Synthetic oligonucleotide
gcttttgaccgccagg

SEQ ID NO: 882 - DNA, 16 bp, Artificial sequence
  Synthetic oligonucleotide
cagacagaggtatgct

SEQ ID NO: 883 - DNA, 16 bp, Artificial sequence
  Synthetic oligonucleotide
ccagcgagctcccttg

SEQ ID NO: 884 - DNA, 16 bp, Artificial sequence
  Synthetic oligonucleotide
ttcacggaagaacaag

SEQ ID NO: 885 - DNA, 16 bp, Artificial sequence
  Synthetic oligonucleotide
ccagaatttcacggaa

SEQ ID NO: 886 - DNA, 16 bp, Artificial sequence
  Synthetic oligonucleotide
ctccgtcgccgtcctc

SEQ ID NO: 887 - DNA, 16 bp, Artificial sequence
  Synthetic oligonucleotide
cagcgtcggaaggtgg

SEQ ID NO: 888 - DNA, 16 bp, Artificial sequence
  Synthetic oligonucleotide
gcctagacagcgtcgg

SEQ ID NO: 889 - DNA, 16 bp, Artificial sequence
  Synthetic oligonucleotide
actctaatccaggttt

SEQ ID NO: 890 - DNA, 16 bp, Artificial sequence
  Synthetic oligonucleotide
agatgtaactctaatc

SEQ ID NO: 891 - DNA, 16 bp, Artificial sequence
  Synthetic oligonucleotide
tcatccaggagatgta

SEQ ID NO: 892 - DNA, 16 bp, Artificial sequence
  Synthetic oligonucleotide
tgaactaatcatccag

SEQ ID NO: 893 - DNA, 16 bp, Artificial sequence
  Synthetic oligonucleotide
gtccaaacgagtctcc

SEQ ID NO: 894 - DNA, 16 bp, Artificial sequence
  Synthetic oligonucleotide
caaagcaggctcgcag

SEQ ID NO: 895 - DNA, 16 bp, Artificial sequence
  Synthetic oligonucleotide
acgggttccgctcaaa

SEQ ID NO: 896 - DNA, 16 bp, Artificial sequence
  Synthetic oligonucleotide
atgccgatggcctggg

SEQ ID NO: 897 - DNA, 16 bp, Artificial sequence
  Synthetic oligonucleotide
ctccggaatgccgatg

SEQ ID NO: 898 - DNA, 16 bp, Artificial sequence
  Synthetic oligonucleotide
aaccaaatctggaccc

SEQ ID NO: 899 - DNA, 16 bp, Artificial sequence
  Synthetic oligonucleotide
tcccgccggtgctgcc

SEQ ID NO: 900 - DNA, 16 bp, Artificial sequence
  Synthetic oligonucleotide
cagggccgagattccc

SEQ ID NO: 901 - DNA, 16 bp, Artificial sequence
  Synthetic oligonucleotide
cgctttcgccggcctt

SEQ ID NO: 902 - DNA, 16 bp, Artificial sequence
  Synthetic oligonucleotide
gaggagcagggcggtc

SEQ ID NO: 903 - DNA, 16 bp, Artificial sequence
  Synthetic oligonucleotide
ctcaaaggctcggagg

SEQ ID NO: 904 - DNA, 16 bp, Artificial sequence
  Synthetic oligonucleotide
aaagcgatccttctca

SEQ ID NO: 905 - DNA, 16 bp, Artificial sequence
  Synthetic oligonucleotide
gcctggaaagcgatcc

SEQ ID NO: 906 - DNA, 16 bp, Artificial sequence
  Synthetic oligonucleotide
gggcggcgatgcctgg

SEQ ID NO: 907 - DNA, 16 bp, Artificial sequence
  Synthetic oligonucleotide
tcctcccgggcggcga

SEQ ID NO: 908 - DNA, 16 bp, Artificial sequence
  Synthetic oligonucleotide
atcctggactccggga

SEQ ID NO: 909 - DNA, 16 bp, Artificial sequence
  Synthetic oligonucleotide
gcccttcgattctgaa

SEQ ID NO: 910 - DNA, 16 bp, Artificial sequence
  Synthetic oligonucleotide
cgcgggcgccctgcca

SEQ ID NO: 911 - DNA, 16 bp, Artificial sequence
  Synthetic oligonucleotide
ccgcgctgcacaggcc

SEQ ID NO: 912 - DNA, 16 bp, Artificial sequence
  Synthetic oligonucleotide
acccacgagggagcag

SEQ ID NO: 913 - DNA, 16 bp, Artificial sequence
  Synthetic oligonucleotide
aaggcgacccacgagg

SEQ ID NO: 914 - DNA, 16 bp, Artificial sequence
  Synthetic oligonucleotide
gtgggcgaaggcgacc

SEQ ID NO: 915 - DNA, 16 bp, Artificial sequence
  Synthetic oligonucleotide
ccacgcgccggtgtgg

SEQ ID NO: 916 - DNA, 16 bp, Artificial sequence
  Synthetic oligonucleotide
aagccccgttccccac

SEQ ID NO: 917 - DNA, 16 bp, Artificial sequence
  Synthetic oligonucleotide
cgcgcagggcacgtgg

SEQ ID NO: 918 - DNA, 16 bp, Artificial sequence
  Synthetic oligonucleotide
ccccaggcgcgcaggg

SEQ ID NO: 919 - DNA, 16 bp, Artificial sequence
  Synthetic oligonucleotide
agccccctgtgggaga

SEQ ID NO: 920 - DNA, 16 bp, Artificial sequence
  Synthetic oligonucleotide
cacgaaagccccctgt

SEQ ID NO: 921 - DNA, 16 bp, Artificial sequence
  Synthetic oligonucleotide
ccggcgcggcctggct

SEQ ID NO: 922 - DNA, 16 bp, Artificial sequence
  Synthetic oligonucleotide
atccccgcgcgccggg

SEQ ID NO: 923 - DNA, 16 bp, Artificial sequence
  Synthetic oligonucleotide
ggcgaaatccccgcgc

SEQ ID NO: 924 - DNA, 16 bp, Artificial sequence
  Synthetic oligonucleotide
gcggcgtaggcgaaat

SEQ ID NO: 925 - DNA, 16 bp, Artificial sequence
  Synthetic oligonucleotide
ccccgtccggaggagc

SEQ ID NO: 926 - DNA, 16 bp, Artificial sequence
  Synthetic oligonucleotide
ggagagcgccccgtcc

SEQ ID NO: 927 - DNA, 16 bp, Artificial sequence
  Synthetic oligonucleotide
gcttttgcccgggtgc

SEQ ID NO: 928 - DNA, 16 bp, Artificial sequence
  Synthetic oligonucleotide
ccggcaggccgtcgcg

SEQ ID NO: 929 - DNA, 16 bp, Artificial sequence
  Synthetic oligonucleotide
cgcagaggaggcgagc

SEQ ID NO: 930 - DNA, 16 bp, Artificial sequence
  Synthetic oligonucleotide
catgaatggcggtgag

SEQ ID NO: 931 - DNA, 16 bp, Artificial sequence
  Synthetic oligonucleotide
cctcagccggactgtg

SEQ ID NO: 932 - DNA, 16 bp, Artificial sequence
  Synthetic oligonucleotide
aaatcttctataggat

SEQ ID NO: 933 - DNA, 16 bp, Artificial sequence
  Synthetic oligonucleotide
agcgggcccaggctgt

SEQ ID NO: 934 - DNA, 16 bp, Artificial sequence
  Synthetic oligonucleotide
ccccgcttgagcgggc

SEQ ID NO: 935 - DNA, 16 bp, Artificial sequence
  Synthetic oligonucleotide
ccccttggccctgcgg

SEQ ID NO: 936 - DNA, 16 bp, Artificial sequence
  Synthetic oligonucleotide
ggcgcaagcacccctt

SEQ ID NO: 937 - DNA, 16 bp, Artificial sequence
  Synthetic oligonucleotide
accacggactcccctg

SEQ ID NO: 938 - DNA, 16 bp, Artificial sequence
  Synthetic oligonucleotide
gccgccccggcgacct

SEQ ID NO: 939 - DNA, 16 bp, Artificial sequence
  Synthetic oligonucleotide
acgcgggcagagagag

SEQ ID NO: 940 - DNA, 16 bp, Artificial sequence
  Synthetic oligonucleotide
aggcggaggcgtccgg

SEQ ID NO: 941 - DNA, 16 bp, Artificial sequence
  Synthetic oligonucleotide
tgccgcgcggaggcgg

SEQ ID NO: 942 - DNA, 16 bp, Artificial sequence
  Synthetic oligonucleotide
ggcgccgggatgcctt

SEQ ID NO: 943 - DNA, 16 bp, Artificial sequence
  Synthetic oligonucleotide
cacggacggacgcggg

SEQ ID NO: 944 - DNA, 16 bp, Artificial sequence
  Synthetic oligonucleotide
ggcgccggctcctgga

SEQ ID NO: 945 - DNA, 16 bp, Artificial sequence
  Synthetic oligonucleotide
atccagcagcaggccg

SEQ ID NO: 946 - DNA, 16 bp, Artificial sequence
  Synthetic oligonucleotide
cggaatttcacggacg

SEQ ID NO: 947 - DNA, 16 bp, Artificial sequence
  Synthetic oligonucleotide
ctccgggctcgccagg

SEQ ID NO: 948 - DNA, 16 bp, Artificial sequence
  Synthetic oligonucleotide
gcagaaactccgggct

SEQ ID NO: 949 - DNA, 16 bp, Artificial sequence
  Synthetic oligonucleotide
tctaggagaggttgcg

SEQ ID NO: 950 - DNA, 16 bp, Artificial sequence
  Synthetic oligonucleotide
tccgaggcctccagct

SEQ ID NO: 951 - DNA, 16 bp, Artificial sequence
  Synthetic oligonucleotide
ccccggccggaatttc

SEQ ID NO: 952 - DNA, 16 bp, Artificial sequence
  Synthetic oligonucleotide
ttccagcgaggcggcc

SEQ ID NO: 953 - DNA, 16 bp, Artificial sequence
  Synthetic oligonucleotide
gcccggtattcttcct

SEQ ID NO: 954 - DNA, 16 bp, Artificial sequence
  Synthetic oligonucleotide
taaagctcctccagca

SEQ ID NO: 955 - DNA, 16 bp, Artificial sequence
  Synthetic oligonucleotide
cgtccccgggcttccg

SEQ ID NO: 956 - DNA, 16 bp, Artificial sequence
  Synthetic oligonucleotide
agacagaggtatgctt

SEQ ID NO: 957 - DNA, 16 bp, Artificial sequence
  Synthetic oligonucleotide
cagcgagctcccttgc

SEQ ID NO: 958 - DNA, 16 bp, Artificial sequence
  Synthetic oligonucleotide
cacggaagaacaaggg

SEQ ID NO: 959 - DNA, 16 bp, Artificial sequence
  Synthetic oligonucleotide
cagaatttcacggaag

SEQ ID NO: 960 - DNA, 16 bp, Artificial sequence
  Synthetic oligonucleotide
agcgtcggaaggtggg

SEQ ID NO: 961 - DNA, 16 bp, Artificial sequence
  Synthetic oligonucleotide
cctagacagcgtcgga

SEQ ID NO: 962 - DNA, 16 bp, Artificial sequence
  Synthetic oligonucleotide
ctctaatccaggtttg

SEQ ID NO: 963 - DNA, 16 bp, Artificial sequence
  Synthetic oligonucleotide
gatgtaactctaatcc

SEQ ID NO: 964 - DNA, 16 bp, Artificial sequence
  Synthetic oligonucleotide
catccaggagatgtaa

SEQ ID NO: 965 - DNA, 16 bp, Artificial sequence
  Synthetic oligonucleotide
gaactaatcatccagg

SEQ ID NO: 966 - DNA, 16 bp, Artificial sequence
  Synthetic oligonucleotide
tccaaacgagtctccg

SEQ ID NO: 967 - DNA, 16 bp, Artificial sequence
  Synthetic oligonucleotide
aaagcaggctcgcagg

SEQ ID NO: 968 - DNA, 16 bp, Artificial sequence
  Synthetic oligonucleotide
gttccgctcaaagcag

SEQ ID NO: 969 - DNA, 16 bp, Artificial sequence
  Synthetic oligonucleotide
gccgatggcctgggcc

SEQ ID NO: 970 - DNA, 16 bp, Artificial sequence
  Synthetic oligonucleotide
tccggaatgccgatgg

SEQ ID NO: 971 - DNA, 16 bp, Artificial sequence
  Synthetic oligonucleotide
accaaatctggaccct

SEQ ID NO: 972 - DNA, 16 bp, Artificial sequence
  Synthetic oligonucleotide
cccgccggtgctgcct

SEQ ID NO: 973 - DNA, 16 bp, Artificial sequence
  Synthetic oligonucleotide
agggccgagattcccg

SEQ ID NO: 974 - DNA, 16 bp, Artificial sequence
  Synthetic oligonucleotide
ctttcgccggccttct

SEQ ID NO: 975 - DNA, 16 bp, Artificial sequence
  Synthetic oligonucleotide
gagcagggcggtctgg

SEQ ID NO: 976 - DNA, 16 bp, Artificial sequence
  Synthetic oligonucleotide
tcaaaggctcggagga

SEQ ID NO: 977 - DNA, 16 bp, Artificial sequence
  Synthetic oligonucleotide
aagcgatccttctcaa

SEQ ID NO: 978 - DNA, 16 bp, Artificial sequence
  Synthetic oligonucleotide
cctggaaagcgatcct

SEQ ID NO: 979 - DNA, 16 bp, Artificial sequence
  Synthetic oligonucleotide
ggcggcgatgcctgga

SEQ ID NO: 980 - DNA, 16 bp, Artificial sequence
  Synthetic oligonucleotide
cctcccgggcggcgat

SEQ ID NO: 981 - DNA, 16 bp, Artificial sequence
  Synthetic oligonucleotide
gactccgggaggcccg

SEQ ID NO: 982 - DNA, 16 bp, Artificial sequence
  Synthetic oligonucleotide
ccttcgattctgaaac

SEQ ID NO: 983 - DNA, 16 bp, Artificial sequence
  Synthetic oligonucleotide
cgggcgccctgccacc

SEQ ID NO: 984 - DNA, 16 bp, Artificial sequence
  Synthetic oligonucleotide
ctgcacaggccgcctg

SEQ ID NO: 985 - DNA, 16 bp, Artificial sequence
  Synthetic oligonucleotide
cccacgagggagcagg

SEQ ID NO: 986 - DNA, 16 bp, Artificial sequence
  Synthetic oligonucleotide
aggcgacccacgaggg

SEQ ID NO: 987 - DNA, 16 bp, Artificial sequence
  Synthetic oligonucleotide
gggcgaaggcgaccca

SEQ ID NO: 988 - DNA, 16 bp, Artificial sequence
  Synthetic oligonucleotide
cacgcgccggtgtggg

SEQ ID NO: 989 - DNA, 16 bp, Artificial sequence
  Synthetic oligonucleotide
agccccgttccccacg

SEQ ID NO: 990 - DNA, 16 bp, Artificial sequence
  Synthetic oligonucleotide
gtgcgggaagccccgt

SEQ ID NO: 991 - DNA, 16 bp, Artificial sequence
  Synthetic oligonucleotide
ccaggcgcgcagggca

SEQ ID NO: 992 - DNA, 16 bp, Artificial sequence
  Synthetic oligonucleotide
cccctgtgggagagcc

SEQ ID NO: 993 - DNA, 16 bp, Artificial sequence
  Synthetic oligonucleotide
acgaaagccccctgtg

SEQ ID NO: 994 - DNA, 16 bp, Artificial sequence
  Synthetic oligonucleotide
gcggccctcgctgcct

SEQ ID NO: 995 - DNA, 16 bp, Artificial sequence
  Synthetic oligonucleotide
caggttgggagatccc

SEQ ID NO: 996 - DNA, 16 bp, Artificial sequence
  Synthetic oligonucleotide
gcgaaatccccgcgcg

SEQ ID NO: 997 - DNA, 16 bp, Artificial sequence
  Synthetic oligonucleotide
cggcgtaggcgaaatc

SEQ ID NO: 998 - DNA, 16 bp, Artificial sequence
  Synthetic oligonucleotide
ccgtccggaggagccg

SEQ ID NO: 999 - DNA, 16 bp, Artificial sequence
  Synthetic oligonucleotide
gagcgccccgtccgga

SEQ ID NO: 1000 - DNA, 16 bp, Artificial sequence
  Synthetic oligonucleotide
ttgcccgggtgcggag

SEQ ID NO: 1001 - DNA, 16 bp, Artificial sequence
  Synthetic oligonucleotide
caggccgtcgcgctgc

SEQ ID NO: 1002 - DNA, 16 bp, Artificial sequence
  Synthetic oligonucleotide
cagaggaggcgagccg

SEQ ID NO: 1003 - DNA, 16 bp, Artificial sequence
  Synthetic oligonucleotide
atgaatggcggtgagc

SEQ ID NO: 1004 - DNA, 16 bp, Artificial sequence
  Synthetic oligonucleotide
cagccggactgtgcac

SEQ ID NO: 1005 - DNA, 16 bp, Artificial sequence
  Synthetic oligonucleotide
taggatccacagggag

SEQ ID NO: 1006 - DNA, 16 bp, Artificial sequence
  Synthetic oligonucleotide
cgggcccaggctgtgc

SEQ ID NO: 1007 - DNA, 16 bp, Artificial sequence
  Synthetic oligonucleotide
cccgcttgagcgggcc

SEQ ID NO: 1008 - DNA, 16 bp, Artificial sequence
  Synthetic oligonucleotide
ctgcggccccgcttga

SEQ ID NO: 1009 - DNA, 16 bp, Artificial sequence
  Synthetic oligonucleotide
gcgcaagcaccccttg

SEQ ID NO: 1010 - DNA, 16 bp, Artificial sequence
  Synthetic oligonucleotide
ccacggactcccctgg

SEQ ID NO: 1011 - DNA, 16 bp, Artificial sequence
  Synthetic oligonucleotide
ccgccccggcgacctg

SEQ ID NO: 1012 - DNA, 16 bp, Artificial sequence
  Synthetic oligonucleotide
cgcgggcagagagagg

SEQ ID NO: 1013 - DNA, 16 bp, Artificial sequence
  Synthetic oligonucleotide
tcccacgccgccccgg

SEQ ID NO: 1014 - DNA, 16 bp, Artificial sequence
  Synthetic oligonucleotide
gacggacgcgggcaga

SEQ ID NO: 1015 - DNA, 16 bp, Artificial sequence
  Synthetic oligonucleotide
gccgcgcggaggcgga

SEQ ID NO: 1016 - DNA, 16 bp, Artificial sequence
  Synthetic oligonucleotide
gcgccgggatgccttg

SEQ ID NO: 1017 - DNA, 16 bp, Artificial sequence
  Synthetic oligonucleotide
gcgccggctcctggag

SEQ ID NO: 1018 - DNA, 16 bp, Artificial sequence
  Synthetic oligonucleotide
gcagaccagggcgccg

SEQ ID NO: 1019 - DNA, 16 bp, Artificial sequence
  Synthetic oligonucleotide
ggaatttcacggacgg

SEQ ID NO: 1020 - DNA, 16 bp, Artificial sequence
  Synthetic oligonucleotide
ccgggctcgccaggag

SEQ ID NO: 1021 - DNA, 16 bp, Artificial sequence
  Synthetic oligonucleotide
cagaaactccgggctc

SEQ ID NO: 1022 - DNA, 16 bp, Artificial sequence
  Synthetic oligonucleotide
ctaggagaggttgcgc

SEQ ID NO: 1023 - DNA, 16 bp, Artificial sequence
  Synthetic oligonucleotide
ctccgtttctaggaga

SEQ ID NO: 1024 - DNA, 16 bp, Artificial sequence
  Synthetic oligonucleotide
cccggccggaatttca

SEQ ID NO: 1025 - DNA, 16 bp, Artificial sequence
  Synthetic oligonucleotide
ccagcgaggcggcctc

SEQ ID NO: 1026 - DNA, 16 bp, Artificial sequence
  Synthetic oligonucleotide
cccggtattcttcctc

SEQ ID NO: 1027 - DNA, 16 bp, Artificial sequence
  Synthetic oligonucleotide
ccagcagagcccggta

SEQ ID NO: 1028 - DNA, 16 bp, Artificial sequence
  Synthetic oligonucleotide
gtccccgggcttccgc

SEQ ID NO: 1029 - DNA, 16 bp, Artificial sequence
  Synthetic oligonucleotide
cagaggtatgcttttg

SEQ ID NO: 1030 - DNA, 16 bp, Artificial sequence
  Synthetic oligonucleotide
gaagcgggcaaagaca

SEQ ID NO: 1031 - DNA, 16 bp, Artificial sequence
  Synthetic oligonucleotide
agcgagctcccttgca

SEQ ID NO: 1032 - DNA, 16 bp, Artificial sequence
  Synthetic oligonucleotide
acggaagaacaagggc

SEQ ID NO: 1033 - DNA, 16 bp, Artificial sequence
  Synthetic oligonucleotide
agaatttcacggaaga

SEQ ID NO: 1034 - DNA, 16 bp, Artificial sequence
  Synthetic oligonucleotide
gacattcagccagaat

SEQ ID NO: 1035 - DNA, 16 bp, Artificial sequence
  Synthetic oligonucleotide
ctagacagcgtcggaa

SEQ ID NO: 1036 - DNA, 16 bp, Artificial sequence
  Synthetic oligonucleotide
tctaatccaggtttgc

SEQ ID NO: 1037 - DNA, 16 bp, Artificial sequence
  Synthetic oligonucleotide
atgtaactctaatcca

SEQ ID NO: 1038 - DNA, 16 bp, Artificial sequence
  Synthetic oligonucleotide
atccaggagatgtaac

SEQ ID NO: 1039 - DNA, 16 bp, Artificial sequence
  Synthetic oligonucleotide
aactaatcatccagga

SEQ ID NO: 1040 - DNA, 16 bp, Artificial sequence
  Synthetic oligonucleotide
ccaaacgagtctccgt

SEQ ID NO: 1041 - DNA, 16 bp, Artificial sequence
  Synthetic oligonucleotide
aagcaggctcgcaggg

SEQ ID NO: 1042 - DNA, 16 bp, Artificial sequence
  Synthetic oligonucleotide
ttccgctcaaagcagg

SEQ ID NO: 1043 - DNA, 16 bp, Artificial sequence
  Synthetic oligonucleotide
ggccagccgttctctg

SEQ ID NO: 1044 - DNA, 16 bp, Artificial sequence
  Synthetic oligonucleotide
ccggaatgccgatggc

SEQ ID NO: 1045 - DNA, 16 bp, Artificial sequence
  Synthetic oligonucleotide
ccaaatctggaccctg

SEQ ID NO: 1046 - DNA, 16 bp, Artificial sequence
  Synthetic oligonucleotide
cctcagctggcgtgac

SEQ ID NO: 1047 - DNA, 16 bp, Artificial sequence
  Synthetic oligonucleotide
gggccgagattcccgc

SEQ ID NO: 1048 - DNA, 16 bp, Artificial sequence
  Synthetic oligonucleotide
ttcgccggccttctgg

SEQ ID NO: 1049 - DNA, 16 bp, Artificial sequence
  Synthetic oligonucleotide
gatccggtgacggcgg

SEQ ID NO: 1050 - DNA, 16 bp, Artificial sequence
  Synthetic oligonucleotide
caaaggctcggaggag

SEQ ID NO: 1051 - DNA, 16 bp, Artificial sequence
  Synthetic oligonucleotide
agcgatccttctcaaa

SEQ ID NO: 1052 - DNA, 16 bp, Artificial sequence
  Synthetic oligonucleotide
ctggaaagcgatcctt

SEQ ID NO: 1053 - DNA, 16 bp, Artificial sequence
  Synthetic oligonucleotide
gcggcgatgcctggaa

SEQ ID NO: 1054 - DNA, 16 bp, Artificial sequence
  Synthetic oligonucleotide
ctcccgggcggcgatg

SEQ ID NO: 1055 - DNA, 16 bp, Artificial sequence
  Synthetic oligonucleotide
ctccgggaggcccgtc

SEQ ID NO: 1056 - DNA, 16 bp, Artificial sequence
  Synthetic oligonucleotide
cttcgattctgaaacc

SEQ ID NO: 1057 - DNA, 16 bp, Artificial sequence
  Synthetic oligonucleotide
gggcgccctgccaccc

SEQ ID NO: 1058 - DNA, 16 bp, Artificial sequence
  Synthetic oligonucleotide
cctgcgcgggcgccct

SEQ ID NO: 1059 - DNA, 16 bp, Artificial sequence
  Synthetic oligonucleotide
gagcagggtgaccccc

SEQ ID NO: 1060 - DNA, 16 bp, Artificial sequence
  Synthetic oligonucleotide
ggcgacccacgaggga

SEQ ID NO: 1061 - DNA, 16 bp, Artificial sequence
  Synthetic oligonucleotide
ggcgaaggcgacccac

SEQ ID NO: 1062 - DNA, 16 bp, Artificial sequence
  Synthetic oligonucleotide
acgcgccggtgtgggc

SEQ ID NO: 1063 - DNA, 16 bp, Artificial sequence
  Synthetic oligonucleotide
gccccgttccccacgc

SEQ ID NO: 1064 - DNA, 16 bp, Artificial sequence
  Synthetic oligonucleotide
tgcgggaagccccgtt

SEQ ID NO: 1065 - DNA, 16 bp, Artificial sequence
  Synthetic oligonucleotide
caggcgcgcagggcac

SEQ ID NO: 1066 - DNA, 16 bp, Artificial sequence
  Synthetic oligonucleotide
agagccccaggcgcgc

SEQ ID NO: 1067 - DNA, 16 bp, Artificial sequence
  Synthetic oligonucleotide
cgaaagccccctgtgg

SEQ ID NO: 1068 - DNA, 16 bp, Artificial sequence
  Synthetic oligonucleotide
ggctcacgaaagcccc

SEQ ID NO: 1069 - DNA, 16 bp, Artificial sequence
  Synthetic oligonucleotide
tgggagatcccctctg

SEQ ID NO: 1070 - DNA, 16 bp, Artificial sequence
  Synthetic oligonucleotide
cgaaatccccgcgcgc

SEQ ID NO: 1071 - DNA, 16 bp, Artificial sequence
  Synthetic oligonucleotide
ggcgtaggcgaaatcc

SEQ ID NO: 1072 - DNA, 16 bp, Artificial sequence
  Synthetic oligonucleotide
cgtccggaggagccgg

SEQ ID NO: 1073 - DNA, 16 bp, Artificial sequence
  Synthetic oligonucleotide
agcgccccgtccggag

SEQ ID NO: 1074 - DNA, 16 bp, Artificial sequence
  Synthetic oligonucleotide
tgcccgggtgcggagg

SEQ ID NO: 1075 - DNA, 16 bp, Artificial sequence
  Synthetic oligonucleotide
ggccgtcgcgctgcgg

SEQ ID NO: 1076 - DNA, 16 bp, Artificial sequence
  Synthetic oligonucleotide
cctgctgcagaaactc

SEQ ID NO: 1077 - DNA, 16 bp, Artificial sequence
  Synthetic oligonucleotide
gaggttgcgcctgctg

SEQ ID NO: 1078 - DNA, 16 bp, Artificial sequence
  Synthetic oligonucleotide
agaggttgcgcctgct

SEQ ID NO: 1079 - DNA, 16 bp, Artificial sequence
  Synthetic oligonucleotide
ggtattcttcctcgct

SEQ ID NO: 1080 - DNA, 16 bp, Artificial sequence
  Synthetic oligonucleotide
cggtgtggagtctctc

SEQ ID NO: 1081 - DNA, 16 bp, Artificial sequence
  Synthetic oligonucleotide
tccgcggtgtggagtc

SEQ ID NO: 1082 - DNA, 16 bp, Artificial sequence
  Synthetic oligonucleotide
gttctccgcggtgtgg

SEQ ID NO: 1083 - DNA, 16 bp, Artificial sequence
  Synthetic oligonucleotide
agttctccgcggtgtg

SEQ ID NO: 1084 - DNA, 16 bp, Artificial sequence
  Synthetic oligonucleotide
cagttctccgcggtgt

SEQ ID NO: 1085 - DNA, 16 bp, Artificial sequence
  Synthetic oligonucleotide
gcagttctccgcggtg

SEQ ID NO: 1086 - DNA, 16 bp, Artificial sequence
  Synthetic oligonucleotide
ggcagttctccgcggt

SEQ ID NO: 1087 - DNA, 16 bp, Artificial sequence
  Synthetic oligonucleotide
atggcagttctccgcg

SEQ ID NO: 1088 - DNA, 16 bp, Artificial sequence
  Synthetic oligonucleotide
gctgtccgagggtgtc

SEQ ID NO: 1089 - DNA, 16 bp, Artificial sequence
  Synthetic oligonucleotide
ggtgctgtccgagggt

SEQ ID NO: 1090 - DNA, 16 bp, Artificial sequence
  Synthetic oligonucleotide
gagggtgctgtccgag

SEQ ID NO: 1091 - DNA, 16 bp, Artificial sequence
  Synthetic oligonucleotide
ggagggtgctgtccga

SEQ ID NO: 1092 - DNA, 16 bp, Artificial sequence
  Synthetic oligonucleotide
gacggtggcgcggggg

SEQ ID NO: 1093 - DNA, 16 bp, Artificial sequence
  Synthetic oligonucleotide
aggctgcaggggcccg

SEQ ID NO: 1094 - DNA, 16 bp, Artificial sequence
  Synthetic oligonucleotide
tatgcttttgaccgcc

SEQ ID NO: 1095 - DNA, 16 bp, Artificial sequence
  Synthetic oligonucleotide
ggtatgcttttgaccg

SEQ ID NO: 1096 - DNA, 16 bp, Artificial sequence
  Synthetic oligonucleotide
ggaagcgggcaaagac

SEQ ID NO: 1097 - DNA, 16 bp, Artificial sequence
  Synthetic oligonucleotide
actgcgcgcaggtcta

SEQ ID NO: 1098 - DNA, 16 bp, Artificial sequence
  Synthetic oligonucleotide
cactgcgcgcaggtct

SEQ ID NO: 1099 - DNA, 16 bp, Artificial sequence
  Synthetic oligonucleotide
cggggtgcgcactgcg

SEQ ID NO: 1100 - DNA, 16 bp, Artificial sequence
  Synthetic oligonucleotide
ccggggtgcgcactgc

SEQ ID NO: 1101 - DNA, 16 bp, Artificial sequence
  Synthetic oligonucleotide
gccggggtgcgcactg

SEQ ID NO: 1102 - DNA, 16 bp, Artificial sequence
  Synthetic oligonucleotide
acgtcagccggggtgc

SEQ ID NO: 1103 - DNA, 16 bp, Artificial sequence
  Synthetic oligonucleotide
cacgtcagccggggtg

SEQ ID NO: 1104 - DNA, 16 bp, Artificial sequence
  Synthetic oligonucleotide
gcacgtcagccggggt

SEQ ID NO: 1105 - DNA, 16 bp, Artificial sequence
  Synthetic oligonucleotide
cttgcacgtcagccgg

SEQ ID NO: 1106 - DNA, 16 bp, Artificial sequence
  Synthetic oligonucleotide
ccttgcacgtcagccg

SEQ ID NO: 1107 - DNA, 16 bp, Artificial sequence
  Synthetic oligonucleotide
cccttgcacgtcagcc

SEQ ID NO: 1108 - DNA, 16 bp, Artificial sequence
  Synthetic oligonucleotide
tcccttgcacgtcagc

SEQ ID NO: 1109 - DNA, 16 bp, Artificial sequence
  Synthetic oligonucleotide
gctcccttgcacgtca

SEQ ID NO: 1110 - DNA, 16 bp, Artificial sequence
  Synthetic oligonucleotide
gagctcccttgcacgt

SEQ ID NO: 1111 - DNA, 16 bp, Artificial sequence
  Synthetic oligonucleotide
cgagctcccttgcacg

SEQ ID NO: 1112 - DNA, 16 bp, Artificial sequence
  Synthetic oligonucleotide
gcgagctcccttgcac

SEQ ID NO: 1113 - DNA, 16 bp, Artificial sequence
  Synthetic oligonucleotide
gagaggccagcgagct

SEQ ID NO: 1114 - DNA, 16 bp, Artificial sequence
  Synthetic oligonucleotide
cagagaggccagcgag

SEQ ID NO: 1115 - DNA, 16 bp, Artificial sequence
  Synthetic oligonucleotide
agacattcagccagaa

SEQ ID NO: 1116 - DNA, 16 bp, Artificial sequence
  Synthetic oligonucleotide
gggagacattcagcca

SEQ ID NO: 1117 - DNA, 16 bp, Artificial sequence
  Synthetic oligonucleotide
ggggagacattcagcc

SEQ ID NO: 1118 - DNA, 16 bp, Artificial sequence
  Synthetic oligonucleotide
tccgtcgccgtcctcg

SEQ ID NO: 1119 - DNA, 16 bp, Artificial sequence
  Synthetic oligonucleotide
cgtcggaaggtggggg

SEQ ID NO: 1120 - DNA, 16 bp, Artificial sequence
  Synthetic oligonucleotide
gcgtcggaaggtgggg

SEQ ID NO: 1121 - DNA, 16 bp, Artificial sequence
  Synthetic oligonucleotide
gacagcgtcggaaggt

SEQ ID NO: 1122 - DNA, 16 bp, Artificial sequence
  Synthetic oligonucleotide
ggtttgcctagacagc

SEQ ID NO: 1123 - DNA, 16 bp, Artificial sequence
  Synthetic oligonucleotide
ccaggtttgcctagac

SEQ ID NO: 1124 - DNA, 16 bp, Artificial sequence
  Synthetic oligonucleotide
tccaggtttgcctaga

SEQ ID NO: 1125 - DNA, 16 bp, Artificial sequence
  Synthetic oligonucleotide
atatatctctgaacta

SEQ ID NO: 1126 - DNA, 16 bp, Artificial sequence
  Synthetic oligonucleotide
aatatatctctgaact

SEQ ID NO: 1127 - DNA, 16 bp, Artificial sequence
  Synthetic oligonucleotide
agggggcattttaata

SEQ ID NO: 1128 - DNA, 16 bp, Artificial sequence
  Synthetic oligonucleotide
gagggggcattttaat

SEQ ID NO: 1129 - DNA, 16 bp, Artificial sequence
  Synthetic oligonucleotide
aggctcgcagggcctc

SEQ ID NO: 1130 - DNA, 16 bp, Artificial sequence
  Synthetic oligonucleotide
ggttccgctcaaagca

SEQ ID NO: 1131 - DNA, 16 bp, Artificial sequence
  Synthetic oligonucleotide
tctcattctgaaacca

SEQ ID NO: 1132 - DNA, 16 bp, Artificial sequence
  Synthetic oligonucleotide
gtgacctctcattctg

SEQ ID NO: 1133 - DNA, 16 bp, Artificial sequence
  Synthetic oligonucleotide
gctggcgtgacctctc

SEQ ID NO: 1134 - DNA, 16 bp, Artificial sequence
  Synthetic oligonucleotide
ggtgctgcctcagctg

SEQ ID NO: 1135 - DNA, 16 bp, Artificial sequence
  Synthetic oligonucleotide
cttctcaaaggctcgg

SEQ ID NO: 1136 - DNA, 16 bp, Artificial sequence
  Synthetic oligonucleotide
tccttctcaaaggctc

SEQ ID NO: 1137 - DNA, 16 bp, Artificial sequence
  Synthetic oligonucleotide
gctcctcccgggcggc

SEQ ID NO: 1138 - DNA, 16 bp, Artificial sequence
  Synthetic oligonucleotide
tctggccagctcctcc

SEQ ID NO: 1139 - DNA, 16 bp, Artificial sequence
  Synthetic oligonucleotide
cgtctctctggccagc

SEQ ID NO: 1140 - DNA, 16 bp, Artificial sequence
  Synthetic oligonucleotide
ccgtctctctggccag

SEQ ID NO: 1141 - DNA, 16 bp, Artificial sequence
  Synthetic oligonucleotide
tgaatcctggactccg

SEQ ID NO: 1142 - DNA, 16 bp, Artificial sequence
  Synthetic oligonucleotide
cagatctgaatcctgg

SEQ ID NO: 1143 - DNA, 16 bp, Artificial sequence
  Synthetic oligonucleotide
ccagatctgaatcctg

SEQ ID NO: 1144 - DNA, 16 bp, Artificial sequence
  Synthetic oligonucleotide
tcgattctgaaaccag

SEQ ID NO: 1145 - DNA, 16 bp, Artificial sequence
  Synthetic oligonucleotide
ggtgcctggcccttcg

SEQ ID NO: 1146 - DNA, 16 bp, Artificial sequence
  Synthetic oligonucleotide
aaagagaggccacggc

SEQ ID NO: 1147 - DNA, 16 bp, Artificial sequence
  Synthetic oligonucleotide
tgaccgccaggagctc

SEQ ID NO: 1148 - DNA, 16 bp, Artificial sequence
  Synthetic oligonucleotide
aagagaggccacggcc

SEQ ID NO: 1149 - DNA, 16 bp, Artificial sequence
  Synthetic oligonucleotide
accgccaggagctccg

SEQ ID NO: 1150 - DNA, 16 bp, Artificial sequence
  Synthetic oligonucleotide
cacggccctgccccga

SEQ ID NO: 1151 - DNA, 16 bp, Artificial sequence
  Synthetic oligonucleotide
ccgccaggagctccgc

SEQ ID NO: 1152 - DNA, 16 bp, Artificial sequence
  Synthetic oligonucleotide
cgaaagagaggccacg

SEQ ID NO: 1153 - DNA, 16 bp, Artificial sequence
  Synthetic oligonucleotide
cttttgaccgccagga

SEQ ID NO: 1154 - DNA, 16 bp, Artificial sequence
  Synthetic oligonucleotide
gaaagagaggccacgg

SEQ ID NO: 1155 - DNA, 16 bp, Artificial sequence
  Synthetic oligonucleotide
tttgaccgccaggagc

SEQ ID NO: 1156 - DNA, 16 bp, Artificial sequence
  Synthetic oligonucleotide
ttgaccgccaggagct

SEQ ID NO: 1157 - DNA, 16 bp, Artificial sequence
  Synthetic oligonucleotide
caggtctagtcaggaa

SEQ ID NO: 1158 - DNA, 16 bp, Artificial sequence
  Synthetic oligonucleotide
gtctagtcaggaagcg

SEQ ID NO: 1159 - DNA, 16 bp, Artificial sequence
  Synthetic oligonucleotide
ctagtcaggaagcggg

SEQ ID NO: 1160 - DNA, 16 bp, Artificial sequence
  Synthetic oligonucleotide
tagtcaggaagcgggc

SEQ ID NO: 1161 - DNA, 16 bp, Artificial sequence
  Synthetic oligonucleotide
tgcgcgcaggtctagt

SEQ ID NO: 1162 - DNA, 16 bp, Artificial sequence
  Synthetic oligonucleotide
agtcaggaagcgggca

SEQ ID NO: 1163 - DNA, 16 bp, Artificial sequence
  Synthetic oligonucleotide
cgcgcaggtctagtca

SEQ ID NO: 1164 - DNA, 16 bp, Artificial sequence
  Synthetic oligonucleotide
gcgcaggtctagtcag

SEQ ID NO: 1165 - DNA, 16 bp, Artificial sequence
  Synthetic oligonucleotide
gtcaggaagcgggcaa

SEQ ID NO: 1166 - DNA, 16 bp, Artificial sequence
  Synthetic oligonucleotide
tctagtcaggaagcgg

SEQ ID NO: 1167 - DNA, 16 bp, Artificial sequence
  Synthetic oligonucleotide
ggtctagtcaggaagc

SEQ ID NO: 1168 - DNA, 16 bp, Artificial sequence
  Synthetic oligonucleotide
aggtctagtcaggaag

SEQ ID NO: 1169 - DNA, 16 bp, Artificial sequence
  Synthetic oligonucleotide
gcaggtctagtcagga

SEQ ID NO: 1170 - DNA, 16 bp, Artificial sequence
  Synthetic oligonucleotide
cgcaggtctagtcagg

SEQ ID NO: 1171 - DNA, 16 bp, Artificial sequence
  Synthetic oligonucleotide
gcgcgcaggtctagtc

SEQ ID NO: 1172 - RNA, 25 nt, Artificial sequence
  Synthetic oligonucleotide
aaagcgauccuucucaaaggcucgg

SEQ ID NO: 1173 - RNA, 25 nt, Artificial sequence
  Synthetic oligonucleotide
ccgagccuuugagaaggaucgcuuu

SEQ ID NO: 1174 - RNA, 19 nt, Artificial sequence
  Synthetic oligonucleotide
uuccgcucaaagcaggcuc

SEQ ID NO: 1175 - RNA, 19 nt, Artificial sequence
  Synthetic oligonucleotide
gagccugcuuugagcggaa

SEQ ID NO: 1176 - RNA, 23 nt, Artificial sequence
  Synthetic oligonucleotide
uuuaauauaucucugaacuaauc

SEQ ID NO: 1177 - RNA, 23 nt, Artificial sequence
  Synthetic oligonucleotide
aucucugaacuaaucauccagga

SEQ ID NO: 1178 - RNA, 23 nt, Artificial sequence
  Synthetic oligonucleotide
acuaaucauccaggagauguaac

SEQ ID NO: 1179 - RNA, 23 nt, Artificial sequence
  Synthetic oligonucleotide
uccaggagauguaacucuaaucc

SEQ ID NO: 1180 - RNA, 23 nt, Artificial sequence
  Synthetic oligonucleotide
auguaacucuaauccagguuugc

SEQ ID NO: 1181 - RNA, 23 nt, Artificial sequence
  Synthetic oligonucleotide
uagacagcgucggaagguggggg

SEQ ID NO: 1182 - RNA, 23 nt, Artificial sequence
  Synthetic oligonucleotide
agacauucagccagaauuucacg

SEQ ID NO: 1183 - RNA, 23 nt, Artificial sequence
  Synthetic oligonucleotide
agccagaauuucacggaagaaca

SEQ ID NO: 1184 - RNA, 23 nt, Artificial sequence
  Synthetic oligonucleotide
uuucacggaagaacaagggcaca

SEQ ID NO: 1185 - RNA, 23 nt, Artificial sequence
  Synthetic oligonucleotide
aagaacaagggcacagagaggcc

SEQ ID NO: 1186 - RNA, 23 nt, Artificial sequence
  Synthetic oligonucleotide
agaggccagcgagcucccuugca

SEQ ID NO: 1187 - RNA, 23 nt, Artificial sequence
  Synthetic oligonucleotide
ugggccggcucugggauccccgg

SEQ ID NO: 1188 - RNA, 23 nt, Artificial sequence
  Synthetic oligonucleotide
uccccgggaugcccaggaaagaa

SEQ ID NO: 1189 - RNA, 23 nt, Artificial sequence
  Synthetic oligonucleotide
uccuccagcagagcccgguauuc

SEQ ID NO: 1190 - RNA, 23 nt, Artificial sequence
  Synthetic oligonucleotide
uccucgcugaggggugcuuccag

SEQ ID NO: 1191 - RNA, 23 nt, Artificial sequence
  Synthetic oligonucleotide
aggccuccagcucccccggggcc

SEQ ID NO: 1192 - RNA, 23 nt, Artificial sequence
  Synthetic oligonucleotide
agcucccccggggccuccguuuc

SEQ ID NO: 1193 - RNA, 23 nt, Artificial sequence
  Synthetic oligonucleotide
aggagagguugcgccugcugcag

SEQ ID NO: 1194 - RNA, 23 nt, Artificial sequence
  Synthetic oligonucleotide
uugcgccugcugcagaaacuccg

SEQ ID NO: 1195 - RNA, 23 nt, Artificial sequence
  Synthetic oligonucleotide
aacuccgggcucgccaggagcuc

SEQ ID NO: 1196 - RNA, 23 nt, Artificial sequence
  Synthetic oligonucleotide
uccagcagcaggccgcaggggag

SEQ ID NO: 1197 - RNA, 23 nt, Artificial sequence
  Synthetic oligonucleotide
aggggagugcagaccagggcgcc

SEQ ID NO: 1198 - RNA, 23 nt, Artificial sequence
  Synthetic oligonucleotide
agcgccugggagggcgccgggau

SEQ ID NO: 1199 - RNA, 23 nt, Artificial sequence
  Synthetic oligonucleotide
agguggagcugccccggcuuggg

SEQ ID NO: 1200 - RNA, 23 nt, Artificial sequence
  Synthetic oligonucleotide
uucccacgccgccccggcgaccu

SEQ ID NO: 1201 - RNA, 23 nt, Artificial sequence
  Synthetic oligonucleotide
ugccaccgcgcaggggcccggca

SEQ ID NO: 1202 - RNA, 23 nt, Artificial sequence
  Synthetic oligonucleotide
ucccgguccucccggcuuuugcc

SEQ ID NO: 1203 - RNA, 23 nt, Artificial sequence
  Synthetic oligonucleotide
ugagggugggagagcgccccguc

SEQ ID NO: 1204 - RNA, 23 nt, Artificial sequence
  Synthetic oligonucleotide
aaauccccgcgcgccggggcagg

SEQ ID NO: 1205 - RNA, 23 nt, Artificial sequence
  Synthetic oligonucleotide
ugggagauccccucugccggcgc

SEQ ID NO: 1206 - RNA, 23 nt, Artificial sequence
  Synthetic oligonucleotide
aaagcccccugugggagagcccc

SEQ ID NO: 1207 - RNA, 23 nt, Artificial sequence
  Synthetic oligonucleotide
acgcgccggugugggcgaaggcg

SEQ ID NO: 1208 - RNA, 23 nt, Artificial sequence
  Synthetic oligonucleotide
ugcgcgggcgcccugccacccug

SEQ ID NO: 1209 - RNA, 23 nt, Artificial sequence
  Synthetic oligonucleotide
uucgauucugaaaccagaucuga

SEQ ID NO: 1210 - RNA, 23 nt, Artificial sequence
  Synthetic oligonucleotide
ugaaaccagaucugaauccugga

SEQ ID NO: 1211 - RNA, 23 nt, Artificial sequence
  Synthetic oligonucleotide
uccuggacuccgggaggcccguc

SEQ ID NO: 1212 - RNA, 23 nt, Artificial sequence
  Synthetic oligonucleotide
uccgggaggcccgucucucuggc

SEQ ID NO: 1213 - RNA, 23 nt, Artificial sequence
  Synthetic oligonucleotide
agcuccucccgggcggcgaugcc

SEQ ID NO: 1214 - RNA, 23 nt, Artificial sequence
  Synthetic oligonucleotide
auccuucucaaaggcucggagga

SEQ ID NO: 1215 - RNA, 23 nt, Artificial sequence
  Synthetic oligonucleotide
ucugggauccggugacggcgguc

SEQ ID NO: 1216 - RNA, 23 nt, Artificial sequence
  Synthetic oligonucleotide
uggcgggccgcgucucccgggcc

SEQ ID NO: 1217 - RNA, 23 nt, Artificial sequence
  Synthetic oligonucleotide
auucccgccggugcugccucagc

SEQ ID NO: 1218 - RNA, 23 nt, Artificial sequence
  Synthetic oligonucleotide
aaaucuggacccugggcuccgga

SEQ ID NO: 1219 - RNA, 23 nt, Artificial sequence
  Synthetic oligonucleotide
acccugggcuccggaaugccgau

SEQ ID NO: 1220 - RNA, 23 nt, Artificial sequence
  Synthetic oligonucleotide
ugccgauggccugggccagccgu

SEQ ID NO: 1221 - RNA, 23 nt, Artificial sequence
  Synthetic oligonucleotide
ucaaagcaggcucgcagggccuc

SEQ ID NO: 1222 - RNA, 23 nt, Artificial sequence
  Synthetic oligonucleotide
aaacgagucuccgucgccguccu

SEQ ID NO: 1223 - RNA, 23 nt, Artificial sequence
  Synthetic oligonucleotide
ugcuguccgagggugucgggagg

SEQ ID NO: 1224 - RNA, 23 nt, Artificial sequence
  Synthetic oligonucleotide
auuucacggacggacgcgggcag

SEQ ID NO: 1225 - RNA, 23 nt, Artificial sequence
  Synthetic oligonucleotide
agccagccagccagccgcccuug

SEQ ID NO: 1226 - RNA, 23 nt, Artificial sequence
  Synthetic oligonucleotide
agccagccgcccuuguaaaggcc

SEQ ID NO: 1227 - RNA, 23 nt, Artificial sequence
  Synthetic oligonucleotide
agggggcauuuuaauauaucucu

SEQ ID NO: 1228 - RNA, 23 nt, Artificial sequence
  Synthetic oligonucleotide
aauccagguuugccuagacagcg

SEQ ID NO: 1229 - RNA, 23 nt, Artificial sequence
  Synthetic oligonucleotide
acagcgucggaagguggggggag

SEQ ID NO: 1230 - RNA, 23 nt, Artificial sequence
  Synthetic oligonucleotide
aagggcacagagaggccagcgag

SEQ ID NO: 1231 - RNA, 23 nt, Artificial sequence
  Synthetic oligonucleotide
agcgagcucccuugcacgucagc

SEQ ID NO: 1232 - RNA, 23 nt, Artificial sequence
  Synthetic oligonucleotide
ugggauccccgggaugcccagga

SEQ ID NO: 1233 - RNA, 23 nt, Artificial sequence
  Synthetic oligonucleotide
uucuccgcggaguggagucucuc

SEQ ID NO: 1234 - RNA, 23 nt, Artificial sequence
  Synthetic oligonucleotide
ucucucaccgggccuagaccuag

SEQ ID NO: 1235 - RNA, 23 nt, Artificial sequence
  Synthetic oligonucleotide
accuagaaggcaggaaucccagg

SEQ ID NO: 1236 - RNA, 23 nt, Artificial sequence
  Synthetic oligonucleotide
uagccagccagguguuccccgcg

SEQ ID NO: 1237 - RNA, 23 nt, Artificial sequence
  Synthetic oligonucleotide
acugcgcgcaggucugcugguac

SEQ ID NO: 1238 - RNA, 23 nt, Artificial sequence
  Synthetic oligonucleotide
augcccaggaaagaauggcaguu

SEQ ID NO: 1239 - RNA, 23 nt, Artificial sequence
  Synthetic oligonucleotide
uccgcgguguggagucucucacc

SEQ ID NO: 1240 - RNA, 23 nt, Artificial sequence
  Synthetic oligonucleotide
uuuugcccgggugcggaggccag

SEQ ID NO: 1241 - RNA, 23 nt, Artificial sequence
  Synthetic oligonucleotide
aggccagcgaggagccugagggu

SEQ ID NO: 1242 - RNA, 21 nt, Artificial sequence
  Synthetic oligonucleotide
uuaguucagagauauauuaaa

SEQ ID NO: 1243 - RNA, 21 nt, Artificial sequence
  Synthetic oligonucleotide
cuggaugauuaguucagagau

SEQ ID NO: 1244 - RNA, 21 nt, Artificial sequence
  Synthetic oligonucleotide
uacaucuccuggaugauuagu

SEQ ID NO: 1245 - RNA, 21 nt, Artificial sequence
  Synthetic oligonucleotide
auuagaguuacaucuccugga

SEQ ID NO: 1246 - RNA, 21 nt, Artificial sequence
  Synthetic oligonucleotide
aaaccuggauuagaguuacau

SEQ ID NO: 1247 - RNA, 21 nt, Artificial sequence
  Synthetic oligonucleotide
cccaccuuccgacgcugucua

SEQ ID NO: 1248 - RNA, 21 nt, Artificial sequence
  Synthetic oligonucleotide
ugaaauucuggcugaaugucu

SEQ ID NO: 1249 - RNA, 21 nt, Artificial sequence
  Synthetic oligonucleotide
uucuuccgugaaauucuggcu

SEQ ID NO: 1250 - RNA, 21 nt, Artificial sequence
  Synthetic oligonucleotide
ugcccuuguucuuccgugaaa

SEQ ID NO: 1251 - RNA, 21 nt, Artificial sequence
  Synthetic oligonucleotide
ccucucugugcccuuguucuu

SEQ ID NO: 1252 - RNA, 21 nt, Artificial sequence
  Synthetic oligonucleotide
caagggagcucgcuggccucu

SEQ ID NO: 1253 - RNA, 21 nt, Artificial sequence
  Synthetic oligonucleotide
ggggaucccagagccggccca

SEQ ID NO: 1254 - RNA, 21 nt, Artificial sequence
  Synthetic oligonucleotide
cuuuccugggcaucccgggga

SEQ ID NO: 1255 - RNA, 21 nt, Artificial sequence
  Synthetic oligonucleotide
auaccgggcucugcuggagga

SEQ ID NO: 1256 - RNA, 21 nt, Artificial sequence
  Synthetic oligonucleotide
ggaagcaccccucagcgagga

SEQ ID NO: 1257 - RNA, 21 nt, Artificial sequence
  Synthetic oligonucleotide
ccccgggggagcuggaggccu

SEQ ID NO: 1258 - RNA, 21 nt, Artificial sequence
  Synthetic oligonucleotide
aacggaggccccgggggagcu

SEQ ID NO: 1259 - RNA, 21 nt, Artificial sequence
  Synthetic oligonucleotide
gcagcaggcgcaaccucuccu

SEQ ID NO: 1260 - RNA, 21 nt, Artificial sequence
  Synthetic oligonucleotide
gaguuucugcagcaggcgcaa

SEQ ID NO: 1261 - RNA, 21 nt, Artificial sequence
  Synthetic oligonucleotide
gcuccuggcgagcccggaguu

SEQ ID NO: 1262 - RNA, 21 nt, Artificial sequence
  Synthetic oligonucleotide
ccccugcggccugcugcugga

SEQ ID NO: 1263 - RNA, 21 nt, Artificial sequence
  Synthetic oligonucleotide
cgcccuggucugcacuccccu

SEQ ID NO: 1264 - RNA, 21 nt, Artificial sequence
  Synthetic oligonucleotide
cccggcgcccucccaggcgcu

SEQ ID NO: 1265 - RNA, 21 nt, Artificial sequence
  Synthetic oligonucleotide
caagccggggcagcuccaccu

SEQ ID NO: 1266 - RNA, 21 nt, Artificial sequence
  Synthetic oligonucleotide
gucgccggggcggcgugggaa

SEQ ID NO: 1267 - RNA, 21 nt, Artificial sequence
  Synthetic oligonucleotide
ccgggccccugcgcgguggca

SEQ ID NO: 1268 - RNA, 21 nt, Artificial sequence
  Synthetic oligonucleotide
caaaagccgggaggaccggga

SEQ ID NO: 1269 - RNA, 21 nt, Artificial sequence
  Synthetic oligonucleotide
cggggcgcucucccacccuca

SEQ ID NO: 1270 - RNA, 21 nt, Artificial sequence
  Synthetic oligonucleotide
ugccccggcgcgcggggauuu

SEQ ID NO: 1271 - RNA, 21 nt, Artificial sequence
  Synthetic oligonucleotide
gccggcagaggggaucuccca

SEQ ID NO: 1272 - RNA, 21 nt, Artificial sequence
  Synthetic oligonucleotide
ggcucucccacagggggcuuu

SEQ ID NO: 1273 - RNA, 21 nt, Artificial sequence
  Synthetic oligonucleotide
ccuucgcccacaccggcgcgu

SEQ ID NO: 1274 - RNA, 21 nt, Artificial sequence
  Synthetic oligonucleotide
ggguggcagggcgcccgcgca

SEQ ID NO: 1275 - RNA, 21 nt, Artificial sequence
  Synthetic oligonucleotide
agaucugguuucagaaucgaa

SEQ ID NO: 1276 - RNA, 21 nt, Artificial sequence
  Synthetic oligonucleotide
caggauucagaucugguuuca

SEQ ID NO: 1277 - RNA, 21 nt, Artificial sequence
  Synthetic oligonucleotide
cgggccucccggaguccagga

SEQ ID NO: 1278 - RNA, 21 nt, Artificial sequence
  Synthetic oligonucleotide
cagagagacgggccucccgga

SEQ ID NO: 1279 - RNA, 21 nt, Artificial sequence
  Synthetic oligonucleotide
caucgccgcccgggaggagcu

SEQ ID NO: 1280 - RNA, 21 nt, Artificial sequence
  Synthetic oligonucleotide
cuccgagccuuugagaaggau

SEQ ID NO: 1281 - RNA, 21 nt, Artificial sequence
  Synthetic oligonucleotide
ccgccgucaccggaucccaga

SEQ ID NO: 1282 - RNA, 21 nt, Artificial sequence
  Synthetic oligonucleotide
cccgggagacgcggcccgcca

SEQ ID NO: 1283 - RNA, 21 nt, Artificial sequence
  Synthetic oligonucleotide
ugaggcagcaccggcgggaau

SEQ ID NO: 1284 - RNA, 21 nt, Artificial sequence
  Synthetic oligonucleotide
cggagcccaggguccagauuu

SEQ ID NO: 1285 - RNA, 21 nt, Artificial sequence
  Synthetic oligonucleotide
cggcauuccggagcccagggu

SEQ ID NO: 1286 - RNA, 21 nt, Artificial sequence
  Synthetic oligonucleotide
ggcuggcccaggccaucggca

SEQ ID NO: 1287 - RNA, 21 nt, Artificial sequence
  Synthetic oligonucleotide
ggcccugcgagccugcuuuga

SEQ ID NO: 1288 - RNA, 21 nt, Artificial sequence
  Synthetic oligonucleotide
gacggcgacggagacucguuu

SEQ ID NO: 1289 - RNA, 21 nt, Artificial sequence
  Synthetic oligonucleotide
ucccgacacccucggacagca

SEQ ID NO: 1290 - RNA, 21 nt, Artificial sequence
  Synthetic oligonucleotide
gcccgcguccguccgugaaau

SEQ ID NO: 1291 - RNA, 21 nt, Artificial sequence
  Synthetic oligonucleotide
agggcggcuggcuggcuggcu

SEQ ID NO: 1292 - RNA, 21 nt, Artificial sequence
  Synthetic oligonucleotide
ccuuuacaagggcggcuggcu

SEQ ID NO: 1293 - RNA, 21 nt, Artificial sequence
  Synthetic oligonucleotide
agauauauuaaaaugcccccu

SEQ ID NO: 1294 - RNA, 21 nt, Artificial sequence
  Synthetic oligonucleotide
cugucuaggcaaaccuggauu

SEQ ID NO: 1295 - RNA, 21 nt, Artificial sequence
  Synthetic oligonucleotide
ccccccaccuuccgacgcugu

SEQ ID NO: 1296 - RNA, 21 nt, Artificial sequence
  Synthetic oligonucleotide
cgcuggccucucugugcccuu

SEQ ID NO: 1297 - RNA, 21 nt, Artificial sequence
  Synthetic oligonucleotide
ugacgugcaagggagcucgcu

SEQ ID NO: 1298 - RNA, 21 nt, Artificial sequence
  Synthetic oligonucleotide
cugggcaucccggggauccca

SEQ ID NO: 1299 - RNA, 21 nt, Artificial sequence
  Synthetic oligonucleotide
gagacuccacuccgcggagaa

SEQ ID NO: 1300 - RNA, 21 nt, Artificial sequence
  Synthetic oligonucleotide
aggucuaggcccggugagaga

SEQ ID NO: 1301 - RNA, 21 nt, Artificial sequence
  Synthetic oligonucleotide
ugggauuccugccuucuaggu

SEQ ID NO: 1302 - RNA, 21 nt, Artificial sequence
  Synthetic oligonucleotide
cggggaacaccuggcuggcua

SEQ ID NO: 1303 - RNA, 21 nt, Artificial sequence
  Synthetic oligonucleotide
accagcagaccugcgcgcagu

SEQ ID NO: 1304 - RNA, 21 nt, Artificial sequence
  Synthetic oligonucleotide
cugccauucuuuccugggcau

SEQ ID NO: 1305 - RNA, 21 nt, Artificial sequence
  Synthetic oligonucleotide
ugagagacuccacaccgcgga

SEQ ID NO: 1306 - RNA, 21 nt, Artificial sequence
  Synthetic oligonucleotide
ggccuccgcacccgggcaaaa

SEQ ID NO: 1307 - RNA, 21 nt, Artificial sequence
  Synthetic oligonucleotide
ccucaggcuccucgcuggccu

SEQ ID NO: 1308 - RNA, 23 nt, Artificial sequence
  Synthetic oligonucleotide
ucucaccgggccuagaccccgcg

SEQ ID NO: 1309 - RNA, 21 nt, Artificial sequence
  Synthetic oligonucleotide
cggggucuaggcccggugaga

SEQ ID NO: 1310 - RNA, 23 nt, Artificial sequence
  Synthetic oligonucleotide
auaauccagguuugccuagacag

SEQ ID NO: 1311 - RNA, 23 nt, Artificial sequence
  Synthetic oligonucleotide
aguuugccuagacagcgucggaa

SEQ ID NO: 1312 - RNA, 23 nt, Artificial sequence
  Synthetic oligonucleotide
aucggaagguggggggagacauu

SEQ ID NO: 1313 - RNA, 23 nt, Artificial sequence
  Synthetic oligonucleotide
auggggggagacauucagccaga

SEQ ID NO: 1314 - RNA, 23 nt, Artificial sequence
  Synthetic oligonucleotide
aggcacagagaggccagcgagcu

SEQ ID NO: 1315 - RNA, 23 nt, Artificial sequence
  Synthetic oligonucleotide
acgagcucccuugcacgucagcc

SEQ ID NO: 1316 - RNA, 23 nt, Artificial sequence
  Synthetic oligonucleotide
acuugcacgucagccggggugcg

SEQ ID NO: 1317 - RNA, 23 nt, Artificial sequence
  Synthetic oligonucleotide
aucagccggggugcgcacugcgc

SEQ ID NO: 1318 - RNA, 23 nt, Artificial sequence
  Synthetic oligonucleotide
aggugcgcacugcgcgcaggucu

SEQ ID NO: 1319 - RNA, 23 nt, Artificial sequence
  Synthetic oligonucleotide
augguaccugggccggcucuggg

SEQ ID NO: 1320 - RNA, 23 nt, Artificial sequence
  Synthetic oligonucleotide
aucugggauccccgggaugccca

SEQ ID NO: 1321 - RNA, 23 nt, Artificial sequence
  Synthetic oligonucleotide
auggagucucucaccgggccuag

SEQ ID NO: 1322 - RNA, 23 nt, Artificial sequence
  Synthetic oligonucleotide
acccgcguccuaaagcuccucca

SEQ ID NO: 1323 - RNA, 23 nt, Artificial sequence
  Synthetic oligonucleotide
acuaaagcuccuccagcagagcc

SEQ ID NO: 1324 - RNA, 23 nt, Artificial sequence
  Synthetic oligonucleotide
aagagcccgguauucuuccucgc

SEQ ID NO: 1325 - RNA, 23 nt, Artificial sequence
  Synthetic oligonucleotide
aguauucuuccucgcugaggggu

SEQ ID NO: 1326 - RNA, 23 nt, Artificial sequence
  Synthetic oligonucleotide
aaggggugcuuccagcgaggcgg

SEQ ID NO: 1327 - RNA, 23 nt, Artificial sequence
  Synthetic oligonucleotide
auuccagcgaggcggccucuucc

SEQ ID NO: 1328 - RNA, 23 nt, Artificial sequence
  Synthetic oligonucleotide
aaggcggccucuuccgaggccuc

SEQ ID NO: 1329 - RNA, 23 nt, Artificial sequence
  Synthetic oligonucleotide
aucuuccgaggccuccagcuccc

SEQ ID NO: 1330 - RNA, 23 nt, Artificial sequence
  Synthetic oligonucleotide
aggggccuccguuucuaggagag

SEQ ID NO: 1331 - RNA, 23 nt, Artificial sequence
  Synthetic oligonucleotide
acguuucuaggagagguugcgcc

SEQ ID NO: 1332 - RNA, 23 nt, Artificial sequence
  Synthetic oligonucleotide
acugcagaaacuccgggcucgcc

SEQ ID NO: 1333 - RNA, 23 nt, Artificial sequence
  Synthetic oligonucleotide
acucgccaggagcucauccagca

SEQ ID NO: 1334 - RNA, 23 nt, Artificial sequence
  Synthetic oligonucleotide
agagcucauccagcagcaggccg

SEQ ID NO: 1335 - RNA, 23 nt, Artificial sequence
  Synthetic oligonucleotide
aaggccgcaggggagugcagacc

SEQ ID NO: 1336 - RNA, 23 nt, Artificial sequence
  Synthetic oligonucleotide
acagaccagggcgccggcuccug

SEQ ID NO: 1337 - RNA, 23 nt, Artificial sequence
  Synthetic oligonucleotide
aggcgccggcuccuggagcgccu

SEQ ID NO: 1338 - RNA, 23 nt, Artificial sequence
  Synthetic oligonucleotide
acuccuggagcgccugggagggc

SEQ ID NO: 1339 - RNA, 23 nt, Artificial sequence
  Synthetic oligonucleotide
agagggcgccgggaugccuugca

SEQ ID NO: 1340 - RNA, 23 nt, Artificial sequence
  Synthetic oligonucleotide
acgggaugccuugcaucugcccc

SEQ ID NO: 1341 - RNA, 23 nt, Artificial sequence
  Synthetic oligonucleotide
acuugcaucugccccugccgcgc

SEQ ID NO: 1342 - RNA, 23 nt, Artificial sequence
  Synthetic oligonucleotide
augccccugccgcgcggaggcgg

SEQ ID NO: 1343 - RNA, 23 nt, Artificial sequence
  Synthetic oligonucleotide
accgcgcggaggcggaggcgucc

SEQ ID NO: 1344 - RNA, 23 nt, Artificial sequence
  Synthetic oligonucleotide
aaggcggaggcguccgggggcgc

SEQ ID NO: 1345 - RNA, 23 nt, Artificial sequence
  Synthetic oligonucleotide
agcguccgggggcgcgggcuggg

SEQ ID NO: 1346 - RNA, 23 nt, Artificial sequence
  Synthetic oligonucleotide
agggcgcgggcuggggaggugga

SEQ ID NO: 1347 - RNA, 23 nt, Artificial sequence
  Synthetic oligonucleotide
agcuggggagguggagcugcccc

SEQ ID NO: 1348 - RNA, 23 nt, Artificial sequence
  Synthetic oligonucleotide
augccccggcuugggguucccac

SEQ ID NO: 1349 - RNA, 23 nt, Artificial sequence
  Synthetic oligonucleotide
acuugggguucccacgccgcccc

SEQ ID NO: 1350 - RNA, 23 nt, Artificial sequence
  Synthetic oligonucleotide
acgccccggcgaccuggggaccc

SEQ ID NO: 1351 - RNA, 23 nt, Artificial sequence
  Synthetic oligonucleotide
acgaccuggggaccccggcccca

SEQ ID NO: 1352 - RNA, 23 nt, Artificial sequence
  Synthetic oligonucleotide
aggaccccggccccagccccacc

SEQ ID NO: 1353 - RNA, 23 nt, Artificial sequence
  Synthetic oligonucleotide
agccccagccccaccacggacuc

SEQ ID NO: 1354 - RNA, 23 nt, Artificial sequence
  Synthetic oligonucleotide
acccaccacggacuccccuggga

SEQ ID NO: 1355 - RNA, 23 nt, Artificial sequence
  Synthetic oligonucleotide
aggacuccccugggacgugggug

SEQ ID NO: 1356 - RNA, 23 nt, Artificial sequence
  Synthetic oligonucleotide
acugggacguggguggcgcaagc

SEQ ID NO: 1357 - RNA, 23 nt, Artificial sequence
  Synthetic oligonucleotide
auggguggcgcaagcaccccuug

SEQ ID NO: 1358 - RNA, 23 nt, Artificial sequence
  Synthetic oligonucleotide
agcaagcaccccuuggcccugcg

SEQ ID NO: 1359 - RNA, 23 nt, Artificial sequence
  Synthetic oligonucleotide
acccuuggcccugcggccccgcu

SEQ ID NO: 1360 - RNA, 23 nt, Artificial sequence
  Synthetic oligonucleotide
accugcggccccgcuugagcggg

SEQ ID NO: 1361 - RNA, 23 nt, Artificial sequence
  Synthetic oligonucleotide
acccgcuugagcgggcccaggcu

SEQ ID NO: 1362 - RNA, 23 nt, Artificial sequence
  Synthetic oligonucleotide
aagcgggcccaggcugugccacc

SEQ ID NO: 1363 - RNA, 23 nt, Artificial sequence
  Synthetic oligonucleotide
acaggcugugccaccgcgcaggg

SEQ ID NO: 1364 - RNA, 23 nt, Artificial sequence
  Synthetic oligonucleotide
agcaggggcccggcaggccgucg

SEQ ID NO: 1365 - RNA, 23 nt, Artificial sequence
  Synthetic oligonucleotide
accggcaggccgucgcgcugcgg

SEQ ID NO: 1366 - RNA, 23 nt, Artificial sequence
  Synthetic oligonucleotide
accgucgcgcugcgggucccggu

SEQ ID NO: 1367 - RNA, 23 nt, Artificial sequence
  Synthetic oligonucleotide
acugcgggucccgguccucccgg

SEQ ID NO: 1368 - RNA, 23 nt, Artificial sequence
  Synthetic oligonucleotide
aucccggcuuuugcccgggugcg

SEQ ID NO: 1369 - RNA, 23 nt, Artificial sequence
  Synthetic oligonucleotide
aaggagccugagggugggagagc

SEQ ID NO: 1370 - RNA, 23 nt, Artificial sequence
  Synthetic oligonucleotide
agagagcgccccguccggaggag

SEQ ID NO: 1371 - RNA, 23 nt, Artificial sequence
  Synthetic oligonucleotide
acccguccggaggagccggggcg

SEQ ID NO: 1372 - RNA, 23 nt, Artificial sequence
  Synthetic oligonucleotide
agaggagccggggcggcguaggc

SEQ ID NO: 1373 - RNA, 23 nt, Artificial sequence
  Synthetic oligonucleotide
aggggcggcguaggcgaaauccc

SEQ ID NO: 1374 - RNA, 23 nt, Artificial sequence
  Synthetic oligonucleotide
aguaggcgaaauccccgcgcgcc

SEQ ID NO: 1375 - RNA, 23 nt, Artificial sequence
  Synthetic oligonucleotide
acgcgccggggcagguugggaga

SEQ ID NO: 1376 - RNA, 23 nt, Artificial sequence
  Synthetic oligonucleotide
aggcagguugggagauccccucu

SEQ ID NO: 1377 - RNA, 23 nt, Artificial sequence
  Synthetic oligonucleotide
acccucugccggcgcggccuggc

SEQ ID NO: 1378 - RNA, 23 nt, Artificial sequence
  Synthetic oligonucleotide
acggcgcggccuggcugggcugc

SEQ ID NO: 1379 - RNA, 23 nt, Artificial sequence
  Synthetic oligonucleotide
accuggcugggcugcagcgcggg

SEQ ID NO: 1380 - RNA, 23 nt, Artificial sequence
  Synthetic oligonucleotide
aggcugcagcgcgggggcggccc

SEQ ID NO: 1381 - RNA, 23 nt, Artificial sequence
  Synthetic oligonucleotide
acgcgggggcggcccucgcugcc

SEQ ID NO: 1382 - RNA, 23 nt, Artificial sequence
  Synthetic oligonucleotide
acggcccucgcugccuggcucac

SEQ ID NO: 1383 - RNA, 23 nt, Artificial sequence
  Synthetic oligonucleotide
agcugccuggcucacgaaagccc

SEQ ID NO: 1384 - RNA, 23 nt, Artificial sequence
  Synthetic oligonucleotide
agcucacgaaagcccccuguggg

SEQ ID NO: 1385 - RNA, 23 nt, Artificial sequence
  Synthetic oligonucleotide
augugggagagccccaggcgcgc

SEQ ID NO: 1386 - RNA, 23 nt, Artificial sequence
  Synthetic oligonucleotide
aagccccaggcgcgcagggcacg

SEQ ID NO: 1387 - RNA, 23 nt, Artificial sequence
  Synthetic oligonucleotide
agcgcgcagggcacguggggugc

SEQ ID NO: 1388 - RNA, 23 nt, Artificial sequence
  Synthetic oligonucleotide
aggcacguggggugcgggaagcc

SEQ ID NO: 1389 - RNA, 23 nt, Artificial sequence
  Synthetic oligonucleotide
agggugcgggaagccccguuccc

SEQ ID NO: 1390 - RNA, 23 nt, Artificial sequence
  Synthetic oligonucleotide
agaagccccguuccccacgcgcc

SEQ ID NO: 1391 - RNA, 23 nt, Artificial sequence
  Synthetic oligonucleotide
aguuccccacgcgccgguguggg

SEQ ID NO: 1392 - RNA, 23 nt, Artificial sequence
  Synthetic oligonucleotide
augugggcgaaggcgacccacga

SEQ ID NO: 1393 - RNA, 23 nt, Artificial sequence
  Synthetic oligonucleotide
aaaggcgacccacgagggagcag

SEQ ID NO: 1394 - RNA, 23 nt, Artificial sequence
  Synthetic oligonucleotide
accacgagggagcagggugaccc

SEQ ID NO: 1395 - RNA, 23 nt, Artificial sequence
  Synthetic oligonucleotide
agagcagggugacccccgccggg

SEQ ID NO: 1396 - RNA, 23 nt, Artificial sequence
  Synthetic oligonucleotide
augacccccgccgggggccgcgc

SEQ ID NO: 1397 - RNA, 23 nt, Artificial sequence
  Synthetic oligonucleotide
agccgggggccgcgcugcacagg

SEQ ID NO: 1398 - RNA, 23 nt, Artificial sequence
  Synthetic oligonucleotide
accgcgcugcacaggccgccugc

SEQ ID NO: 1399 - RNA, 23 nt, Artificial sequence
  Synthetic oligonucleotide
acacaggccgccugccugcgcgg

SEQ ID NO: 1400 - RNA, 23 nt, Artificial sequence
  Synthetic oligonucleotide
agccugccugcgcgggcgcccug

SEQ ID NO: 1401 - RNA, 23 nt, Artificial sequence
  Synthetic oligonucleotide
agcccugccacccugucccgggu

SEQ ID NO: 1402 - RNA, 23 nt, Artificial sequence
  Synthetic oligonucleotide
aacccugucccgggugccuggcc

SEQ ID NO: 1403 - RNA, 23 nt, Artificial sequence
  Synthetic oligonucleotide
accgggugccuggcccuucgauu

SEQ ID NO: 1404 - RNA, 23 nt, Artificial sequence
  Synthetic oligonucleotide
acuggcccuucgauucugaaacc

SEQ ID NO: 1405 - RNA, 23 nt, Artificial sequence
  Synthetic oligonucleotide
aaucugaauccuggacuccggga

SEQ ID NO: 1406 - RNA, 23 nt, Artificial sequence
  Synthetic oligonucleotide
acccgucucucuggccagcuccu

SEQ ID NO: 1407 - RNA, 23 nt, Artificial sequence
  Synthetic oligonucleotide
aucuggccagcuccucccgggcg

SEQ ID NO: 1408 - RNA, 23 nt, Artificial sequence
  Synthetic oligonucleotide
acgggcggcgaugccuggaaagc

SEQ ID NO: 1409 - RNA, 23 nt, Artificial sequence
  Synthetic oligonucleotide
agaugccuggaaagcgauccuuc

SEQ ID NO: 1410 - RNA, 23 nt, Artificial sequence
  Synthetic oligonucleotide
agaaagcgauccuucucaaaggc

SEQ ID NO: 1411 - RNA, 23 nt, Artificial sequence
  Synthetic oligonucleotide
aaaaggcucggaggagcagggcg

SEQ ID NO: 1412 - RNA, 23 nt, Artificial sequence
  Synthetic oligonucleotide
aggaggagcagggcggucuggga

SEQ ID NO: 1413 - RNA, 23 nt, Artificial sequence
  Synthetic oligonucleotide
aagggcggucugggauccgguga

SEQ ID NO: 1414 - RNA, 23 nt, Artificial sequence
  Synthetic oligonucleotide
acggugacggcgguccgcuuucg

SEQ ID NO: 1415 - RNA, 23 nt, Artificial sequence
  Synthetic oligonucleotide
agcgguccgcuuucgccggccuu

SEQ ID NO: 1416 - RNA, 23 nt, Artificial sequence
  Synthetic oligonucleotide
acuuucgccggccuucuggcggg

SEQ ID NO: 1417 - RNA, 23 nt, Artificial sequence
  Synthetic oligonucleotide
aggccuucuggcgggccgcgucu

SEQ ID NO: 1418 - RNA, 23 nt, Artificial sequence
  Synthetic oligonucleotide
agcgucucccgggccagggccga

SEQ ID NO: 1419 - RNA, 23 nt, Artificial sequence
  Synthetic oligonucleotide
acgggccagggccgagauucccg

SEQ ID NO: 1420 - RNA, 23 nt, Artificial sequence
  Synthetic oligonucleotide
aggccgagauucccgccggugcu

SEQ ID NO: 1421 - RNA, 23 nt, Artificial sequence
  Synthetic oligonucleotide
aggugcugccucagcuggcguga

SEQ ID NO: 1422 - RNA, 23 nt, Artificial sequence
  Synthetic oligonucleotide
acucagcuggcgugaccucucau

SEQ ID NO: 1423 - RNA, 23 nt, Artificial sequence
  Synthetic oligonucleotide
agcgugaccucucauucugaaac

SEQ ID NO: 1424 - RNA, 23 nt, Artificial sequence
  Synthetic oligonucleotide
aucucauucugaaaccaaaucug

SEQ ID NO: 1425 - RNA, 23 nt, Artificial sequence
  Synthetic oligonucleotide
augaaaccaaaucuggacccugg

SEQ ID NO: 1426 - RNA, 23 nt, Artificial sequence
  Synthetic oligonucleotide
auccggaaugccgauggccuggg

SEQ ID NO: 1427 - RNA, 23 nt, Artificial sequence
  Synthetic oligonucleotide
accugggccagccguucucuggu

SEQ ID NO: 1428 - RNA, 23 nt, Artificial sequence
  Synthetic oligonucleotide
aagccguucucugguggcgaugc

SEQ ID NO: 1429 - RNA, 23 nt, Artificial sequence
  Synthetic oligonucleotide
aucugguggcgaugcccggguac

SEQ ID NO: 1430 - RNA, 23 nt, Artificial sequence
  Synthetic oligonucleotide
acgaugcccggguacggguuccg

SEQ ID NO: 1431 - RNA, 23 nt, Artificial sequence
  Synthetic oligonucleotide
aggguacggguuccgcucaaagc

SEQ ID NO: 1432 - RNA, 23 nt, Artificial sequence
  Synthetic oligonucleotide
aguuccgcucaaagcaggcucgc

SEQ ID NO: 1433 - RNA, 23 nt, Artificial sequence
  Synthetic oligonucleotide
agcucgcagggccucgcuuuggc

SEQ ID NO: 1434 - RNA, 23 nt, Artificial sequence
  Synthetic oligonucleotide
aggccucgcuuuggcucgggguc

SEQ ID NO: 1435 - RNA, 23 nt, Artificial sequence
  Synthetic oligonucleotide
auuuggcucgggguccaaacgag

SEQ ID NO: 1436 - RNA, 23 nt, Artificial sequence
  Synthetic oligonucleotide
agggguccaaacgagucuccguc

SEQ ID NO: 1437 - RNA, 23 nt, Artificial sequence
  Synthetic oligonucleotide
auccgucgccguccucguccccg

SEQ ID NO: 1438 - RNA, 23 nt, Artificial sequence
  Synthetic oligonucleotide
acguccucguccccgggcuuccg

SEQ ID NO: 1439 - RNA, 23 nt, Artificial sequence
  Synthetic oligonucleotide
auccccgggcuuccgcggggagg

SEQ ID NO: 1440 - RNA, 23 nt, Artificial sequence
  Synthetic oligonucleotide
acuuccgcggggagggugcuguc

SEQ ID NO: 1441 - RNA, 23 nt, Artificial sequence
  Synthetic oligonucleotide
agggagggugcuguccgagggug

SEQ ID NO: 1442 - RNA, 23 nt, Artificial sequence
  Synthetic oligonucleotide
aagggugucgggagggccaucgc

SEQ ID NO: 1443 - RNA, 23 nt, Artificial sequence
  Synthetic oligonucleotide
agggagggccaucgcggugagcc

SEQ ID NO: 1444 - RNA, 23 nt, Artificial sequence
  Synthetic oligonucleotide
acaucgcggugagccccggccgg

SEQ ID NO: 1445 - RNA, 23 nt, Artificial sequence
  Synthetic oligonucleotide
augagccccggccggaauuucac

SEQ ID NO: 1446 - RNA, 23 nt, Artificial sequence
  Synthetic oligonucleotide
aggccggaauuucacggacggac

SEQ ID NO: 1447 - RNA, 23 nt, Artificial sequence
  Synthetic oligonucleotide
aacggacgcgggcagagagaggc

SEQ ID NO: 1448 - RNA, 23 nt, Artificial sequence
  Synthetic oligonucleotide
agggcagagagaggccggcgggc

SEQ ID NO: 1449 - RNA, 23 nt, Artificial sequence
  Synthetic oligonucleotide
aagaggccggcgggcucccgugc

SEQ ID NO: 1450 - RNA, 23 nt, Artificial sequence
  Synthetic oligonucleotide
agcgggcucccgugcaccucagc

SEQ ID NO: 1451 - RNA, 23 nt, Artificial sequence
  Synthetic oligonucleotide
accgugcaccucagccggacugu

SEQ ID NO: 1452 - RNA, 23 nt, Artificial sequence
  Synthetic oligonucleotide
acucagccggacugugcacugcg

SEQ ID NO: 1453 - RNA, 23 nt, Artificial sequence
  Synthetic oligonucleotide
agacugugcacugcggcaggugc

SEQ ID NO: 1454 - RNA, 23 nt, Artificial sequence
  Synthetic oligonucleotide
aacugcggcaggugcagccagga

SEQ ID NO: 1455 - RNA, 23 nt, Artificial sequence
  Synthetic oligonucleotide
aaggugcagccaggaggccugcc

SEQ ID NO: 1456 - RNA, 23 nt, Artificial sequence
  Synthetic oligonucleotide
accaggaggccugcccggacagc

SEQ ID NO: 1457 - RNA, 23 nt, Artificial sequence
  Synthetic oligonucleotide
accugcccggacagccagccagc

SEQ ID NO: 1458 - RNA, 23 nt, Artificial sequence
  Synthetic oligonucleotide
agacagccagccagccagccagc

SEQ ID NO: 1459 - RNA, 23 nt, Artificial sequence
  Synthetic oligonucleotide
auauaggauccacagggaggggg

SEQ ID NO: 1460 - RNA, 23 nt, Artificial sequence
  Synthetic oligonucleotide
acaggagauguaacucuaaucca

SEQ ID NO: 1461 - RNA, 23 nt, Artificial sequence
  Synthetic oligonucleotide
agggagacauucagccagaauuu

SEQ ID NO: 1462 - RNA, 23 nt, Artificial sequence
  Synthetic oligonucleotide
aaauuucacggaagaacaagggc

SEQ ID NO: 1463 - RNA, 23 nt, Artificial sequence
  Synthetic oligonucleotide
aguaccugggccggcucugggau

SEQ ID NO: 1464 - RNA, 23 nt, Artificial sequence
  Synthetic oligonucleotide
acaggaaagaaaggcaguucucc

SEQ ID NO: 1465 - RNA, 23 nt, Artificial sequence
  Synthetic oligonucleotide
accaggccggucagcccggugga

SEQ ID NO: 1466 - RNA, 23 nt, Artificial sequence
  Synthetic oligonucleotide
aguggagggggcggggcggagac

SEQ ID NO: 1467 - RNA, 23 nt, Artificial sequence
  Synthetic oligonucleotide
agagacacgccccuccguagcca

SEQ ID NO: 1468 - RNA, 23 nt, Artificial sequence
  Synthetic oligonucleotide
accgcgaaagagaggccaccgcc

SEQ ID NO: 1469 - RNA, 23 nt, Artificial sequence
  Synthetic oligonucleotide
aaggucugcugguaccugggccg

SEQ ID NO: 1470 - RNA, 23 nt, Artificial sequence
  Synthetic oligonucleotide
aaaagaauggcaguucuccgcgg

SEQ ID NO: 1471 - RNA, 23 nt, Artificial sequence
  Synthetic oligonucleotide
agcaguucuccgcgguguggagu

SEQ ID NO: 1472 - RNA, 23 nt, Artificial sequence
  Synthetic oligonucleotide
aggugcggaggccagcgaggagc

SEQ ID NO: 1473 - RNA, 23 nt, Artificial sequence
  Synthetic oligonucleotide
augcgcgcaggucugcugguacc

SEQ ID NO: 1474 - RNA, 21 nt, Artificial sequence
  Synthetic oligonucleotide
gucuaggcaaaccuggauuau

SEQ ID NO: 1475 - RNA, 21 nt, Artificial sequence
  Synthetic oligonucleotide
ccgacgcugucuaggcaaacu

SEQ ID NO: 1476 - RNA, 21 nt, Artificial sequence
  Synthetic oligonucleotide
ugucuccccccaccuuccgau

SEQ ID NO: 1477 - RNA, 21 nt, Artificial sequence
  Synthetic oligonucleotide
uggcugaaugucuccccccau

SEQ ID NO: 1478 - RNA, 21 nt, Artificial sequence
  Synthetic oligonucleotide
cucgcuggccucucugugccu

SEQ ID NO: 1479 - RNA, 21 nt, Artificial sequence
  Synthetic oligonucleotide
cugacgugcaagggagcucgu

SEQ ID NO: 1480 - RNA, 21 nt, Artificial sequence
  Synthetic oligonucleotide
caccccggcugacgugcaagu

SEQ ID NO: 1481 - RNA, 21 nt, Artificial sequence
  Synthetic oligonucleotide
gcagugcgcaccccggcugau

SEQ ID NO: 1482 - RNA, 21 nt, Artificial sequence
  Synthetic oligonucleotide
accugcgcgcagugcgcaccu

SEQ ID NO: 1483 - RNA, 21 nt, Artificial sequence
  Synthetic oligonucleotide
cagagccggcccagguaccau

SEQ ID NO: 1484 - RNA, 21 nt, Artificial sequence
  Synthetic oligonucleotide
ggcaucccggggaucccagau

SEQ ID NO: 1485 - RNA, 21 nt, Artificial sequence
  Synthetic oligonucleotide
aggcccggugagagacuccau

SEQ ID NO: 1486 - RNA, 21 nt, Artificial sequence
  Synthetic oligonucleotide
gaggagcuuuaggacgcgggu

SEQ ID NO: 1487 - RNA, 21 nt, Artificial sequence
  Synthetic oligonucleotide
cucugcuggaggagcuuuagu

SEQ ID NO: 1488 - RNA, 21 nt, Artificial sequence
  Synthetic oligonucleotide
gaggaagaauaccgggcucuu

SEQ ID NO: 1489 - RNA, 21 nt, Artificial sequence
  Synthetic oligonucleotide
cccucagcgaggaagaauacu

SEQ ID NO: 1490 - RNA, 21 nt, Artificial sequence
  Synthetic oligonucleotide
gccucgcuggaagcaccccuu

SEQ ID NO: 1491 - RNA, 21 nt, Artificial sequence
  Synthetic oligonucleotide
aagaggccgccucgcuggaau

SEQ ID NO: 1492 - RNA, 21 nt, Artificial sequence
  Synthetic oligonucleotide
ggccucggaagaggccgccuu

SEQ ID NO: 1493 - RNA, 21 nt, Artificial sequence
  Synthetic oligonucleotide
gagcuggaggccucggaagau

SEQ ID NO: 1494 - RNA, 21 nt, Artificial sequence
  Synthetic oligonucleotide
cuccuagaaacggaggccccu

SEQ ID NO: 1495 - RNA, 21 nt, Artificial sequence
  Synthetic oligonucleotide
cgcaaccucuccuagaaacgu

SEQ ID NO: 1496 - RNA, 21 nt, Artificial sequence
  Synthetic oligonucleotide
cgagcccggaguuucugcagu

SEQ ID NO: 1497 - RNA, 21 nt, Artificial sequence
  Synthetic oligonucleotide
cuggaugagcuccuggcgagu

SEQ ID NO: 1498 - RNA, 21 nt, Artificial sequence
  Synthetic oligonucleotide
gccugcugcuggaugagcucu

SEQ ID NO: 1499 - RNA, 21 nt, Artificial sequence
  Synthetic oligonucleotide
ucugcacuccccugcggccuu

SEQ ID NO: 1500 - RNA, 21 nt, Artificial sequence
  Synthetic oligonucleotide
ggagccggcgcccuggucugu

SEQ ID NO: 1501 - RNA, 21 nt, Artificial sequence
  Synthetic oligonucleotide
gcgcuccaggagccggcgccu

SEQ ID NO: 1502 - RNA, 21 nt, Artificial sequence
  Synthetic oligonucleotide
ccucccaggcgcuccaggagu

SEQ ID NO: 1503 - RNA, 21 nt, Artificial sequence
  Synthetic oligonucleotide
caaggcaucccggcgcccucu

SEQ ID NO: 1504 - RNA, 21 nt, Artificial sequence
  Synthetic oligonucleotide
ggcagaugcaaggcaucccgu

SEQ ID NO: 1505 - RNA, 21 nt, Artificial sequence
  Synthetic oligonucleotide
gcggcaggggcagaugcaagu

SEQ ID NO: 1506 - RNA, 21 nt, Artificial sequence
  Synthetic oligonucleotide
gccuccgcgcggcaggggcau

SEQ ID NO: 1507 - RNA, 21 nt, Artificial sequence
  Synthetic oligonucleotide
acgccuccgccuccgcgcggu

SEQ ID NO: 1508 - RNA, 21 nt, Artificial sequence
  Synthetic oligonucleotide
gcccccggacgccuccgccuu

SEQ ID NO: 1509 - RNA, 21 nt, Artificial sequence
  Synthetic oligonucleotide
cagcccgcgcccccggacgcu

SEQ ID NO: 1510 - RNA, 21 nt, Artificial sequence
  Synthetic oligonucleotide
caccuccccagcccgcgcccu

SEQ ID NO: 1511 - RNA, 21 nt, Artificial sequence
  Synthetic oligonucleotide
ggcagcuccaccuccccagcu

SEQ ID NO: 1512 - RNA, 21 nt, Artificial sequence
  Synthetic oligonucleotide
gggaaccccaagccggggcau

SEQ ID NO: 1513 - RNA, 21 nt, Artificial sequence
  Synthetic oligonucleotide
ggcggcgugggaaccccaagu

SEQ ID NO: 1514 - RNA, 21 nt, Artificial sequence
  Synthetic oligonucleotide
guccccaggucgccggggcgu

SEQ ID NO: 1515 - RNA, 21 nt, Artificial sequence
  Synthetic oligonucleotide
gggccgggguccccaggucgu

SEQ ID NO: 1516 - RNA, 21 nt, Artificial sequence
  Synthetic oligonucleotide
uggggcuggggccgggguccu

SEQ ID NO: 1517 - RNA, 21 nt, Artificial sequence
  Synthetic oligonucleotide
guccgugguggggcuggggcu

SEQ ID NO: 1518 - RNA, 21 nt, Artificial sequence
  Synthetic oligonucleotide
ccaggggaguccguggugggu

SEQ ID NO: 1519 - RNA, 21 nt, Artificial sequence
  Synthetic oligonucleotide
cccacgucccaggggaguccu

SEQ ID NO: 1520 - RNA, 21 nt, Artificial sequence
  Synthetic oligonucleotide
uugcgccacccacgucccagu

SEQ ID NO: 1521 - RNA, 21 nt, Artificial sequence
  Synthetic oligonucleotide
aggggugcuugcgccacccau

SEQ ID NO: 1522 - RNA, 21 nt, Artificial sequence
  Synthetic oligonucleotide
cagggccaaggggugcuugcu

SEQ ID NO: 1523 - RNA, 21 nt, Artificial sequence
  Synthetic oligonucleotide
cggggccgcagggccaagggu

SEQ ID NO: 1524 - RNA, 21 nt, Artificial sequence
  Synthetic oligonucleotide
cgcucaagcggggccgcaggu

SEQ ID NO: 1525 - RNA, 21 nt, Artificial sequence
  Synthetic oligonucleotide
ccugggcccgcucaagcgggu

SEQ ID NO: 1526 - RNA, 21 nt, Artificial sequence
  Synthetic oligonucleotide
uggcacagccugggcccgcuu

SEQ ID NO: 1527 - RNA, 21 nt, Artificial sequence
  Synthetic oligonucleotide
cugcgcgguggcacagccugu

SEQ ID NO: 1528 - RNA, 21 nt, Artificial sequence
  Synthetic oligonucleotide
acggccugccgggccccugcu

SEQ ID NO: 1529 - RNA, 21 nt, Artificial sequence
  Synthetic oligonucleotide
gcagcgcgacggccugccggu

SEQ ID NO: 1530 - RNA, 21 nt, Artificial sequence
  Synthetic oligonucleotide
cgggacccgcagcgcgacggu

SEQ ID NO: 1531 - RNA, 21 nt, Artificial sequence
  Synthetic oligonucleotide
gggaggaccgggacccgcagu

SEQ ID NO: 1532 - RNA, 21 nt, Artificial sequence
  Synthetic oligonucleotide
cacccgggcaaaagccgggau

SEQ ID NO: 1533 - RNA, 21 nt, Artificial sequence
  Synthetic oligonucleotide
ucucccacccucaggcuccuu

SEQ ID NO: 1534 - RNA, 21 nt, Artificial sequence
  Synthetic oligonucleotide
ccuccggacggggcgcucucu

SEQ ID NO: 1535 - RNA, 21 nt, Artificial sequence
  Synthetic oligonucleotide
ccccggcuccuccggacgggu

SEQ ID NO: 1536 - RNA, 21 nt, Artificial sequence
  Synthetic oligonucleotide
cuacgccgccccggcuccucu

SEQ ID NO: 1537 - RNA, 21 nt, Artificial sequence
  Synthetic oligonucleotide
gauuucgccuacgccgccccu

SEQ ID NO: 1538 - RNA, 21 nt, Artificial sequence
  Synthetic oligonucleotide
cgcgcggggauuucgccuacu

SEQ ID NO: 1539 - RNA, 21 nt, Artificial sequence
  Synthetic oligonucleotide
ucccaaccugccccggcgcgu

SEQ ID NO: 1540 - RNA, 21 nt, Artificial sequence
  Synthetic oligonucleotide
aggggaucucccaaccugccu

SEQ ID NO: 1541 - RNA, 21 nt, Artificial sequence
  Synthetic oligonucleotide
caggccgcgccggcagagggu

SEQ ID NO: 1542 - RNA, 21 nt, Artificial sequence
  Synthetic oligonucleotide
agcccagccaggccgcgccgu

SEQ ID NO: 1543 - RNA, 21 nt, Artificial sequence
  Synthetic oligonucleotide
cgcgcugcagcccagccaggu

SEQ ID NO: 1544 - RNA, 21 nt, Artificial sequence
  Synthetic oligonucleotide
gccgcccccgcgcugcagccu

SEQ ID NO: 1545 - RNA, 21 nt, Artificial sequence
  Synthetic oligonucleotide
cagcgagggccgcccccgcgu

SEQ ID NO: 1546 - RNA, 21 nt, Artificial sequence
  Synthetic oligonucleotide
gagccaggcagcgagggccgu

SEQ ID NO: 1547 - RNA, 21 nt, Artificial sequence
  Synthetic oligonucleotide
gcuuucgugagccaggcagcu

SEQ ID NO: 1548 - RNA, 21 nt, Artificial sequence
  Synthetic oligonucleotide
cacagggggcuuucgugagcu

SEQ ID NO: 1549 - RNA, 21 nt, Artificial sequence
  Synthetic oligonucleotide
gcgccuggggcucucccacau

SEQ ID NO: 1550 - RNA, 21 nt, Artificial sequence
  Synthetic oligonucleotide
ugcccugcgcgccuggggcuu

SEQ ID NO: 1551 - RNA, 21 nt, Artificial sequence
  Synthetic oligonucleotide
accccacgugcccugcgcgcu

SEQ ID NO: 1552 - RNA, 21 nt, Artificial sequence
  Synthetic oligonucleotide
cuucccgcaccccacgugccu

SEQ ID NO: 1553 - RNA, 21 nt, Artificial sequence
  Synthetic oligonucleotide
gaacggggcuucccgcacccu

SEQ ID NO: 1554 - RNA, 21 nt, Artificial sequence
  Synthetic oligonucleotide
cgcguggggaacggggcuucu

SEQ ID NO: 1555 - RNA, 21 nt, Artificial sequence
  Synthetic oligonucleotide
cacaccggcgcguggggaacu

SEQ ID NO: 1556 - RNA, 21 nt, Artificial sequence
  Synthetic oligonucleotide
gugggucgccuucgcccacau

SEQ ID NO: 1557 - RNA, 21 nt, Artificial sequence
  Synthetic oligonucleotide
gcucccucgugggucgccuuu

SEQ ID NO: 1558 - RNA, 21 nt, Artificial sequence
  Synthetic oligonucleotide
gucacccugcucccucguggu

SEQ ID NO: 1559 - RNA, 21 nt, Artificial sequence
  Synthetic oligonucleotide
cggcgggggucacccugcucu

SEQ ID NO: 1560 - RNA, 21 nt, Artificial sequence
  Synthetic oligonucleotide
gcggcccccggcgggggucau

SEQ ID NO: 1561 - RNA, 21 nt, Artificial sequence
  Synthetic oligonucleotide
ugugcagcgcggcccccggcu

SEQ ID NO: 1562 - RNA, 21 nt, Artificial sequence
  Synthetic oligonucleotide
aggcggccugugcagcgcggu

SEQ ID NO: 1563 - RNA, 21 nt, Artificial sequence
  Synthetic oligonucleotide
gcgcaggcaggcggccugugu

SEQ ID NO: 1564 - RNA, 21 nt, Artificial sequence
  Synthetic oligonucleotide
gggcgcccgcgcaggcaggcu

SEQ ID NO: 1565 - RNA, 21 nt, Artificial sequence
  Synthetic oligonucleotide
ccgggacaggguggcagggcu

SEQ ID NO: 1566 - RNA, 21 nt, Artificial sequence
  Synthetic oligonucleotide
ccaggcacccgggacaggguu

SEQ ID NO: 1567 - RNA, 21 nt, Artificial sequence
  Synthetic oligonucleotide
ucgaagggccaggcacccggu

SEQ ID NO: 1568 - RNA, 21 nt, Artificial sequence
  Synthetic oligonucleotide
uuucagaaucgaagggccagu

SEQ ID NO: 1569 - RNA, 21 nt, Artificial sequence
  Synthetic oligonucleotide
ccggaguccaggauucagauu

SEQ ID NO: 1570 - RNA, 21 nt, Artificial sequence
  Synthetic oligonucleotide
gagcuggccagagagacgggu

SEQ ID NO: 1571 - RNA, 21 nt, Artificial sequence
  Synthetic oligonucleotide
cccgggaggagcuggccagau

SEQ ID NO: 1572 - RNA, 21 nt, Artificial sequence
  Synthetic oligonucleotide
uuuccaggcaucgccgcccgu

SEQ ID NO: 1573 - RNA, 21 nt, Artificial sequence
  Synthetic oligonucleotide
aggaucgcuuuccaggcaucu

SEQ ID NO: 1574 - RNA, 21 nt, Artificial sequence
  Synthetic oligonucleotide
cuuugagaaggaucgcuuucu

SEQ ID NO: 1575 - RNA, 21 nt, Artificial sequence
  Synthetic oligonucleotide
cccugcuccuccgagccuuuu

SEQ ID NO: 1576 - RNA, 21 nt, Artificial sequence
  Synthetic oligonucleotide
ccagaccgcccugcuccuccu

SEQ ID NO: 1577 - RNA, 21 nt, Artificial sequence
  Synthetic oligonucleotide
accggaucccagaccgcccuu

SEQ ID NO: 1578 - RNA, 21 nt, Artificial sequence
  Synthetic oligonucleotide
aaagcggaccgccgucaccgu

SEQ ID NO: 1579 - RNA, 21 nt, Artificial sequence
  Synthetic oligonucleotide
ggccggcgaaagcggaccgcu

SEQ ID NO: 1580 - RNA, 21 nt, Artificial sequence
  Synthetic oligonucleotide
cgccagaaggccggcgaaagu

SEQ ID NO: 1581 - RNA, 21 nt, Artificial sequence
  Synthetic oligonucleotide
acgcggcccgccagaaggccu

SEQ ID NO: 1582 - RNA, 21 nt, Artificial sequence
  Synthetic oligonucleotide
ggcccuggcccgggagacgcu

SEQ ID NO: 1583 - RNA, 21 nt, Artificial sequence
  Synthetic oligonucleotide
ggaaucucggcccuggcccgu

SEQ ID NO: 1584 - RNA, 21 nt, Artificial sequence
  Synthetic oligonucleotide
caccggcgggaaucucggccu

SEQ ID NO: 1585 - RNA, 21 nt, Artificial sequence
  Synthetic oligonucleotide
acgccagcugaggcagcaccu

SEQ ID NO: 1586 - RNA, 21 nt, Artificial sequence
  Synthetic oligonucleotide
gagaggucacgccagcugagu

SEQ ID NO: 1587 - RNA, 21 nt, Artificial sequence
  Synthetic oligonucleotide
uucagaaugagaggucacgcu

SEQ ID NO: 1588 - RNA, 21 nt, Artificial sequence
  Synthetic oligonucleotide
gauuugguuucagaaugagau

SEQ ID NO: 1589 - RNA, 21 nt, Artificial sequence
  Synthetic oligonucleotide
aggguccagauuugguuucau

SEQ ID NO: 1590 - RNA, 21 nt, Artificial sequence
  Synthetic oligonucleotide
caggccaucggcauuccggau

SEQ ID NO: 1591 - RNA, 21 nt, Artificial sequence
  Synthetic oligonucleotide
cagagaacggcuggcccaggu

SEQ ID NO: 1592 - RNA, 21 nt, Artificial sequence
  Synthetic oligonucleotide
aucgccaccagagaacggcuu

SEQ ID NO: 1593 - RNA, 21 nt, Artificial sequence
  Synthetic oligonucleotide
acccgggcaucgccaccagau

SEQ ID NO: 1594 - RNA, 21 nt, Artificial sequence
  Synthetic oligonucleotide
gaacccguacccgggcaucgu

SEQ ID NO: 1595 - RNA, 21 nt, Artificial sequence
  Synthetic oligonucleotide
uuugagcggaacccguacccu

SEQ ID NO: 1596 - RNA, 21 nt, Artificial sequence
  Synthetic oligonucleotide
gagccugcuuugagcggaacu

SEQ ID NO: 1597 - RNA, 21 nt, Artificial sequence
  Synthetic oligonucleotide
caaagcgaggcccugcgagcu

SEQ ID NO: 1598 - RNA, 21 nt, Artificial sequence
  Synthetic oligonucleotide
ccccgagccaaagcgaggccu

SEQ ID NO: 1599 - RNA, 21 nt, Artificial sequence
  Synthetic oligonucleotide
cguuuggaccccgagccaaau

SEQ ID NO: 1600 - RNA, 21 nt, Artificial sequence
  Synthetic oligonucleotide
cggagacucguuuggaccccu

SEQ ID NO: 1601 - RNA, 21 nt, Artificial sequence
  Synthetic oligonucleotide
gggacgaggacggcgacggau

SEQ ID NO: 1602 - RNA, 21 nt, Artificial sequence
  Synthetic oligonucleotide
gaagcccggggacgaggacgu

SEQ ID NO: 1603 - RNA, 21 nt, Artificial sequence
  Synthetic oligonucleotide
uccccgcggaagcccggggau

SEQ ID NO: 1604 - RNA, 21 nt, Artificial sequence
  Synthetic oligonucleotide
cagcacccuccccgcggaagu

SEQ ID NO: 1605 - RNA, 21 nt, Artificial sequence
  Synthetic oligonucleotide
cccucggacagcacccucccu

SEQ ID NO: 1606 - RNA, 21 nt, Artificial sequence
  Synthetic oligonucleotide
gauggcccucccgacacccuu

SEQ ID NO: 1607 - RNA, 21 nt, Artificial sequence
  Synthetic oligonucleotide
cucaccgcgauggcccucccu

SEQ ID NO: 1608 - RNA, 21 nt, Artificial sequence
  Synthetic oligonucleotide
ggccggggcucaccgcgaugu

SEQ ID NO: 1609 - RNA, 21 nt, Artificial sequence
  Synthetic oligonucleotide
gaaauuccggccggggcucau

SEQ ID NO: 1610 - RNA, 21 nt, Artificial sequence
  Synthetic oligonucleotide
ccguccgugaaauuccggccu

SEQ ID NO: 1611 - RNA, 21 nt, Artificial sequence
  Synthetic oligonucleotide
cucucucugcccgcguccguu

SEQ ID NO: 1612 - RNA, 21 nt, Artificial sequence
  Synthetic oligonucleotide
ccgccggccucucucugcccu

SEQ ID NO: 1613 - RNA, 21 nt, Artificial sequence
  Synthetic oligonucleotide
acgggagcccgccggccucuu

SEQ ID NO: 1614 - RNA, 21 nt, Artificial sequence
  Synthetic oligonucleotide
ugaggugcacgggagcccgcu

SEQ ID NO: 1615 - RNA, 21 nt, Artificial sequence
  Synthetic oligonucleotide
aguccggcugaggugcacggu

SEQ ID NO: 1616 - RNA, 21 nt, Artificial sequence
  Synthetic oligonucleotide
cagugcacaguccggcugagu

SEQ ID NO: 1617 - RNA, 21 nt, Artificial sequence
  Synthetic oligonucleotide
accugccgcagugcacagucu

SEQ ID NO: 1618 - RNA, 21 nt, Artificial sequence
  Synthetic oligonucleotide
cuggcugcaccugccgcaguu

SEQ ID NO: 1619 - RNA, 21 nt, Artificial sequence
  Synthetic oligonucleotide
caggccuccuggcugcaccuu

SEQ ID NO: 1620 - RNA, 21 nt, Artificial sequence
  Synthetic oligonucleotide
uguccgggcaggccuccuggu

SEQ ID NO: 1621 - RNA, 21 nt, Artificial sequence
  Synthetic oligonucleotide
uggcuggcuguccgggcaggu

SEQ ID NO: 1622 - RNA, 21 nt, Artificial sequence
  Synthetic oligonucleotide
uggcuggcuggcuggcugucu

SEQ ID NO: 1623 - RNA, 21 nt, Artificial sequence
  Synthetic oligonucleotide
cccucccuguggauccuauau

SEQ ID NO: 1624 - RNA, 21 nt, Artificial sequence
  Synthetic oligonucleotide
gauuagaguuacaucuccugu

SEQ ID NO: 1625 - RNA, 21 nt, Artificial sequence
  Synthetic oligonucleotide
auucuggcugaaugucucccu

SEQ ID NO: 1626 - RNA, 21 nt, Artificial sequence
  Synthetic oligonucleotide
ccuuguucuuccgugaaauuu

SEQ ID NO: 1627 - RNA, 21 nt, Artificial sequence
  Synthetic oligonucleotide
cccagagccggcccagguacu

SEQ ID NO: 1628 - RNA, 21 nt, Artificial sequence
  Synthetic oligonucleotide
agaacugccuuucuuuccugu

SEQ ID NO: 1629 - RNA, 21 nt, Artificial sequence
  Synthetic oligonucleotide
caccgggcugaccggccuggu

SEQ ID NO: 1630 - RNA, 21 nt, Artificial sequence
  Synthetic oligonucleotide
cuccgccccgcccccuccacu

SEQ ID NO: 1631 - RNA, 21 nt, Artificial sequence
  Synthetic oligonucleotide
gcuacggaggggcgugucucu

SEQ ID NO: 1632 - RNA, 21 nt, Artificial sequence
  Synthetic oligonucleotide
cgguggccucucuuucgcggu

SEQ ID NO: 1633 - RNA, 21 nt, Artificial sequence
  Synthetic oligonucleotide
gcccagguaccagcagaccuu

SEQ ID NO: 1634 - RNA, 21 nt, Artificial sequence
  Synthetic oligonucleotide
gcggagaacugccauucuuuu

SEQ ID NO: 1635 - RNA, 21 nt, Artificial sequence
  Synthetic oligonucleotide
uccacaccgcggagaacugcu

SEQ ID NO: 1636 - RNA, 21 nt, Artificial sequence
  Synthetic oligonucleotide
uccucgcuggccuccgcaccu

SEQ ID NO: 1637 - RNA, 21 nt, Artificial sequence
  Synthetic oligonucleotide
uaccagcagaccugcgcgcau

SEQ ID NO: 1638 - RNA, 23 nt, Artificial sequence
  Synthetic oligonucleotide
agccuagaccccgcguccuaaag

SEQ ID NO: 1639 - RNA, 21 nt, Artificial sequence
  Synthetic oligonucleotide
uuaggacgcggggucuaggcu